SEQUENCE LISTING (17 sequences)

SEQ ID NO: 1 - DNA, 22 bp, synthetic construct
  misc_feature 1..22: note = primer
  source 1..22: mol_type = other DNA
ggagcgcgcactgcgccagtcg

SEQ ID NO: 2 - DNA, 22 bp, synthetic construct
  misc_feature 1..22: note = primer
  source 1..22: mol_type = other DNA
acgactggcgcagtgcgcgctc

SEQ ID NO: 3 - DNA, 29 bp, synthetic construct
  misc_feature 1..29: note = primer
  source 1..29: mol_type = other DNA
cccgatacgctgagagtggtttgcggatc

SEQ ID NO: 4 - DNA, 29 bp, synthetic construct
  misc_feature 1..29: note = primer
  source 1..29: mol_type = other DNA
gatccgcaaaccactctcagcgtatcggg

SEQ ID NO: 5 - protein, 165 aa, Homo sapiens
DDWRCARSMHEFSAKDIDGHMVNLDKYRGFVCIVTNVASQCGKTEVNYTQLVDLHARYAECGLRILAFPCNQFGKQEPGSNEEIKEFAAGYNVKFDMFSKICVNGDDAHPLWKWMKIQPKGKGILGNAIKWNFTKFLIDKNGCVVKRYGPMEEPLVIEKDLPHYF

SEQ ID NO: 6 - DNA, 29 bp, synthetic construct
  misc_feature 1..29: note = primer
  source 1..29: mol_type = other DNA
ctgcgtggtgaagcactacggacccatgg

SEQ ID NO: 7 - DNA, 29 bp, synthetic construct
  misc_feature 1..29: note = primer
  source 1..29: mol_type = other DNA
ccatgggtccgtagtgcttcaccacgcag

SEQ ID NO: 8 - DNA, 34 bp, synthetic construct
  misc_feature 1..34: note = primer
  source 1..34: mol_type = other DNA
ggcctcccagtgaggcgcgaccgaagtaaactac

SEQ ID NO: 9 - DNA, 34 bp, synthetic construct
  misc_feature 1..34: note = primer
  source 1..34: mol_type = other DNA
gtagtttacttcggtcgcgcctcactgggaggcc

SEQ ID NO: 10 - DNA, 36 bp, synthetic construct
  misc_feature 1..36: note = primer
  source 1..36: mol_type = other DNA
tggatgaagatccaacccaggggcaggggcatcctg

SEQ ID NO: 11 - DNA, 36 bp, synthetic construct
  misc_feature 1..36: note = primer
  source 1..36: mol_type = other DNA
caggatgcccctgcccctgggttggatcttcatcca

SEQ ID NO: 12 - DNA, 29 bp, synthetic construct
  misc_feature 1..29: note = primer
  source 1..29: mol_type = other DNA
ctgcgtggtgaagcactacggacccatgg

SEQ ID NO: 13 - DNA, 29 bp, synthetic construct
  misc_feature 1..29: note = primer
  source 1..29: mol_type = other DNA
ccatgggtccgtagtgcttcaccacgcag

SEQ ID NO: 14 - DNA, 34 bp, synthetic construct
  misc_feature 1..34: note = primer
  source 1..34: mol_type = other DNA
ggcctcccagtgtggcgcgaccgaagtaaactac

SEQ ID NO: 15 - DNA, 34 bp, synthetic construct
  misc_feature 1..34: note = primer
  source 1..34: mol_type = other DNA
gtagtttacttcggtcgcgccacactgggaggcc

SEQ ID NO: 16 - DNA, 41 bp, synthetic construct
  misc_feature 1..41: note = primer
  source 1..41: mol_type = other DNA
cgtggcctcccagtgtggcctaaccgaagtaaactacactc

SEQ ID NO: 17 - DNA, 41 bp, synthetic construct
  misc_feature 1..41: note = primer
  source 1..41: mol_type = other DNA
gagtgtagtttacttcggttaggccacactgggaggccacg

(19) United States
(12) Patent Application Publication
(10) Pub. No.: US 2023/0167058 A1
(43) Pub. Date: Jun. 1, 2023

(54) GPX4 COMPOUNDS AND COMPOSITIONS AND METHODS OF TREATMENT USING SAME

(71) Applicant: THE TRUSTEES OF COLUMBIA UNIVERSITY IN THE CITY OF NEW YORK, New York, NY (US)

(72) Inventors: Brent R. STOCKWELL, New York, NY (US); Hengrui LIU, New York, NY (US); Farhad FOROUHAR, New York, NY (US); Qian WANG, New York, NY (US)

(21) Appl. No.: 17/931,341

(22) Filed: Sep. 12, 2022

Related U.S. Application Data
(63) Continuation of application No. PCT/US2021/022143, filed on Mar. 12, 2021.
(60) Provisional application No. 63/122,143, filed on Dec. 7, 2020.
(60) Provisional application No. 62/989,425, filed on Mar. 13, 2020.

(51) Int. Cl. C07D 209/48; A61P 35/00; C07D 295/16; C07D 487/04; C07F 7/22; C07D 409/04; C07D 277/42; C07D 403/06; C07D 455/02

(52) U.S. Cl. CPC C07D 209/48; A61P 35/00; C07D 295/16; C07D 487/04; C07F 7/2224; C07D 409/04; C07D 277/42; C07D 403/06; C07D 455/02

(57) ABSTRACT

The present disclosure provides, inter alia, compounds to modulate GPX4 activity. Also provided are pharmaceutical compositions containing same compounds. Further provided are methods for treating or ameliorating the effects of a cancer in a subject, methods of modulating GPX activity in a subject, methods of inducing ferroptosis in a cell, and methods for treating or ameliorating the effects of a cancer in a subject using the compounds or composition in combination with other therapeutic agents.

### CROSS REFERENCE TO RELATED APPLICATIONS

The present application is a continuation of PCT International Application No. PCT/US2021/022143, filed Mar. 12, 2021, which claims benefit of U.S. Provisional Patent Application Ser. No. 63/122,143, filed on Dec. 7, 2020, and U.S. Provisional Patent Application Ser. No. 62/989,425, filed on Mar. 13, 2020, which applications are incorporated by reference herein in their entireties.

### GOVERNMENT FUNDING

This disclosure was made with government support under grant no. CA209896, awarded by National Institutes of Health. The government has certain rights in the disclosure.

### FIELD OF DISCLOSURE

The present disclosure provides, inter alia, compounds to modulate GPX4 activity. Also provided are pharmaceutical compositions containing same compounds, as well as methods of using such compounds and compositions.

### INCORPORATION BY REFERENCE OF SEQUENCE LISTING

This application contains references to amino acids and/or nucleic acid sequences that have been filed concurrently herewith as sequence listing XML file “CU21144-seq26.xml”, file size of 22 KB, created on Sep. 11, 2022. The aforementioned sequence listing is hereby incorporated by reference in its entirety pursuant to 37 C.F.R. § 1.52(e)(5).

### BACKGROUND OF THE DISCLOSURE

Cancer cells are dependent on their lipid composition for establishing and modulating membrane structural integrity, morphology, metabolism, migration, invasiveness, and other functions. For example, among the thousands of lipid species that compose eukaryotic cell membranes, the abundance and localization of polyunsaturated-fatty-acid-(PUFAs)-containing phospholipids (PUFA-PLs) is a major factor in determining the fluidity of cell membranes (Agmon et al. 2017). Since the cis conformation of double bonds in PUFA-PLs hinders efficient stacking of fatty acyl tails, elevated levels of PUFA-PLs contribute to increasing membrane fluidity and thinning (Agmon et al. 2018). PUFA-PLs are, however, susceptible to peroxidation via iron-catalyzed reaction with molecular oxygen at bis-allylic positions, catalyzed by lipoxygenases and labile iron (Yang et al. 2016). Thus, some cancer cells depend on a critical network of proteins to eliminate their PUFA-PL peroxides; a key protein at the center of this defense network is the selenoprotein glutathione peroxidase 4 (GPX4). When GPX4 activity is compromised, lipid peroxidation can cause ferroptosis (Stockwell et al. 2017), an oxidative, iron-dependent form of non-apoptotic cell death (Dixon et al. 2012). Ferroptosis acts as a natural tumor suppressive and immune surveillance mechanism, and can be induced by exogenous agents in cells that are addicted to GPX4 (Dixon and Stockwell, 2019). Cancer cells from tissues of diverse origins have been screened for their sensitivity to ferroptosis-inducing compounds (Viswanathan et al. 2017). It has been found that ferroptosis inducers, including GPX4 inhibitors, selectively target cancers with a mesenchymal or otherwise drug-resistant signature (Viswanathan et al. 2017). Consistent with the mesenchymal state being associated with drug resistance, an independent study on persister cancer cells, which are proposed to escape from conventional cytotoxic treatment through a dormant state and then revive to cause tumor relapse, revealed a similar selective dependency on GPX4 (Hangauer et al. 2017).

Examination of persister cells also revealed upregulation of mesenchymal markers and downregulation of epithelial markers (Hangauer et al. 2017). Overexpression of mesenchymal state genes is associated with epithelial-mesenchymal transition (EMT). Since EMT increases motility of tumor cells and enables the invasion of primary tumors to distant sites, EMT is a key step in metastasis. EMT also renders cancer cells resistant to apoptosis and chemotherapy (Viswanathan et al. 2017). EMT requires plasma membrane remodeling to increase fluidity, which is associated with elevated biosynthesis of PUFA-PLs. Given that PUFA-PLs are more susceptible to peroxidation than saturated or mono-unsaturated fatty acid PLs, cells in an EMT state have increased dependency on GPX4 to remove these lipid peroxides (Viswanathan et al. 2017). Therefore, cancer cells undergoing EMT that acquire resistance to apoptosis become vulnerable to lipid peroxidation and ferroptosis induced by GPX4 inhibition (Viswanathan et al. 2017). As cancer cells evolve into a high-mesenchymal drug-resistant state and become resistant to apoptosis, one may selectively target such cells through ferroptosis; the most effective compounds in this context are GPX4 inhibitors (Viswanathan et al. 2017). For example, in-vivo xenografts of GPX4-knockout high-mesenchymal therapy-resistant melanoma regressed after cessation of ferrostatin-1 (a lipophilic antioxidant discovered in the Stockwell Lab that suppresses the loss of GPX4) and did not relapse after ceasing dabrafenib and trametinib treatment, while wt GPX4 xenografts continued to grow in both experiments (Viswanathan et al. 2017). GPX4 inhibitors are selectively lethal to persister and EMT cancer cells, with minimal effects on parental cells and non-transformed cells, suggesting that addiction to GPX4 creates a large therapeutic window.

Accordingly, there is a need for developing GPX4 inhibitors for the treatment of aggressive drug-resistance cancers and other GPX4-associated diseases. This disclosure is directed to meeting these and other needs.

### SUMMARY OF THE DISCLOSURE

One of the most pressing problems in oncology is metastatic, drug-resistant cancers. Indeed, most deaths of cancer patients are caused by aggressive, metastatic, drug-resistant cancers. A surprising finding is that as cancers evolve into aggressive and drug-resistant forms, they acquire an exquisite sensitivity to GPX4 inhibition. These data provide the tantalizing possibility that the most aggressive neoplastic diseases can be treated through the use of GPX4 inhibitors, and that the ideal patients for treatment with these inhibitors are end-stage patients that have exhausted other therapeutic options. In 2012, we reported the existence of a new form of tumor suppressive cell death, ferroptosis (Dixon et al. 2012). In 2014, we discovered that the key negative regulator of ferroptosis was the lipid repair enzyme GPX4, demonstrating that GPX4 functions in ferroptosis in manner analogous to how the oncogene Bcl-2 functions in apoptosis (Yang et al. 2014). We discovered the first GPX4 inhibitor—the nanomolar potency small molecule RSL3 (Yang et al. 2014). In 2017, we reported that cancer cells that have undergone epithelial-to-mesenchymal (EMT) transition become hypersensitive to ferroptosis, and to GPX4 inhibitors (Viswanathan et al. 2017). We also discovered how RSL3 inhibits GPX4, obtaining a co-crystal structure of RSL3 bound to GPX4, which revealed a novel drug-binding site on GPX4. We propose, inter alia, to exploit this finding to discover drug-like GPX4 inhibitors with favorable ADMET properties that can be developed as a first in class GPX4 inhibitors for drug-resistant cancers having a high EMT gene expression signature.

Accordingly, one embodiment of the present disclosure is a compound according to formula (1):

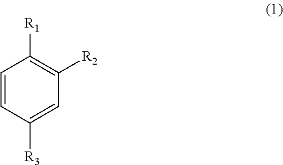

wherein:
R₁, R₂, and R₃ are independently selected from the group consisting of H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof, with the proviso that the compound is not

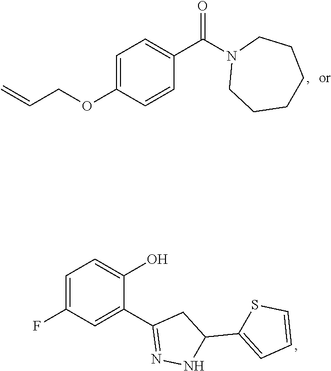 [SMILES: C=CCOc1ccc(C(=O)N2CCCCCC2)cc1 ; Oc1ccc(F)cc1C1=NNC(c2cccs2)C1]

or an N-oxide, crystalline form, hydrate, or pharmaceutically acceptable salt thereof.

Another embodiment of the present disclosure is a compound according to formula (2):

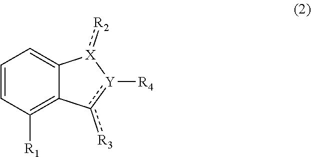

wherein:
a dashed line indicates the presence of an optional double bond;
X and Y are independently selected from the group consisting of C, N, S and O;
R₁, R₂, R₃, and R₄ are independently selected from the group consisting of no atom, H, D, O, N, halo, ether, ester, amide, C(O), (O)C(R), C(O)C, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

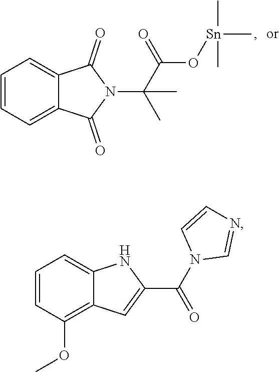 [SMILES: CC(C)(C(=O)[O][Sn]([CH3])([CH3])[CH3])N1C(=O)c2ccccc2C1=O ; COc1cccc2[nH]c(C(=O)n3ccnc3)cc12]

or an N-oxide, crystalline form, hydrate, or pharmaceutically acceptable salt thereof.

In some embodiments, the compound has a structure selected from the group consisting of:

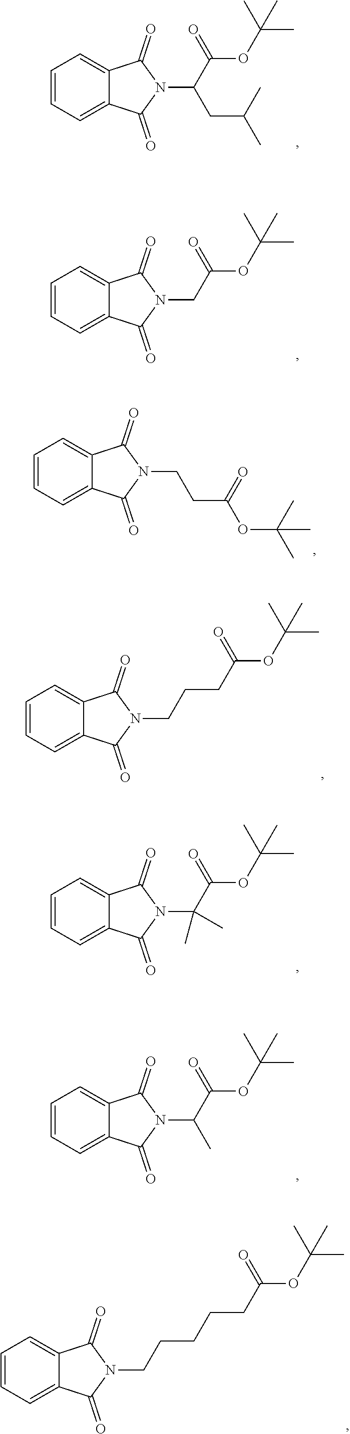

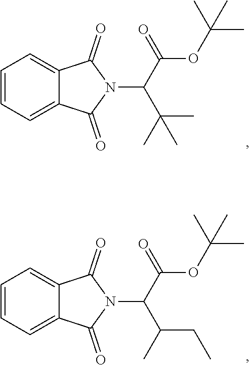 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

or an N-oxide, crystalline form, hydrate, or pharmaceutically acceptable salt thereof.

Another embodiment of the present disclosure is a compound according to formula (3):

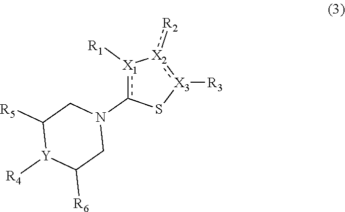

wherein:
a dashed line indicates the presence of an optional double bond;
X₁, X₂, X₃ and Y are independently selected from the group consisting of C, N, S and O;
R₁, R₂, R₃, R₄, R₅, and R₆ are independently selected from the group consisting of no atom H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

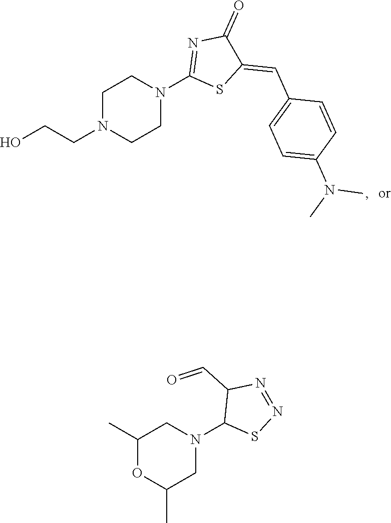

or an N-oxide, crystalline form, hydrate, or pharmaceutically acceptable salt thereof.

Another embodiment of the present disclosure is a compound according to formula (4):

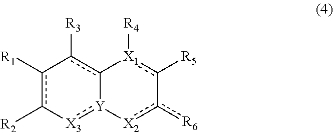

wherein:
a dashed line indicates the presence of an optional double bond;
X₁, X₂, and X₃ are independently selected from the group consisting of C, N, S and O;

### Y is C or N;

R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₂carbocycle that may be optionally substituted with an atom or a group selected from the group consisting of O, N, halo, C₁₋₄alkyl, CF₃, and combinations thereof;
R₃ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
R₄ and R₅ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₄ and R₅ may together form a C₃₋₁₂carbocycle that may be optionally substituted with an atom or a group selected from the group consisting of O, N, halo, C₁₋₄alkyl, CF₃, and combinations thereof;
R₆ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

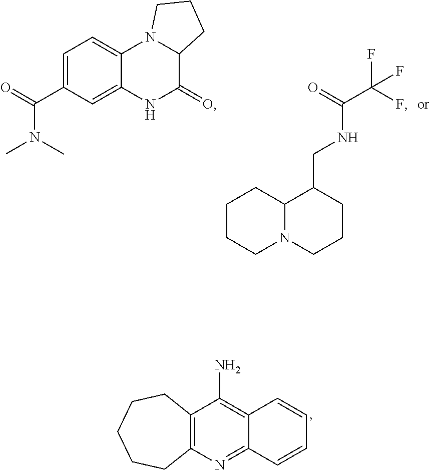 [SMILES: CN(C)C(=O)c1ccc2c(c1)NC(=O)C1CCCN21 ; O=C(NCC1CCCN2CCCCC12)C(F)(F)F ; "or" ; Nc1c2c(nc3ccccc13)CCCCC2]

or an N-oxide, crystalline form, hydrate, or pharmaceutically acceptable salt thereof.

Another embodiment of the present disclosure is a composition, including pharmaceutical compositions, comprising one or more compounds disclosed herein and a pharmaceutically acceptable carrier, adjuvant or vehicle.

Another embodiment of the present disclosure is a method for treating or ameliorating the effects of a glutathione peroxidase 4 (GPX4)-associated disease in a subject in need thereof, comprising administering to the subject an effective amount of one or more compounds disclosed herein or the compositions disclosed herein.

Another embodiment of the present disclosure is a method for modulating the activity of glutathione peroxidase 4 (GPX4) in a subject in need thereof, comprising administering to the subject an effective amount of one or more compounds disclosed herein or the compositions disclosed herein.

Another embodiment of the present disclosure is a method for increasing the level of peroxide in a subject in need thereof, comprising administering to the subject an effective amount of one or more compounds disclosed herein or the compositions disclosed herein.

A further embodiment of the present disclosure is a method for inducing ferroptosis in a cell, comprising contacting the cell with an effective amount of one or more compounds disclosed herein or the compositions disclosed herein.

Another embodiment of the present disclosure is a method for treating or ameliorating the effects of a cancer in a subject in need thereof, comprising administering to the subject i) an effective amount of a first anti-cancer agent, which is one or more compounds disclosed herein or the compositions disclosed herein, and ii) an effective amount of a second anti-cancer agent.

An additional embodiment of the present disclosure is a kit for treating or ameliorating the effects of a glutathione peroxidase 4 (GPX4)-associated disease in a subject in need thereof, comprising an effective amount of one or more compounds disclosed herein or the compositions disclosed herein, packaged with its instructions for use.

Another embodiment of the present disclosure is a method for treating or ameliorating the effects of a glutathione peroxidase 4 (GPX4)-associated disease in a subject in need thereof, comprising administering to the subject an effective amount of one or more compounds having a structure selected from the group consisting of:

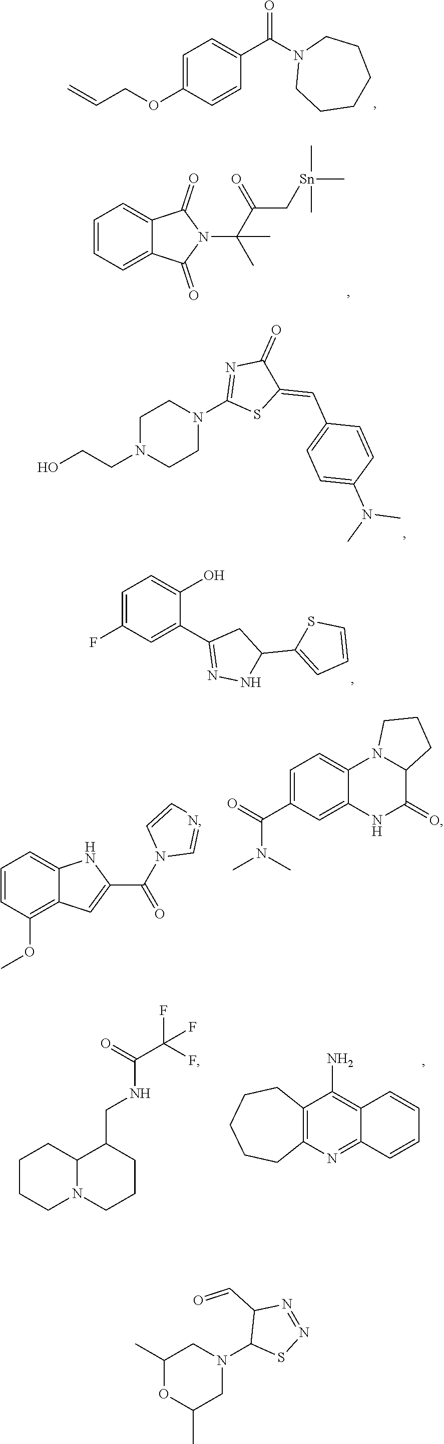

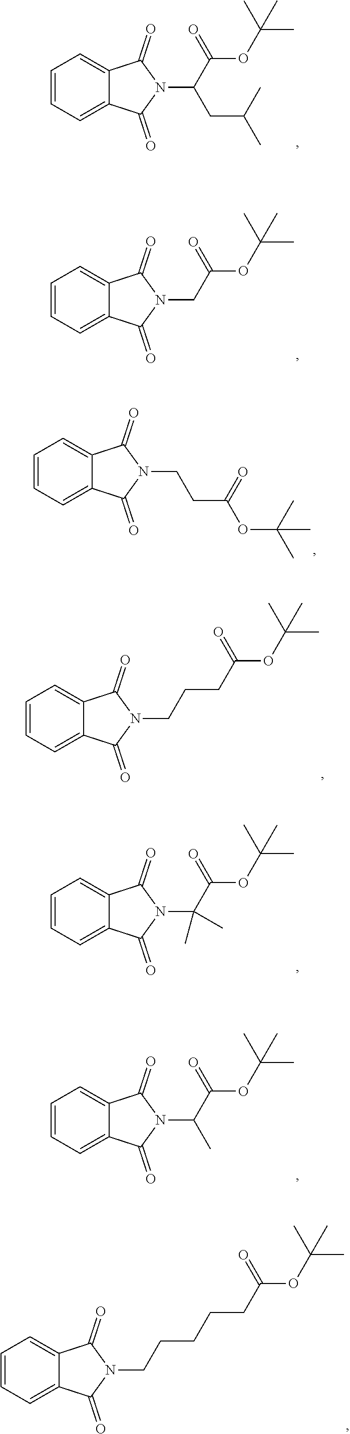

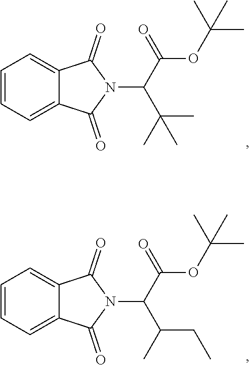 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for treating or ameliorating the effects of a glutathione peroxidase 4 (GPX4)-associated disease in a subject in need thereof, comprising administering to the subject an effective amount of a compound having the structure of:

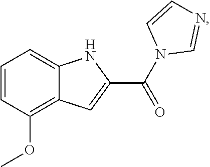 [SMILES: COc1cccc2[nH]c(C(=O)n3ccnc3)cc12]

or an N-oxide, crystalline form, hydrate, or pharmaceutically acceptable salt thereof.

Another embodiment of the present disclosure is a method for increasing the level of peroxide in a subject in need thereof, comprising administering to the subject an effective amount of one or more compounds having a structure selected from the group consisting of:

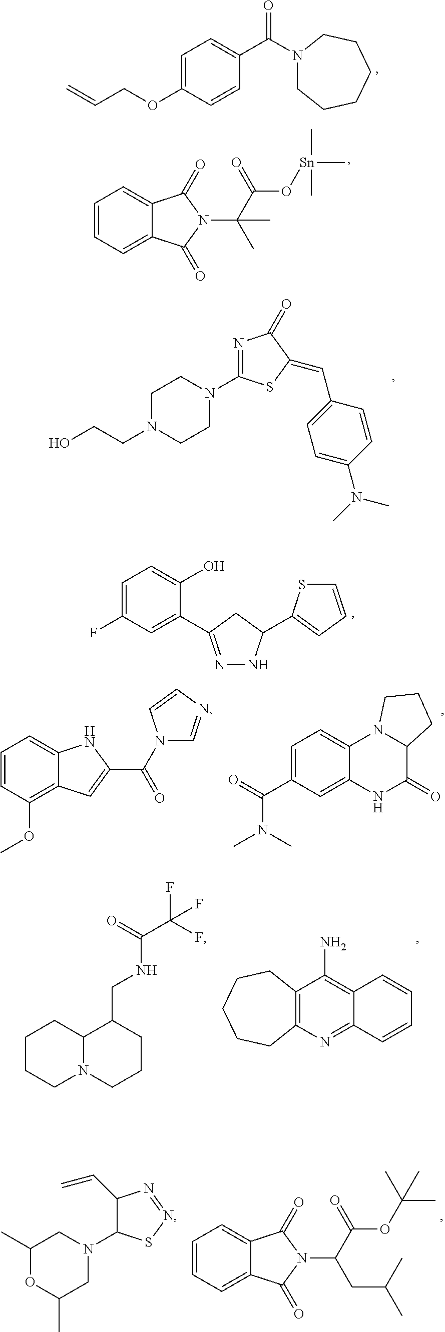

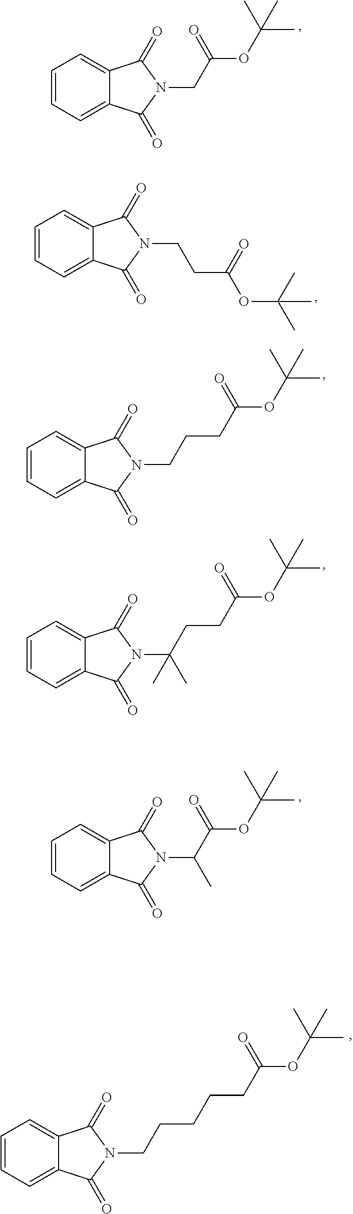

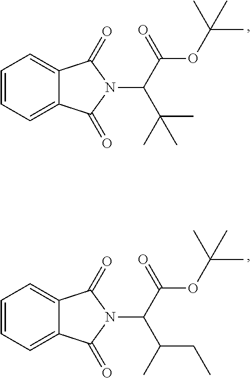 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for inducing ferroptosis in a cell, comprising contacting the cell with an effective amount of one or more compounds having a structure selected from the group consisting of:

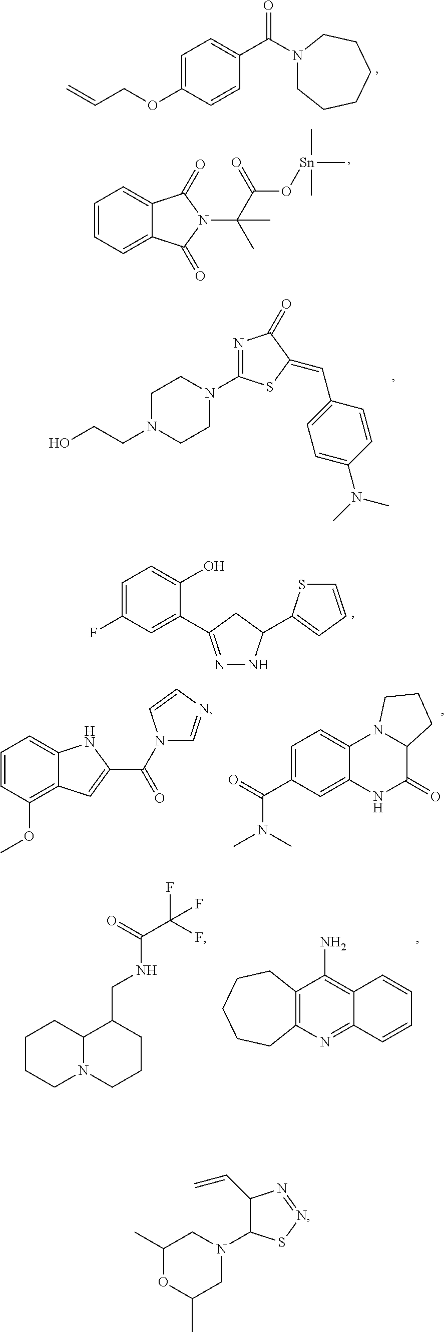

and combinations thereof, or an N-oxide, crystalline form, hydrate, or pharmaceutically acceptable salt thereof.

Another embodiment of the present disclosure is a method for treating or ameliorating the effects of a cancer in a subject in need thereof, comprising administering to the subject i) an effective amount of a first anti-cancer agent, and ii) an effective amount of a second anti-cancer agent, wherein the first anti-cancer agent is one or more compounds having a structure selected from the group consisting of:

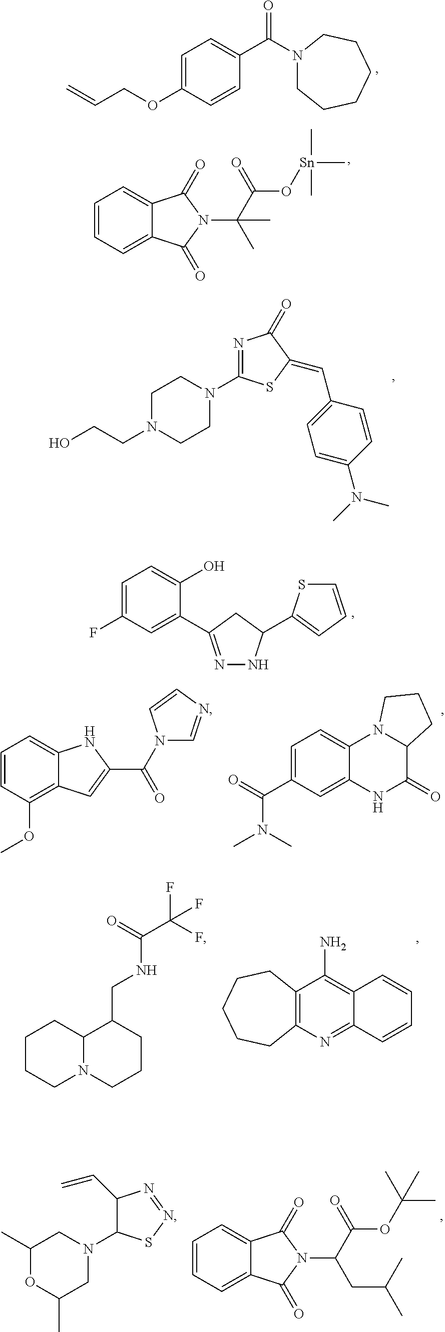

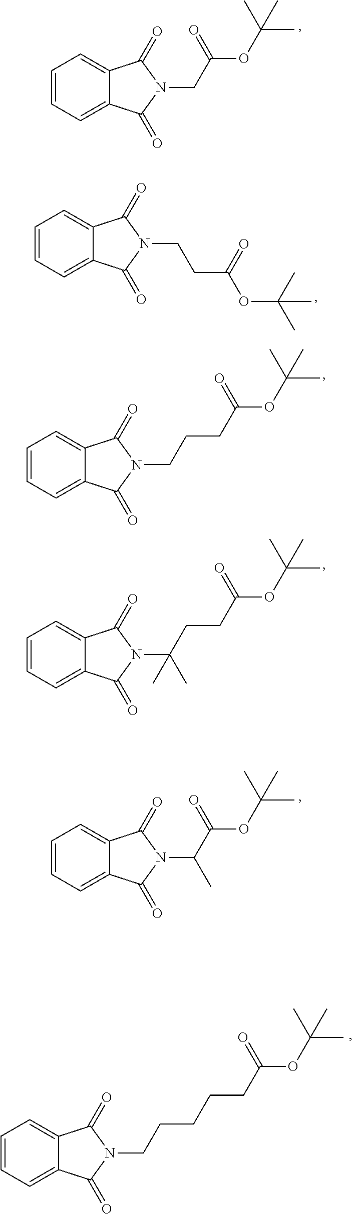

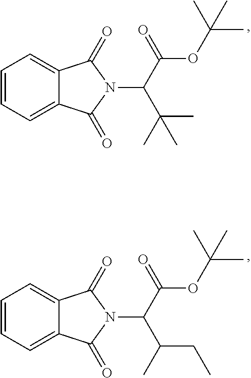 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for treating or ameliorating the effects of a cancer in a subject in need thereof, comprising administering to the subject i) an effective amount of a first anti-cancer agent, and ii) an effective amount of a second anti-cancer agent, wherein the first anti-cancer agent is a compound having the structure of:

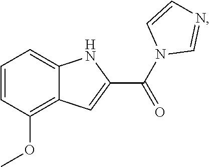 [SMILES: COc1cccc2[nH]c(C(=O)n3ccnc3)cc12]

or an N-oxide, crystalline form, hydrate, or pharmaceutically acceptable salt thereof.

### BRIEF DESCRIPTION OF THE DRAWINGS

The application file contains at least one photograph executed in color. Copies of this patent application with color photographs will be provided by the Office upon request and payment of the necessary fee.

FIGS. 1A and 1B show that the hepatocellular carcinoma therapies sorafenib and lenvatinib induce ferroptosis. In FIG. 1A, HT-1080 cells treated with 10 μM sorafenib or erastin for 24 h show loss of viability that is suppressed by co-treatment with the ferroptosis inhibitors beta-mercaptoethanol (β-ME), ferrostatin-1 (fer-1) or deferoxamine (DFO). In FIG. 1B, SKHEP1 HCC cells treated with 12.5 μM lenvatinib for 72 h show a reduction in cell number that is suppressed by co-treatment with either of the ferroptosis inhibitors fer-1 or liproxstatin.

FIG. 2 shows the structures of reported GPX4 inhibitors. None of these reported compounds are suitable for development as drugs; hence, new scaffolds are needed.

FIG. 3 shows the structure of ML162 bound to GPX4^{C66S} (Eaton et al. 2019). It reveals that ML162 binds at the active site.

FIG. 4 shows the co-crystal structure of GPX4 bound to RSL3.

FIGS. 5A-5C show that MALDI mass spectra demonstrating RSL3 does not bind in the active site. In FIG. 5A, MALDI mass spectra demonstrating binding of RSL3 to GPX4^{U46C} in vitro is shown. In FIGS. 5B and 5C, we generated an all-cys mutant of GPX4 in which all cysteines were mutated to alanine or serine, and the active site selenocysteine was mutated to cysteine to enable protein expression. While ML162 was verified to bind to this U46C, allCys(−) mutant of GPX4, RSL3 showed no binding to this mutant.

FIGS. 6A-6E show the GPX4 inhibitor discovery. FIG. 6A shows the in silico docking into binding sites on GPX4. FIG. 6B shows the validation of binding by MST.

FIG. 6C shows the analysis of binding by SPR. FIG. 6D shows the examination of enzymatic inhibitory effects by a GPX4-specific activity assay. FIG. 6E shows the GPX4 product/substrate ratio calculated based on data in FIG. 6D. Note: LOC1 (LOC880), LOC2 (LOC957), LOC3 (LOC1886) and LOC4 (LOC4873).

FIGS. 7A and 7B show that HSQC NMR data suggesting that the hit compound binds to the RSL3-binding site. FIG. 7A shows the HSQC NMR of GPX4 alone or with RSL3. FIG. 7B shows the HSQC NMR of GPX4 alone or with LOC1886.

FIGS. 8A-8I show that cysteine 66 is a binding site of RSL3 and ML162 on GPX4. FIG. 8A shows the RSL3 and co-crystal structure of GPX4 with RSL3. FIG. 8B shows the ML162 and co-crystal structure of GPX4 with ML162. FIG. 8C shows that overexpression of GFPtagged-GPX4^{C66S}, which lacks the cys66 binding site, protected HT-1080 cells from ferroptosis induced by RSL3 and ML162 to a greater extent than the overexpression of GFP-tagged-GPX4^{W}, suggesting binding of RSL3 and ML162 to cys66 of GPX4 in a cellular context. FIG. 8D shows that overexpression of tag-free GPX4^{C66S}, which lacks the cys66 binding site, also protected HT-1080 cells from ferroptosis induced by RSL3 and ML162 to a greater extent than overexpression of tag-free GPX4^{WT}, excluding the possibility of GFP tag interference. FIG. 8E shows that overexpression of GFP-tagged-GPX4^{C66S} provided equivalent protection as GFP-tagged-GPX4^{WT} against FIN56 and FINO₂, confirming specific protection against RSL3 and ML162 due to loss of the covalent binding site at cys66. FIG. 8F shows the construction of GFP-tagged AllCys(−), and AllCys(−) A66C GPX4 to verify the Cys66 binding bite. FIG. 8G shows that overexpression of enzymatically inactive AllCys(−) A66C GPX4 provided significant protection against RSL3 and ML162 as compared to AllCys(−) GPX4, suggesting Cys66 bound to RSL3 and ML162 and therefore shielded the endogenous active GPX4 in G401 cells from the inhibitors. FIG. 8H shows that, as expected, overexpression of enzymatically inactive AllCys(−) A66C GPX4 exhibited no effects on FIN56 and FINO₂ lethality, as compared to AllCys(−) GPX4. FIG. 8I shows that while AllCys(−) GPX4 cannot be covalently modified by RSL3 and therefore is resistant to RSL3-induced degradation observed with WT GPX4, AllCys(−) A66C GPX4 with the Cys66 binding site is vulnerable to the RSL3-induced degradation, based on the Western-blotting of G401 overexpressing AllCys(−) or AllCys(−) A66C GPX4 treated with RSL3.

FIGS. 9A-9F show that RSL3 and ML162 specifically bind to Sec46 and Cys66 of GPX4. FIG. 9A shows the construction of GFPtagged AllCys(−) A10C, AllCys(−) A46C, AllCys(−) A46U, AllCys(−) A107C, and AllCys(−) A148C GPX4, which individually carries only one surface (selenol)cysteine. FIG. 9B shows that overexpression of WT GPX4, AllCys(−) A46U GPX4, or AllCys(−) A66C GPX4 significantly protected G401 cells from RSL3 as compared with AllCys(−), while overexpression of GPX4 with other cysteines didn't. FIG. 9C shows that overexpression of WT GPX4, AllCys(−) A46C GPX4, AllCys(−) A46U GPX4, or AllCys(−) A66C GPX4 significantly protected G401 cells from ML162 as compared with AllCys(−), while overexpression of GPX4 with other cysteines didn't. FIG. 9D shows that WT GPX4, AllCys(−) A46U GPX4, and AllCys(−) A66C GPX4 were prone to RSL3-induced degradation, while GPX4 with other cysteines were not, confirming the selectivity towards selenocysteine 46 and cysteine 66. FIG. 9E shows that overexpression of GPX4^{U46C_C66S} which has a lower enzymatic activity than WT, exhibited no effects against treatment with FIN56 or FINO₂. FIG. 9F shows that, as expected, overexpression of GPX4^{U46C_C66S}, which has a lower enzymatic activity than WT but is devoid of both binding sites, significantly protected HT1080 cells from ferroptosis induced by RSL3 and ML162.

FIGS. 10A-10G show that Cys66 and Cys10 are involved in modulating the dual function of GPX4. FIG. 10A shows that G401 cells overexpressing GFP-tagged WT, AllCys(−), AllCys(−) A10C, AllCys(−) A46C, AllCys(−) A46U, AllCys(−) A66C, AllCys(−) A107C, and AllCys(−) A148C GPX4 were tested for IKE sensitivity. FIG. 10B shows that in our hypothesis, in addition to the canonical glutathione-dependent catalytic cycle of GPX4 (I), GPX4 may utilize the thiol on Cys10 of a second GPX4 protein as reductant to form a dimer intermediate, which will then be regenerated with the thiol on Cys66 of a third GPX4 protein to complete the catalytic cycle (II). FIG. 10C shows the enzymatic activity of endogenous GPX4 in regular G401 and G401 cells overexpressing AllCys(−), AllCys(−) A10C, AllCys(−) A66C, and AllCys(−) A148C GPX4. FIG. 10D shows that in the packing patterns of GPX4 crystals in diverse space groups (P3121, P21, and P1), C₁₀, C₄₆, and C₆₆ are consistently in close proximities to each other. FIG. 10E and FIG. 10F show that HT1080 cells overexpressing GFP-tagged WT or C10S-C66S GPX4 were tested for IKE, ML162, FIN56, and IKE sensitivity, as compared to HT1080 transfected with empty vector. FIG. 10G shows the enzymatic activity of GPX4 in HT1080 cells overexpressing GFP-tagged WT or C10S-C66S GPX4, as compared to HT1080 transfected with empty vector.

FIGS. 11A-11F show that Cys66 is an allosteric binding site of RSL3 on GPX4. In FIG. 11A, intact protein MALDI MS showed RSL3 1:1 covalently bound to GPX4^{U46C} in vitro. FIG. 11B shows that RSL3 didn't covalently bind to cysteine 46 of GPX4^{AllCys(-)-A46C} in vitro, while ML162 did. FIG. 11C shows that binding of RSL3 to Cys66 inhibited the capability of GPX4 to reduce phospholipid hydroperoxides. FIG. 11D shows that backbone resonance assignments of ¹H, ¹⁵N-HSQC-NMR spectrum for ¹⁵N-GPX4^{U46C} were solved. FIG. 11E shows that binding of RSL3 to the Cys66 allosteric site was confirmed with the overlap of ¹H, ¹⁵N-HSQC-NMR spectrum of ¹⁵N-GPX4^{U46C} alone and its complex with RSL3. FIG. 11F shows that residues interacting with RSL3 in the co-crystal structure of GPX4^{U46C}-RSL3, as shown in the 2D-interaction diagram, exhibited chemical shifts in the HSQC-NMR spectrum.

FIGS. 12A-12I show that screening of Lead-Optimized-Compound library identified lead compound binding to the Cys66 allosteric site. FIGS. 12A-12C show that co-crystal structure of GPX4 with CDS9, TMT10, or MAC5576 revealed that these inhibitors all bound to cysteine 66 of GPX4. FIG. 12D shows that thermal shift assay was applied to screen 9,719 compounds in the Lead-Optimized-Compound library for in vitro binders of GPX4^{U46C}, which would shift the melting temperature of GPX4^{U46C} (|ΔTₘ|>2° C.). FIG. 12E shows that ¹H, ¹⁵N-HSQC-NMR spectrum suggested that LOC1886, a DSF screening top hit compound, interacted with the Cys66 binding site and resulted in a global conformational change of GPX4^{U46C}. FIG. 12F shows the structure of LOC1886. FIG. 12G shows that intact protein MALDI MS analysis of GPX4^{U46C} preincubated with DMSO or LOC1886 revealed that LOC1886 covalently bound to GPX4. FIG. 12H shows that LOC1886 inhibited the capability of cellular GPX4 in HT1080 cell lysate to reduce phospholipid hydroperoxides. FIG. 12I shows that LOC1886 induced ferroptosis to HT1080, which can be rescued by fer-1, a ferroptosisspecific inhibitor.

FIGS. 13A-13D show that in silico analysis of the impact of R152H mutation on GPX4 structure predicted significantly conformational change and increased flexibility of local loop and the active site. FIG. 13A shows that computational analysis of R152H mutation predicted an alternation of the local protein surface, which caused the loss of a hydrophobic pocket. Overlap of the R152H variant with wild-type suggested a major conformational change in the loop around R152H. FIG. 13B shows that the alternation of surface mainly derived from an outstanding conformational change of the loop between Gln123 and Thr139, with which the side chains of Arg152 formed multiple hydrogen bonds in the wild-type, but not His152 in the mutant. FIG. 13C shows that MD simulation based on the modeled structure predicted extra flexibilities of the local loop and the active site of the mutant. FIG. 13D shows that distances between Cys46 and its catalytic partners Gln81/Trp136 were increased and presented a considerably wide distribution in the MD simulation of GPX4^{R152H}, as compared to GPX4^{R152R}.

FIGS. 14A-14E show the characterization of GPX4^{R152H} in cell models. In FIG. 14A, NADPH-coupled GPX4 activity assay suggested that cellular GPX4^{R152H} has a much lower enzymatic activity than WT GPX4. Using HT1080 transfected with pBP empty vector, activity of the GFP-GPX4 was acquired and then normalized with the WB intensity of GFP-GPX4. In FIG. 14B, cell lines with the knockout of endogenous GPX4 and the overexpression of the annotated GPX4^{WT} or GPX4^{R152H} were monitored for viability in media without a-Tocopherol. In FIGS. 14C and 14D, cell lines with the knockout of endogenous GPX4 and the overexpression of the annotated GPX4^{WT} or GPX4^{R152H} were tested for GPX4 enzymatic activity, either when they were cultured in media with 100 μM α-Tocopherol, or when they had been cultured in media without α-Tocopherol for 5 days. FIG. 14E shows that overexpression of GPX4^{R152H} is less protective against ferroptosis induced by RSL3, ML162, and, IKE, as compared to GPX4^{WT}.

FIGS. 15A-15C show the characterization of GPX4^{R152H} in molecular models. FIG. 15A shows the crystal structures of GPX4^{R152H_U46C} at 1.5 Å resolution. The surrounding loop (124-133) that Arg152 forms multiple H-bonds with is completely disordered. Overlap with the wild-type also revealed a conformational change of Lys48, which is around the active site. FIG. 15B shows that distances between the catalytic triad in R152H variant were increased as compared to GPX4^{U46C} (PDB:2OBI). FIG. 15C shows that Lys48 is significantly shifted away from the active site in the GPX4^{R152H} structure.

FIGS. 16A-16J show that Lys48 modulates the enzymatic function of GPX4. FIG. 16A shows that cellular GPX4^{K48A} mutant completely lost its enzymatic activity. FIG. 16B shows that overexpression of GPX4^{K48A} is not protective at all against ferroptosis induced by RSL3, ML162, and IKE. In FIG. 16C, the crystal structure of GPX4^{U46C_K48A} aligned well with of GPX4^{U46C}. A minor difference is at the loop124-133. In FIG. 16D, the distances between catalytic residues Cys46 and Trp136 were recorded throughout multiple 100 ns MD simulation of WT, K48A, and K48L GPX4. FIG. 16E shows that in the crystal structure of oxidized GPX4^{U46C} (Cys46-SO₃H), Lys48 is in close proximity to the oxidized Cys46. FIG. 16F shows that the distances between catalytic residues Cys46 and Trp136 were recorded throughout multiple 100 ns MD simulation of oxidized WT, K48A, and K48L GPX4 (Cys46-SO₃H). In FIG. 16G, crystal structures of GPX4^{U46C_K48L} aligned well with of GPX4^{U46C}. A minor difference is at the loop124-133. FIG. 16H shows that cellular GPX4^{K48L} exhibited a higher enzymatic activity than WT GPX4. FIG. 16I shows that overexpression of GPX4^{K48L} is more protective than WT GPX4 against ferroptosis induced by RSL3. FIG. 16J shows that overexpression of GPX4^{K48L} is not protective at all against ferroptosis induced by IKE.

FIGS. 17A-17G show that resistance of GPX4^{R152H} to degradation induced by GPX4 inhibitor revealed the Ubiquitin/Proteosome-dependent mechanism of the GPX4 degradation induced by FIN56/RSL3. FIG. 17A shows that overexpression of GPX4^{R152H} is especially protective against FIN56. FIG. 17B shows that the endogenous GPX4 in both HT1080 OE GFP-WT-GPX4 and HT1080 OE GFP-R152H-GPX4 are both vulnerable to the degradation induced by RSL3/ML162/FIN56. FIG. 17C shows that GFP-WT-GPX4 is vulnerable to the degradation induced by RSL3/ML162/FIN56, while GPX4^{R152H} is resistant to degradation induced by RSL3/ML162/FIN56. FIG. 17D shows that in HT1080 OE GFP-K125R-K127R-GPX4, GFP-GPX4^{K125R_K127R} is resistant to the degradation induced by RSL3/ML162/FIN56, while the endogenous GPX4 is vulnerable. FIG. 17E shows the Western blot of HT1080 after treatments with RSL3 or/and MG132. FIG. 17F shows the GPX4 enzymatic activity of HT1080 after treatments with RSL3 or/and MG132. FIG. 17G shows the Western blot of A673 after treatments with RSL3 or/and MG132.

FIGS. 18A-18G show that supplementations of selenium-containing compounds and cysteine were tested as a proof-of-concept treatment on HT1080 cells overexpressing GFP-GPX4^{R152H}. In FIGS. 18A-18D, rescue effect of seleniumcontaining compounds and cysteine was tested in HT1080 OE GFP-R152H-GPX4 treated with 0.25 μM RSL3, 0.6 μM IKE, 5 μM FIN56, or 5 μM FINO₂. In FIG. 18E, rescue effect of sodium selenite was tested at extended concentrations against RSL3, IKE, FIN56, and FINO₂. FIG. 18F shows the toxicity test of the selenium-containing compounds and N-acetyl-cysteine on HT1080. In FIG. 18G, the suppression of selenium toxicity by IKE was also reproduced in regular HT1080.

FIGS. 19A-19G show that pathology analysis was validated in the patient fibroblasts. FIG. 19A shows that RAG01 (patient, with homozygous R152H mutation in GPX4) and RAG02 (parent of patient, with heterozygous R152H mutation in GPX4) express an undistinguishable level of GPX4 protein. FIG. 19B shows that RAG01 has a lower GPX4 enzymatic activity than RAG02. FIG. 19C shows that RAG01 is more sensitive to lipid peroxidation and ferroptosis induced by RSL3, ML162, IKE, and FIN56 than RAG02. FIG. 19D shows that GPX4^{R152H} is more resistant than GPX4^{WT} to degradation induced by RSL3 and ML162. E, Mean cellular immunofluorescence of GPX4. FIG. 19F shows ratio of mean cytoplasm immunofluorescence of GPX4 over mean nuclear immunofluorescence of GPX4. In FIG. 19G, nine representative immunofluorescence images for each cell line were presented, showing DAPI and GPX4 fluorescence.

FIGS. 20A-20K show the proof-of-concept treatments on patient fibroblast. Supplementations of selenium-containing compounds, cysteine, antioxidants, and ferroptosis inhibitors were tested as proof-of-concept treatments on the patient fibroblasts.

FIGS. 21A-21B show that proof-of-concept treatments were validated in Pfa1 cells, which were knocked out of endogenous GPX4 and transfected to overexpress human (FIG. 21A) or murine (FIG. 21B) mScarlet GPX4^{WT} (red) or GPX4^{R152H} (blue).

### DETAILED DESCRIPTION OF THE DISCLOSURE

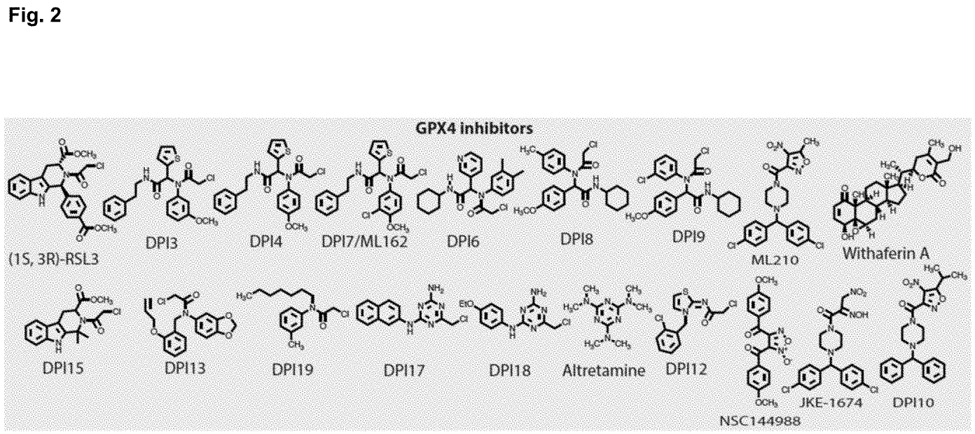

In the present disclosure, GPX4 inhibitors were developed, inter alia, for cancers with a high-mesenchymal-state gene expression signature. All known GPX4 inhibitors (FIG. 2) are or are assumed to be covalent inhibitors that react with the active site selenocysteine of GPX4; developing selective compounds for GPX4 over other selenocysteine active sites, such as in GPX1, GPX2, GPX3 and GPX6 may be challenging. In an exciting recent development, we identified allosteric sites on GPX4 that allow inactivation of GPX4 without targeting the active site selenocysteine. In particular, we solved the x-ray structure of RSL3 bound to GPX4, revealing an allosteric site distant from the active site. This new allosteric site allows for greater selectivity, and structure-based design of the first allosteric GPX4 inhibitors. This represents an exciting, potentially game-changing advance in the search for GPX4-targeted therapeutics.

Accordingly, one embodiment of the present disclosure is a compound according to formula (1):

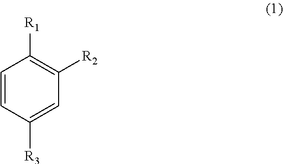

wherein:
R₁, R₂, and R₃ are independently selected from the group consisting of H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

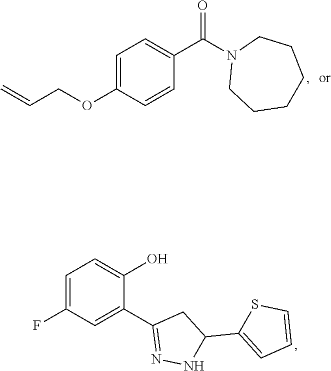 [SMILES: C=CCOc1ccc(C(=O)N2CCCCCC2)cc1 ; Oc1ccc(F)cc1C1=NNC(c2cccs2)C1]

or an N-oxide, crystalline form, hydrate, or pharmaceutically acceptable salt thereof.

Another embodiment of the present disclosure is a compound according to formula (2):

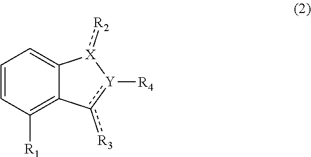

wherein:
a dashed line indicates the presence of an optional double bond;
X and Y are independently selected from the group consisting of C, N, S and O;
R₁, R₂, R₃, and R₄ are independently selected from the group consisting of no atom, H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

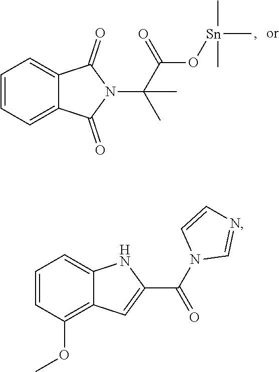 [SMILES: CC(C)(C(=O)[O][Sn]([CH3])([CH3])[CH3])N1C(=O)c2ccccc2C1=O ; COc1cccc2[nH]c(C(=O)n3ccnc3)cc12]

or an N-oxide, crystalline form, hydrate, or pharmaceutically acceptable salt thereof.

Another embodiment of the present disclosure is a compound according to formula (3):

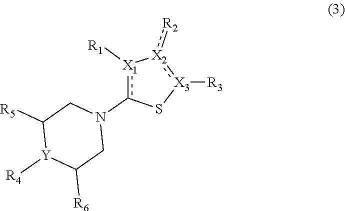

wherein:
a dashed line indicates the presence of an optional double bond;
X₁, X₂, X₃ and Y are independently selected from the group consisting of C, N, S and O;
R₁, R₂, R₃, R₄, R₅, and R₆ are independently selected from the group consisting of no atom H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

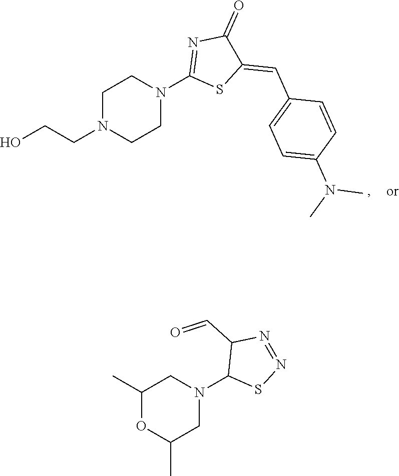

or an N-oxide, crystalline form, hydrate, or pharmaceutically acceptable salt thereof.

Another embodiment of the present disclosure is a compound according to formula (4):

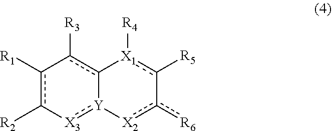

wherein:
a dashed line indicates the presence of an optional double bond;
X₁, X₂, and X₃ are independently selected from the group consisting of C, N, S and O;

### Y is C or N;

R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₂carbocycle that may be optionally substituted with an atom or a group selected from the group consisting of O, N, halo, C₁₋₄alkyl, CF₃, and combinations thereof;
R₃ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
R₄ and R₅ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₄ and R₅ may together form a C₃₋₁₂carbocycle that may be optionally substituted with an atom or a group selected from the group consisting of O, N, halo, C₁₋₄alkyl, CF₃, and combinations thereof;
R₆ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

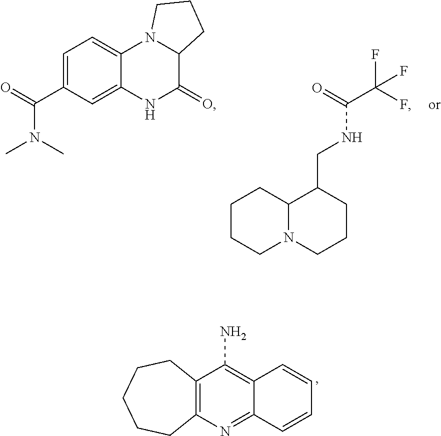

or an N-oxide, crystalline form, hydrate, or pharmaceutically acceptable salt thereof.

Another embodiment of the present disclosure is a composition, including pharmaceutical compositions, comprising one or more compounds disclosed herein and a pharmaceutically acceptable carrier, adjuvant or vehicle.

Another embodiment of the present disclosure is a method for treating or ameliorating the effects of a glutathione peroxidase 4 (GPX4)-associated disease in a subject in need thereof, comprising administering to the subject an effective amount of one or more compounds disclosed herein or the compositions disclosed herein.

In some embodiments, the GPX4-associated disease is selected from the group consisting of a cancer, a neurotic disorder, a neurodegenerative disorder, spondylometaphyseal dysplasia, mixed cerebral palsy, pontocerebellar hypoplasia, and male infertility.

In some embodiments, the GPX4-associated disease is a cancer. Non-limiting examples of cancer include hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the subject is a mammal. In some embodiments, the mammal is selected from the group consisting of humans, veterinary animals, and agricultural animals. In some embodiments, the subject is a human.

In some embodiments, the cancer is metastatic. In some embodiments, the cancer is under epithelial-to-mesenchymal (EMT) transition. In some embodiments, the cancer is hypersensitive to ferroptosis and/or addicted to GPX4. In some embodiments, the cancer is refractory to standard cancer treatment. Non-limiting examples of standard cancer treatment include chemotherapy, radiation therapy, targeted therapy, immunotherapy, and combinations thereof.

Another embodiment of the present disclosure is a method for modulating the activity of glutathione peroxidase 4 (GPX4) in a subject in need thereof, comprising administering to the subject an effective amount of one or more compounds disclosed herein or the compositions disclosed herein. In some embodiments, the modulation comprises inhibiting GPX4 activity.

Another embodiment of the present disclosure is a method for increasing the level of peroxide in a subject in need thereof, comprising administering to the subject an effective amount of one or more compounds disclosed herein or the compositions disclosed herein. Non-limiting examples of peroxide include hydrogen peroxide, organic hydroperoxide, lipid peroxide, and combinations thereof.

A further embodiment of the present disclosure is a method for inducing ferroptosis in a cell, comprising contacting the cell with an effective amount of one or more compounds disclosed herein or the compositions disclosed herein.

In some embodiments, the cell has abberant lipid accumulation. In some embodiments, the cell is a cancer cell. In some embodiments, the cancer cell is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the cell is a human cell. In some embodiments, wherein the cancer cell is metastatic. In some embodiments, the cancer cell is under epithelial-to-mesenchymal (EMT) transition. In some embodiments, the cancer cell is hypersensitive to ferroptosis and/or addicted to GPX4. In some embodiments, the hypersensitivity to ferropotosis is identified by NADPH abundance, GCH1 expression, NF2-YAP activity, EMT signature, and GPX4 expression. In some embodiments, the cancer cell is refractory to standard cancer treatment. Non-limiting examples of standard cancer treatment includes chemotherapy, radiation therapy, targeted therapy, immunotherapy, and combinations thereof.

Another embodiment of the present disclosure is a method for treating or ameliorating the effects of a cancer in a subject in need thereof, comprising administering to the subject i) an effective amount of a first anti-cancer agent, which is one or more compounds disclosed herein or the compositions disclosed herein, and ii) an effective amount of a second anti-cancer agent.

In some embodiments, the second anti-cancer agent is selected from the group consisting of chemotherapy, radiation therapy, targeted therapy, immunotherapy, and combinations thereof. In some embodiments, the second anti-cancer agent is an immunotherapy, such as checkpoint inhibitor therapy including PD-1 and CTLA-4 inhibitor therapy. Non-limiting examples of immunotherapy include ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab, and combinations thereof.

In some embodiments, the subject is a human.

In some embodiments, the cancer is metastatic. In some embodiments, the cancer is under epithelial-to-mesenchymal (EMT) transition. In some embodiments, the cancer is hypersensitive to ferroptosis and/or addicted to GPX4. In some embodiments, the cancer is refractory to standard cancer treatment.

In some embodiments, the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the first anti-cancer agent is administered to the subject before, concurrently with, or after the second anti-cancer agent.

An additional embodiment of the present disclosure is a kit for treating or ameliorating the effects of a glutathione peroxidase 4 (GPX4)-associated disease in a subject in need thereof, comprising an effective amount of one or more compounds disclosed herein or the compositions disclosed herein, packaged with its instructions for use.

The kits may also include suitable storage containers, e.g., ampules, vials, tubes, etc., for each compound of the present disclosure (which, e.g., may be in the form of pharmaceutical compositions) and other reagents, e.g., buffers, balanced salt solutions, etc., for use in administering the active agents to subjects. The compounds and/or pharmaceutical compositions of the disclosure and other reagents may be present in the kits in any convenient form, such as, e.g., in a solution or in a powder form. The kits may further include a packaging container, optionally having one or more partitions for housing the compounds and/or pharmaceutical compositions and other optional reagents.

Another embodiment of the present disclosure is a method for treating or ameliorating the effects of a glutathione peroxidase 4 (GPX4)-associated disease in a subject in need thereof, comprising administering to the subject an effective amount of one or more compounds having a structure selected from the group consisting of:

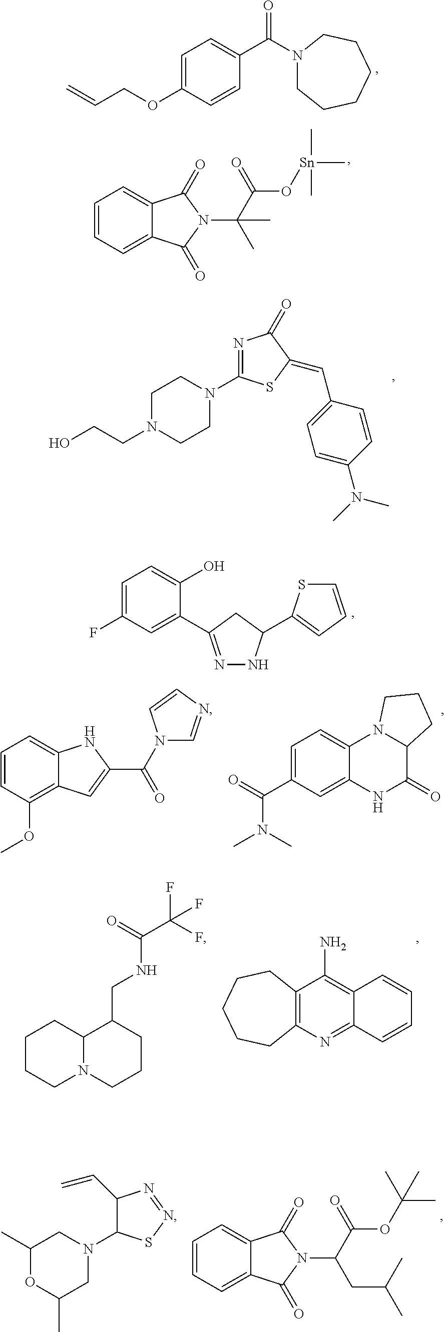

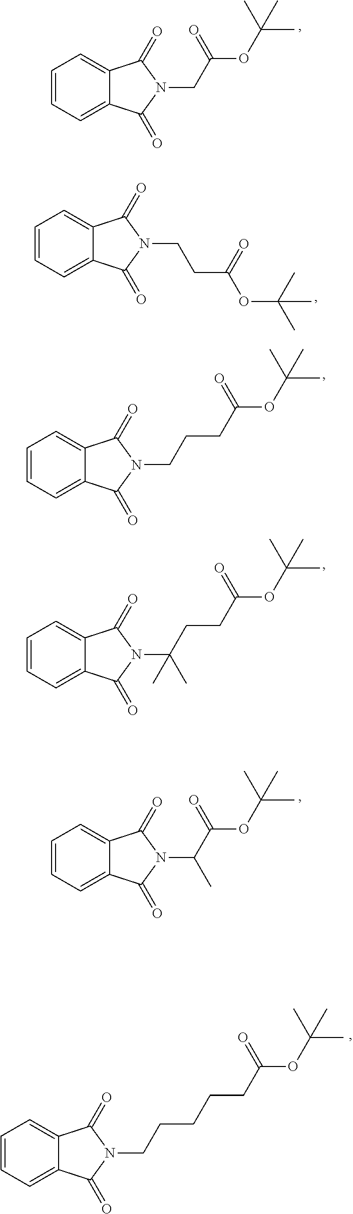

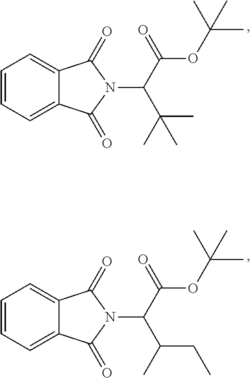 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for treating or ameliorating the effects of a glutathione peroxidase 4 (GPX4)-associated disease in a subject in need thereof, comprising administering to the subject an effective amount of a compound having the structure of:

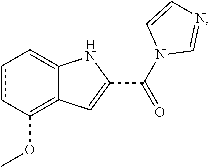

or an N-oxide, crystalline form, hydrate, or pharmaceutically acceptable salt thereof.

Another embodiment of the present disclosure is a method for increasing the level of peroxide in a subject in need thereof, comprising administering to the subject an effective amount of one or more compounds having a structure selected from the group consisting of:

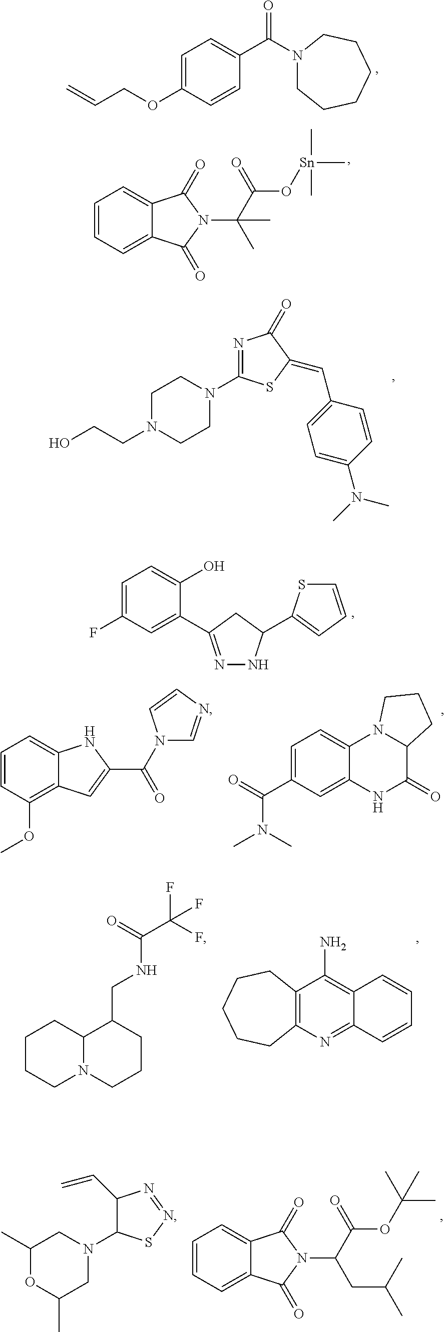

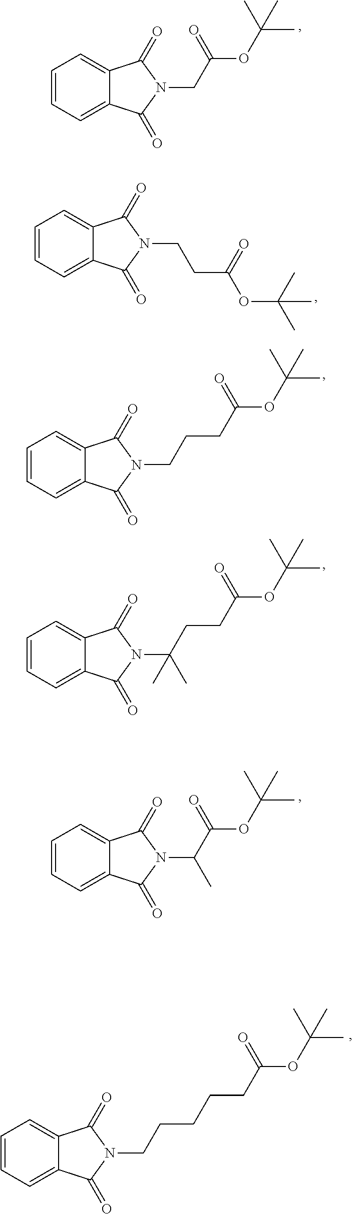

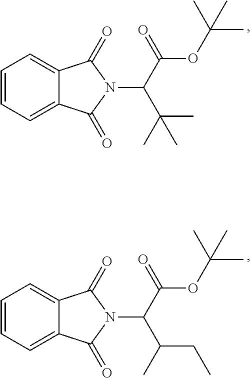 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for inducing ferroptosis in a cell, comprising contacting the cell with an effective amount of one or more compounds having a structure selected from the group consisting of:

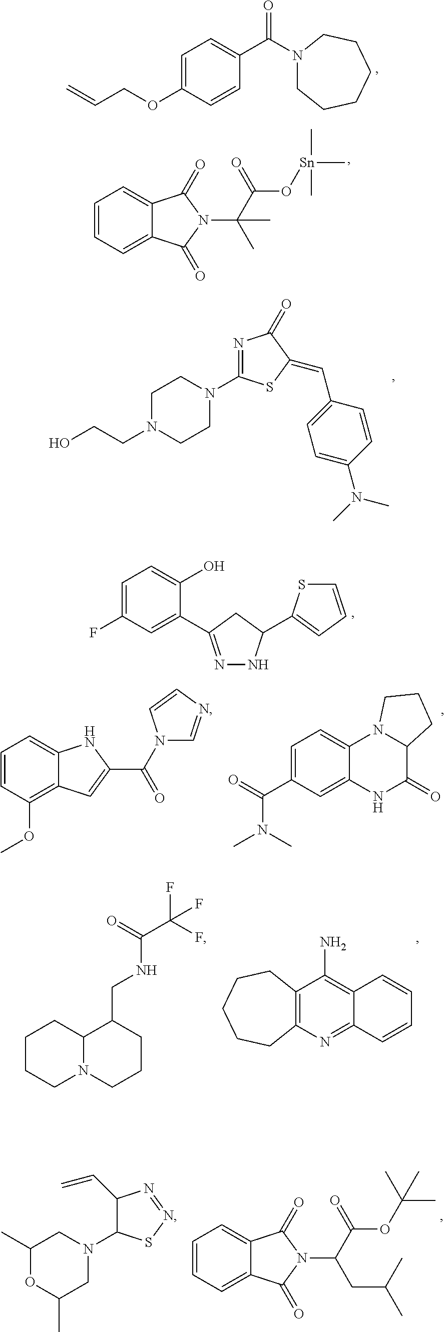

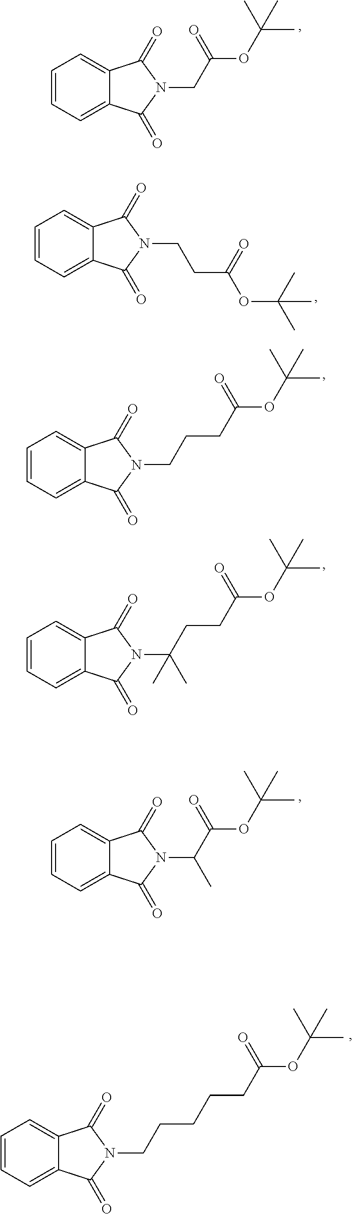

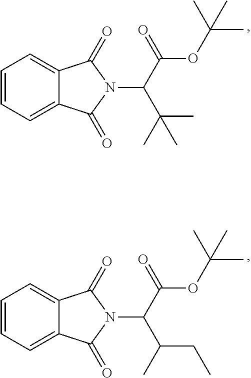 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for treating or ameliorating the effects of a cancer in a subject in need thereof, comprising administering to the subject i) an effective amount of a first anti-cancer agent, and ii) an effective amount of a second anti-cancer agent, wherein the first anti-cancer agent is one or more compounds having a structure selected from the group consisting of:

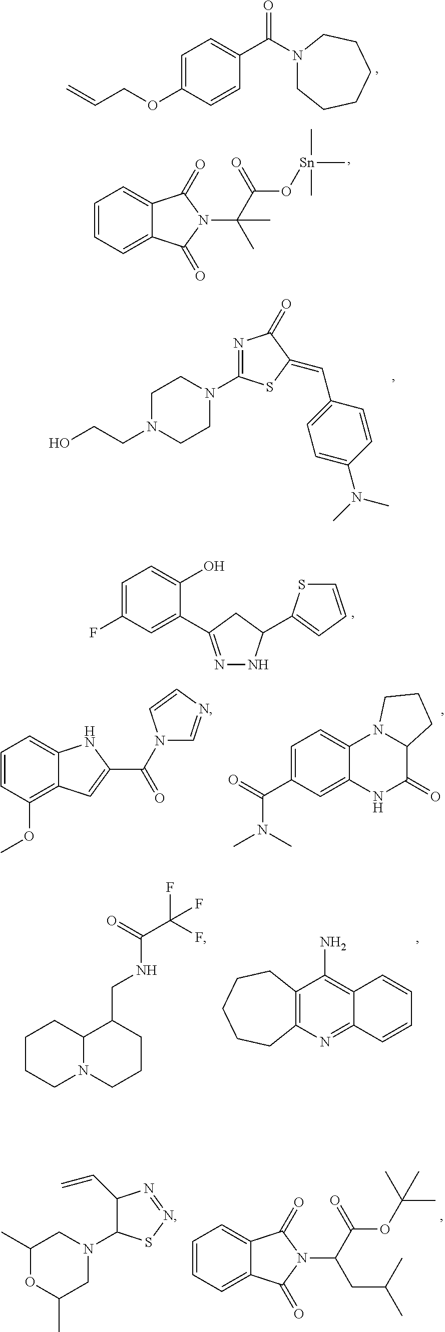

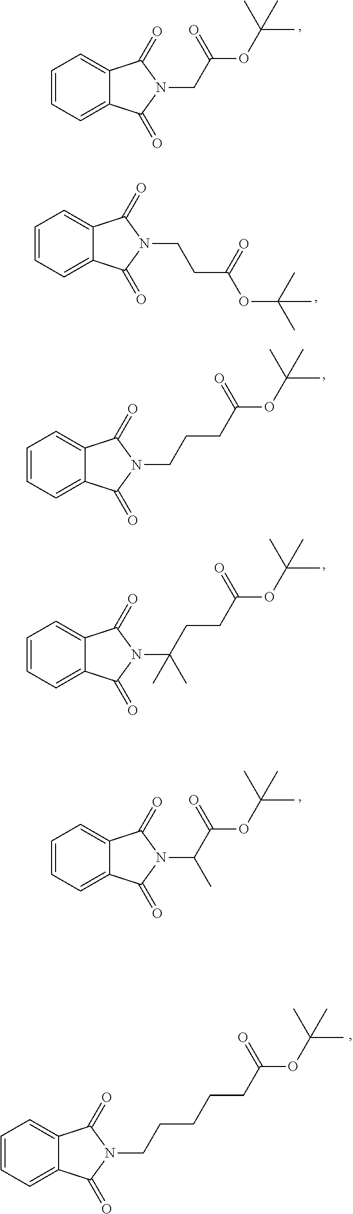

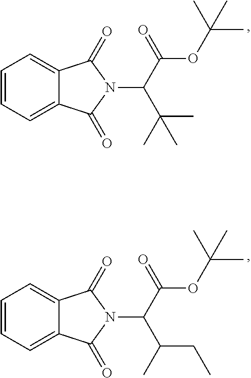 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]

and combinations thereof, or an N-oxide, crystalline form, hydrate, or pharmaceutically acceptable salt thereof.

Another embodiment of the present disclosure is a method for treating or ameliorating the effects of a cancer in a subject in need thereof, comprising administering to the subject i) an effective amount of a first anti-cancer agent, and ii) an effective amount of a second anti-cancer agent, wherein the first anti-cancer agent is a compound having the structure of:

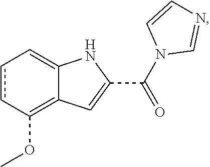

or an N-oxide, crystalline form, hydrate, or pharmaceutically acceptable salt thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. in the context of the present disclosure, the phrase “a subject in need thereof” means a subject in need of treatment for a GPX4-associated disorder, such as, e.g., a cancer. Alternatively, the phrase “a subject in need thereof” means a subject diagnosed with a GPX4-associated disorder, such as, e.g., a cancer.

As used herein, “lipid peroxidation” means the oxidative degradation of fats, oils, waxes, sterols, triglycerides, and the like. Lipid peroxidation has been linked with many degenerative diseases, such as atherosclerosis, ischemia-reperfusion, heart failure, Alzheimer's disease, rheumatic arthritis, cancer, and other immunological disorders. (Ramana et al., 2013).

As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.)

As used herein, the terms “modulate”, “modulating”, “modulator” and grammatical variations thereof mean to change, such as increasing or decreasing the activity of GPX4. In this embodiment, “contacting” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

In the present disclosure, an “effective amount” or “therapeutically effective amount” of a compound or pharmaceutical composition is an amount of such a compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or pharmaceutical composition according to the disclosure will be that amount of the compound or composition, which is the lowest dose effective to produce the desired effect with no or minimal side effects. The effective dose of a compound or pharmaceutical composition according to the present disclosure may be administered as two, three, four, five, six or more sub-doses, administered separately at appropriate intervals throughout the day.

A suitable, non-limiting example of a dosage of a compound or pharmaceutical composition according to the present disclosure or a composition comprising such a compound, is from about 1 ng/kg to about 1000 mg/kg, such as from about 1 mg/kg to about 100 mg/kg, including from about 5 mg/kg to about 50 mg/kg. Other representative dosages of a compound or a pharmaceutical composition of the present disclosure include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

A compound, composition, or pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a compound, composition, or pharmaceutical composition of the present disclosure may be administered in conjunction with other treatments. A compound, composition, or pharmaceutical composition of the present disclosure may be encapsulated or otherwise protected against gastric or other secretions, if desired.

The compositions or pharmaceutical compositions of the disclosure are pharmaceutically acceptable and comprise one or more active ingredients in admixture with one or more pharmaceutically-acceptable carriers or diluents and, optionally, one or more other compounds, drugs, ingredients and/or materials. Regardless of the route of administration selected, the compounds/compositions/pharmaceutical compositions of the present disclosure are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.). More generally, “pharmaceutically acceptable” means that which is useful in preparing a composition that is generally safe, non-toxic, and neither biologically nor otherwise undesirable and includes that which is acceptable for veterinary use as well as human pharmaceutical use.

Pharmaceutically acceptable carriers and diluents are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The compositions or pharmaceutical compositions of the disclosure may, optionally, contain additional ingredients and/or materials commonly used in such compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compounds, compositions or pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers or diluents and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier or diluent. The ointments, pastes, creams and gels may contain excipients. Powders and sprays may contain excipients and propellants.

Compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier or diluent, for example water for injection, immediately prior to use. Extemporaneous injection solutions and suspensions may be prepared from sterile powders, granules and tablets of the type described above.

The following examples are provided to further illustrate the methods of the present disclosure. These examples are illustrative only and are not intended to limit the scope of the disclosure in any way.

### EXAMPLES

### Example 1

### Indications of GPX4 Inhibitors

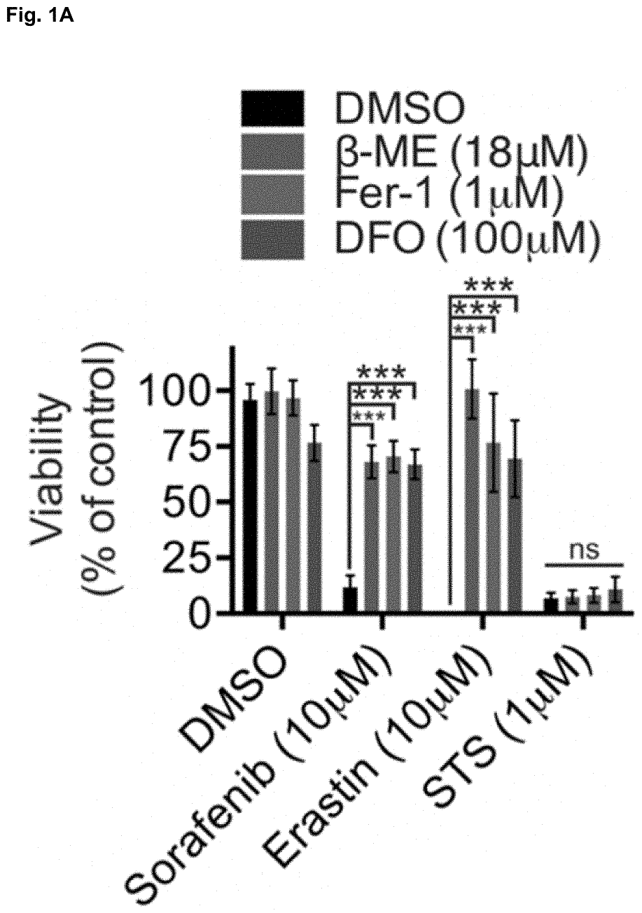

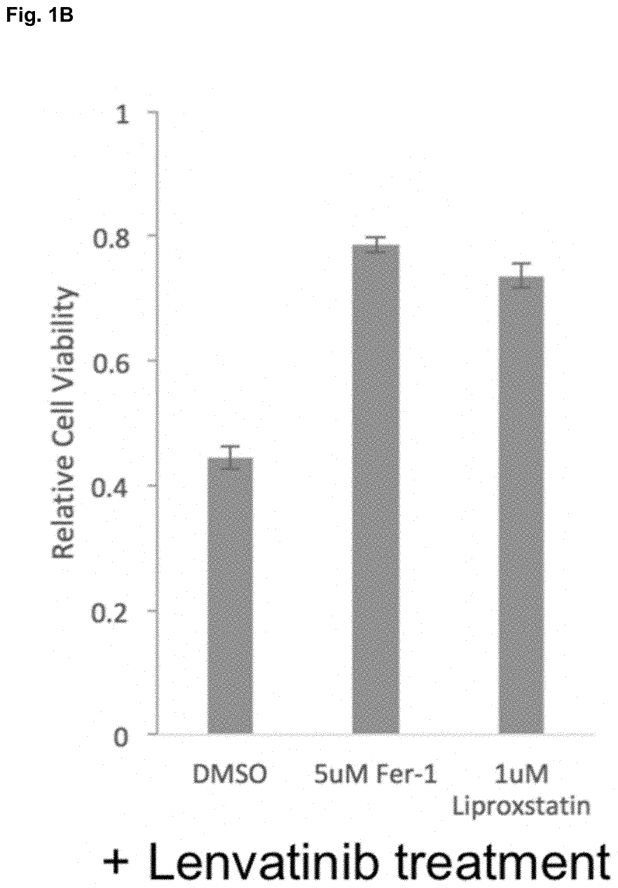

We propose to target hepatocellular carcinoma as a first indication for several reasons. First, we showed that the FDA-approved multi-targeted kinase inhibitor sorafenib is one of the very few kinase inhibitors that induce ferroptosis (FIG. 1) in cell and animal models (Lachaier et al. 2014; Louandre et al. 2013; Dixon et al. 2014); given that the primary indication for sorafenib is hepatocellular carcinoma, this suggests that pro-ferroptotic activity may contribute to the clinical efficacy of sorafenib, especially as sorafenib does not induce apoptosis and inhibits necroptosis (Martens et al. 2017; Feldmann et al. 2017). Second, we recently discovered that a recently approved targeted therapy approved for treatment of HCC, lenvatinib (lelasi et al. 2019), also induces ferroptosis, as evidenced by the ability of both ferrostatin-1 and liproxstatin-1 to suppress lenvatinib lethality in SKHEP1 HCC cells (FIG. 1).

Hepatocellular carcinoma (HCC) is an important unmet medical need, as it is one of the most aggressive, heterogeneous, and difficult to treat cancers (Medavaram and Zhang, 2018), causing >700,000 annual deaths globally (Singh et al. 2018). HCC affects children and adults of all ages, and has genetic and environmental origins. In addition, patients with hereditary tyrosinemia due to inactivation of the last step of the tyrosine catabolism pathway often develop HCC; such patients accumulate 4-fumarylacetoacetate (FAA), which, is an electrophilic Michael acceptor that depletes liver glutathione, a central inhibitor of ferroptosis (Yang et al. 2014). Exogenous glutathione suppresses much of the toxicity of FAA in cell culture and in a genetic mouse model of the disease (Singh et al. 2018), suggesting that GSH depletion can drive HCC, but also collateral sensitivity to ferroptosis (Yang et al. 2014). FAA is converted into succinylacetone, which inhibits porphyrin biosynthesis, leading to accumulation of liver iron and contributing to HCC pathogenesis (Singh et al. 2018), driving collateral sensitivity to ferroptosis (Dixon et al. 2012). Thus, GSH depletion and iron overload, key drivers of sensitivity to ferroptosis (Stockwell et al. 2017), are genetically linked to HCC through hereditary tyrosinemia patients and mouse models. Moreover, as noted above, the first targeted therapy approved for advanced HCC was the multi-targeted kinase inhibitor sorafenib, in 2007 (Medavaram and Zhang, 2018), which induces ferroptosis by inhibiting system x_{c}⁻ (Dixon et al. 2014). Many other therapies that don't induce ferroptosis have been explored in HCC, with few positive clinical results (Medavaram and Zhang, 2018). This suggests that ferroptosis may be a uniquely effective therapeutic approach to HCC.

In addition to GSH depletion and iron accumulation seen in familial-tyrosinemia-driven HCC, additional factors that predispose to HCC drive sensitivity to ferroptosis: excess alcohol metabolism in the liver depletes GSH (Zhou et al. 2019), and fatty liver in NASH patients is reported to trigger ferroptosis (Tsurusaki et al. 2019). These observations suggest that HCC tumors may harbor an intrinsic sensitivity to ferroptosis inducers, which we found to be the case in HCC cell lines (FIG. 1). As noted above, ferroptosis induction is generally tumor suppressive, as the ferroptosis inhibitor vitamin E suppresses lipid peroxidation and accelerates tumorigenesis in numerous mouse models and in cancer patients (Sayin et al. 2014). Several oncogenes and tumor suppressors appear to function in part by modulating lipid peroxidation and ferroptosis sensitivity (e.g., NRF2, KEAP1, BAP1, P53, and HIF1a) (Cao et al. 2019; Sun et al. 2015; Jung et al. 2013; Jiang et al. 2015; Speer et al. 2013). For all of these reasons, we suspect that HCC will be a good first indication for GPX4 inhibitors.

Additional indications where there is evidence for sensitivity to GPX4 inhibitors and/or ferroptosis include sarcomas, gliomas, renal cell carcinomas, ovarian cancers, DLBCLs (lymphomas), lung cancers, and breast cancers, as well as drug-resistant metastatic and EMT cancers from additional lineages (Hangauer et al. 2017).

We identified several biomarkers that predict sensitivity to ferroptosis inducers in general and GPX4 inhibitors specifically, including NADPH abundance (Shimada et al. 2015), GCH1 expression (Kraft et al. 2020), NF2-YAP activity (Wu et al. 2019), EMT signature (Viswanathan et al. 2017), and GPX4 expression itself (unpublished). We will examine each of these biomarkers and determine which combination of biomarkers is the best predictor of sensitivity to the GPX4 inhibitors we create for HCC and other target indications.

### Example 2

### Discovery of Novel GPX4 Inhibitors

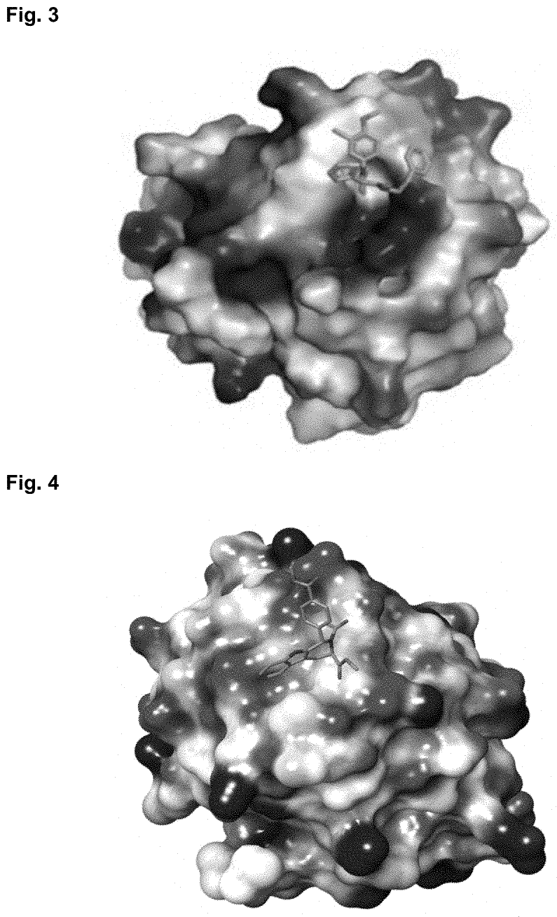

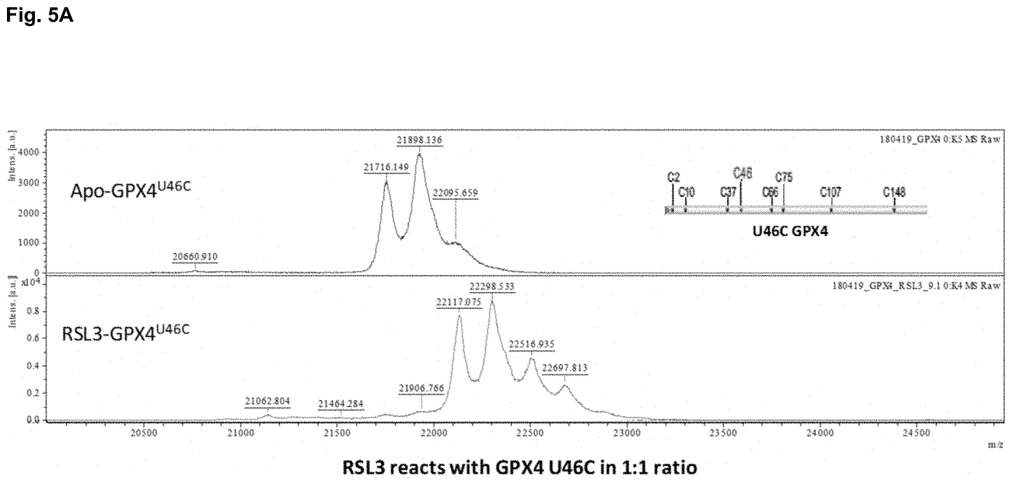

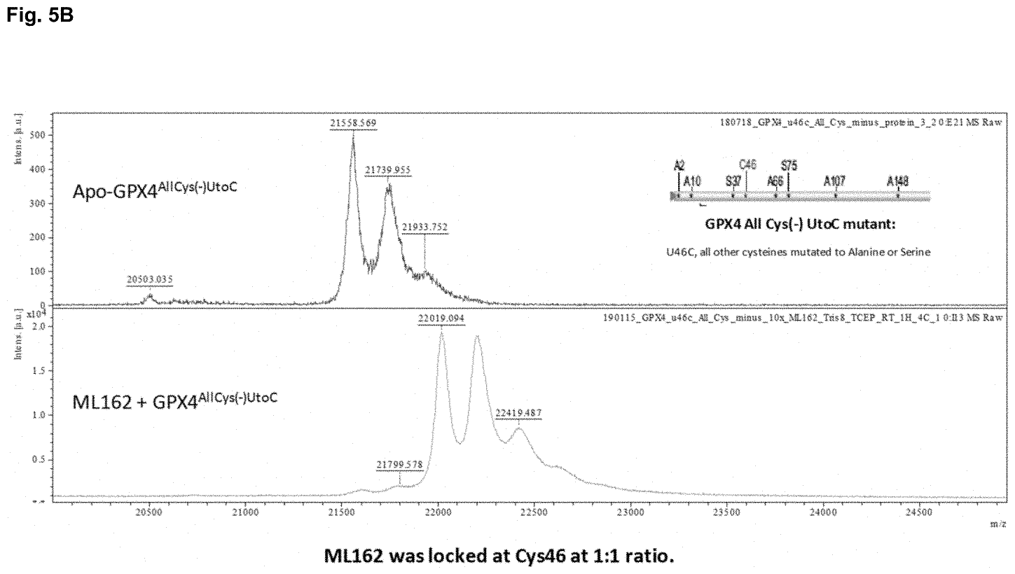

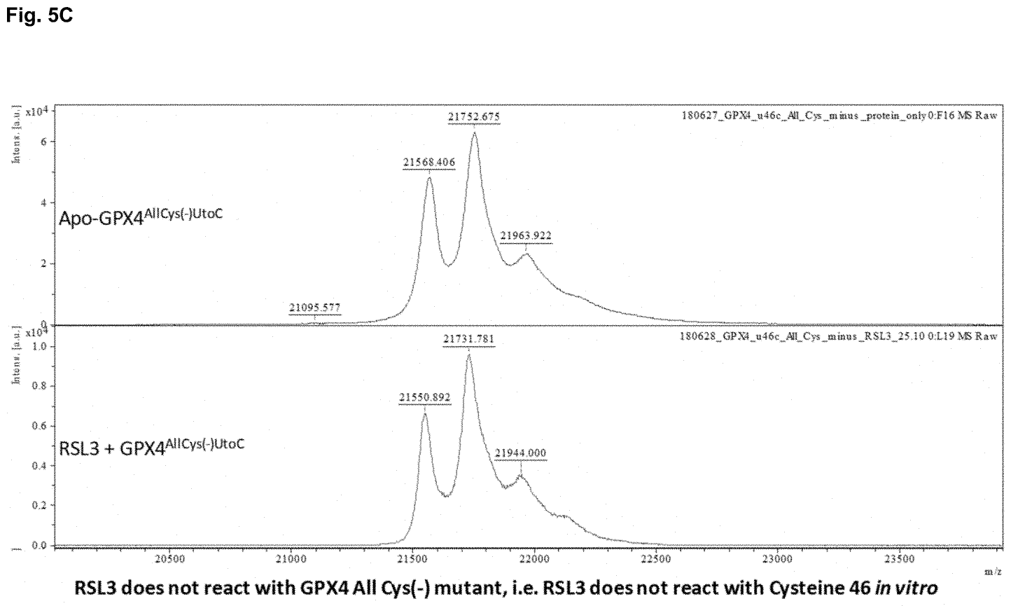

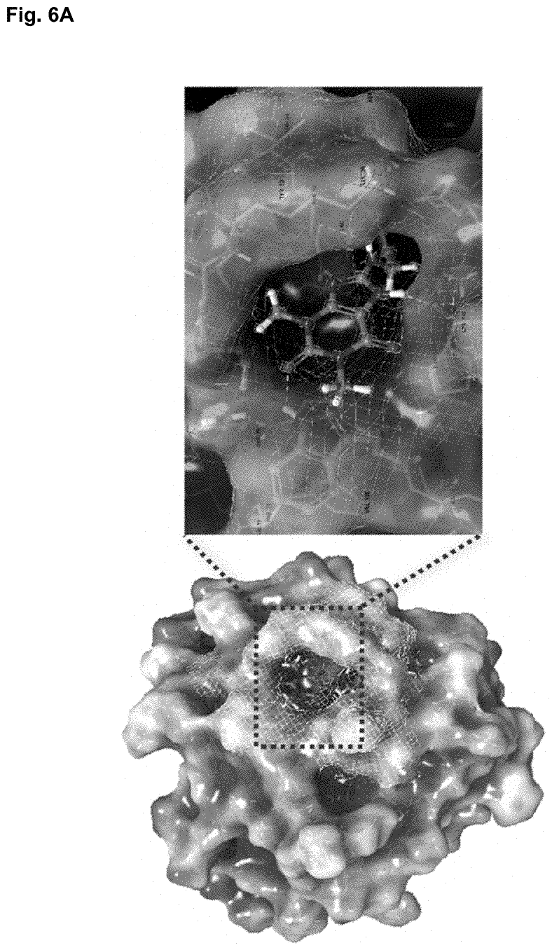

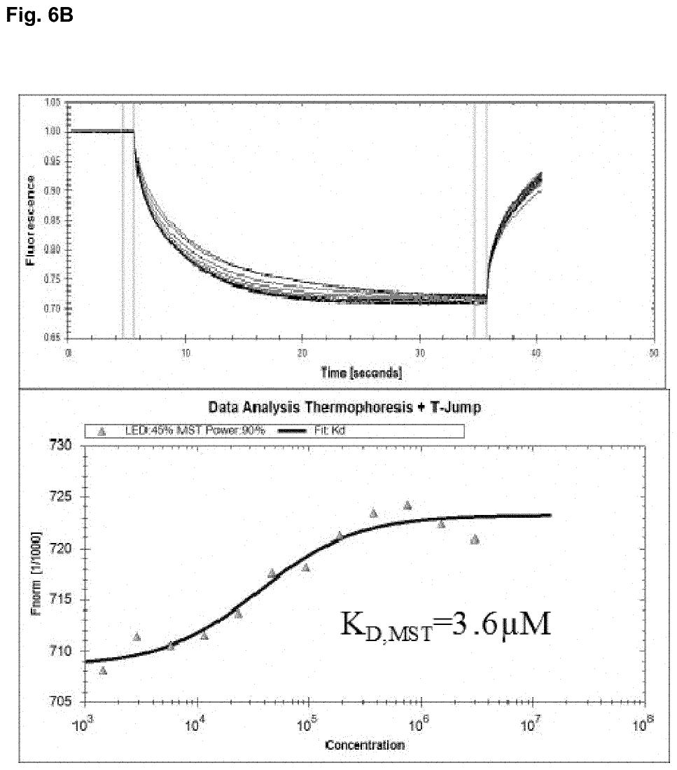

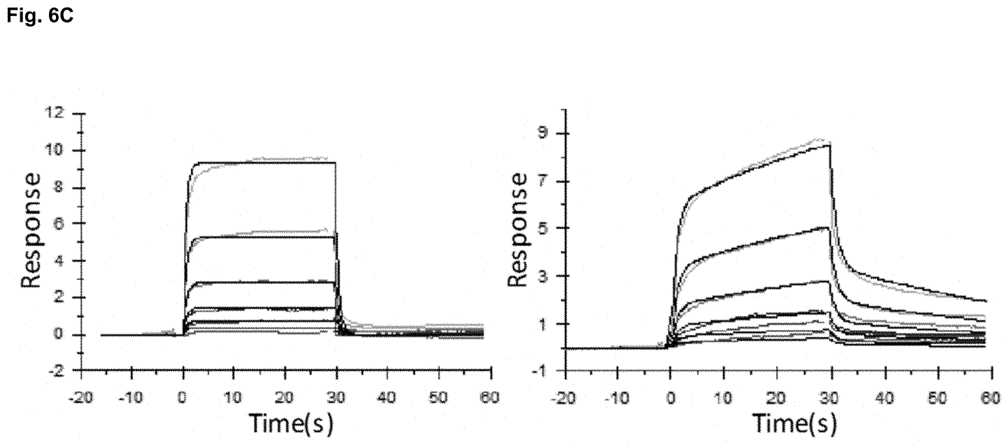

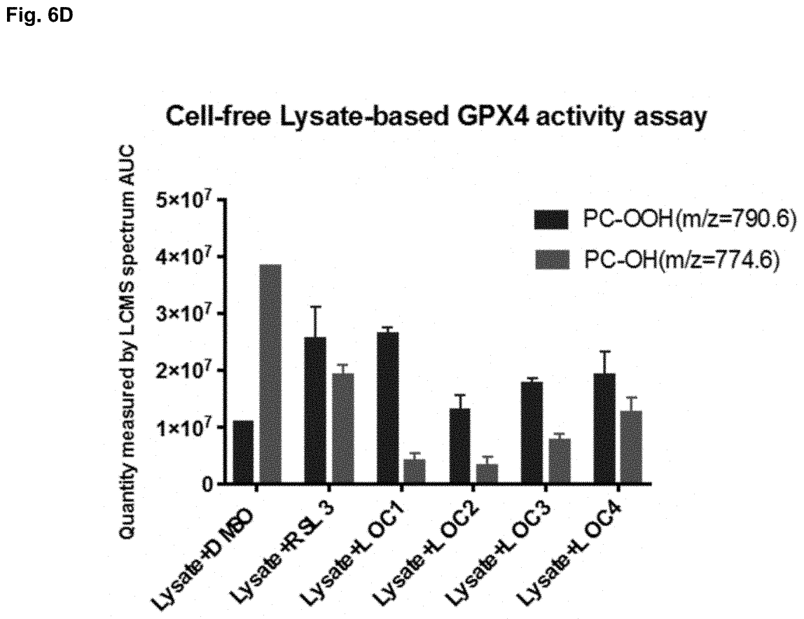

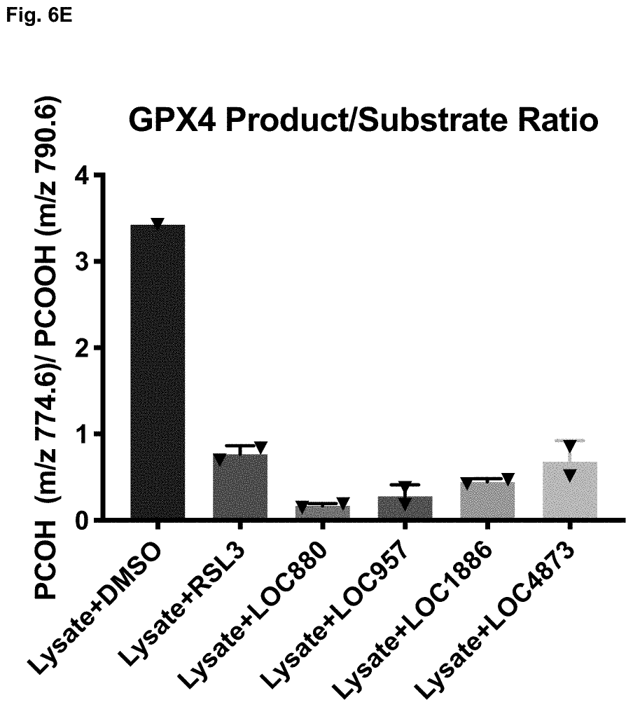

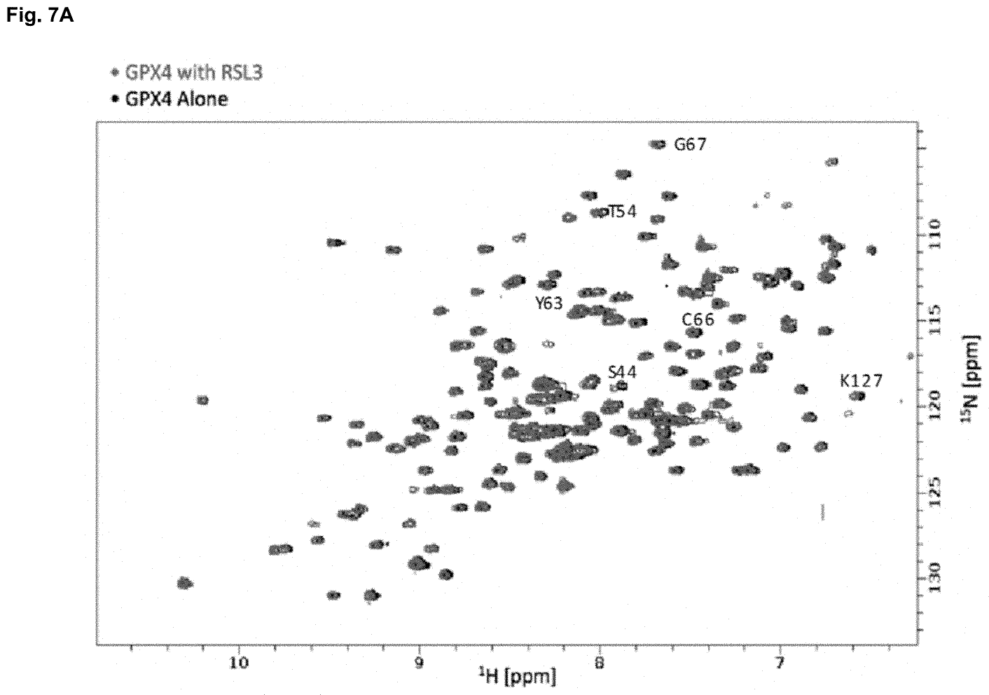

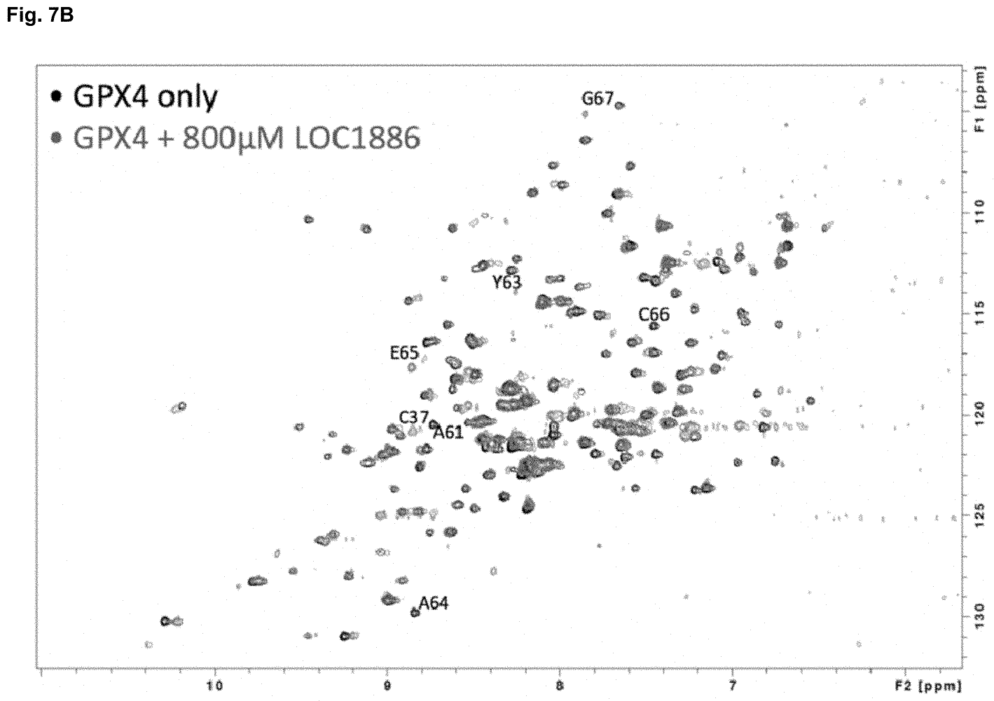

Starting from deciphering the nanomolar potency of the proof-of-concept GPX4 inhibitors, we solved the co-crystal structure of GPX4 with its covalent inhibitors RSL3 and ML162, which revealed that RSL3 binds to an alternative allosteric site rather than the expected active site as ML162 does (FIGS. 3 and 4). While our MALDI MS data confirmed that RSL3 binds to GPX4, and ML162 binds to a GPX4 mutant in which all other cysteines except the active site were mutated to serine or alanine, RSL3 was unable to bind to this mutant, demonstrating that RSL3 does not bind in the active site of GPX4 (FIGS. 5A-5C). These x-ray structures enabled further optimization of current inhibitors and the design of novel inhibitors targeting various binding sites on GPX4. Therefore, we applied a strategy of computer-facilitated design, based on MD simulation (Desmond) and Glide docking (Schrödinger Suite), and experimental high-throughput screening of a lead-optimized compound library of 10,000 compounds (FIGS. 6A-6E). All candidates were first evaluated by thermal denaturation assay and microscale thermophoresis (MST) to validate binding to GPX4 (Table 1). Surface plasmon resonance (SPR) and isothermal titration calorimetry (ITC) were then applied to precisely measure their binding kinetics and affinities, while ¹H-¹⁵N heteronuclear single-quantum coherence (HSQC) NMR spectroscopy informed us of binding sites. We optimized two GPX4-specific activity assays to examine enzymatic inhibitory effects of hit compounds—LC-MS-based substrate/product monitoring and an NADPH-coupled assay. It is noteworthy that our data suggested one of the hit compounds, LOC1886, non-covalently binds to the RSL3-binding site, induces a global conformational change of GPX4 and causes a suppression of its enzymatic activity (FIGS. 7A-7B). Together, these data suggest that we identified druggable allosteric sites on GPX4, and compounds that bind in these sites (Table 1). Some L880 analogues were also synthesized and tested (Table 2).

TABLE 1
Fragments/leads verified to bind to GPX4. These compounds have been tested
in biophysical and biochemical assays, but not in cell-based assays.

| | | | | | | | GPX4 |
|---|---|---|---|---|---|---|---|
| | | | | | | | enzymatic |
| Name | Thermal shift | MST | MST Kd | HSQC assay | Binding site | SPR | assay |
| LOC1886 | positive | positive | 100 μM | positive | Allosteric Site 1 | positive | positive |
| | | | | | (RSL3 site) | | |
| LOC577 | positive | positive | 5.4 μM | positive | Allosteric Site 2 | Not yet | Not yet tested |
| | | | | | | tested | |
| LOC880 | positive | positive | 6.7 μM | positive | Allosteric Site 2 | positive | positive |
| LOC957 | positive | positive | 6.8 μM | positive | Allosteric Site 2 | positive | positive |
| LOC4873 | positive | positive | 40 μM | positive | Allosteric Site 2 | positive | positive |
| LOC5893 | positive | positive | 100 μM | positive | Allosteric Site 2 | Not yet | Not yet tested |
| | | | | | | tested | |
| LOC1250 | positive | positive | 3 μM | positive | Allosteric Site 3 | Not yet | Not yet tested |
| | | | | | | tested | |

| Name | Structure |
|---|---|
| LOC577 | 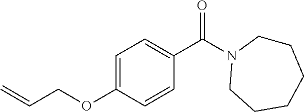 [SMILES: C=CCOc1ccc(C(=O)N2CCCCCC2)cc1] |
| LOC4873 | 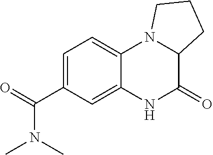 [SMILES: CN(C)C(=O)c1ccc2c(c1)NC(=O)C1CCCN21] |
| LOC880 | 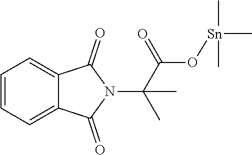 [SMILES: CC(C)(C(=O)[O][Sn]([CH3])([CH3])[CH3])N1C(=O)c2ccccc2C1=O] |
| LOC1250 | 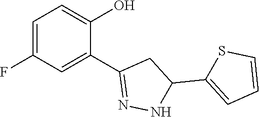 [SMILES: Oc1ccc(F)cc1C1=NNC(c2cccs2)C1] |
| LOC957 | 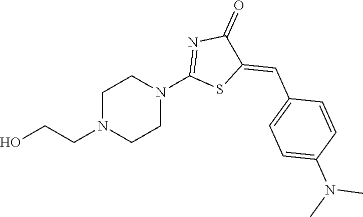 [SMILES: CN(C)c1ccc(/C=C2\SC(N3CCN(CCO)CC3)=NC2=O)cc1] |
| LOC1886 | 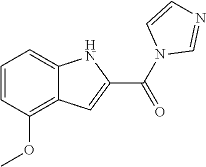 [SMILES: COc1cccc2[nH]c(C(=O)n3ccnc3)cc12] |
| LOC5893 | 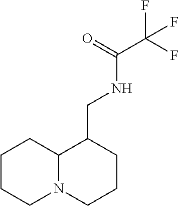 [SMILES: O=C(NCC1CCCN2CCCCC12)C(F)(F)F] |

TABLE 2
Some LOC880 analogues synthesized and tested.

| | | Molecular | | |
|---|---|---|---|---|
| Name | Structure | Weight | MST K_{d} | |
| QW-003 | 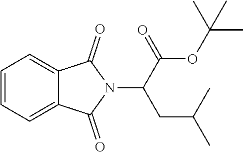 [SMILES: CC(C)CC(C(=O)OC(C)(C)C)N1C(=O)c2ccccc2C1=O] | 317.39 | Not determined | |
| QW-010 | 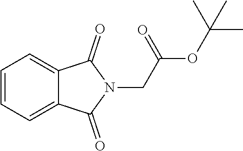 [SMILES: CC(C)(C)OC(=O)CN1C(=O)c2ccccc2C1=O] | 261.28 | 2.9 | μM |
| QW-011 | 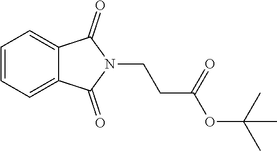 [SMILES: CC(C)(C)OC(=O)CCN1C(=O)c2ccccc2C1=O] | 275.30 | 926 | nM |
| QW-017 | 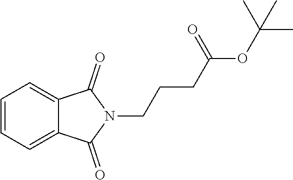 [SMILES: CC(C)(C)OC(=O)CCCN1C(=O)c2ccccc2C1=O] | 289.33 | 399 | nM |
| QW-020 | 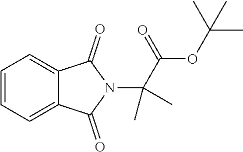 [SMILES: CC(C)(C)OC(=O)C(C)(C)N1C(=O)c2ccccc2C1=O] | 289.33 | 2.14 | μM |
| QW-023 | 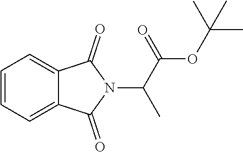 [SMILES: CC(C(=O)OC(C)(C)C)N1C(=O)c2ccccc2C1=O] | 275.30 | 1.78 | μM |
| QW-029 | 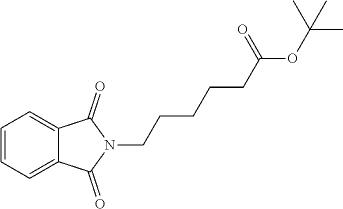 [SMILES: CC(C)(C)OC(=O)CCCCCN1C(=O)c2ccccc2C1=O] | 317.39 | 2.1 | μM |
| QW-034 | 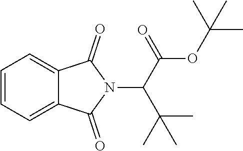 [SMILES: CC(C)(C)OC(=O)C(N1C(=O)c2ccccc2C1=O)C(C)(C)C] | 317.39 | 979 | nM |
| QW-050 | 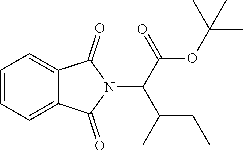 [SMILES: CCC(C)C(C(=O)OC(C)(C)C)N1C(=O)c2ccccc2C1=O] | 317.39 | 2.43 | μM |

### Example 3

### Large-Scale Screen for Novel Compounds that Bind in the RSL3-Binding Site of GPX4

The x-ray structure of the potent GPX4 inhibitor RSL3 showed that this compound binds to an allosteric site distinct from the active site. Thus, we will, in parallel to the above efforts with the validated binders, develop a screening assay focused on the RSL3-binding site.

We will perform a high-throughput screen of compounds from our library of ˜10,000 lead-like compounds (selected from 3.3 million compounds for those with favorable lead-like properties) for those that can compete with the binding of a fluorescently-labeled RSL3 probe to its allosteric binding site on GFP-GPX4; activity will be measured by FRET between the probe and the GFP fused to GPX4. Our hypothesis is that this library contains additional binders to the RSL3-binding site, but that these compounds were not detected in the thermal shift assay we used for our previous GPX4 screen—this assay is not that sensitive and can miss many compounds. Positives will be processed through the funnel described above to obtain validated hits. As an additional backup, we will also screen an 8,000 compound covalent fragment library and the Sigma DyNAbind DNA-encoded library of 370,000 fragment compounds. This will provide a diverse set of additional compounds that may be able to target the RSL3-binding site.

### Example 4

### Allosteric Inhibition of GPX4

### Introduction

As the leading causes of deaths in cancer patients, metastatic and drug-resistant cancers stand among the most pressing problems in oncology (Dillekas et al. 2019; Wang et al. 2019). As a key step in metastasis, the epithelial-to-mesenchymal transition (EMT), which increases motility of tumor cells and enables the invasion of primary tumors to distant sites, has been reported to render cancer cells resistant to apoptosis and chemotherapy (Fischer et al. 2015). During the transition, the elevated level of polyunsaturated-fatty-acid-(PUFA)-containing phospholipids (PUFA-PLs) is a major factor to increase the fluidity of cell membranes, as the cis conformation of double bonds in PUFA-PLs hinders efficient stacking of fatty acyl tails (Agmon and Stockwell, 2017). However, as PUFA-PLs are inherently susceptible to peroxidation at bis-allylic positions, cancer cells that have undergone EMT inevitably became more dependent on a key protein, glutathione peroxidase 4 (GPX4), which is the only peroxidase in mammals capable of reducing phospholipid hydroperoxides within cell membranes (Thomas et al. 1990; Kuhn and Borchert, 2002; Zou et al. 2020). When GPX4 activity is compromised, lipid peroxidation can cause ferroptosis, a new form of tumor suppressive cell death (Dixon et al. 2012; Yang et al. 2014). Indeed, as cancers evolve into aggressive and drug-resistant forms with a mesenchymal or other signatures, they simultaneously acquire an exquisite sensitivity to GPX4 inhibition, which indicated a tantalizing possibility that the most aggressive neoplastic diseases can be treated through the use of GPX4 inhibitors (Viswanathan et al. 2017; Hangauer et al. 2017; Zou et al. 2019).

Inhibition of GPX4 with small molecules has been limited by the characteristic flat surface surrounding its active site and the lack of known allosteric regulatory sites (Borchert et al. 2018; Sakamoto et al. 2017). To date, all known GPX4 inhibitors are assumed to be covalent inhibitors that react with the active site selenocysteine of GPX4 (Yang and Stockwell, 2008; Yang et al. 2014; Weiwer et al. 2012; Yang et al. 2016; Eaton et al. 2019; Eaton et al. 2020; Eaton et al. 2020). The top inhibitors, (1S, 3R)-RSL3 (hereinafter referred to as “RSL3”) and ML162, featured nanomolar potencies to induce ferroptosis to cancer cells, but they are limited by poor drug-like properties associated with their chloroacetamide warheads (Yang and Stockwell, 2008; Yang et al. 2014; Weiwer et al. 2012; Yang et al. 2016; Allimuthu and Adams, 2017). Additionally, developing drug-like compounds selectively binding to the flat active site of GPX4 over selenocysteine of other glutathione peroxidases in the GPX family may be challenging. Although extensive medicinal-chemistry efforts have been devoted to the current inhibitors, the potencies have not been further improved (Eaton et al. 2019; Eaton et al. 2020; Eaton et al. 2020). This indicated the necessity of a deeper understanding of the nanomolar potency of current inhibitors or a search for alternative binding site before the adaption of a structure-based approach for the development of improved GPX4 inhibitors.

In addition to its peroxidase function, as a moonlighting protein under specific condition, GPX4 can polymerize into an enzymatically inactive, oxidatively cross-linked, insoluble structural element of the mitochondrial sheath of the midpiece of mature spermatozoa (Ursini et al. 1999; Conrad et al. 2005). This unusual feature may derive from the less restricted dependence on glutathione as reducing substrate and the expression of multiple non-conserved surface cysteines, which are distinct from other members of the GPX family (Conrad et al. 2005; Scheerer et al. 2007). The structural and mechanism basis for this dual function and whether it is involved in the regulation of ferroptosis by GPX4 have not been clearly demonstrated. We hypothesized that further mechanism study of the enzymatically inactive dual function would benefit the discovery of novel approach to GPX4-specific inhibition.

Here, via deciphering the nanomolar potency of known GPX4 inhibitors, we identified an allosteric binding site on GPX4 that allows inactivation of GPX4 without targeting the active site selenocysteine. We then found this site is involved in the dual function of GPX4, based on which we proposed an alternative GPX4 catalytic cycle. After confirmation of the druggability of this allosteric binding sites with multiple proof-of-concept inhibitors, we screened a library of lead-optimized compounds and found a hit compound binding in the allosteric site to inhibit GPX4, which provided a novel approach for therapeutic strategies targeting GPX4.

### Results

### Cysteine 66 is a Binding Site of RSL3 and ML162 on GPX4

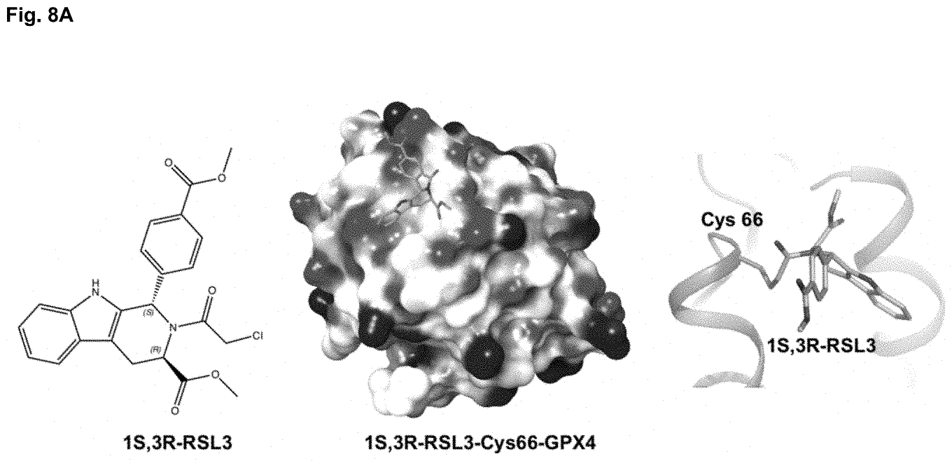

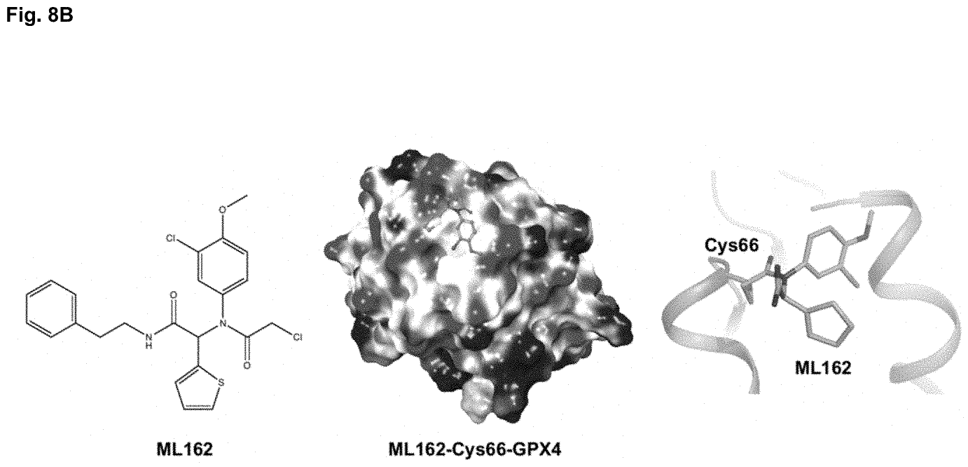

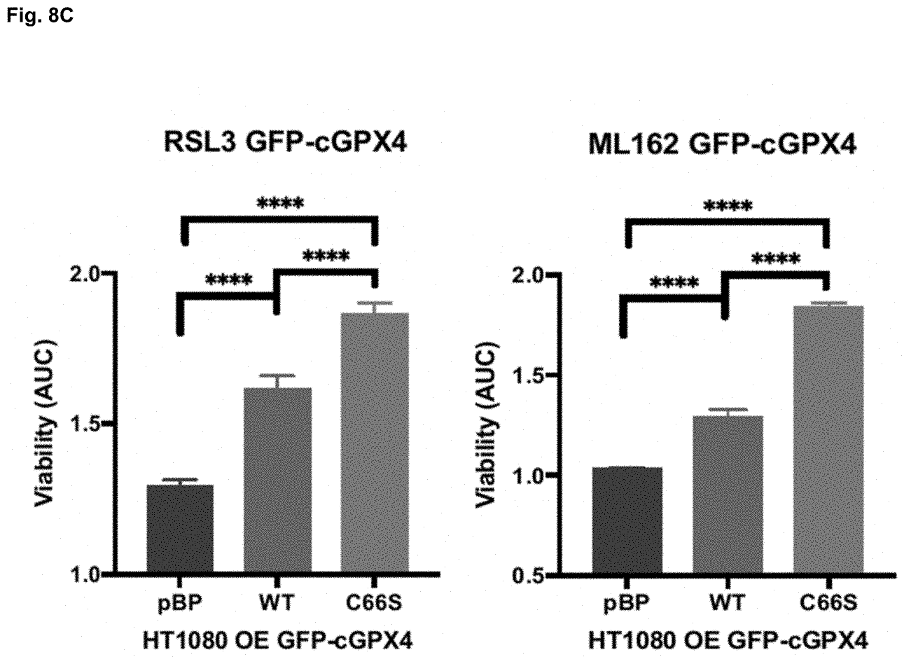

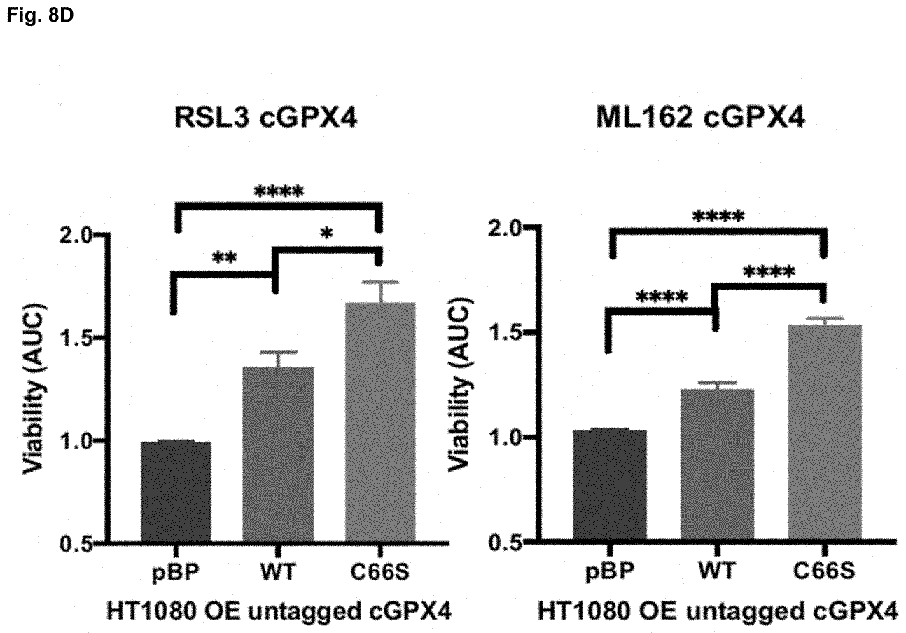

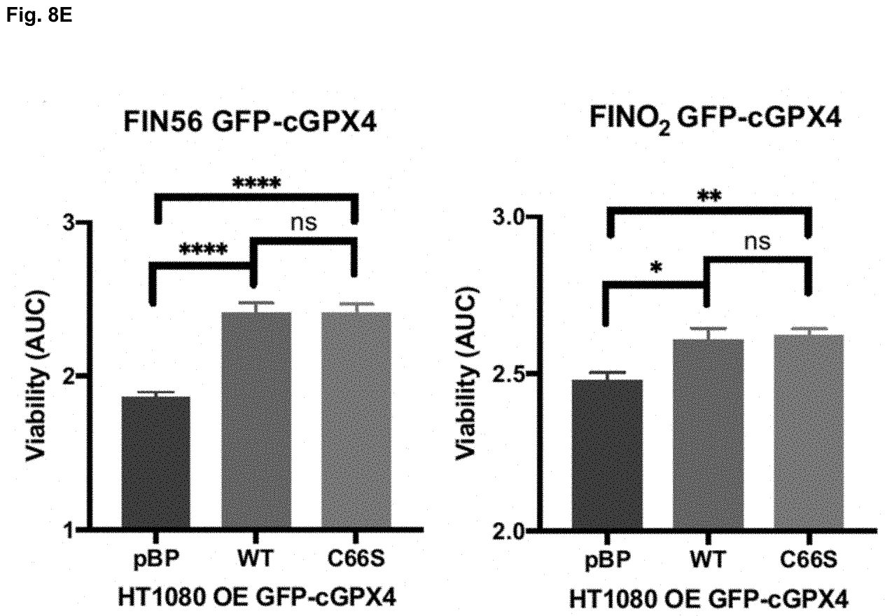

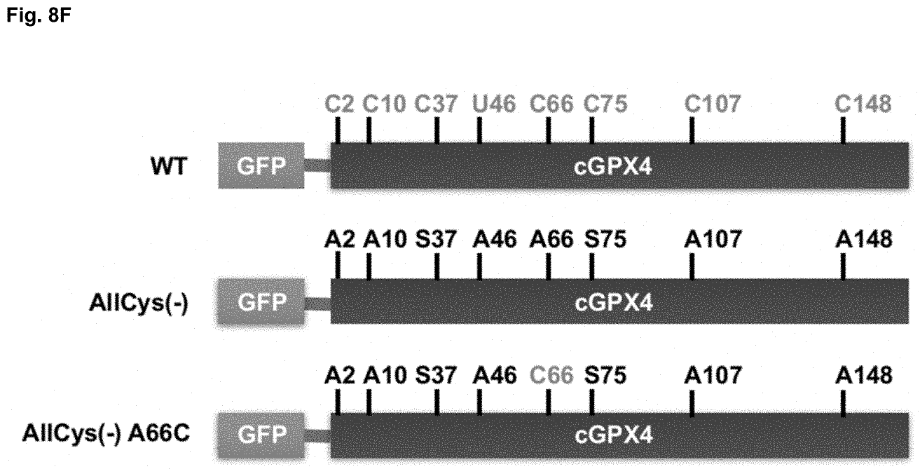

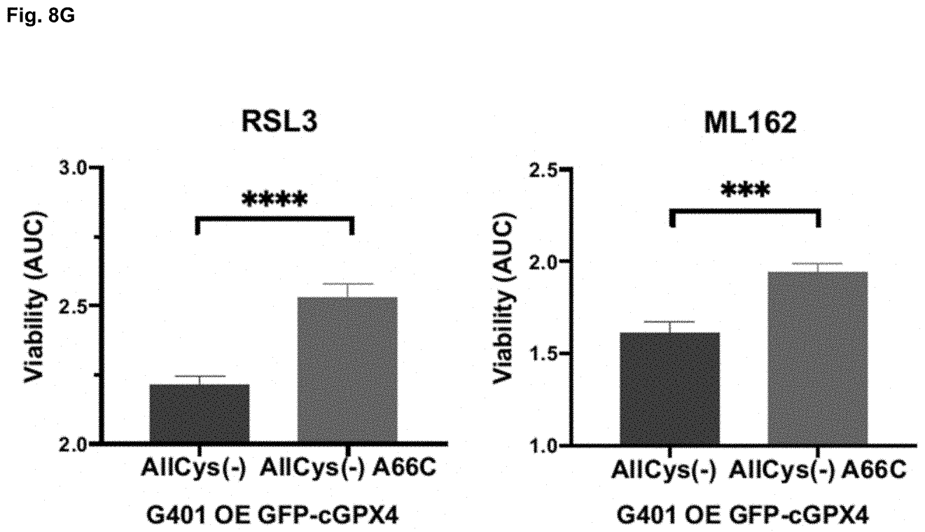

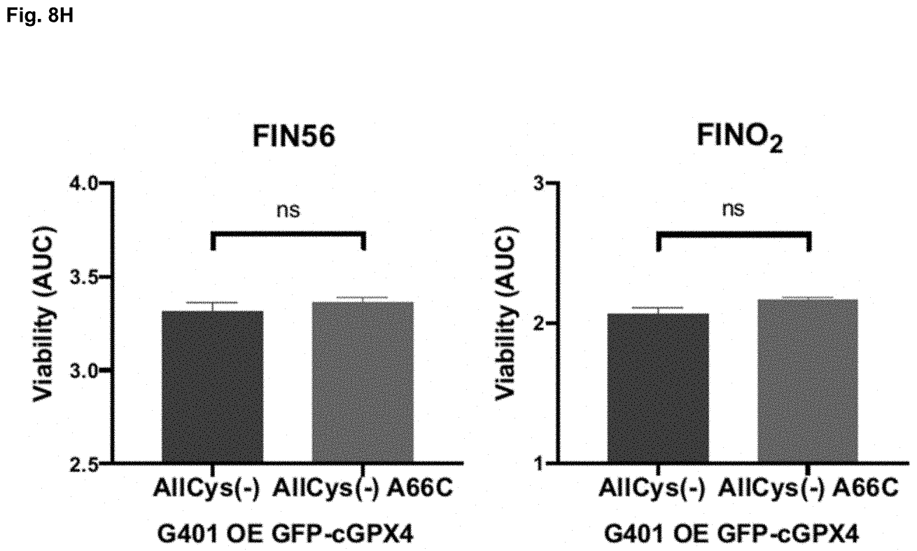

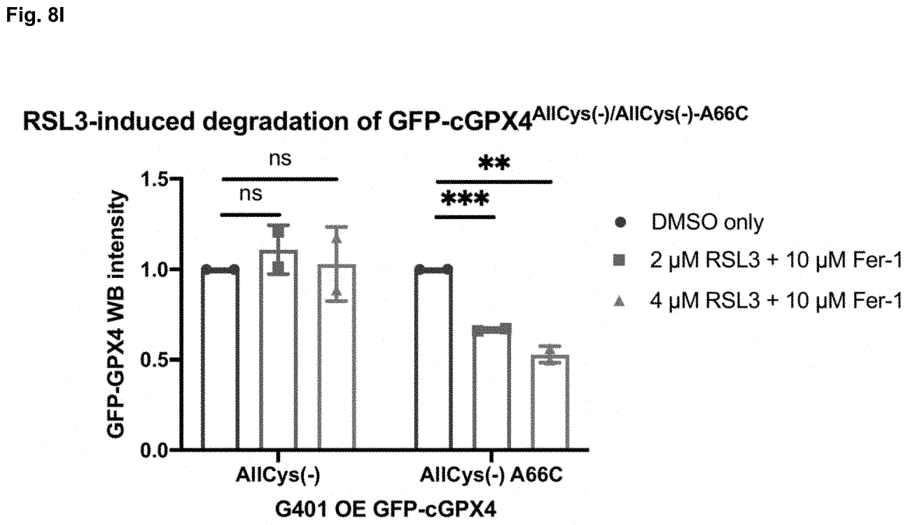

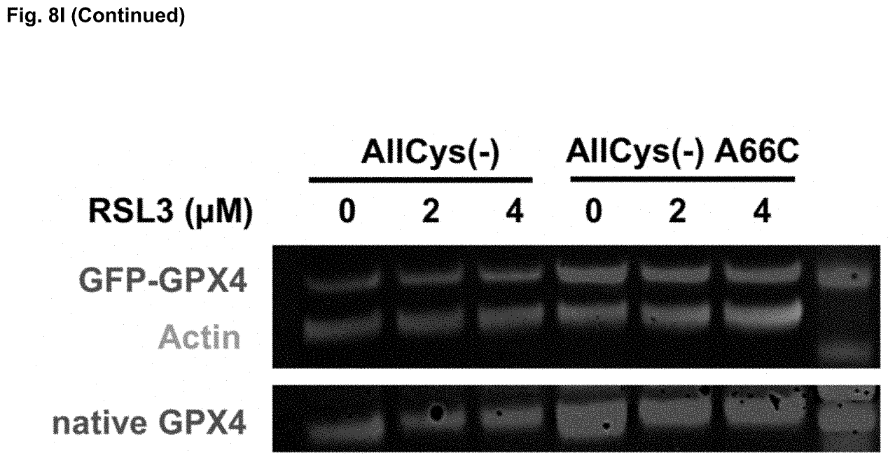

Starting from deciphering the nanomolar potency of the proof-of-concept GPX4 inhibitors, we solved the co-crystal structure of GPX4^{U46C} with its covalent inhibitors RSL3 and ML162, which revealed that RSL3 and ML162 both bind to cys66, an alternative site rather than the expected active site (FIGS. 8A and 8B). It is because that large-scale expression of selenocysteine-containing proteins in recombinant systems is challenging due to inefficient selenocysteine-incorporating machinery, we applied the selenocysteine to cysteine (U to C, inserting a thiol group in place of the selenol group) mutant of GPX4 for the structural study, superposition of which with structure of wild-type GPX4 only revealed minor differences (Scheerer et al. 2007; Borchert et al. 2018). Although the observed binding of the covalent inhibitors to cys66 might be off-target due to the U to C mutation at active site, the recent co-crystal efforts on ML162 with wild-type GPX4 also reported the binding of ML162 to cys66, even in the existence of sec46 (Eaton et al. 2018). To further validate this binding site on wild-type GPX4 in a cellular context, we stably overexpressed either GFP-tagged GPX4^{WT} or GFP-tagged GPX4^{C66S}, a potentially binding-deficient mutant, in HT-1080 fibrosarcoma cells, in which GPX4 functions to protect cells from ferroptosis (Yang et al. 2014). Using HT-1080 cells transfected with empty vector (pBabe-puro) as a control, we found that overexpression of GFP-tagged-GPX4^{C66S}, which lacks the cys66 binding site, protected HT-1080 cells from ferroptosis induced by RSL3 and ML162 to a greater extent than the overexpression of GFP-tagged-GPX4⁷, suggesting binding of RSL3 and ML162 to cys66 of GPX4 and subsequent inhibitions in a cellular context (FIG. 8C).

Moreover, overexpression of tag-free GPX4^{C66S} also protected HT-1080 cells from ferroptosis induced by RSL3 and ML162 to a greater extent than overexpression of tag-free GPX4^{WT}, excluding the possibility of GFP tag interference (FIG. 8D). In addition to GPX4 inhibitors, we also examined whether the inhibitor-binding deficient mutation affected other classes of ferroptosis inducers: FIN56, which depletes GPX4 protein and simultaneously causes depletion of CoQ₁₀, and FINO₂, which oxidizes iron to drive lipid peroxidation and indirect inactivation of GPX4 (Feng and Stockwell, 2018; Shimada et al. 2016; Gaschler et al. 2018). Indeed, overexpression of GFP-tagged-GPX4^{C66S} provided equivalent protection as GFP-tagged-GPX4^{WT} against FIN56 and FINO₂, confirming that specific protection of GPX4^{C66S} against RSL3 and ML162 is likely due to loss of the covalent binding site at cys66 (FIG. 8E).

GPX4 contains one seleno-cysteine at the active site and seven other cysteines, which are all potentially reactive with electrophiles (FIG. 8F). In our previous work attempting to locate the RSL3 binding site, we replaced all the electrophilic residues with Ala or Ser (C2A, C10A, C37S, Sec46A, C66A, C75S, C107A, and C148A) and expressed the mutant GPX4 protein, termed allCys(−), in G401 renal carcinoma cells as a GFP fusion (Yang et al. 2016) (FIG. 8F). Each mutated residue on GFP-allCys(−)-GPX4 was then separately reverted to the original selenol-cysteine or cysteine (Yang et al. 2016). To directly verify the hypothesis that RSL3 binds to cys66 in a cellular context, we examined the ferroptosis sensitivity of allCys(−) and allCys(−) A66C cells, in which Ala66 in allCys(−) GPX4 was reverted to cys66 (FIG. 8F). Accordingly, we found that overexpression of enzymatically inactive allCys(−) A66C GPX4 provided significant protection against RSL3 and ML162 as compared to allCys(−) GPX4, suggesting cys66 on the inactive protein bound to RSL3 and ML162 and therefore shielded the endogenous active GPX4 in G401 cells from the inhibitors (FIG. 8G). As expected, overexpression of enzymatically inactive allCys(−) A66C GPX4 exhibited no effects on FIN56 and FINO₂ lethality as compared to allCys(−) GPX4, confirming a specific protection against RSL3 and ML162 derived from binding of covalent inhibitors to cys66 on allCys(−) A66C GPX4 (FIG. 8H).

In our previous work, sepharose beads coupled with anti-fluorescein antibodies were not able to pull down a detectable amount of allCys(−) A66C GPX4 from G401 allCys(−) A66C cells treated with RSL3-fluorescein (Yang et al. 2016). We hypothesized this could be because RSL3 bound to cys66 of GPX4 and subsequently induced the degradation of allCys(−) A66C GPX4 in the cellular context (Shimada et al. 2016). In line with this hypothesis, we found that, while allCys(−) GPX4 cannot be covalently modified by RSL3 and therefore was resistant to RSL3-induced degradation observed with native WT GPX4, allCys(−) A66C GPX4 with the cys66 binding site is vulnerable to the RSL3-induced degradation, based on the Western-blotting of G401 overexpressing allCys(−) or allCys(−) A66C GPX4 after treatment with RSL3 (FIG. 81). The observed does-dependent degradation of allCys(−) A66C GPX4 induced by RSL3 also indicated a potential role of cys66 binding site in the mechanism of RSL3-induced GPX4 inhibition.

### RSL3 and ML162 Selectively Bind to Sec46 and Cys66 of GPX4

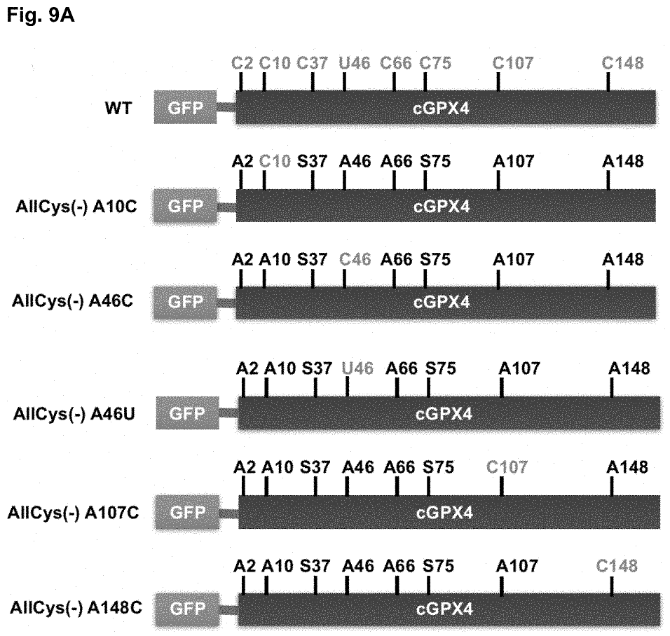

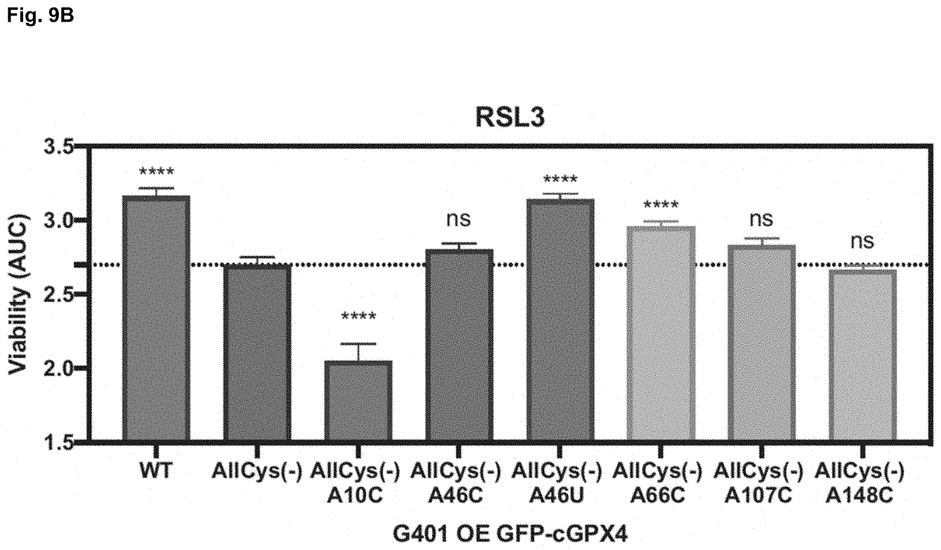

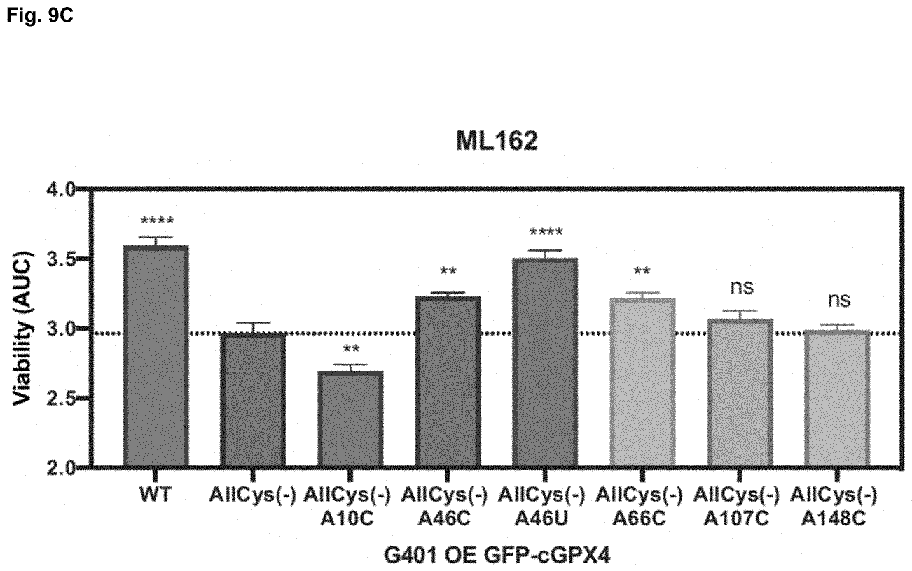

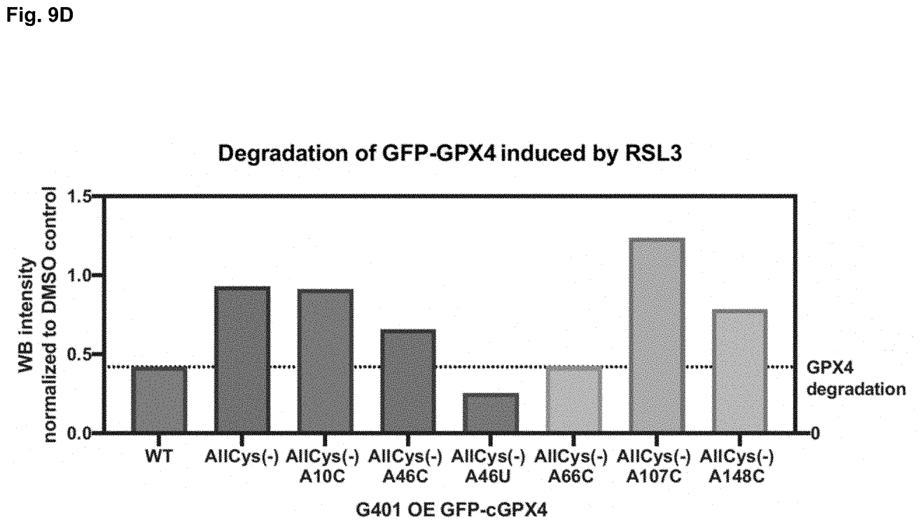

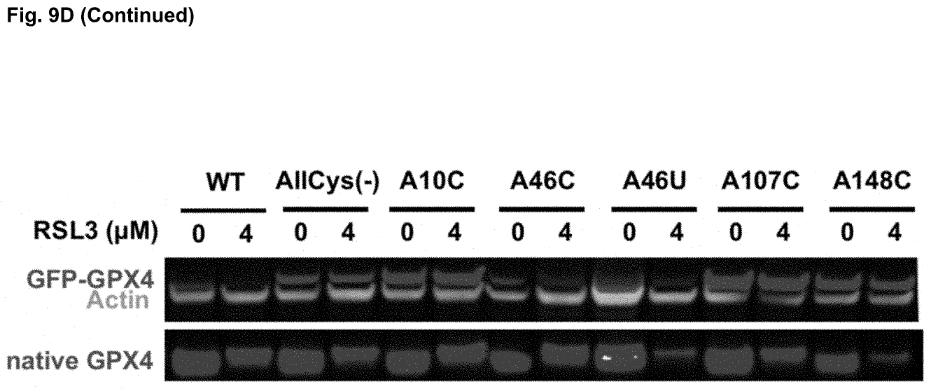

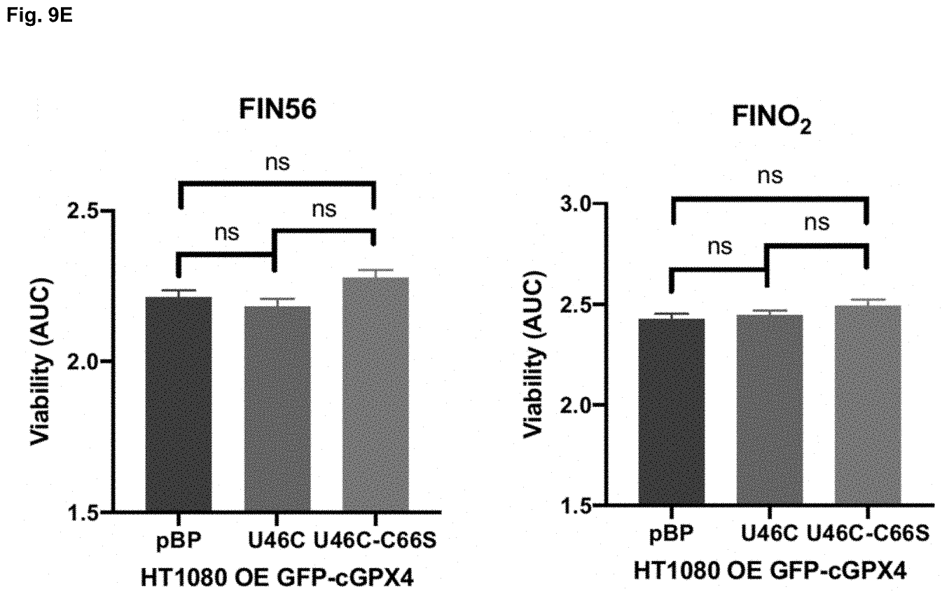

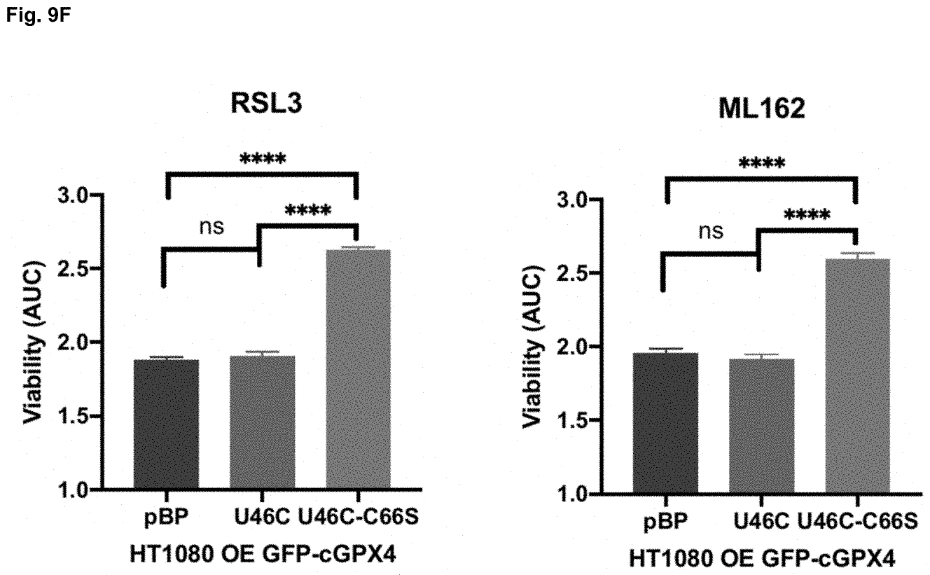

In addition to cys66, other surface cysteines (cys10, cys107, and cys148) of GPX4 may also be amenable to electrophilic attacks by RSL3 and ML162. Moreover, sec46 at the active site is expected to be more reactive towards electrophiles because of its lower pKa values than cysteines (Yang et al. 2016). To test whether sec46 and other surface cysteine residues on GPX4 also contribute to RSL3 binding, we applied G401 cell lines stably expressing corresponding revertants of allCys(−) GPX4 (Yang et al. 2016). Five of such G401 cell lines (A10C, A46C, A46U, A107C, and A148C) were therefore included in the test, along with G401 overexpressing WT, allCys(−), or allCys(−) A66C GPX4, for RSL3 and ML162 sensitivities (FIG. 9A). A46C, in which the A46 in allCys(−) was replaced with cysteine, was also included here to further evaluate the requirement of selenol-cysteine at the active site of GPX4 for covalent binding with RSL3, as in the co-crystal structure we didn't observe binding of RSL3 to cys46 on GPX4^{U46C} protein (Yang et al. 2016). We found that overexpression of WT, allCys(−) A46U, or allCys(−) A66C GPX4 significantly protected G401 cells from both RSL3 and ML162 as compared with allCys(−), while overexpression of GPX4 with other cysteines didn't, suggesting RSL3 and ML162 selectively bound to sec46 and cys66 of GPX4 in a cellular context (FIGS. 9B and 9C). Furthermore, in accordance with the viability analysis, we found that only WT and allCys(−) A46U GPX4 were prone to RSL3-induced degradation, to the extent observed with allCys(−) A66C GPX4, while allCys(−) GPX4 with other cysteines were not, confirming the selectivity towards selenocysteine 46 and cysteine 66 (FIG. 9D).

It is noteworthy that overexpression of allCys(−) A46C GPX4 didn't exhibit a significant protection against RSL3 and only a slight degradation was observed. This further confirmed the reported importance of selenocysteine for the binding of RSL3 to GPX4 active site and explained the absence of inhibitor at the active site of GPX4^{U46C} co-crystal structure (Yang et al. 2016; Gao et al. 2018). Additionally, allCys(−) A46U GPX4 showed the most significant protection and degradation, which indicated a preference of inhibitor binding to sec46, potentially due to its higher activity against electrophiles, and explained why tagged RSL3 pulled down a larger amount of allCys(−) A46U than allCys(−) with any other reverted cysteines (Yang et al. 2016; Gao et al. 2018).

To validate selenocysteine 46 and cysteine 66 as inhibitor binding sites on GPX4 in a cellular context, we stably overexpressed either GFP-tagged GPX4^{U46C_C66S} a double mutant which lacks both binding sites, or GFP-tagged GPX4^{U46C} as control, in HT-1080 cells. Using HT-1080 cells transfected with empty vector (pBabe-puro) as a control, we found that GPX4^{U46C} and GPX4^{U46C_C66S}, both of which have a much lower enzymatic activity than WT, exhibited no effects against treatment with FIN56 or FINO₂ (FIG. 9E). However, as expected, overexpression of GPX4^{U46C_C66S}, which is completely devoid of both binding sites, significantly protected HT1080 cells from ferroptosis induced by RSL3 and ML162, while GPX4^{U46C} with cys66 binding site didn't (FIG. 9F). Together, our data suggested that RSL3 and ML162 specifically bind to sec46 and cys66 of GPX4 in a cellular context.

### Cys66 and Cys10 Modulate the Activity of GPX4

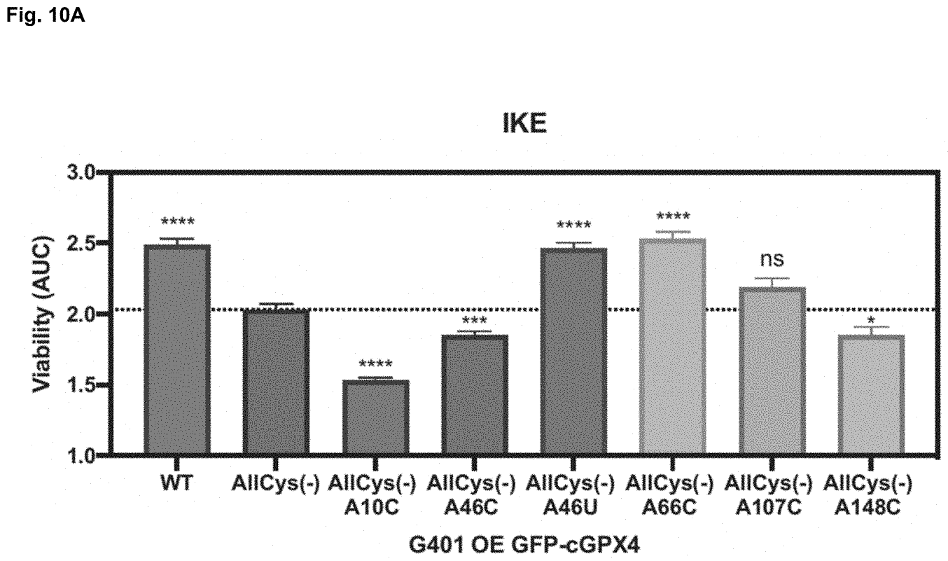

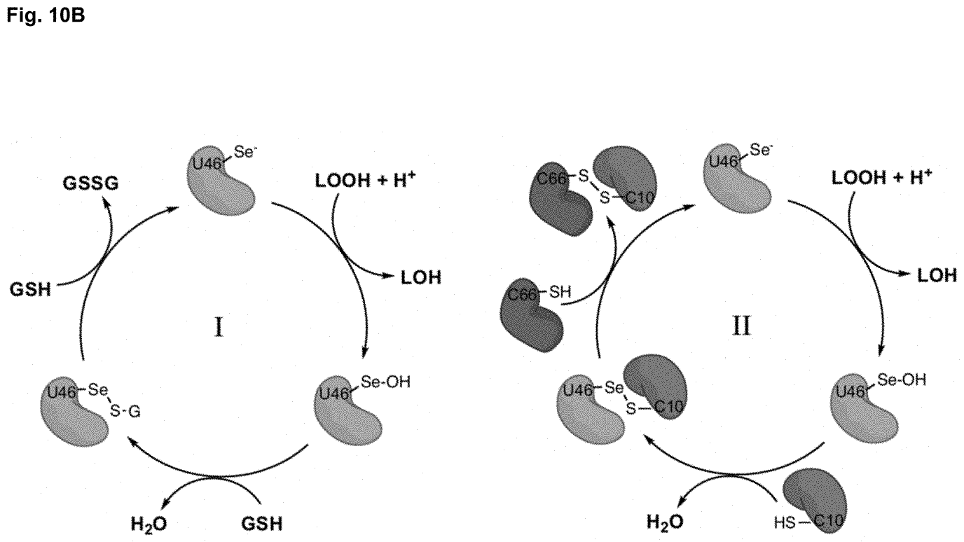

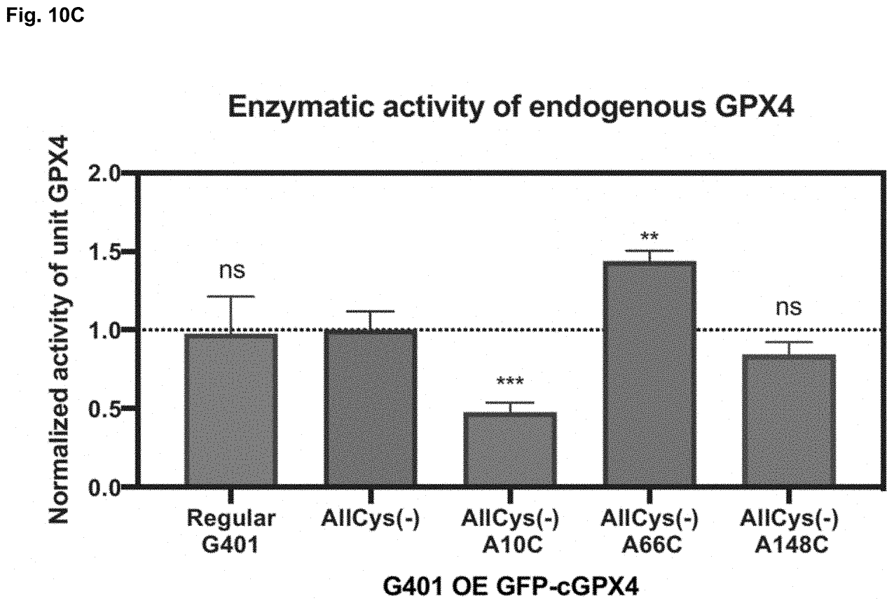

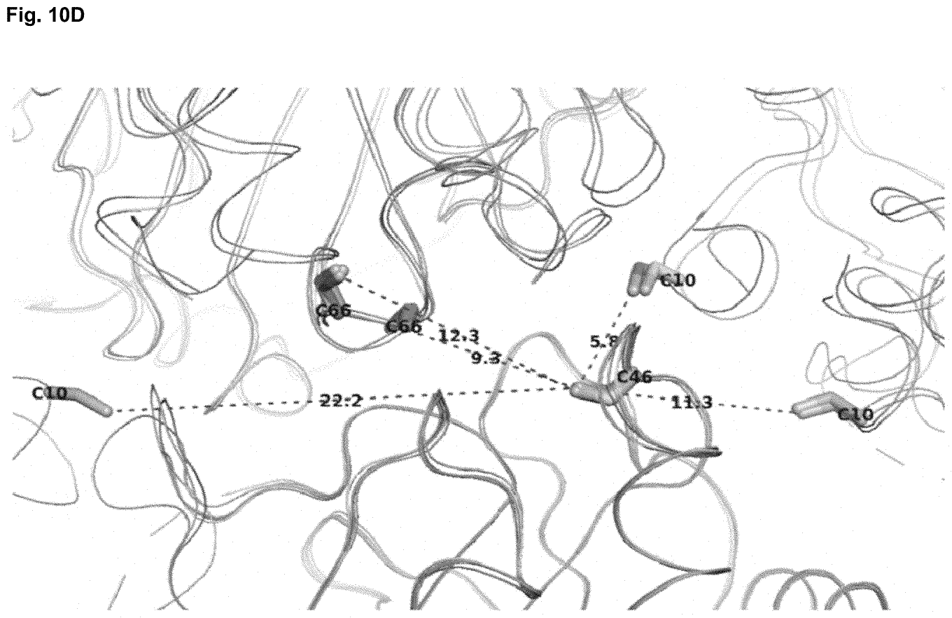

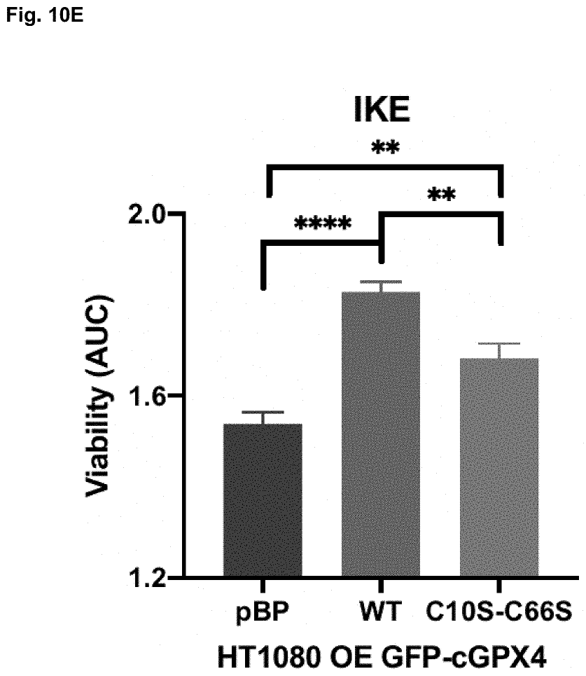

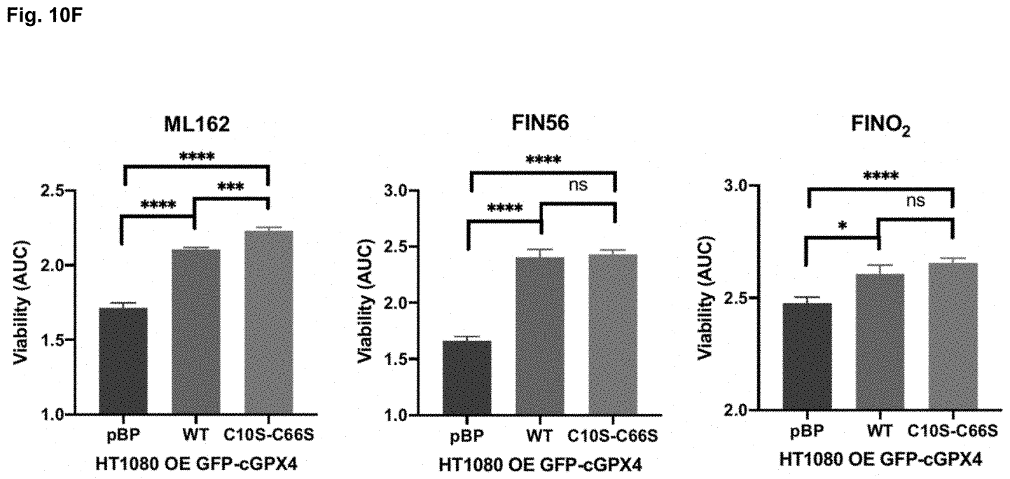

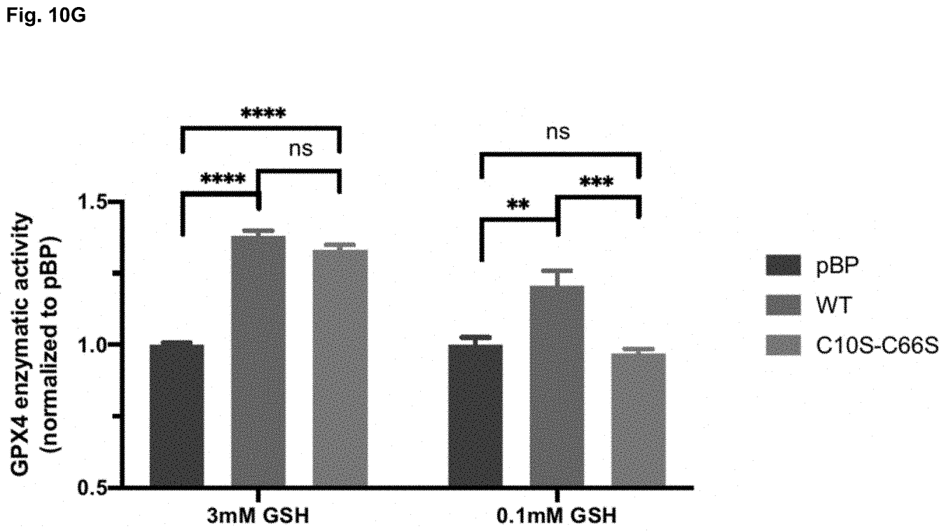

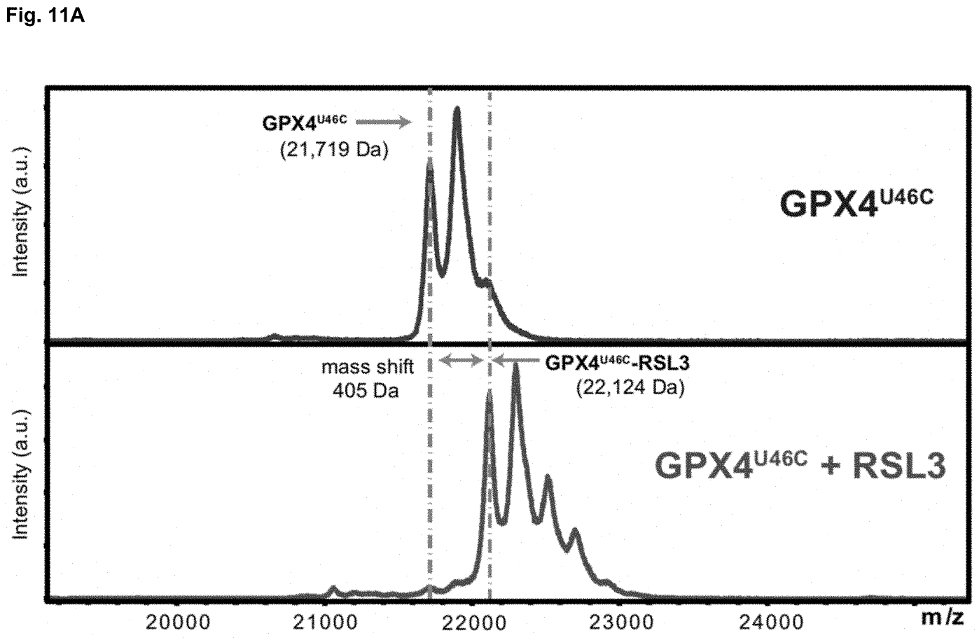

Besides from the three classes of ferroptosis inducers that we have tested, imidazole ketone erastin (IKE), an inhibitor of cystine/glutamate antiporter system x_{c}⁻, represents another class of ferroptosis inhibitors (Feng and Stockwell, 2018). IKE prevents cystine import, which will lead to the depletion of GPX4 cofactor GSH and a loss of GPX4 activity thereafter, to induce ferroptosis (Zhang et al. 2019). When we tested G401 cells overexpressing WT, allCys(−), or individual revertants of allCys(−) GPX4 (A10C, A46C, A46U, A66C, A107C, and A148C) for IKE sensitivities, we found overexpression of enzymatically inactive AllCys(−) A66C GPX4 significantly protected G401 cells from IKE, the protection effect of which is even comparable with the overexpression of enzymatically active WT GPX4 and AllCys(−) A46U GPX4 (FIG. 10A). Furthermore, among all revertants of allCys(−) GPX4, overexpression of enzymatically inactive AllCys(−) A10C GPX4 characteristically sensitized G401 cells to ferroptosis induced by not only IKE, but also RSL3 and ML162 (FIGS. 9B, 9C, and 10A). These observations suggested a potentially intrinsic role of cys66 and cys10 in modulating GPX4 activity, which are independent of GPX4 inhibitor binding.

Previous studies revealed that at low GSH concentrations, GPX4 acted as a protein thiol peroxidase to utilize specific protein thiols as the reductants in its catalytic cycle and structurally crosslink proteins, which is also known as the dual function of GPX4 (Ursini et al. 1999; Conrad et al. 2005). The reported low GSH conditions coincidentally matched the IKE-treated cellular conditions in our experiments (Ursini et al. 1999).

We therefore propose that, in addition to the canonical glutathione-dependent catalytic cycle of GPX4 (I), GPX4 may utilize the thiol of cys10 on a second GPX4 protein as reductant to form a dimer intermediate, which will then be regenerated with the thiol of cys66 on a third GPX4 protein to complete the catalytic cycle (II, FIG. 10B). In this scenario, given that the oxidatively cross-linked GPX4 was reported to be enzymatically inactive, an overwhelming quantity of inactive GPX4 with Cys10 may lock the active selenocysteine-containing GPX4 into inactive states if reductants (GSH or cys66-SH) are not readily available (Ursini et al. 1999). On the contrary, although also enzymatically inactive, GPX4 with cys66 may accelerate the catalytic cycle via pushing the oxidized GPX4 (GPX4-Se—S-G or GPX4-Se—S-cys10) into a regenerated active state (Ingold et al. 2018). A previous mutagenesis study, which showed a role of U46, C10, and C66 in GPX4 polymerization, also supported this model (Scheerer et al. 2007).

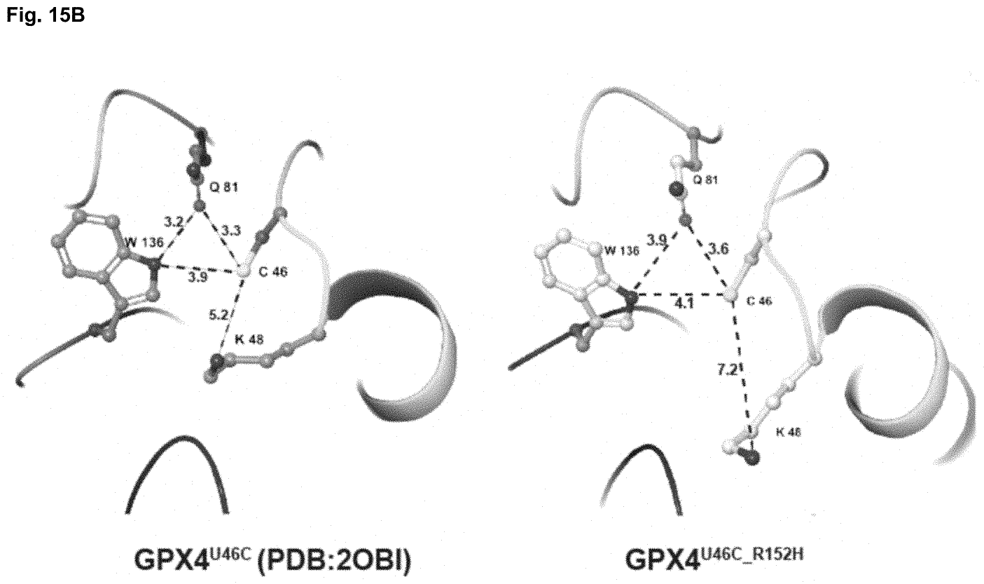

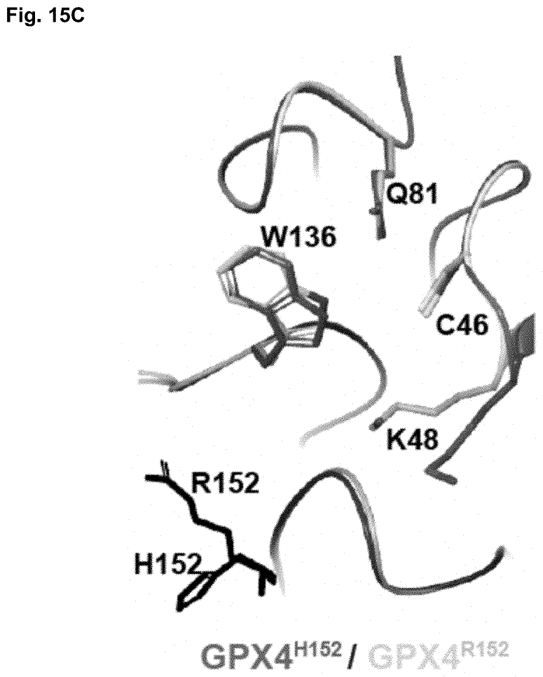

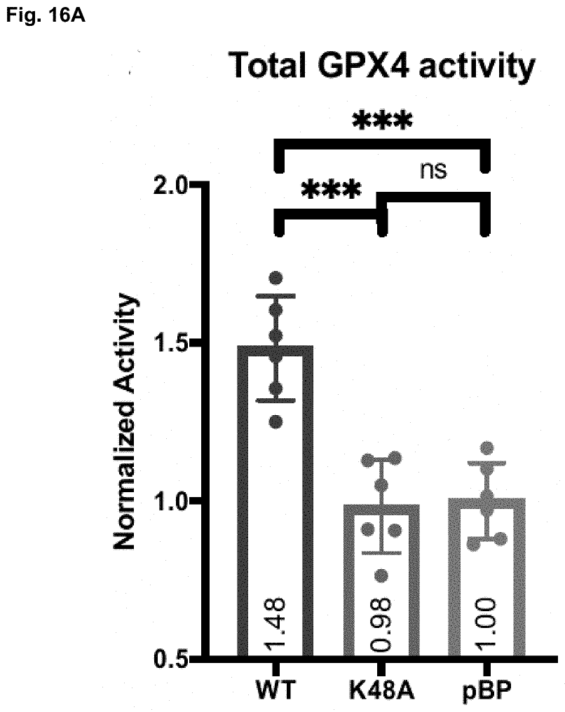

In accordance with the model, we found that overexpression of enzymatically inactive AllCys(−) A66C GPX4 in G401 cells boosted the enzymatic activity of endogenous WT GPX4, while AllCys(−) A10C GPX4 significantly suppressed the activity (FIG. 15C). By comparison, overexpression of AllCys(−) or AllCys(−) A148C GPX4 exhibited no significant effects on the enzymatic activity of endogenous GPX4. From another aspect, in the packing patterns of multiple ligand-free GPX4^{U46C} crystals that we solved in diverse space groups (P3121, P21, and P1), as well as the previously reported GPX4 structures, C10, C46, and C66 are consistently in close proximity to each other, suggesting a structural foundation of the proposed model (Scheerer et al. 2007) (FIG. 15D).

To further verify the potential role of cys66 and cys10 in modulating GPX4 activity, especially at limited GSH levels, we stably overexpressed GFP-tagged GPX4^{C10S_C66S}, which is devoid of the cys66 and cys10 cross-linking sites, in HT-1080 cells and then tested the cells against IKE. Using HT-1080 cells either overexpressing GFP-tagged GPX4^{WT} or transfected with empty vector (pBabe-puro) as controls, we found that overexpression of GPX4^{C10S_C66S}, which lacks the linking sites, still protected HT1080 cells from ferroptosis induced by IKE, but to a significantly lower extent than the overexpression of GPX4WT, suggesting a role of cys10 and cys66 in modulating GPX4 function at low GSH concentrations (FIG. 15E). Additionally, as expected, overexpression of GPX4^{C10S-C66S} protected HT1080 cells from other classes of ferroptosis inducers to an extent greater than (ML162, likely due to loss of cys66 binding site), or indistinguishable (FIN56 and FINO₂) to the overexpression of GPX4^{WT} (FIG. 15F). To further dissect the origin of the different IKE sensitivities observed, we tested the GPX4 activity of the three cell lines in vitro at different GSH concentrations. Although GPX4^{C10S_C66S} and GPX4WT exhibited comparable activity at 3 mM GSH, GPX4^{WT} was more active to reduce phospholipids hydroperoxides than GPX4^{C10S_C66S} at 0.1 mM GSH (FIG. 15G). Together, our observations supported the role of cys66 and cys10 in modulating the activity of GPX4, especially at lower GSH concentrations.

### Cys66 is an Allosteric Binding Site of RSL3 on GPX4

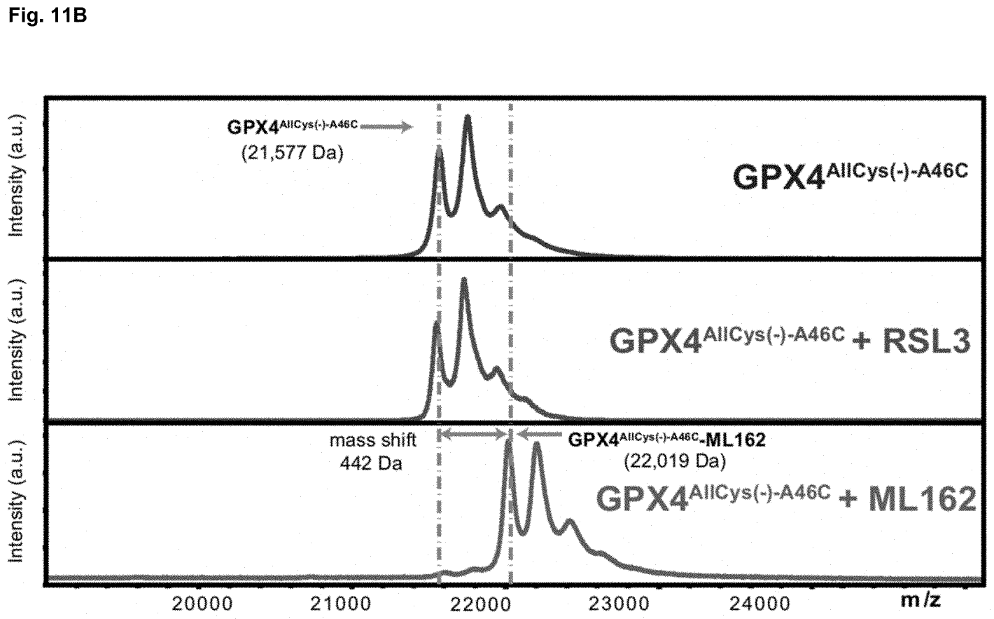

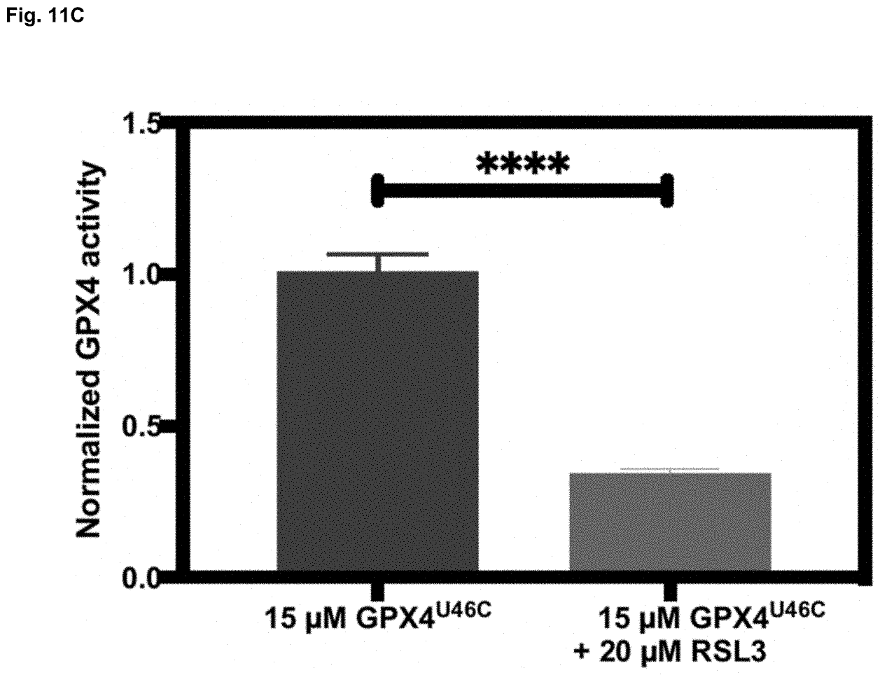

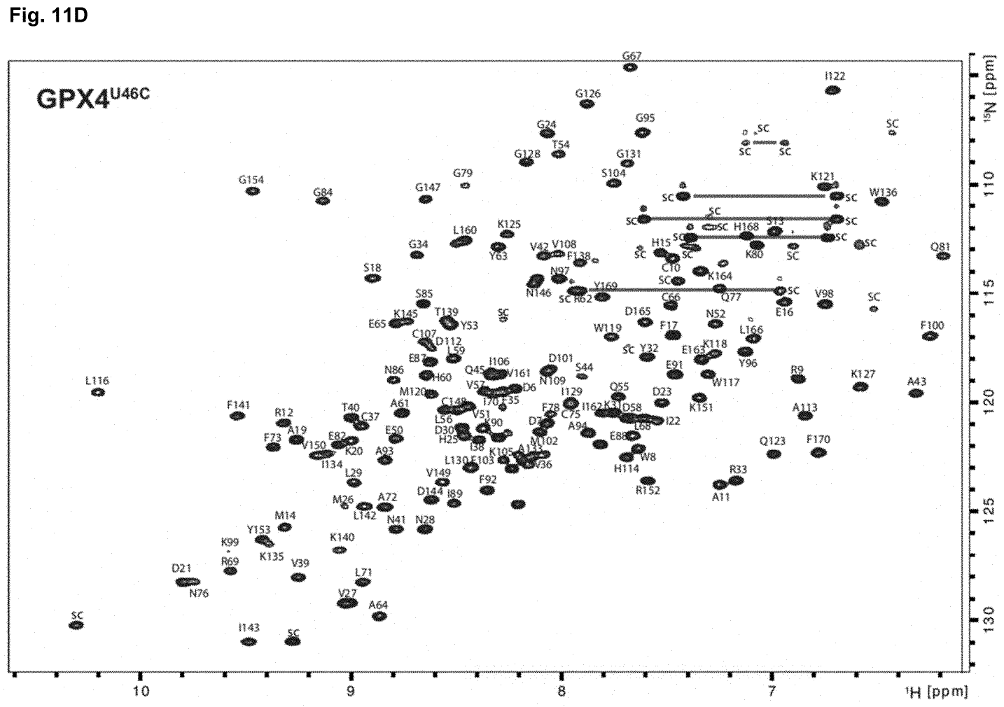

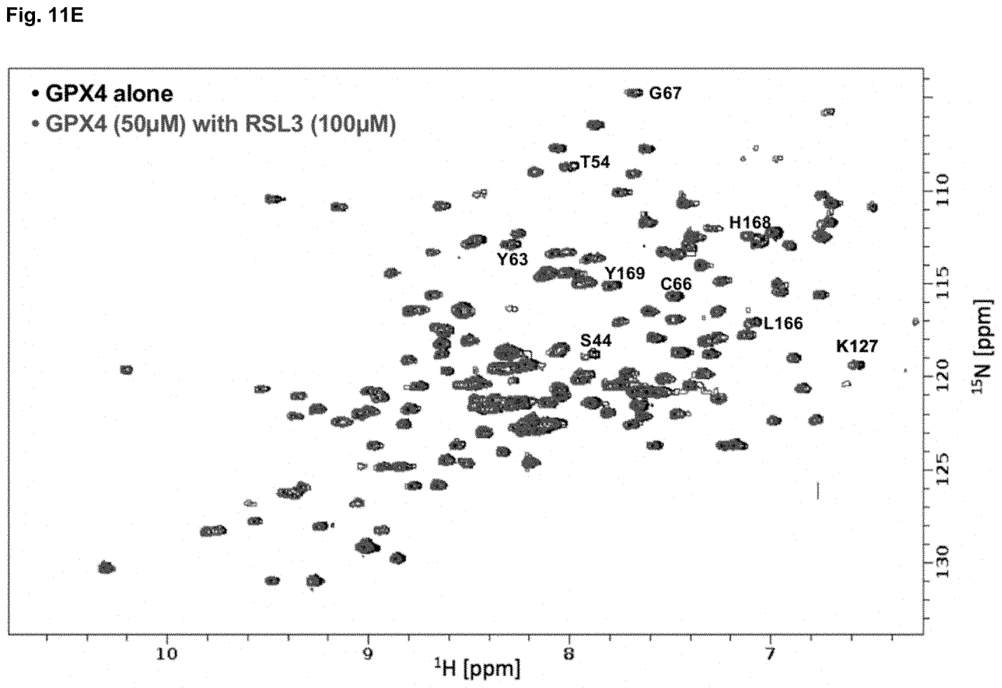

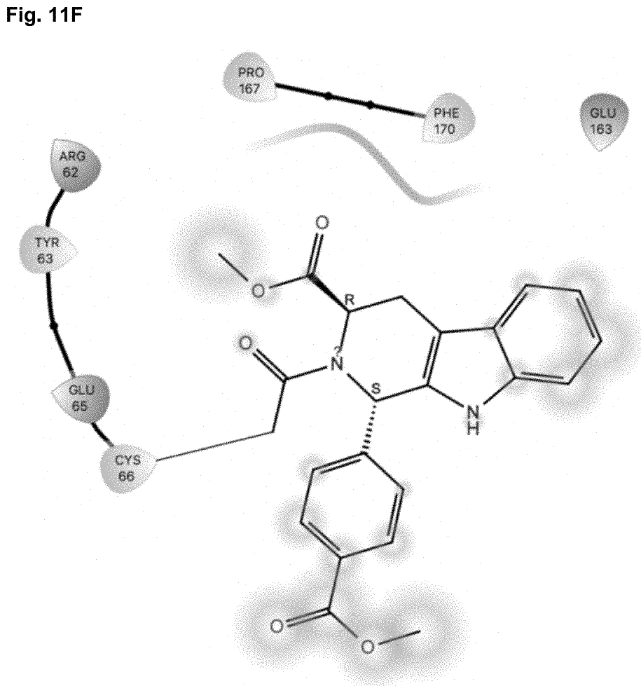

As our results demonstrated that RSL3, the proof-of-concept GPX4 inhibitor with nanomolar potency, not only blocked sec46 at the active site, but also specifically bound to cys66, a positive modulator of GPX4 activity, and subsequentially induced degradation of GPX4 in a cellular context, the potential of taking advantage of cys66 binding site for the design of novel drugs targeting GPX4 was indicated. To specifically examine the potency of cys66 as an inhibitor binding site, in vitro purified GPX4^{U46C} protein was applied, as our cellular data and co-crystal structure indicated RSL3 solely bound to cys66 of GPX4^{U46C}. To further verify this selective binding to cys66, we conducted intact protein MALDI MS analysis of GPX4^{U46C} preincubated with an excess amount of RSL3, which showed 1:1 covalent binding of RSL3 to GPX4^{U46C} (FIG. 11A).

To demonstrate the single covalent modification by RSL3 is not on cys46, we expressed and purified tag-free GPX4^{allCys(-) A46C} protein, in which ala46 in allCys(−) GPX4 was reverted to cys46 (FIG. 9A). While control compound ML162 was shown to be able to modify cys46 and shift the mass of GPX4^{allCys(-) A46C}, which is consistent with the increased viability of G401 AllCys(−) A46C over G401 AllCys(−) when treated with ML162, we observed no mass shift induced by RSL3 in parallel, confirming no RSL3 modification on cys46 (FIGS. 9C and 11B). With our co-crystal structure of GPX4^{U46C}-RSL3 also being considered, we confirmed that RSL3 solely bound to cys66 when selenocysteine 46 is mutated to cysteine in GPX4^{U46C} (FIG. 8A).

To evaluate the impact on GPX4 after RSL3 binds to cys66, we measured the enzymatic activity of GPX4^{U46C} after an incubation with RSL3 or DMSO, which revealed that binding of RSL3 to cys66 inhibited the capability of GPX4 to reduce phospholipid hydroperoxides (FIG. 11C). Aiming to gain more structural insight of the inhibition in solution, we previously reported the ¹H, ¹⁵N-heteronuclear single quantum coherence (HSQC) NMR spectrum of GPX4 in the presence and absence of RSL3, but limited by the lack of HSQC resonance spectrum assignments to each GPX4 amino acid residue at that time, we can only probe binding without specifically knowing its structural binding mode on GPX4 (Gaschler et al. 2018). To address the previous limitation and benefit future study of GPX4 inhibitors, we recently solved the backbone resonance assignments of ¹H, ¹⁵N-HSQC-NMR spectrum for ¹⁵N isotope-labeled GPX4^{U46C}, which enabled a rapid investigation of the specific binding modes of GPX4 inhibitors in solutions (FIG. 11D). With the spectrum assignments, the binding mode of RSL3 at the cys66 site in solution was confirmed with the overlap of ¹H, ¹⁵N-HSQC-NMR spectrum of ¹⁵N-GPX4^{U46C} alone and its complex with RSL3 (FIG. 11E). Residues (Y63, C66, and H168 etc.) interacting with RSL3 in the co-crystal structure of GPX4^{U46C}-RSL3, as shown in the 2D-interaction diagram, consistently exhibited chemical shifts in the HSQC-NMR spectrum (FIG. 11F). Additionally, the significant chemical shifts of S44, T54, and K127, which are distant from the cys66 binding site, suggested a global conformational change induced by binding of RSL3 to cys66. Accordingly, we summarized that cys66 is an allosteric binding site of RSL3 on GPX4. Furthermore, we proposed that the application of ¹H, ¹⁵N-HSQC-NMR in GPX4 inhibitor study, especially along with its spectrum assignments, would enable a rapid investigation into the binding modes and potentially the allosteric effects of future GPX4 inhibitors.

### Screening of Lead-Optimized-Compound Library Identified Lead Compound Binding to the Cys66 Allosteric Site

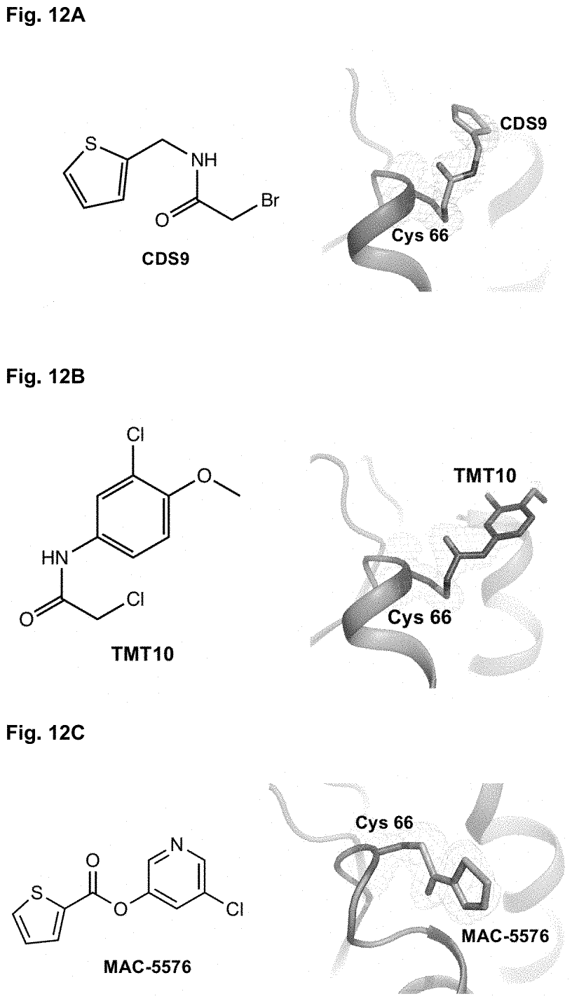

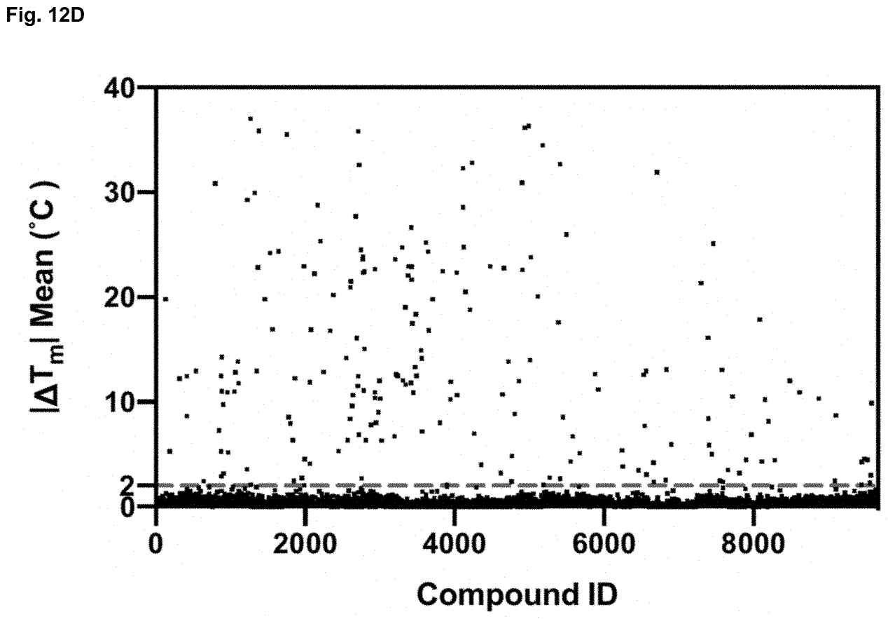

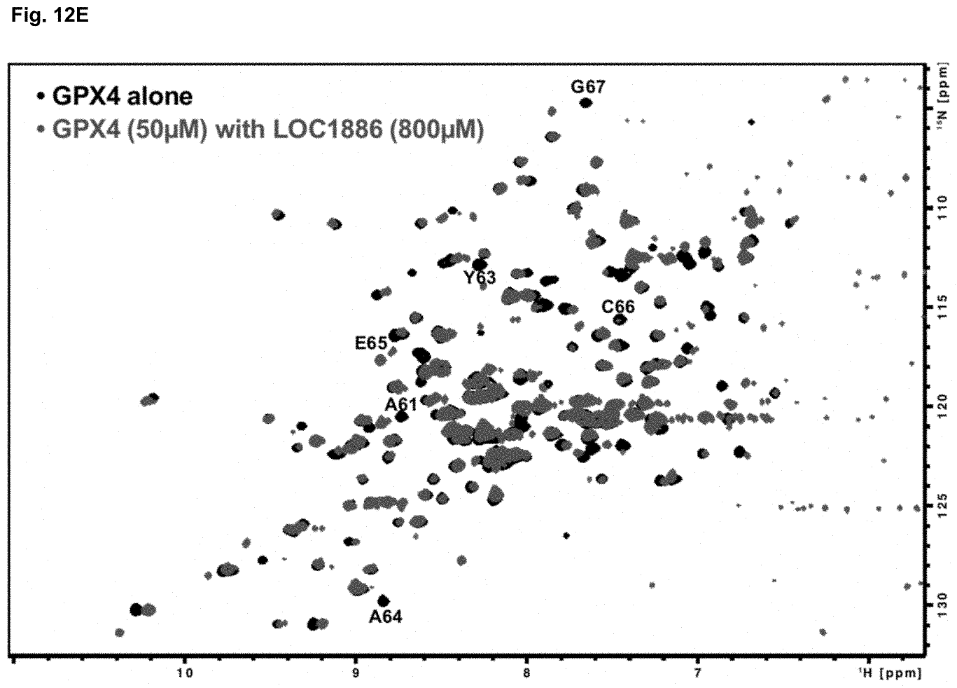

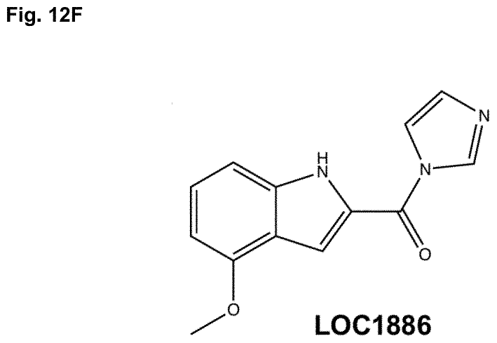

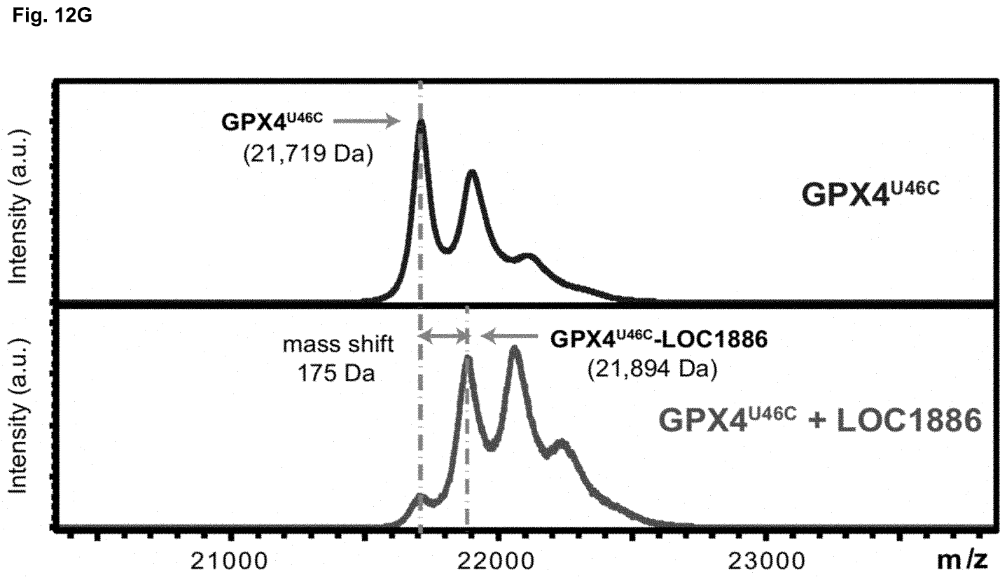

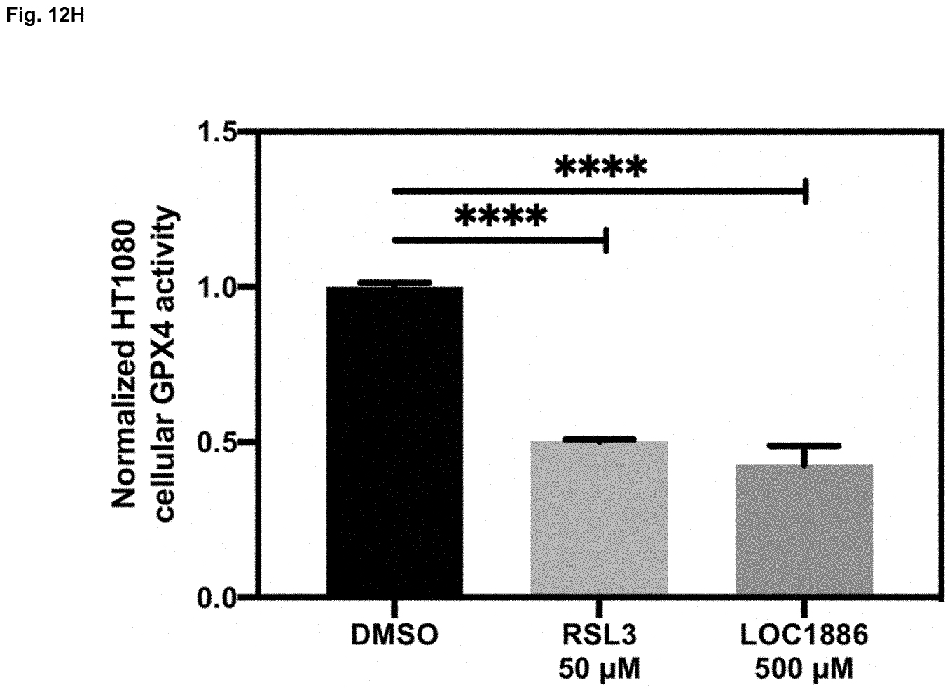

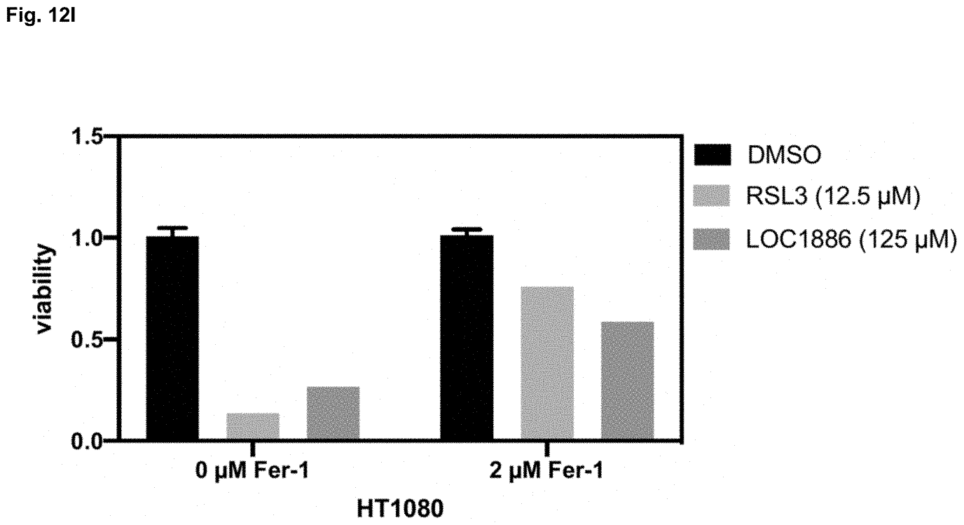

Since we demonstrated that cys66 is an allosteric binding site of RSL3 on GPX4, we then initiated a search for additional compounds that also bind to this site, to validate the druggability of the site and provide foundations for the future design of novel therapeutics targeting GPX4. We started with two fragments of ML162, CDS9 and TMT10, which share similar warheads and structures as RSL3 and ML162. As intact protein MALDI MS analysis of GPX4^{U46C} preincubated with CDS9 and TMT10 suggested covalent bindings, we proceeded to solve the co-crystal structures of GPX4^{U46C} with each of the compounds, which showed both compounds selectively bound to the cys66 site (FIGS. 12A and 12B). During our further search, we found that a protein cysteine modifier, MAC-5576 (Blanchard et al. 2004), was able to covalently modify GPX4. Our co-crystal structure of GPX4 with MAC-5576 revealed that it also bound to the cys66 allosteric site (FIG. 12C).

To fully utilize the versatility shown by the cys66 site to accommodate structurally diverse compounds, we applied thermal shift assay to screen 9,719 compounds in the Lead-Optimized-Compound (LOC) library for in vitro binders of GPX4^{U46C}, which would shift the melting temperature of GPX4^{U46C} (|ΔTₘ|>2° C., FIG. 12D) (Kaplan et al. 2015).

The LOC library was assembled in-house via stringently filtering a database of 3,372,615 commercially available small molecules for structurally diverse compounds with desired drug-like properties and suitability for lead development (Kaplan et al. 2015). Top hits from the screening were then tested by the ¹H, ¹⁵N-HSQC-NMR assay that we developed to examine their individual binding modes. Accordingly, we found that LOC1886, a thermal shift screening top hit compound (|ΔTₘ|=3° C.), strongly interacted with the cys66 binding site, based on the significant chemical shifts of the residues around the site, and resulted in a global conformational change of GPX4^{U46C} (FIGS. 12E and 12F). The subsequent intact protein MALDI MS analysis of GPX4^{U46C} preincubated with LOC1886 revealed that it covalently bound to GPX4^{U46C}, which resembled RSL3 and other proof-of-concept GPX4 inhibitors that we have characterized (FIG. 12G). Moreover, LOC1886 inhibited the capability of cellular GPX4 in HT1080 cell lysate to reduce phospholipid hydroperoxides (FIG. 12H). In addition, LOC1886 was able to induce ferroptosis to HT1080 cells, which can be rescued by fer-1, an ferroptosis-specific inhibitor (Dixon et al. 2012) (FIG. 12I). Although LOC1886 along with other GPX4 binders that we identified exhibited lower potencies than RSL3, they represented novel GPX4 inhibitor scaffolds to be further developed. Moreover, the structurally diverse proof-of-concept compounds that bound to the cys66 site validated the potential of this allosteric site to be further developed for the future design of novel therapeutics targeting GPX4.

Together, these data suggested that we identified a druggable allosteric site on GPX4, and compounds that bind in this site.

### Discussion

Our investigation into the binding modes of RSL3 and ML162 revealed that they not only interact with the active site selenocysteine, but also selectively bind to cysteine 66 of GPX4. With the U46C and AllCys(−)-A66C GPX4 constructs, which excluded binding of inhibitors to active site, we specifically showed that binding of inhibitor to cys66 site resulted in the inactivation of GPX4. In particular, using RSL3 as an example, we showed that its binding to cys66 caused a global conformational change, loss of activity, and also a subsequent degradation in the cellular context. The additional proof-of-concept compounds binding to this allosteric site further validated its druggability. Moreover, since cysteine 66 is not conserved across the GPX isoforms and, unlike selenocysteine, is unique for GPX4, we expected inhibitors designed for cys66 allosteric sites to be selective towards GPX4 (Scheerer et al. 2007).

In line with all known GPX4 inhibitors, the proof-of-concept inhibitors that we identified in this study also form covalent bonds with GPX4. This prevalence might derive from the lack of deep, well-defined pockets on the surface of GPX4, even in the co-crystal structures (Eaton et al. 2018). Although covalent inhibitor used to be intentionally avoided during pharmaceutical development due to selectivity concerns, multiple covalent inhibitors constituted important medicines (such as penicillin and aspirin) (Sutanto et al. 2020; Baillie, 2016). As more covalent inhibitors entered clinical trials recently, the interest in applying the power of covalent inhibitors has been growing, especially for the targets undruggable with reversible inhibitors (Janes et al. 2018; Bar-Peled et al. 2017; Backus et al. 2016). Here, we reported the first co-crystal structure of the top GPX4 inhibitor RSL3, along with structural determination of GPX4 in complex with another four structurally diverse compounds, which would provide structural foundations for the development of improved inhibitor. Additionally, the novel warheads in MAC-5576 (2-pyridylester) and LOC1886 (N-acyl imidazole) represent alternative substituent of the promiscuous chloroacetamide warhead. We envision that larger scales of screening followed by structure-based optimization of the binders would lead to the first drug-like allosteric GPX4-specific inhibitors.

### Methods

### Cell Lines

HT-1080 cells were obtained from ATCC and grown in DMEM with glutamine and sodium pyruvate (Corning 10-013) supplemented with 10% FBS (Gibco), 1% non-essential amino acids (Invitrogen) and 1% penicillin-streptomycin mix (Invitrogen). G401 cells were obtained from ATCC and grown in McCoy's 5A medium (Thermo Fisher, #16600108) supplemented with 10% FBS and 1% penicillin-streptomycin mix.

### Expression and Purification of GPX4^{U46C} Protein

Bacterial expression vectors pET15b-His-tagged-c-GPX4^{U46C} and pET15b-His-tagged-c-GPX4^{AllCys(-)A46C} were prepared in the previous work (Yang et al. 2016). The His-tagged-c-GPX4^{U46C} protein was expressed in E. coli and purified according to a published protocol with minor modifications (Scheerer et al. 2007). Isolated colonies of BL21-Gold (DE3) competent cells (Agilent, #230132) with each plasmid were separately transferred to 8 mL of LB medium with 100 μg/mL ampicillin, and the inoculated culture was incubated while being shaken (225 rpm) at 37° C. for 16 h. 3 mL of the starter culture was added to 1 L of fresh LB medium with 100 μg/mL ampicillin. The culture was incubated while being shaken at 37° C. and 225 rpm until the OD600 reached 0.9. The temperature was then decreased to 15° C. Cells were incubated with 1 mM isopropyl β-D-1-thiogalactopyranoside (IPTG) while being shaken at 15° C. and 225 rpm overnight. The next day, the bacteria were harvested by centrifugation at 4000 g for 20 min at 4° C. and the pellet obtained was ready for purification or stored at −20° C. The pellet was resuspended in 25 mL of chilled lysis buffer (100 mM Tris pH 8.0, 300 mM NaCl, 20 mM imidazole, 3 mM TCEP, and protease inhibitor cocktail (Roche-Sigma, #11836170001)). The bacteria were lysed by sonication on ice for 6 min, and the lysate was centrifuged at 10000 rpm for 20 min at 4° C. to remove cell debris. The clarified lysate was incubated with Ni Sepharose 6 Fast Flow beads (GE Life Sciences, via Cytiva #17-5318-01) on a rotator at 4° C. for at least 1 h. The beads were washed with wash buffer (100 mM Tris pH 8.0, 300 mM NaCl, 50 mM imidazole, and 3 mM TCEP) to remove nonspecific binding. The protein was eluted with 100 mM Tris pH 8.0, 300 mM NaCl, 100 mM imidazole, and 3 mM TCEP. The protein was further purified using a gel filtration Superdex 200 column in FPLC buffer containing 100 mM Tris pH 8.0, 300 mM NaCl, and 3 mM TCEP. The fractions containing GPX4 were pooled together and analyzed by sodium dodecyl sulfate-polyacrylamide gel electrophoresis (SDS-PAGE).

### Intact Protein MALDI MS Analysis

GPX4^{U46C} or GPX4^{AllCys(-)-A46C} protein was pre-incubated with DMSO control or the inhibitor to be tested prior to MALDI MS analysis: 50 μM GPX4 protein was incubated with 500 μM inhibitors in FPLC buffer (100 mM Tris pH 8.0, 300 mM NaCl, and 3 mM TCEP) with 5% DMSO at RT for 1 hour before transferring to 4° C. overnight.

1 μl of the ligand-free protein (pre-incubated with DMSO) or protein-inhibitor complex (pre-incubated with the inhibitor to be tested) was mixed with 9 μl of 10 mg/ml sinapinic acid in the matrix solution (70:30 water/acetonitrile, with 0.1% TFA). 1.0 μl of the final mix was deposited onto the target carrier and allowed to air dry. MALDI spectrum was recorded using Bruker ultrafleXtreme MALDI-TOF instrument. The range of m/z detection and suppression was adjusted to accommodate the molecular weight of target protein. 2000 Hz and 50% intensity was applied for the laser setting. For each sample, five cumulative spectra were collected and the sum was recorded for analysis. All MALDI spectra of protein-inhibitor complex were compared with ligand-free protein to determine the mass shift. Mass shifts were aligned with the mass of potential staying group of each inhibitor to conclude covalent binding.

### Protein Crystallography

GPX4^{U46C} was pre-incubated with the covalent inhibitors before crystallization using the following specifically optimized condition.

RSL3 and ML162 condition: 50 μM GPX4^{U46C} incubated with 150 μM RSL3 or ML162 in the reaction buffer (20 mM Tris pH 9.0, 100 mM NaCl, 3 mM TCEP, 1.5% DMSO) at 15° C. for 1 hour before transferring to 4° C. overnight. CDS9 condition: 25 μM GPX4^{U46C} incubated with 2 mM CDS9 in the reaction buffer (100 mM Tris pH 9.0, 300 mM NaCl, 1 mM TCEP, 2% DMSO) at 37° C. for 2 hours before transferring to 4° C. overnight. TMT10 condition: 25 μM GPX4^{U46C} incubated with 2 mM TMT10 in the reaction buffer (100 mM Tris pH 9.0, 300 mM NaCl, 2% glycerol, 2% DMSO) at 37° C. for 4 hours before transferring to 4° C. overnight. MAC-5576 condition: 50 μM GPX4^{U46C} protein was incubated with 500 μM MAC-5576 in the reaction buffer (100 mM Tris pH 9.0, 300 mM NaCl, and 3 mM TCEP, 5% DMSO) at RT for 1 hour before transferring to 4° C. overnight.

After confirmation of covalent binding using intact protein MALDI MS analysis, the protein-inhibitor complex were exchanged into crystallization buffer (10 mM Tris pH 8.0, 5 mM TCEP) and concentrated to 5 mg/ml.

Molecular surface figures and 2D inhibitor-receptor interaction diagram of co-crystal structures were generated with Maestro (Schrodinger, suite 2020-3), while zoom-in figures with protein backbone and inhibitor electron densities were prepared with Pymol.

### Preparation of Cells Expressing Exogenous GPX4

The pBabe-puro vectors incorporated with the cDNA of GFP-tagged-cyto-GPX4^{WT}, tag-free-cyto-GPX4^{WT}, or GFP-tagged-cyto-GPX4^{U46C} were prepared in previous work (Yang et al. 2014). With the vectors as template, the following mutagenesis primers were designed using the Agilent QuikChange Primer Design application: C10S (5′-GGA GCG CGC ACT GCG CCA GTC G-3′ (SEQ ID NO: 1), 5′-ACG ACT GGC GCA GTG CGC GCT C-3′ (SEQ ID NO: 2)) and C₆₆S (5′-CCC GAT ACG CTG AGA GTG GTT TGC GGA TC-3′ (SEQ ID NO: 3), 5′-GAT CCG CAA ACC ACT CTC AGC GTA TCG GG-3′ (SEQ ID NO: 4)). Primers were purchased from Integrated DNA Technologies. Site-directed mutagenesis kit (QuickChange II, Agilent 200521) was then used to acquire pBP-GFP-cGPX4^{C66S}, pBP-GFP-cGPX4^{U46C_C66S}, and pBP-GFP-cGPX4^{C10S_C66S}. All mutations and the resulted plasmids were confirmed by sequencing at GENEWIZ.

HT-1080 cells were seeded into a 6-well dish at a density of 300,000 cells/well the night before lipofection. 2.5 μg DNA (empty pBabe-puro vector and the above GPX4 expressing pBabe-puro vectors, separately), 7.5 μL Lipofectamine 3000 (Invitrogen, L3000015), and 250 μL Opti-MEM were incubated for 5 min at room temperature before adding to the HT-1080 cells. Following transfection, cells were passaged several times in HT1080 media supplemented with 1.5 mg/mL puromycin and grown in this media for all experiments performed. Expression of the exogenous GFP-tagged-GPX4 was confirmed with fluorescence microscope and Western Blot with both GFP and GPX4 antibodies.

The G401 cells overexpressing WT, allCys(−), allCys(−) A10C, allCys(−) A46C, allCys(−) A46U, allCys(−) A66C, allCys(−) A107C, or allCys(−) A148C GPX4 were reported in the previous work (Yang et al. 2016). The G401 cells were cultured in G401 media supplemented with 1.5 mg/mL puromycin.

### Cellular Viability Assay

1000 cells of each HT-1080 or G401 cell line were plated 36 μL per well of a 384-well plate on day 1. The remaining cells were immediately tested for Western Blot (to monitor GPX4 protein overexpression level). For dose response curves, compounds were dissolved in DMSO and a 12-point, twofold dilution series was prepared. The compounds were then diluted 1:50 in media and 4 μL was added to each well of the plates on day 2. Final concentrations of the compounds on the 384-well plate started from 2 μM for RSL3/ML162 and 20 μM for IKE/FIN56/FINO₂. For single-point ferroptosis test, HT1080 cells were treated either with 12.5 μM RSL3 or 125 μM LOC1886, with or without supplementation of 2 μM Fer-1. After 48 h of treatment, the viability of cells was measured using 1:1 dilution of the CellTiter-Glo luminescent reagent (Promega G7573) with media, which was read on a Victor 5 plate reader after 10 min of shaking at room temperature on day 4. The intensity of luminescence was normalized to that of DMSO control. Results were quantified using GraphPad Prism 9. Based on the dose-response curve of viability, area under curves were calculated and reported in bar graph formats with standard errors using GraphPad Prism 9.

### Western Blot Assay

For the GPX4 degradation study, G-401 cells were seeded at 800,000 per well in a 60-mm plate and allowed to adhere overnight. Cells were then treated with 10 μM Fer-1 and 0 (vehicle), 2 or 4 μM RSL3 for 10 h. Cells were harvested with trypsin (Invitrogen, 25200-114), pelleted, and lysed with RIPA buffer.

For the quantification of GPX4 protein level for cellular viability and GPX4-specific activity assay, each cell line subject to the cellular viability and GPX4-specific activity was tested by Western Blot in technical duplicates. In particular, cells were harvested with trypsin (Invitrogen, 25200-114), pelleted, and lysed by LCW lysis buffer (0.5% TritonX-100, 0.5% sodium deoxycholate salt, 150 mM NaCl, 20 mM Tris-HCl pH 7.5, 10 mM EDTA, 30 mM Na-pyrophosphate, and cOmplete protease inhibitor cocktail). While part of the cell lysates was blotted for protein quantification, the other part of lysates was used for the GPX4-specific activity assay. For both experiments, cell lysates were blotted and imaged as previously described (Yang et al. 2014). Antibodies used were GPX4 (Abcam, ab125066, 1:250 dilution) and actin (Cell Signaling, D18C11, 1:3,000 dilution). Results were quantified using a LI-COR Odyssey CLx IR scanner, ImageJ, and GraphPad Prism 9.

### Determination of Cellular GPX4-Specific Activity

We applied a NADPH-coupled cellular GPX4 enzymatic activity assay as previously reported with minor modifications (Roveri et al. 1994). Oxidized glutathione, generated by GPX4 during reducing its specific phospholipid hydroperoxides substrate, was reduced by Glutathione Reductase at the expense of NADPH, the decrease in the characteristic absorbance of which at 340 nm was monitored and quantified as GPX4 activity. The GPX4-specific substrate PCOOH was prepared by enzymatic hydroperoxidation of phosphatidylcholine by soybean lipoxidase type IV: 22 mL of 0.2 M Tris-HCl, pH 8.8, containing 3 mM sodium deoxycholate and 0.3 mM phosphatidylcholine was incubated at room temperature, under continuous stirring, for 30 min with 0.7 mg of soybean lipoxidase type IV. The mixture was loaded on a Sep-Pak CI8 cartridge (Waters-Millipore) washed with methanol and equilibrated with water. After washing with 10 volumes of water, phosphatidylcholine hydroperoxides were eluted in 2 mL of methanol. 50 millions of particular G401 or HT-1080 cells were harvested and lysed by LCW lysis buffer (0.5% TritonX-100, 0.5% sodium deoxycholate salt, 150 mM NaCl, 20 mM Tris-HCl pH 7.5, 10 mM EDTA, 30 mM Na-pyrophosphate, and complete protease inhibitor cocktail). The concentration of protein in the lysate was determined using BCA assay kit using BSA as standards. Then, on a 96-well plate, 250 μL 1.5 μg/μL cell lysate was incubated in the GPX4 activity assay buffer (0.1% Triton X-100, 100 mM Tris-HCl pH 7.4, 10 mM NaN₃, 5 mM EDTA, 0.6 IU/mL Glutathione reductase, 0.5 mM NADPH, and 3 mM GSH unless otherwise noted) at 37° C. for 10 min. For evaluation of GPX4 inhibitors, DMSO, 50 μM RSL3, or 500 μM LOC1886 was added to the 250 μL cell lysate in GPX4 activity assay buffer before the 10 min incubation. PCOOH was then added to the mixture to initiate GPX4 reaction. Absorbance of NADPH at 340 nm was determined kinetically at 1 min interval over the 20 min time. Experiments using lysis buffer instead of cell lysate and controls without addition of PCOOH were also done to measure the particular activity of GPX4 to reduce phospholipid hydroperoxides. Total GPX4 activity of each sample were normalized to their specific GPX4 level based on Western Blot for unit GPX4 enzymatic activity for comparison. Results were quantified using GraphPad Prism 9.

### Inhibition of the Activity of Purified GPX4^{U46C} Protein

Similar to the determination of cellular GPX4-specific activity, by coupling the oxidation of NADPH to NADP+ by oxidized glutathione produced by GPX4 in the presence of glutathione reductase, GPX4^{U46C} activity was assessed by measuring the decrease in NADPH (absorbance at 340 nm). GPX4 reaction buffer was prepared by adding 0.05 U/mL glutathione reductase, 210 μM GSH, 250 μM NADPH, 50 μM Cumene-OOH into 50 mM Tris HCl, pH 8.0, 0.5 mM EDTA. 15 μM His-tagged-GPX4^{U46C} pre-incubated with DMSO (vehicle) or 20 μM RSL3 was added to the GPX4 reaction buffer and the absorbance at 340 nm was monitored for the following 10 min. Results were quantified using GraphPad Prism 9.

### Thermal Shift Assay

Since the binding of small molecules may alter the thermostability of protein, we applied thermal shift assay to screen the LOC library, which determined binding of ligand from the change of the unfolding transition temperature (ΔTₘ) obtained in the presence of ligands relative to that obtained in the absence of ligands (Kaplan et al. 2015; Lo et al. 2004). For higher throughput purpose, we adapted the assay on 384-well PCR plate. With the assistance of Biomek Automated Liquid Handler (Beckman), 2 μl 10 mM stock solution (in 100% DMSO) of the each compound was transferred from 384-well library plates to 38 μl FPLC buffer (100 mM Tris pH 8.0, 300 mM NaCl, 3 mM TCEP) and mixed in each well of a 384-well mother plate. The compound solution was then dispensed, 12 μl per well, into three 384-well PCR plates. GPX4^{U46C} protein and Sypro orange were manually added to the plate, with the final concentrations of 5 μM and 5×, respectively, to make a volume of 20 μl per well right before thermal shift analysis of the specific plate. The thermal shift assay was performed on the ViiA 7 Real-Time PCR system (Thermo Fisher) with the thermal protocol: 25° for 15 s, Increase temp to 99° at a rate of 0.05°/sec, 99° for 15 sec. The fluorescence was recorded and analyzed by Protein Thermal Shift™ software. The mean |ΔTₘ| value for the biological triplicates were calculated and used for hit identification.

¹H, ¹⁵N-HSQC-NMR Spectrum for ¹⁵N Isotope-Labeled GPX4^{U46C}

Uniformly ¹⁵N-labeled GPX4^{U46C} protein with N-terminal His6 tag was prepared. The GPX4^{U46C} construct was expressed in E. coli BL21-Gold (DE3) cells (Stratagene) growing at 37° C. in M9 minimal medium supplemented with 100 mg/mL ampicillin, 2 mM MgSO₄, 100 mM CaCl₂, 1× trace metals, 1×RPMI 1640 vitamin stock (Sigma-Aldrich, #R7256), 10 mg/mL biotin, 10 mg/mL thiamine hydrochloride, 4 g/L glucose, and 3 g/L ¹⁵NH₄Cl as the sole nitrogen source. The following induction, lysis, and protein purification was the same as for the non-isotope-labeled His-tagged GPX4^{U46C} described above, except the N-terminal His6 tag was removed by adding 5 U/mg thrombin before FPLC purification. The thrombin reaction was after a buffer exchange to remove imidazole, which otherwise would inhibit thrombin, and was allowed to proceed overnight at 4° C.

For HSQC of GPX4 with RSL3, since solubility of RSL3 in aqueous solution is low, 10 μM ¹⁵N-labeled GPX4 was preincubated with 100 μM RSL3 in buffer (100 mM MES, 5 mM TCEP, pH 6.5) for 12 h at 4° C. Then the protein solution was concentrated to 50 μM before testing. For HSQC of GPX4 with other inhibitors, 50 μM ¹⁵N-labeled GPX4 was preincubated with 800 μM inhibitor to be tested for κ h at room temperature in buffer (100 mM MES, 5 mM TCEP, pH 6.5). D20 (10%) was then added for the field frequency lock. The ¹H-¹⁵N HSQC spectra were collected on Bruker Avance III 500 Ascend (500 MHz) spectrometers (Columbia University) at 298K. The ¹H carrier frequency was positioned at the water resonance. The ¹⁵N carrier frequency was positioned at 115 ppm. The spectral width in the ¹H dimension was 7,500 Hz and the width in the ¹⁵N dimension was 1,824.6 Hz. Suppression of water signal was accomplished using the WATERGATE sequence. Heteronuclear decoupling was accomplished using GARP decoupling scheme.

3D Protein NMR Experiments for ¹³C, ¹⁵N Isotope-Labeled GPX4^{U46C}

Uniformly ¹³C, ¹⁵N-labeled GPX4^{U46C} protein with N-terminal His6 tag was prepared. The GPX4^{U46C} construct was expressed in E. coli BL21-Gold (DE3) cells (Stratagene) growing at 37° C. in M9 minimal medium supplemented with 100 mg/mL ampicillin, 2 mM MgSO₄, 100 mM CaCl₂), 1× trace metals, 1×RPMI 1640 vitamin stock (Sigma-Aldrich, #R7256), 10 mg/mL biotin, 10 mg/mL thiamine hydrochloride, 4 g/L U-¹³C₆-glucose as the sole carbon source, and 3 g/L ¹⁵NH₄Cl as the sole nitrogen source. The following induction, lysis, protein purification, and tag removal were the same as for uniformly 15N-labeled GPX4^{U46C} protein.

HNCACB and CBCA(CO)NH NMR spectrum of 50 μM uniformly ¹³C, ¹⁵N-labeled GPX4^{U46C} protein in 100 mM MES pH 6.5, with 5 mM TCEP and 5% D20 were collected on 700 MHz Avance III/TS 3.5.6 at NYSBC and applied for HSQC backbone resonance assignments. We performed the assignment based on standard triple resonance backbone assignment (Cα and Cβ) in the practical guide on protein NMR (https://www.protein-nmr.org.uk) and used CCCPNmr Analysis software (v3). To verify the assignment, we also collected HNCO, HN(CA)CO, HNCA, and HN(CO)CA NMR spectrum, and performed Cα and C′ (carbonyl caron) based assignments in parallel as a cross-check. Of the expected 168 amino acid residues of our GPX4^{U46C} construct (175 total residues excluding 7 prolines which has no H attached to its N and therefore does not give signal in HSQC), backbone resonances of 147 residues were assigned. The unassigned residues included a 10-residue N-terminal linker sequence, which was also not visible in the crystal structure of GPX4 (PDB: 2OBI) (Scheerer et al. 2007). In the sequence of our construct which is corresponding to D6-F170 of human GPX4 (NCBI Reference Sequence: NP_001354761.1), DDWRC ARSMHEFSAK DIDGHMVNLD KYRGFVCIVT NVASQCGKTE VNYTQLVDLH ARYAECGLRI LAFPCNQFGK QEPGSNEEIK EFAAGYNVKF DMFSKICVNG DDAHPLWKWM KIQPKGKGIL GNAIKWNFTK FLIDKNGCW KRYGPMEEPL VIEKDLPHYF (SEQ ID NO: 5), the assigned residues were underlined (154/165). The inability to detect and/or assign the remaining backbone peaks may reflect exchange broadening owing to conformational dynamics and mobility inherent to GPX4 in solution, which notably included cysteine 46 at the active site and other residues on the loop surrounding it (Vadrevu et al. 2003).

### Example 5

### Patient-Derived Variant of GPX4 Reveals the Structural Basis for its Catalytic Activity and Degradation Mechanism

### Introduction

Spondylometaphyseal dysplasia (SMD), first described in 1967 by Kozlowski, was defined as a form of congenital bone dysplasia comprising various types of chondrodystrophy (Nural et al. 2006). Sedaghatian-type spondylometaphyseal dysplasia (SSMD) is a rare neonatal lethal disorder characterized by severe metaphyseal chondrodysplasia with mild limb shortening, platyspondyly, cardiorespiratory defects, and central nervous system abnormalities (Elcioglu and Hall, 1998). Most infants with SSMD succumb within days after birth due to respiratory distress (Ipek and Akin, 2016). So far four SSMD-related GPX4 variants have been described: two biallelic splice site variants (c.587+5G>A and c.588-8_588-4del) in one patient, biallelic homozygous p.Tyr127* in the second patient, and biallelic homozygous variant c.153_160del in the third patient (Smith et al. 2014; Fedida et al. 2020). To date, only recessive truncating mutations in the GPX4 gene have been reported to induce SSMD. The severity of biallelic truncation mutations on GPX4 function are likely due to significant loss of enzymatic functioning, but the exact physiological validation is lacking. Furthermore, whether less involved disease exist and possible genotype-phenotype correlations are unknown. Understanding GPX4 mechanisms may expand the actual overall phenotype by elucidating less involved disease states.

In this study, we identified three individuals from 2 unrelated families who were found to harbor a recurrent homozygous point mutation in the GPX4 gene, c.647 G>A (p. R216H), by whole exome sequencing. One child (patient 1, family 1) has typical skeletal features of SSMD in addition to auditory neuropathy, optic nerve hypoplasia, hypotonia and severe motor delays. A second family (patients 2 and 3, family 2) was identified through a rare disease organization (Curegpx4.org) with similar neurological features but with atypical skeletal findings for SSMD as they lacked platyspondyly and rhizomelia. These findings raise the question of whether missense mutations in GPX4 are pathological or not. Therefore, we sought to further examine the potential impact of this variant.

GPX4 is a selenoprotein and a member of the glutathione peroxidase family of enzymes, which share an antioxidant function of reducing peroxides through the use of the co-substrate glutathione (Brigelius-Flohe and Maiorino, 2013). Despite the structural and functional similarities between these enzymes, GPX4 is distinct from other GPx enzymes, being the only enzyme in mammals capable of reducing esterified phospholipid hydroperoxides and cholesterol hydroperoxides within the context of cell membranes (Brigelius-Flohe and Maiorino, 2013). Although oxygenated lipids can serve as signal molecules regulating inflammatory processes, they disrupt membrane architecture and are prone to decompose into reactive species, which can damage biomolecules, such as proteins and nucleic acids (Imai and Nakagawa, 2003). Therefore, when GPX4 activity is compromised, lipid peroxidation products can accumulate, resulting in cell death through ferroptosis, an iron-dependent form of non-apoptotic cell death (Yang et al. 2014). Accordingly, an essential role for GPX4 during embryogenesis and early development has been demonstrated by the failure of GPX4 homozygous knockout mice to survive past early gestation, and by the neonatal lethality of neuronal-specific GPX4 knockout in mice (Seiler et al. 2008; Yant et al. 2003).

Here, we sought to investigate the impact of a human GPX4 variant missense mutation on the structure and function of GPX4 protein to determine potential pathological features of this variant, and guide SSMD patients towards precise treatments at the earliest opportunity. Additionally, GPX4 has been reported to be a promising therapeutic target for drug-resistant and metastatic cancers, based on the elevated dependency of these cancers on the GPX4 lipid peroxide repair pathway during epithelial-mesenchymal transition (EMT) and in the drug-resistant persister and epithelial to mesenchymal transition cell states (Viswanathan et al. 2017; Hangauer et al. 2017; Liu et al. 2018). We hypothesized that greater insight into the basic biochemical mechanisms of GPX4 might emerge from studying a patient-derived variant, providing us with opportunities to modulate GPX4 for therapeutic benefit in clinical contexts.

Large-scale expression of selenocysteine-containing proteins in recombinant systems is challenging, due to inefficient selenocysteine-incorporating machinery; thus, the selenocysteine to cysteine (U to C, inserting a thiol group in place of the selenol group) mutant of GPX4 is widely used for structural studies, despite its lower enzymatic activity compared to the wild-type protein (Scheerer et al. 2007; Sakamoto et al. 2017; Janowski et al. 2016; Li et al. 2019). Since recent studies on selenocysteine-containing GPX4 confirmed the relevance of the catalytic triad and other structural properties discovered in the context of the U to C mutant, we used a GPX4^{U46C} construct for in vitro structural studies and structure-based computational analysis, and simultaneously examined the selenocysteine-containing cytosolic GPX4 in human cells via enzymatic assays and cellular assays (Borchert et al. 2018; Ingold et al. 2018; Yu et al. 2014). In the mature short form GPX4 construct that we used for both in vitro and the cell-based study, the patient-derived variant is denoted R152H on the GPX4 protein (Arai et al. 1996; Liang et al. 2009).

We found that the missense R152H variant resulted in a change in the protein structure, caused substantial loss of enzymatic function, and thus is likely the basis for the pathological features in patients, considering the essential role of gpx4 in the early development of mice. The functional analysis was validated in patient fibroblasts. Our further structural inspection into the origin of reduced enzymatic activity revealed K48 has an essential role in modulating GPX4 function, in addition to the previously reported catalytic triad (Sec46/Gln81/Trp136) and Asn137 (Tosatto et al. 2008). We also found that the R152H variant alters the degradation of GPX4, revealing for the first time the degradation mechanism of GPX4 protein.

### Results

### Patient-Derived R152H Variant in GPX4 Causes Substantial Loss of Function

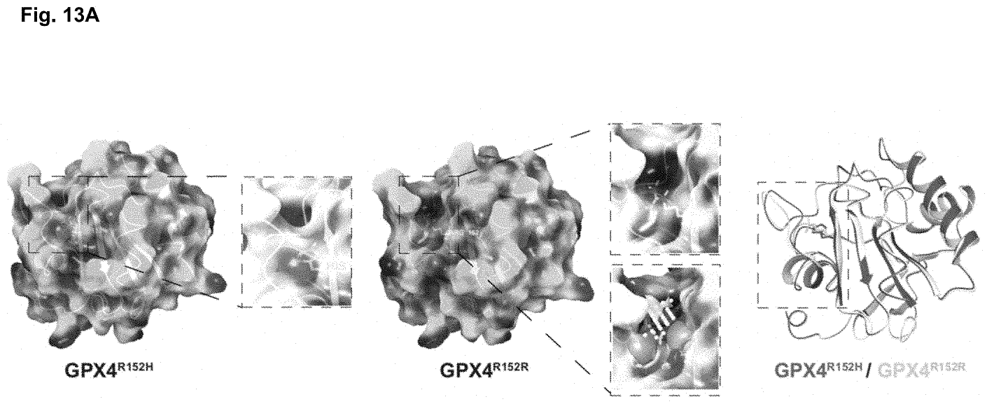

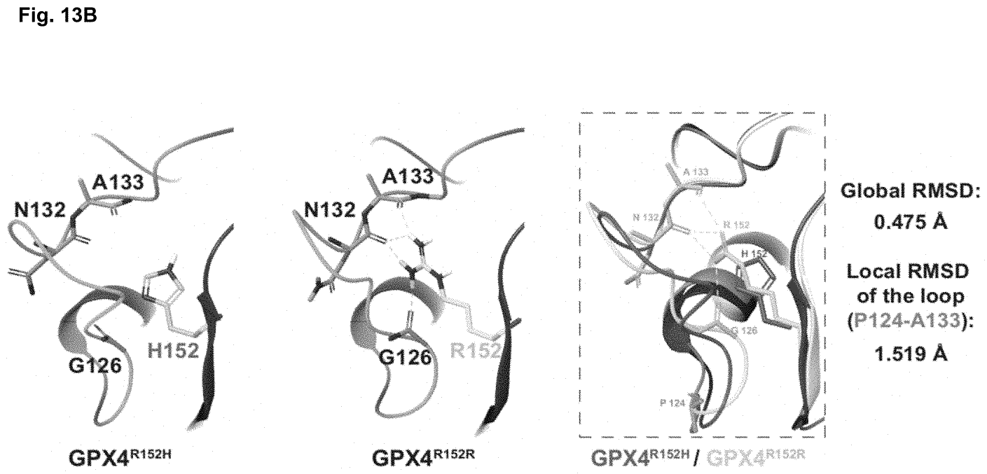

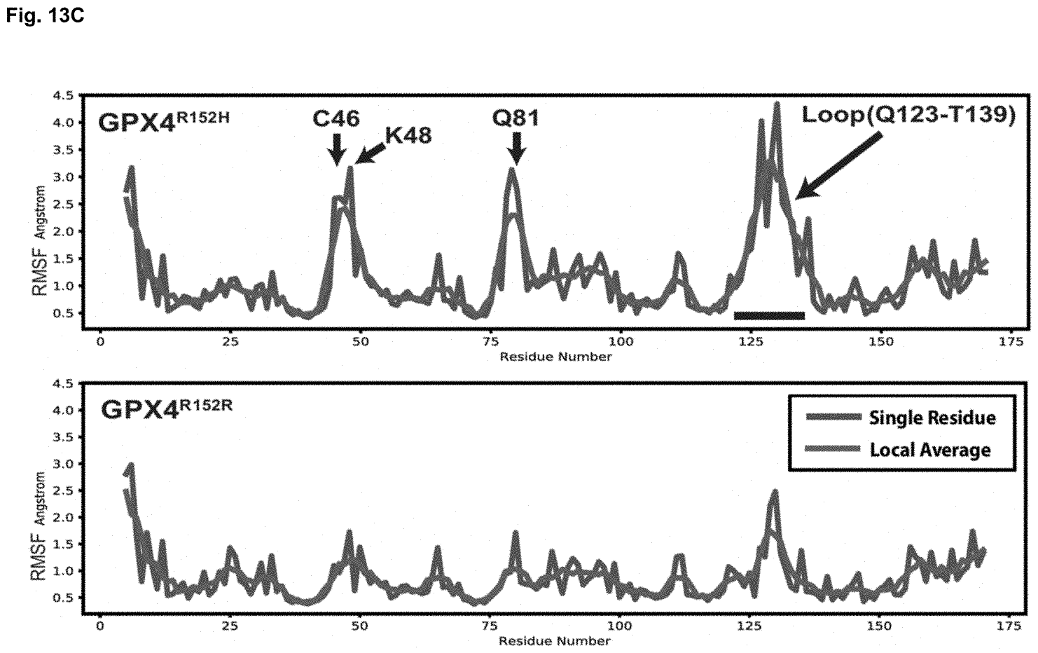

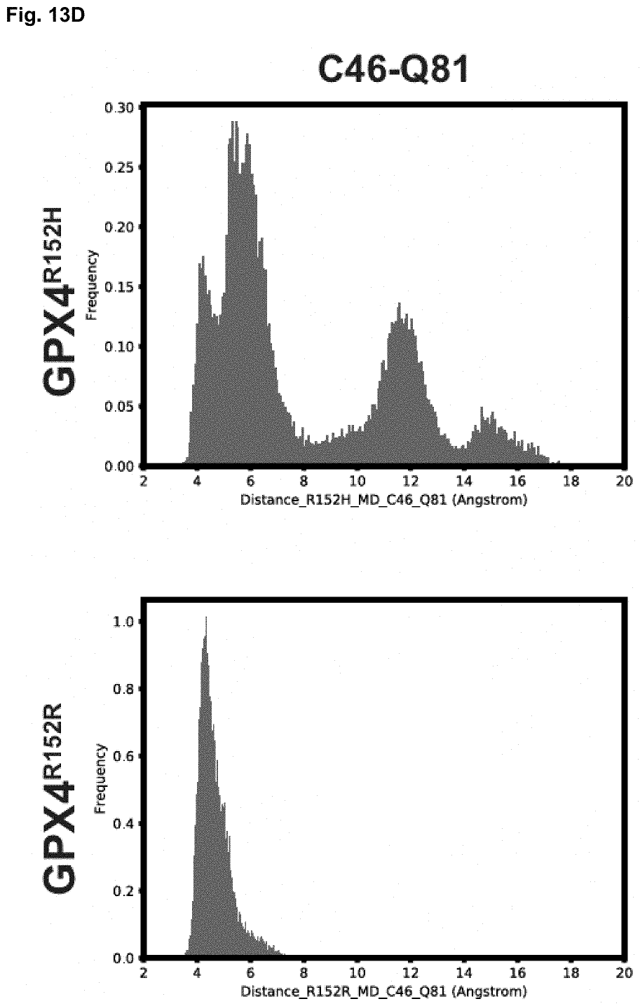

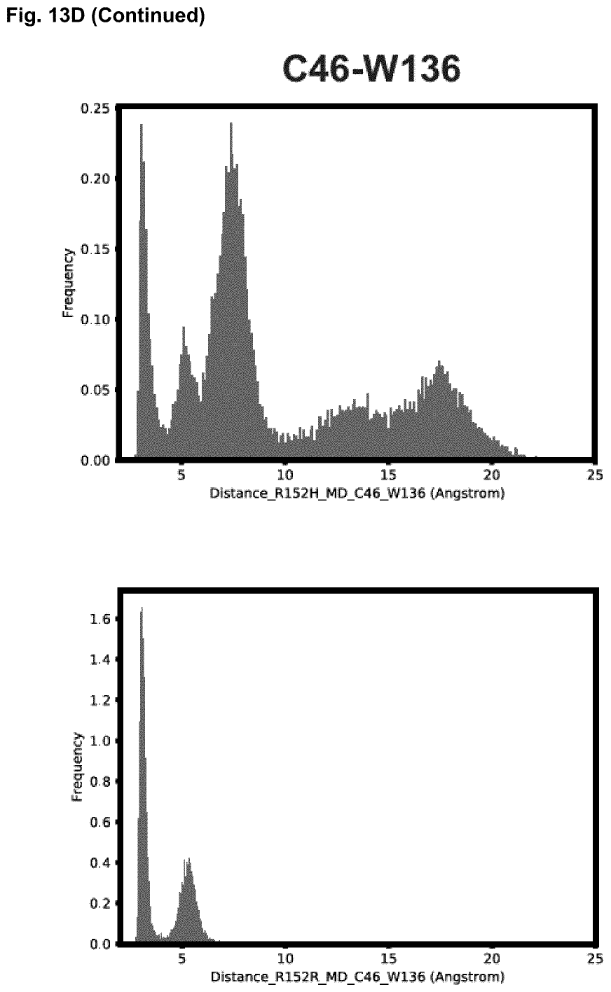

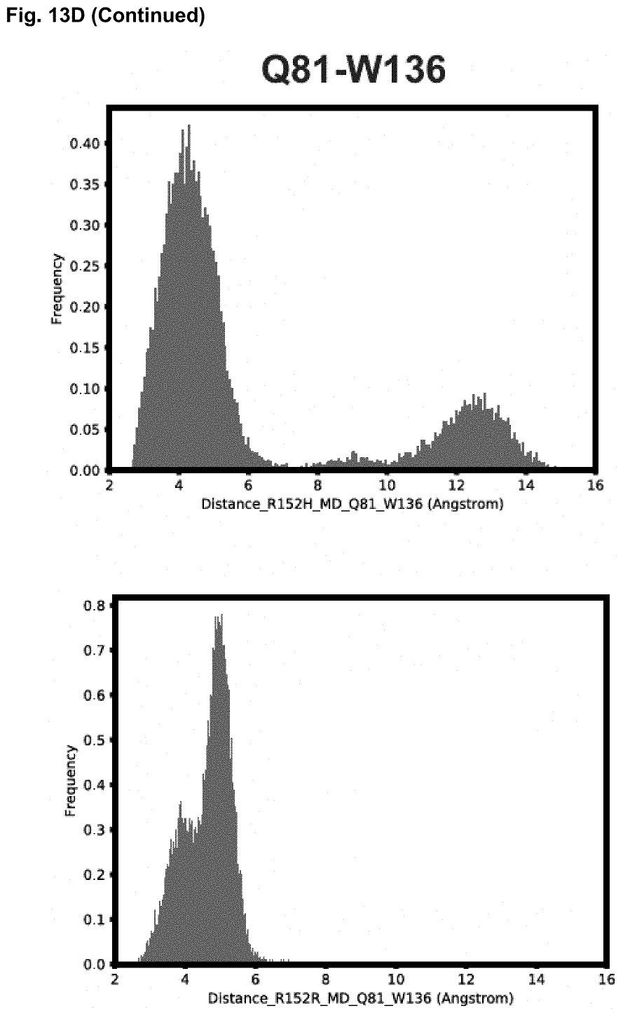

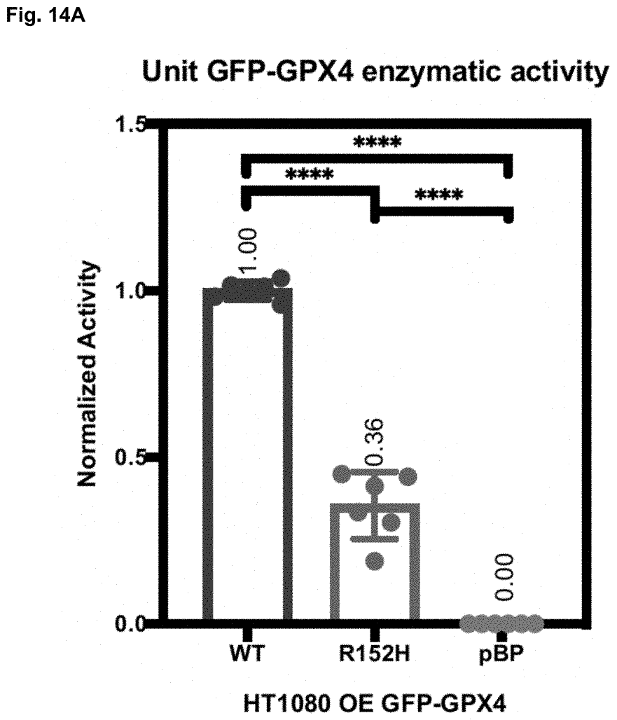

To investigate the impact of the R152H variant on GPX4 structure and function, we began with computational modeling of the GPX4^{R152H} protein structure (FIG. 13). Substitution of the Arg152 residue in the crystal structure of GPX4 (PDB: 2OBI) by His was followed by a global minimization of the whole structure in an implicit solvent to generate a GPX4^{R152H} structural model. As a control, the GPX4^{R152R} was also generated by a synonymous mutation of Arg152 to Arg using the same algorithm and minimization method.

Comparing GPX4^{R152H} with GPX4^{R152R}, we found that the R152H variant significantly altered the surface around residue 152, as evidenced by the loss of a hydrophobic pocket centered on Arg152 in the wild-type protein (FIG. 13A). The change in the surface features mainly derived from an outstanding conformational change of the loop between Gln123 and Thr139, with which the side chains of Arg152 formed multiple hydrogen bonds in the wild-type protein, but not in the His152 variant, which has a more compact side chain and fewer H-bond donors (FIG. 13B). Molecular dynamics (MD) simulations of GPX4^{R152H} and GPX4^{R152R} predicted this loop to be exceptionally mobile in the context of the mutant (FIG. 13C). In addition to the local conformational change, these MD simulations suggested that GPX4^{R152H} exhibits additional flexibility in its active site compared to the wild-type protein (FIG. 13C).

Accordingly, the average distance between the active site catalytic residue Cys46 and its catalytic partners Gln81/Trp136 was significantly increased in GPX4^{R152H} and presented a considerably wide distribution throughout timescale of the dynamics simulated, which indicates a predicted weaker interaction among the catalytic triad (FIG. 13D).

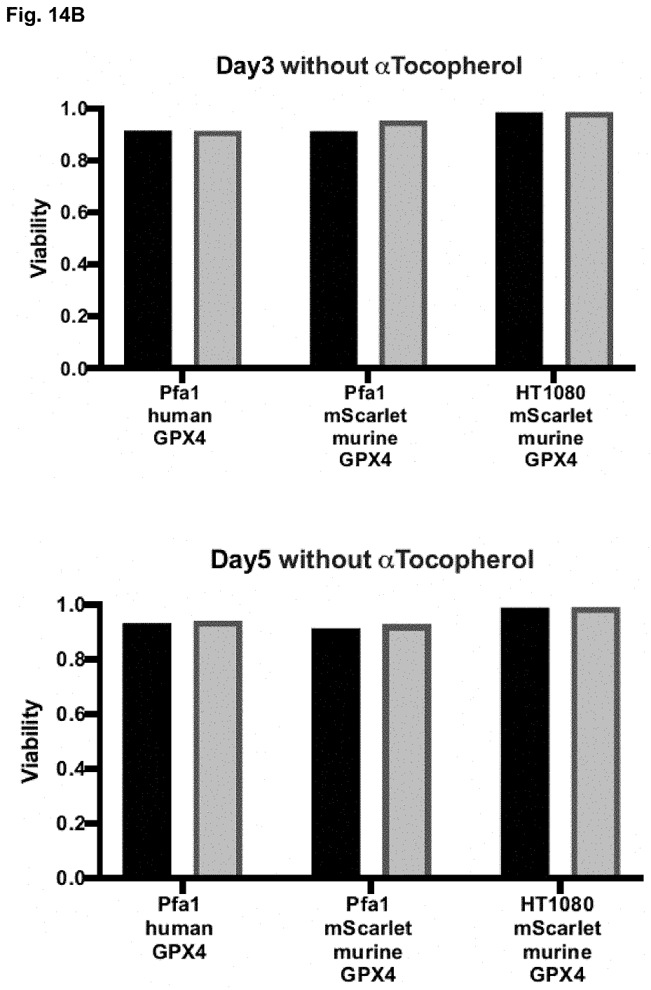

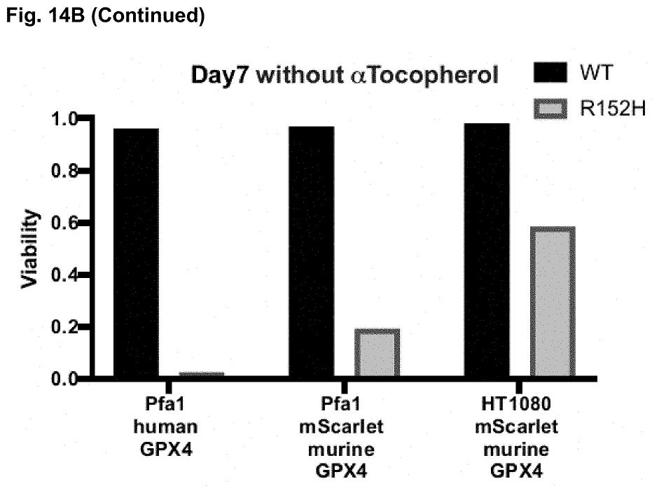

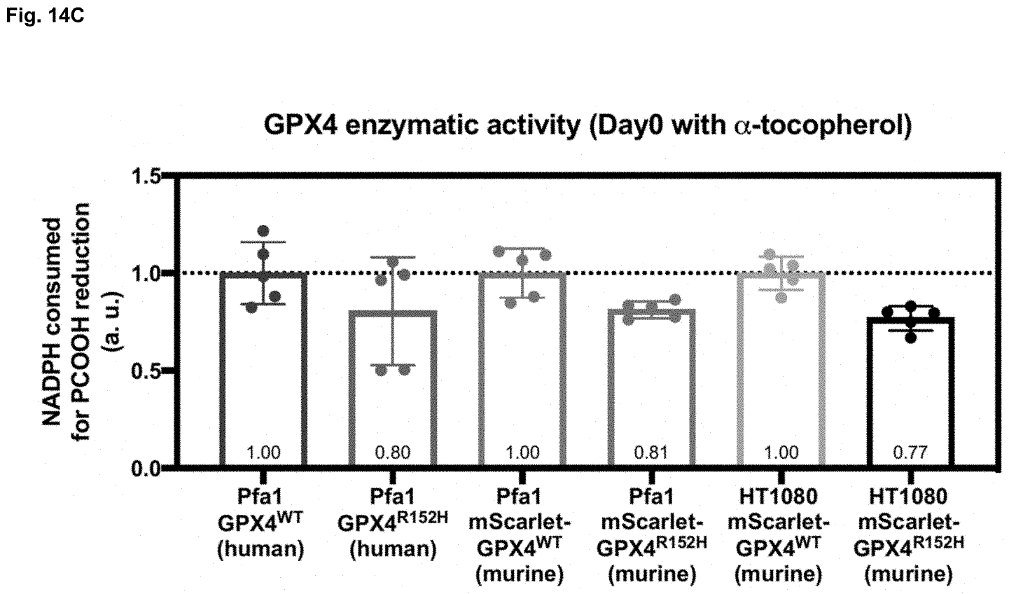

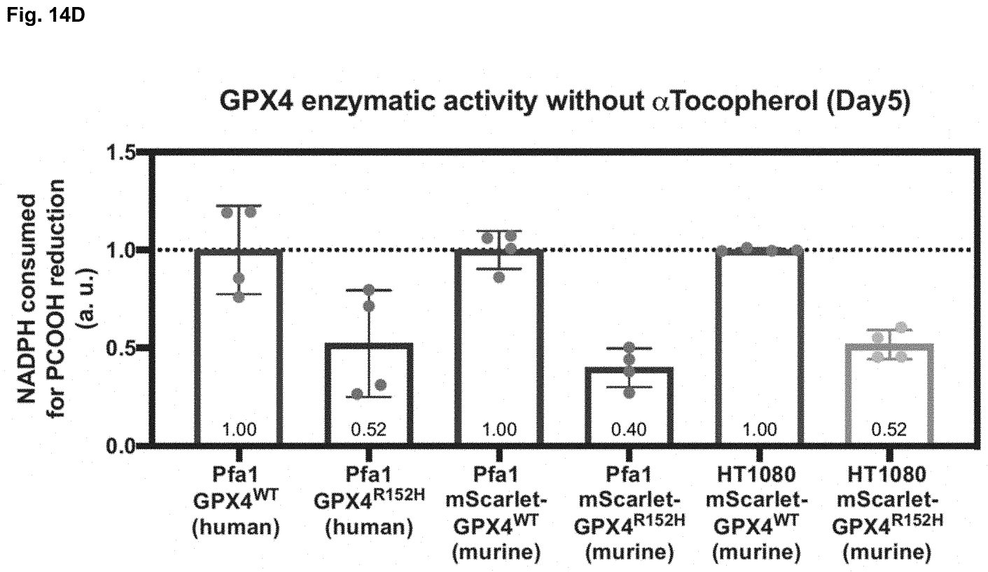

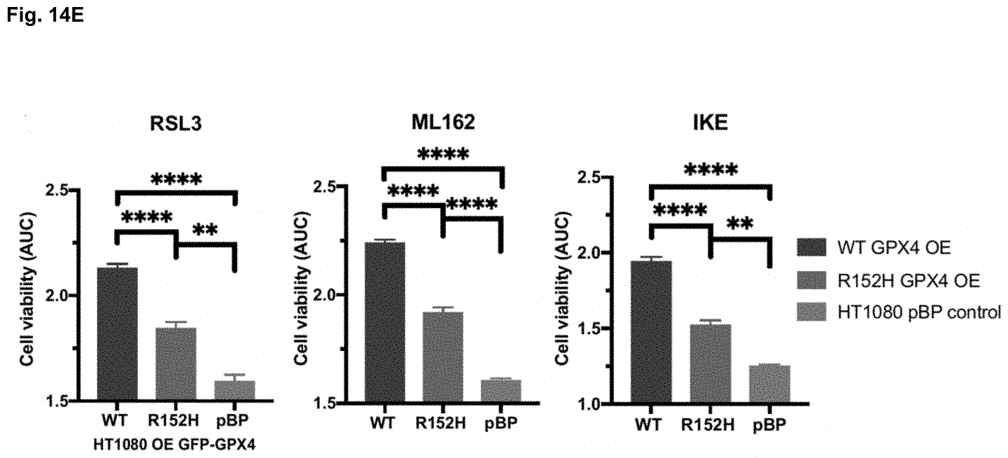

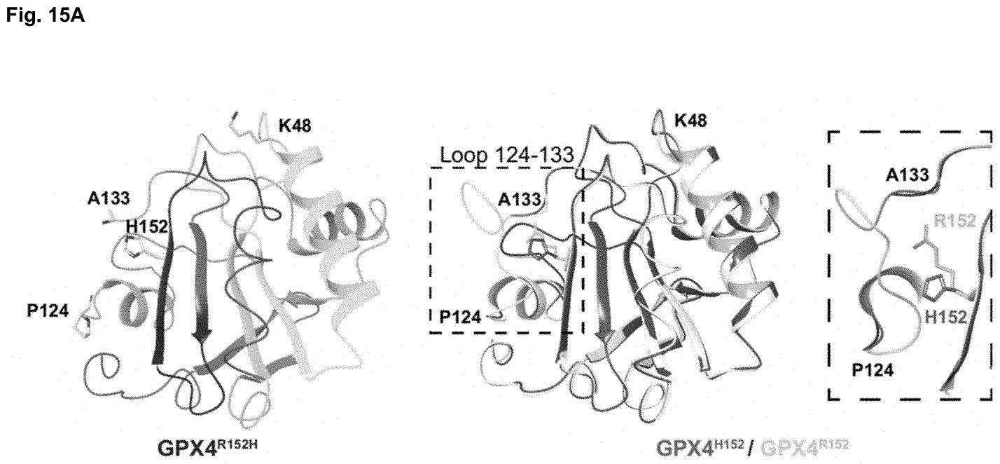

Following the results from computational modeling, we established a cell model of the R152H mutation by stably overexpressing either GFP-tagged GPX4^{WT} or GFP-tagged GPX4^{R152H} in HT-1080 fibrosarcoma cells, in which GPX4 functions to protect cells from ferroptosis, in order to experimentally determine the impact of this variant in a human cell context. Using HT-1080 cells transfected with empty vector (pBabe-puro) as a control, we measured the enzymatic activity of the transfected GFP-GPX4 protein via its ability to reduce a phospholipid hydroperoxide in an NADPH-coupled assay, as reported previously (Roveri et al. 1994). By normalization of the enzyme activity to the protein level, as measured by western blot, we found that one unit of GPX4^{R152H} exhibited 36% of the activity of GPX4^{WT} (FIG. 14A). To further confirm the mutation-induced loss of activity and exclude interferences from the endogenous WT GPX4, we overexpressed exogenous GPX4^{WT} or GPX4^{R152H} in Gpx4-knockout HT1080 and Pfa-1 cells. As we found that Gpx4-knockout cells solely expressing GPX4^{R152H} required α-Tocopherol for normal growth, we measured GPX4 enzymatic activity of the Gpx4-knockout cells cultured in media with or without α-Tocopherol, in which we found a consistently partial loss of activity in each pair of WT/R152H comparisons, especially without interference from α-Tocopherol protection (FIGS. 14B-14D).

We then predicted that the reduction in enzyme activity in R152H would result in impaired resistance to ferroptosis: as the primary enzyme capable of reducing phospholipid hydroperoxides, overexpression of GPX4 protects cells from ferroptosis (Yang et al. 2014).

Accordingly, we tested the ferroptosis sensitivity of HT-1080 cells overexpressing comparable level of GFP-GPX4^{WT} or GFP-GPX4^{R152H}. While both cell lines were more resistant to ferroptosis induced by the GPX4 inhibitors RSL3, ML162 and by the system x_{c}⁻ inhibitor IKE, as compared to the cell line transfected with empty vector, overexpression of GPX4^{R152H} was less protective against ferroptosis inducers than GPX4^{WT}, which was consistent with lower activity of the mutant protein to reduce lipid hydroperoxides (FIG. 14E). Together, these data suggest that the patient-derived R152H variant of GPX4 causes partial loss of its function of phospholipid peroxidase activity and therefore is likely the basis of the pathological features in patients harboring this variant.

### Structural Analysis of GPX4^{R152H} Variant Reveals a Significant Conformational Change

To further understand why the alteration of a single amino acid residue distant from the active site caused a significant loss of enzymatic function in GPX4, we expressed, purified, and solved the x-ray crystal structure of His-tagged GPX4^{U46C} and GPX4^{U46C_R152H} proteins at 1.5 Å resolution. The structure of GPX4^{U46C} that we solved is consistent with the previously reported structure (PDB: 2OBI). While the backbone of GPX4^{U46C_R152H} aligned well with GPX4^{U46C}, the most significant change was in the loop between Pro125 and Ala132, which was intrinsically disordered in GPX4^{R152H}, as evidenced by the loss of electron density corresponding to these residues; this indicates the lack of fixed or ordered three-dimensional structure in this part of the protein (FIG. 15A). As the modeling illustrated, this change is likely due to the loss of multiple hydrogen bonds that Arg152 forms with the backbone carbonyls of Gly126, Asn132 and Ala133, such that the loop between Gln123 and Thr139 became exceptionally flexible, accounting for the missing electron density (FIGS. 13B and 13C).

Moreover, since Trp136 is also on this disordered loop, an examination on the catalytic triad at the active site showed that distances between each pair of the catalytic triad residues were increased, as expected from modeling (FIGS. 13D and 15B). While this observation may partly explain the decrease in catalytic activity, we also noticed in the x-ray structure a significant and unexpected shift of the side chain of Lys48 away from the active site (FIGS. 15A and 15C). The exceptional mobility of Lys48 was also predicted in the MD simulation of GPX4^{R152H} (FIG. 13C). Since the positively-charged Lys48 featured strong interactions with the active site selenium/sulfur anion in the structure of GPX4^{WT} and our MD simulation of GPX4^{U46C}, we reasoned that Lys48 might have an important and previously unknown role in the enzymatic function of GPX4, and its departure from the active site may therefore also impair enzymatic activity (Borchert et al. 2018).

Point Mutation Reveals that Lys48 has an Essential Role in Modulating the Catalytic Activity of GPX4

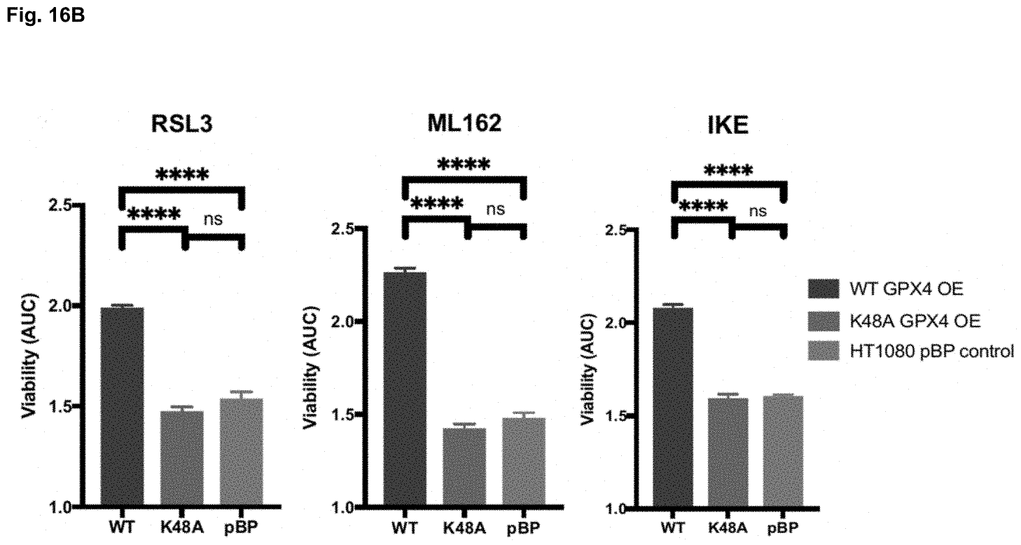

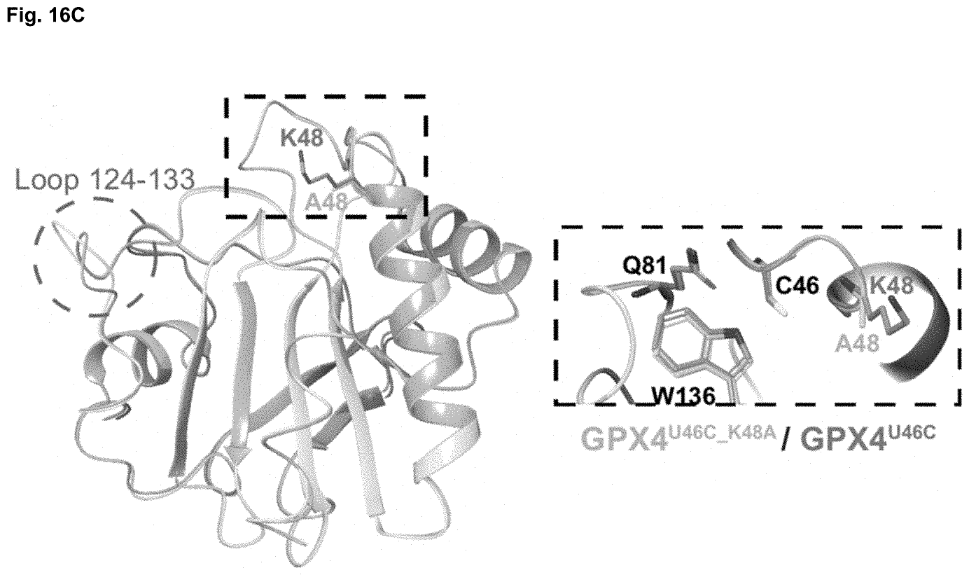

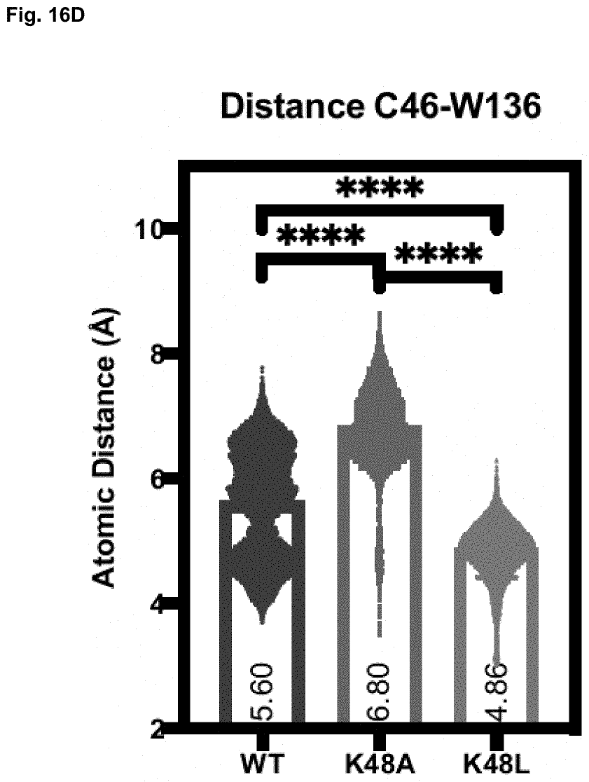

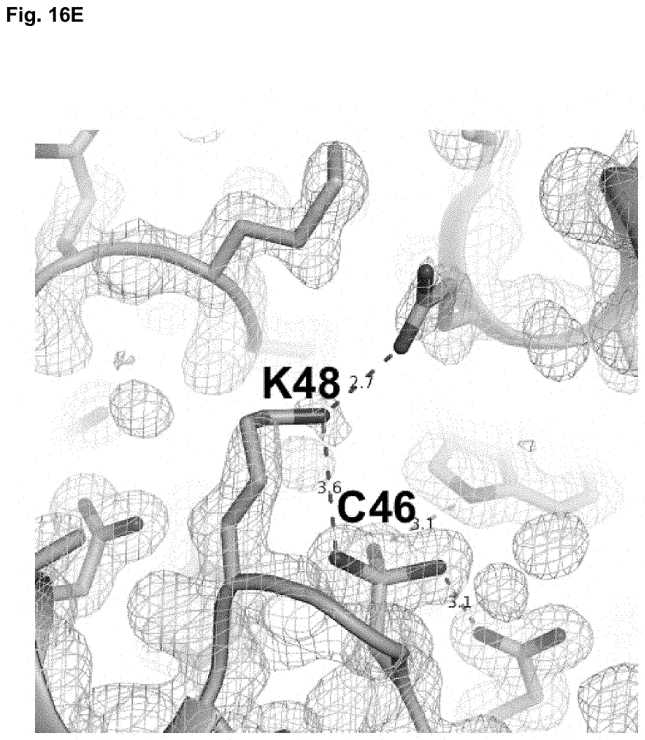

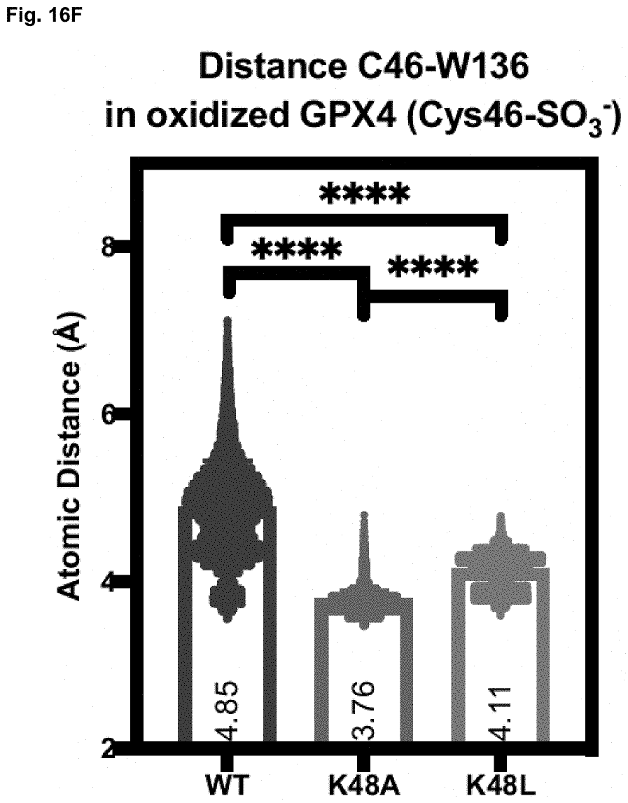

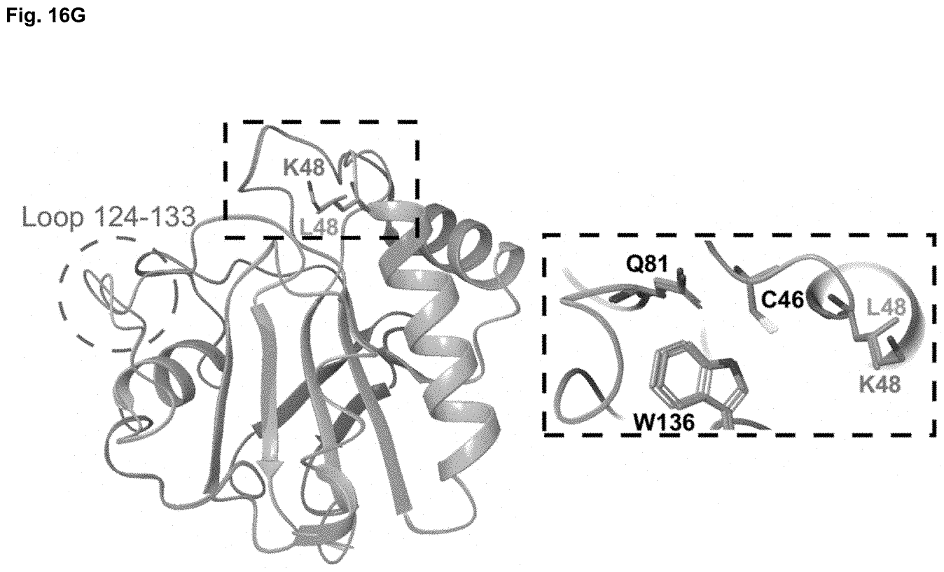

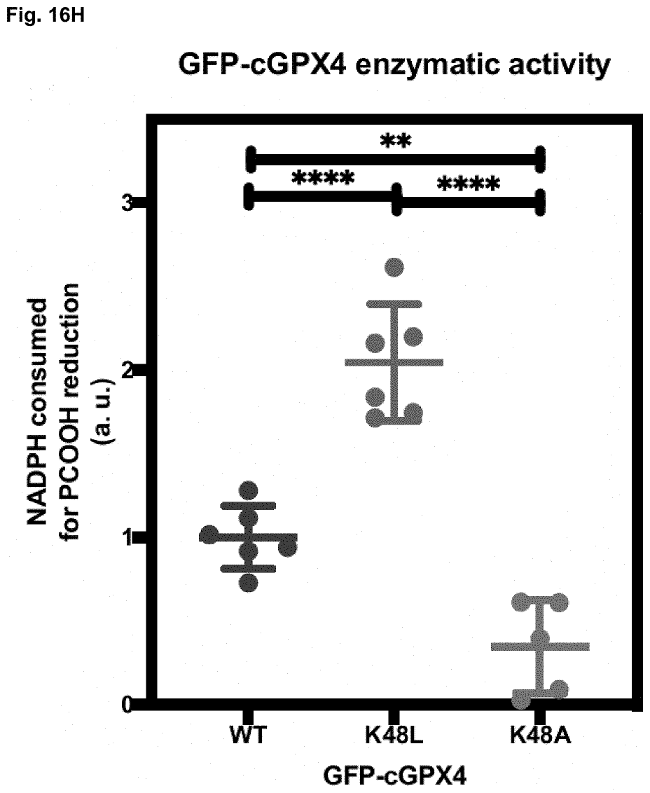

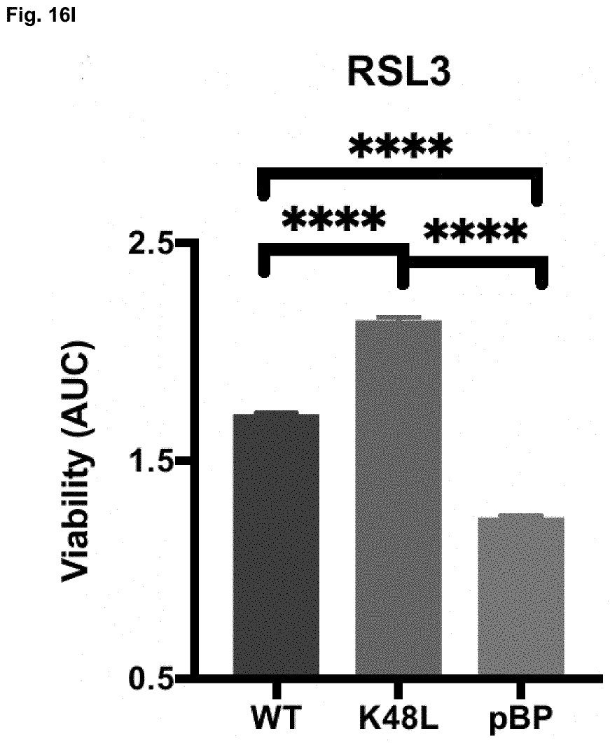

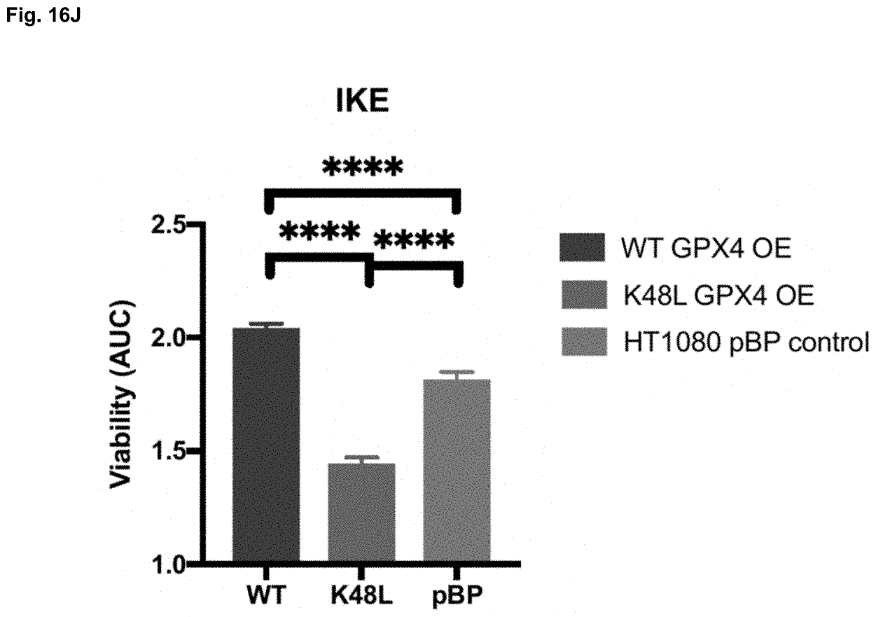

To further investigate the role of Lys48 in the enzymatic function of GPX4, we generated HT-1080 cells stably overexpressing GFP-GPX4^{K48A}. Using HT-1080 cells transfected with GFP-GPX4WT or empty vectors (pBabe-puro) as a control, we measured the enzymatic activity of GFP-GPX4^{K48A}. Strikingly, we found that GPX4^{K48A} had an almost complete loss of activity to reduce phospholipid hydroperoxides (FIG. 16A). Correspondingly, overexpression of GFP-GPX4^{K48A} was not protective at all against ferroptosis induced by RSL3, ML162, or IKE, as compared to a control line (FIG. 16B). These results suggested the unexpected and remarkable hypothesis that Lys48 has an essential role in modulating the enzymatic activity of GPX4.

To illuminate the mechanism for this essential function, we expressed, purified and solved the crystal structure of His-tagged GPX4^{U46C_K48A} protein.

While the GPX4^{U46C_K48A} protein is stable and superimposes well upon the structure of GPX4^{U46C} (FIG. 16C), MD simulations of both structures suggested that GPX4^{U46C_K48A} exhibited additional flexibility in its active site. Accordingly, the average distance between the active site catalytic residue Cys46 and its catalytic partners Trp136 was significantly increased in GPX4^{K48A}, which indicated a weaker interaction (FIG. 16D). This suggested that the interaction between Lys48 and (Seleno-)Cys46 normally stabilizes the active site to be in a more compact and functional state, and that this feature is lost when Lys48 is mutated to Ala.

To further study the role of Lys48 in the context of the canonical catalytic cycle of the GPX4 enzymatic reaction, in which the first step is the oxidation of GPX4 by its hydroperoxides substrates, we solved the crystal structure of fully oxidized GPX4^{U46C}, where the sulfur of the Cys46 was oxidized to sulfonic acid (SO3H). We found that Lys48 is positioned in a close proximity (3.6 Å distance) to the oxidized Cys46, even closer than it was to the reduced Cys46 (5.2 Å distance) in the crystal structure of reduced GPX4^{U46C} (FIG. 16E). This close proximity was also observed in the structure of oxidized selenocysteine-containing GPX4 (Borchert et al. 2018). This suggests a role for Lys48 in the modulation of the active site in the oxidized state of GPX4. To further investigate this interaction, we did MD simulations of fully oxidized GPX4^{U46C} and GPX4^{U46C_K48A} and surprisingly found that oxidized GPX4^{U46C} exhibited additional flexibility in the active site, but oxidized GPX4^{U46C_K48A} featured extreme stability. Accordingly, the average distance between Cys46 and its catalytic partners Gln81/Trp136 was significantly increased in the oxidized GPX4^{U46C} and presented a considerably wide distribution, which suggests the oxidized active site to be in an open state (FIG. 16F). Since the SeO₃⁻/SO₃⁻ state of GPX4 was considered to be inactive and irreversibly over-oxidized, especially for the sulfur variant, the stability of the K48A mutant in this state might contribute to its loss of activity (Ingold et al. 2018).

The second step of the canonical catalytic cycle of GPX4 is the incorporation of its cofactor GSH via the formation of a Se—S bond, which prepares oxidized GPX4 for regeneration to the reduced form. Therefore, we covalently docked GSH into the crystal structures of GPX4^{U46C} and GPX4^{U46C_K48A} in silico using Maestro, and attained better covalent-docking affinities with GPX4^{U46C} (Zhu et al. 2014). This is in line with a previously reported modelling work that proposed a role of Lys48 in interacting with GSH (Mauri et al. 2003). Additionally, the flexible and open active site of the oxidized GPX4^{U46C}, as indicated by its MD simulation in comparison with GPX4^{U46C_K48A}, might also be more accessible for GSH to incorporate and reduce oxidized GPX4.

In summary, these data depict the multi-pronged mechanism of the modulation of GPX4 enzymatic activity by Lys48: to stabilize the active site to be in a more compact and functional state, to modulate the oxidized active site for cycling, and to facilitate the incorporation of the cofactor GSH. Together, this provides an explanation for the loss of activity when this residue was mutated to Ala.

To further dissect the interaction between Lys48 and the active-site residues, we prepared and solved the crystal structure of GPX4^{U46C_K48L} protein, for which the mutation only removed the positively charged E-amino group on the side chain, as compared to the wild-type. While this mutant was stable and superimposes well with the structure of GPX4^{U46C} (FIG. 16G), MD simulations of both structures suggested that GPX4^{U46C_K48L} unexpectedly exhibited enhanced stability in its active site. Accordingly, the average distance between Cys46 and its catalytic partners Trp136 was even lower than in GPX4^{U46C}, which indicated a stronger interaction (FIG. 16D). This is in line with a previous observation that the recombinant K48L mutant of GPX4 featured a higher catalytic activity than WT (Borchert et al. 2018). However, loss of the positively charged ε-amino group in the K48L mutant might have a significant impact on its interaction with the negatively charged oxidized active site and the GSH cofactor. Indeed, we did observe a poor docking affinity of GSH onto the K48L variant, which was indistinguishable from the K48A mutant. The MD simulation of oxidized GPX4^{U46C_K48L} also suggested a moderately stabilized oxidized state, which is comparable to the K48A variant and distinguishable from the wild-type (FIG. 16F). This suggested an elevated susceptibility of K48L to overoxidation, in spite of its higher initial activity. To further verify our analysis of Lys48, we prepared HT-1080 cells stably overexpressing GFP-GPX4^{K48L} Using HT-1080 cells transfected with GFP-GPX4WT or empty vectors (pBabe-puro) as a control, we measured the enzymatic activity of GFP-GPX4^{K48L}. As predicted, we found that GPX4^{K48L} featured an enhanced enzymatic activity to reduce phospholipid hydroperoxides (FIG. 16H). Correspondingly, overexpression of GFP-GPX4^{K48L} was more protective against GPX4 inhibitor RSL3 (FIG. 16I). However, overexpression of GFP-GPX4^{K48L} was not protective at all against IKE, which depleted cellular GSH and may therefore cause overoxidation of GPX4 (FIG. 16J).

The diverse catalytic activities and structural characteristics of the wild-type, K48L (a mutant without the positive charge), and K48A GPX4 (a mutant without both charge and side chain) highlighted that both the positive charge and the long flexible side chain are involved in the function of Lys48. Accordingly, we concluded that the Lys48 has an unexpected and essential role in modulating the enzymatic activity of GPX4 via multiplex interactions with the active site throughout its catalytic cycle. Additionally, we found that in both crystal structures of K48A and K48L, there is a conformational change in the 124-133 loop, as compared to wild-type (FIGS. 16C and 16G). This suggested an allosteric link between the loop and K48 at the active site, which is also the case in the patient-derived R152H variant (FIG. 15A). Therefore, Lys48, as well as the 124-133 loop, would serve as a profound and versatile target for therapeutic strategies aiming at GPX4.

### The Resistance of R152H Mutant to Degradation Implicates the Ubiquitin-Proteasome System

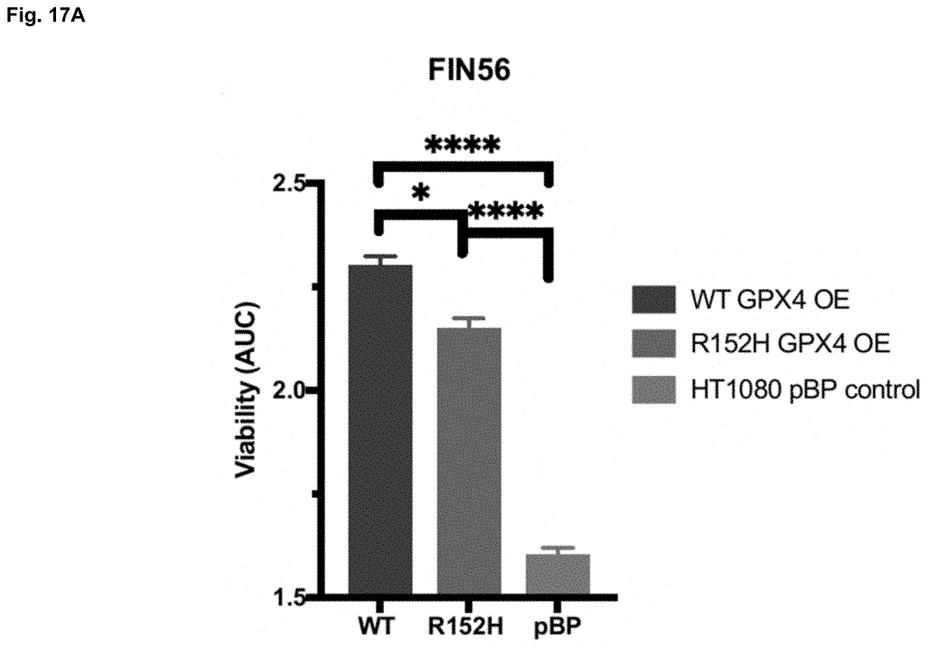

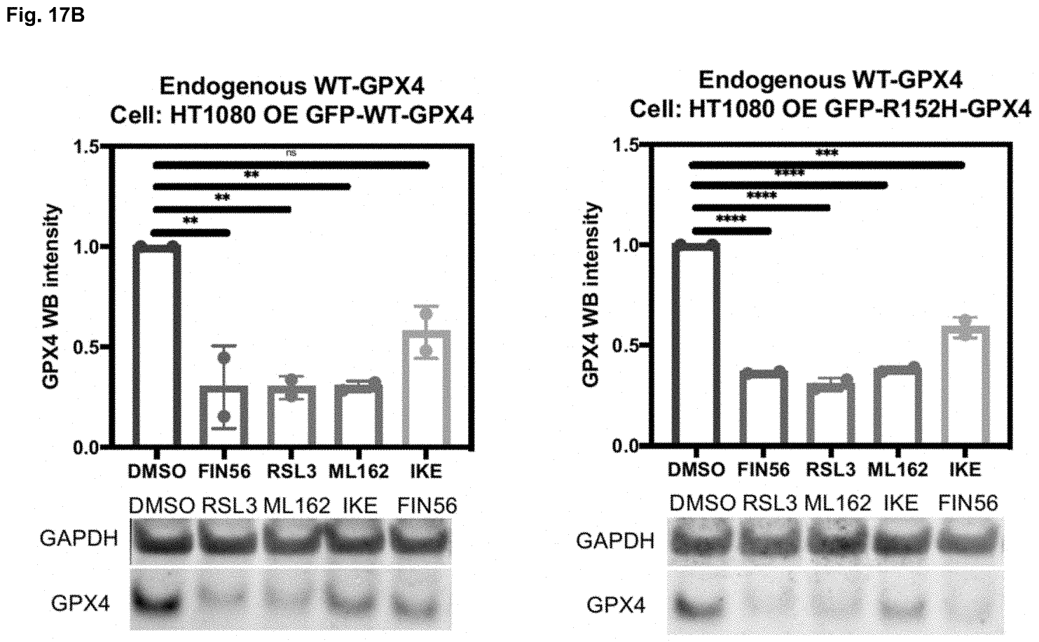

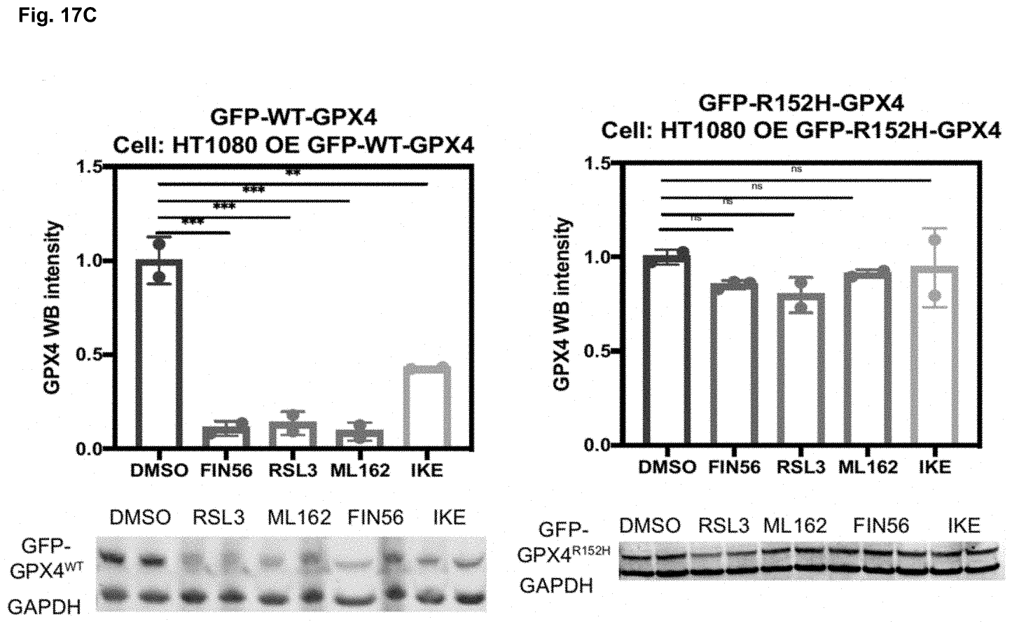

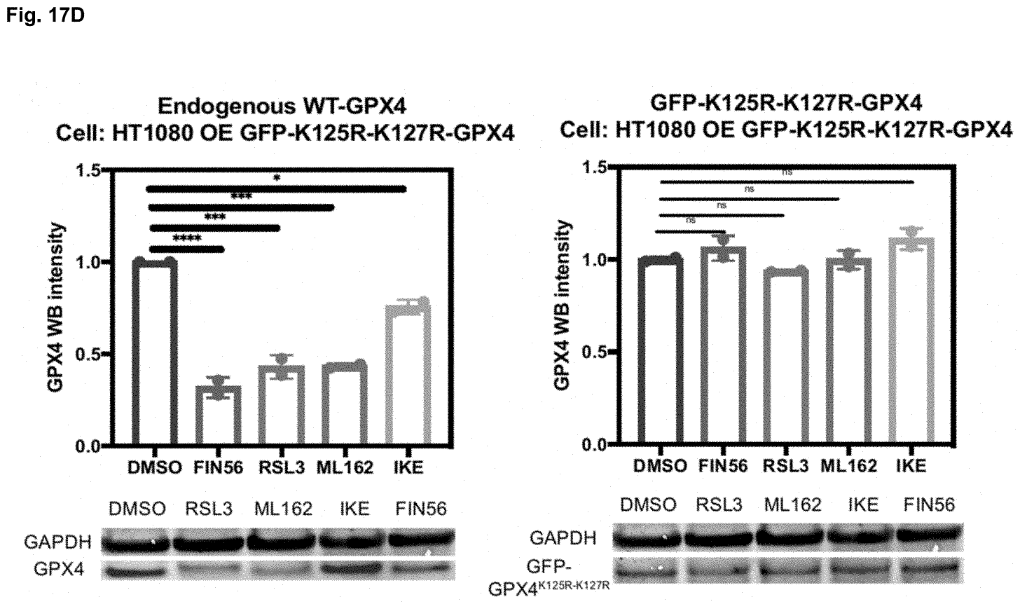

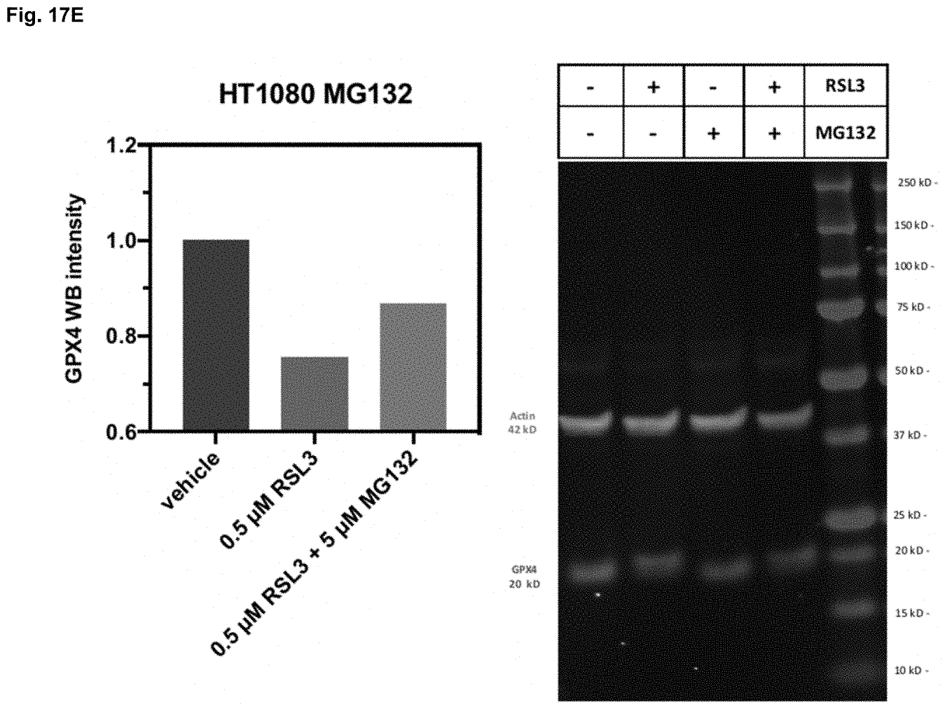

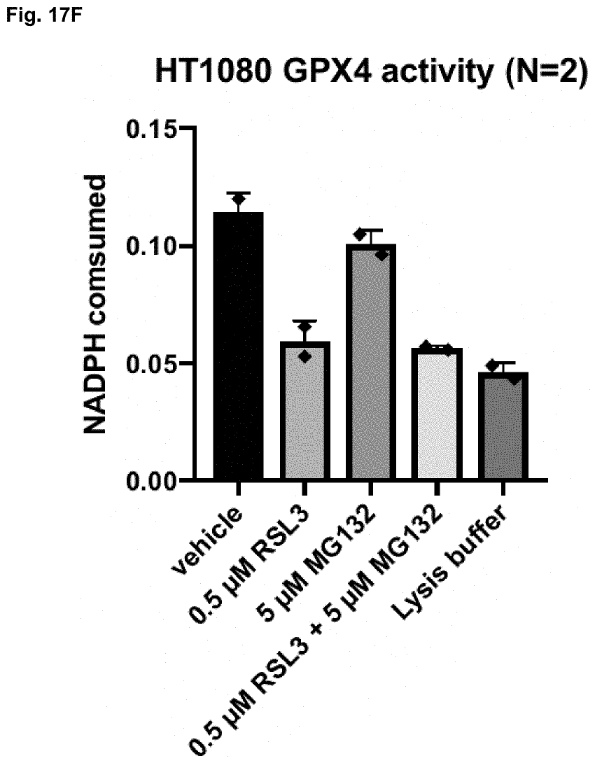

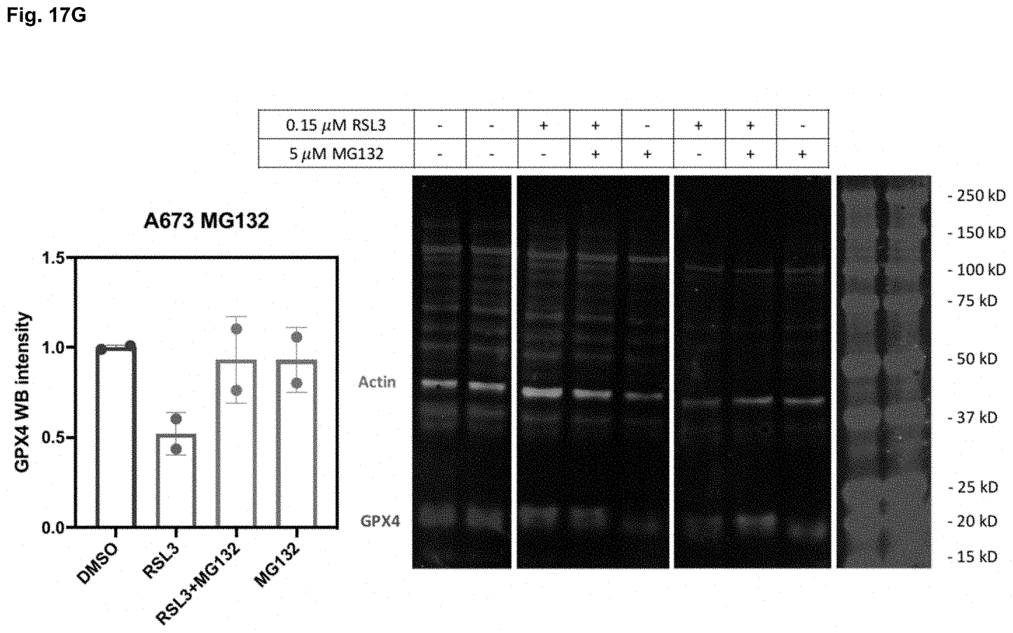

When we tested the ferroptosis sensitivities of HT-1080 cells overexpressing GFP-GPX4^{R152H} or GFP-GPX4^{WT}, we were surprised to find that GPX4^{R152H} provided comparable protection as GPX4^{WT} to the ferroptosis inducer FIN56, despite the insufficient protection against RSL3, ML162 and IKE (FIG. 17A). Since FIN56 was reported to induce ferroptosis via promoting the degradation of GPX4, as well as depleting coenzyme Q₁₀, we speculated that R152H may change the susceptibility of GPX4 to degradation (Shimada et al. 2016). Therefore, we treated HT-1080 cells overexpressing GFP-GPX4^{R152H} or GFP-GPX4^{WT} with various ferroptosis inducers and measured the residual GPX4 protein level after treatment by western blot, using methods reported previously (Gaschler et al. 2018). The endogenous GPX4 protein served as internal control and confirmed that RSL3, ML162 and FIN56 significantly promoted degradation of GPX4 under these treatment conditions (FIG. 17B). Meanwhile, significant degradation of transfected GFP-GPX4^{WT} was induced by RSL3, ML162, and FIN56, as expected, which excluded any interference of the GFP tag on the degradation mechanism (FIG. 17C). We observed that GFP-GPX4^{R152H} was more resistant compared to GFP-GPX4^{WT} to degradation induced by RSL3, ML162, and FIN56; this result confirmed a change in the degradation susceptibility of GPX4 resulted from the R152H mutation (FIG. 17C).

The mechanism of GPX4 degradation induced by RSL3 and FIN56 is not clear, despite some efforts to understand this phenomenon (Shimada et al. 2016; Wu et al. 2019).

Based on our finding that the loop between Pro125 and Ala132, which contains Lys125 and Lys127, became intrinsically disordered in the context of GPX4^{R152H}, we developed a hypothesis that RSL3-induced or FIN56-induced GPX4 degradation involved a proteasome-dependent mechanism, with Lys125 and Lys127 as potential ubiquitination sites on GPX4; in this model, the highly mobile lysines hinder ubiquitination and prevent degradation. We tested this hypothesis by examining GPX4 degradation in HT-1080 cells stably overexpressing GFP-GPX4K125R_K127R, where the proposed sites of ubiquitin ligation were removed by point mutations. While the endogenous GPX4 was degraded upon treatment with RSL3, ML162, or FIN56, we found that GFP-GPX4K125R_K127R was resistant to degradation, even more so than in the context of the R152H mutation (FIG. 17D). Furthermore, we found that MG132, a proteasome inhibitor, suppressed RSL3-induced wild-type GPX4 degradation in HT1080, while having no effect on the decreased GPX4 activity caused by RSL3 (FIGS. 17E and 17F). This implied an induction of degradation following the activity-silencing binding of covalent inhibitors to GPX4. Suppression of RSL3-induced GPX4 degradation by MG132 was also reproduced in A673 cells, which have a different expression level of GPX4 (FIG. 17G). Together, these data suggest that the degradation of GPX4 induced by RSL3 and FIN56 involves a proteasome-dependent mechanism, and that Lys125 and Lys127 of GPX4 are sites of ubiquitin conjugation.

### Selenium Supplementation was Tested as a Proof-of-Concept Treatment

The results described above demonstrated that the patient-derived R152H variant of GPX4 caused partial loss of its function in phospholipid peroxidase activity and therefore is pathological. However, the data also suggested that the overexpression of the partially-active but degradation-resistant GPX4^{R152H} did partially protect cells from ferroptosis induced by lipid peroxidation, though less effectively than wild-type GPX4. Therefore, we proposed a hypothesis that boosting the expression level of the R152H variant of selenoprotein GPX4 via selenium supplementation should be able to compensate the partial loss of function and be helpful to patients. This is also based on the observation that a subtoxic selenium supplementation can significantly increase the expression level of GPX4 (up to 48-fold), as well as another anti-oxidant selenoprotein GPX1 (up to 40-fold), in human cells (Romanowska et al. 2007). Additionally, selenium is readily available from dietary source or as OTC supplement tablets (Bodnar et al. 2016).

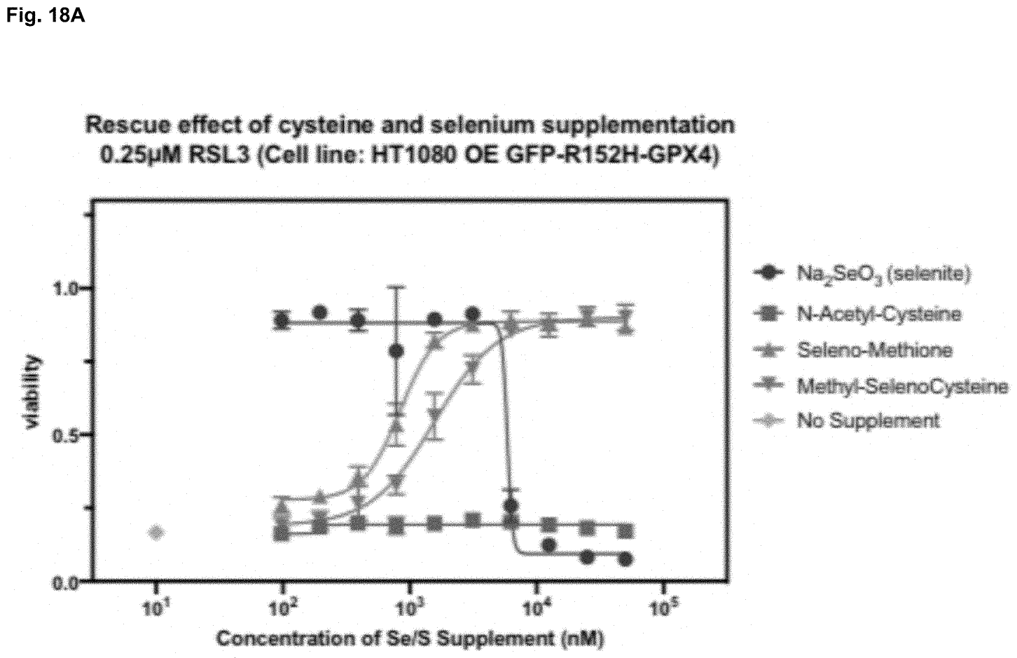

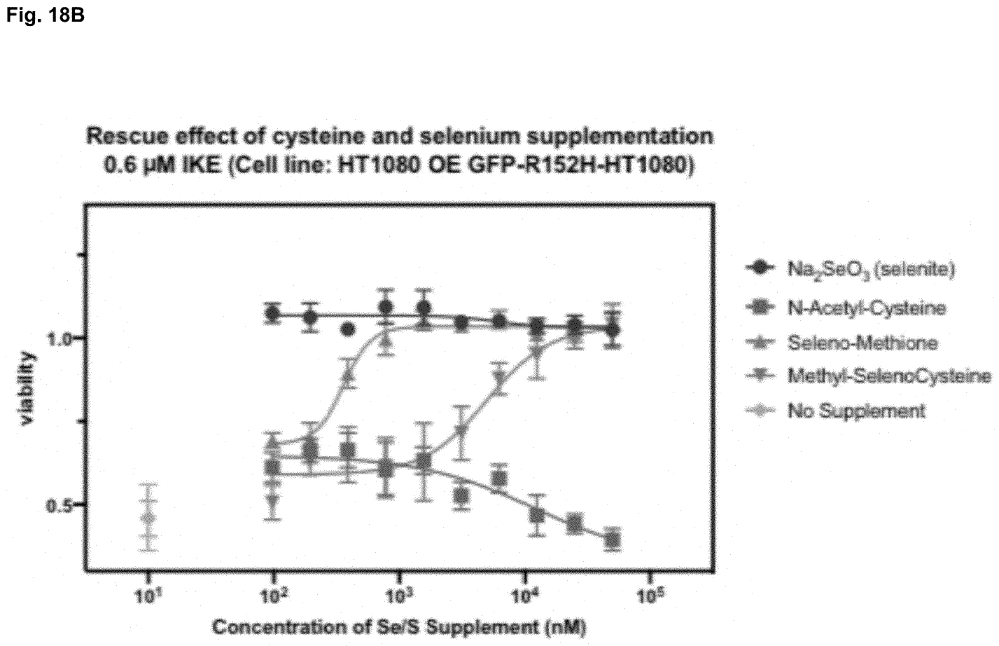

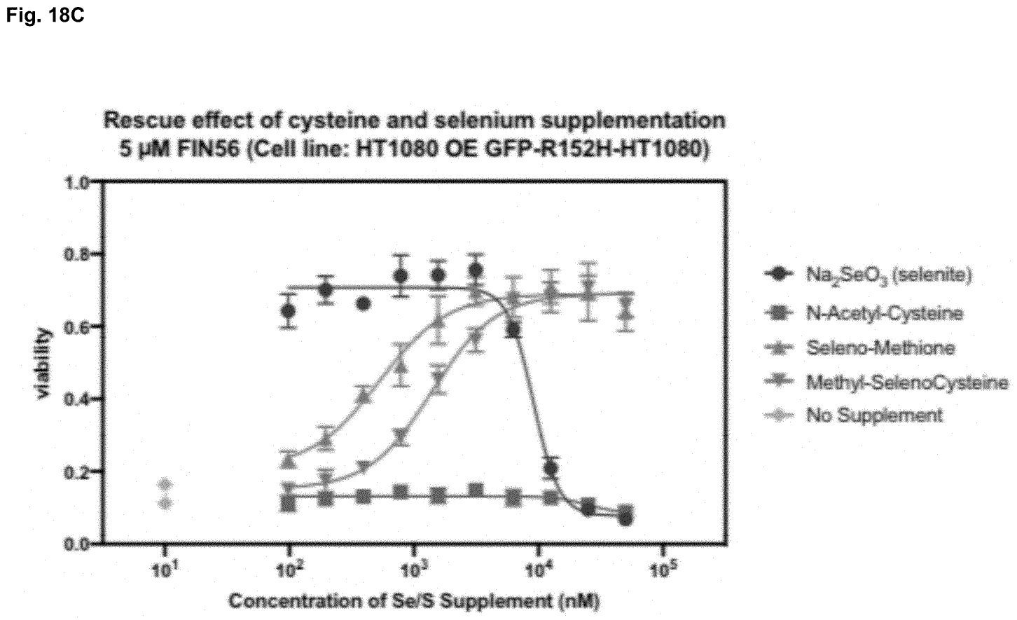

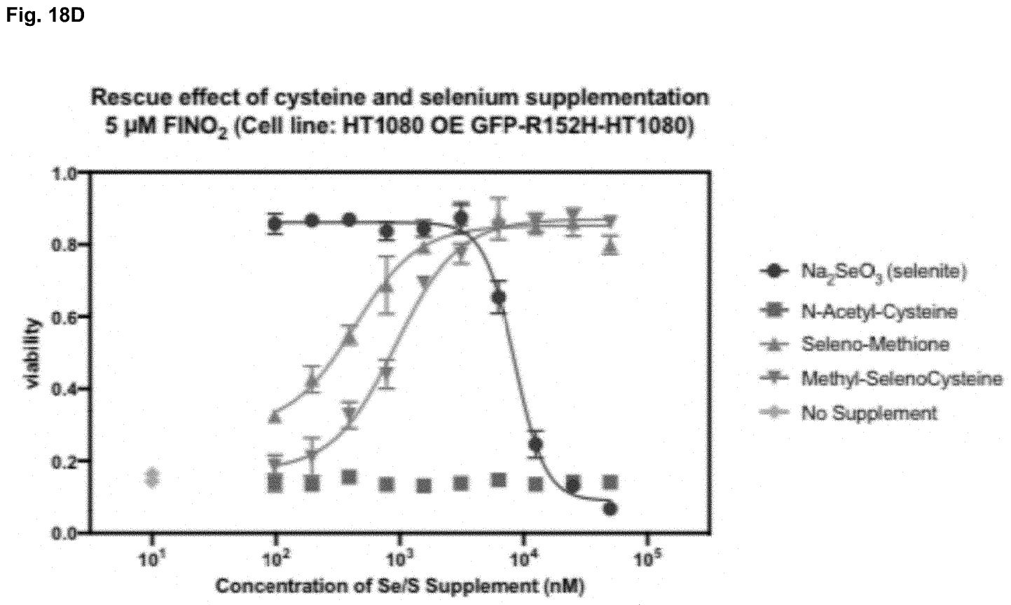

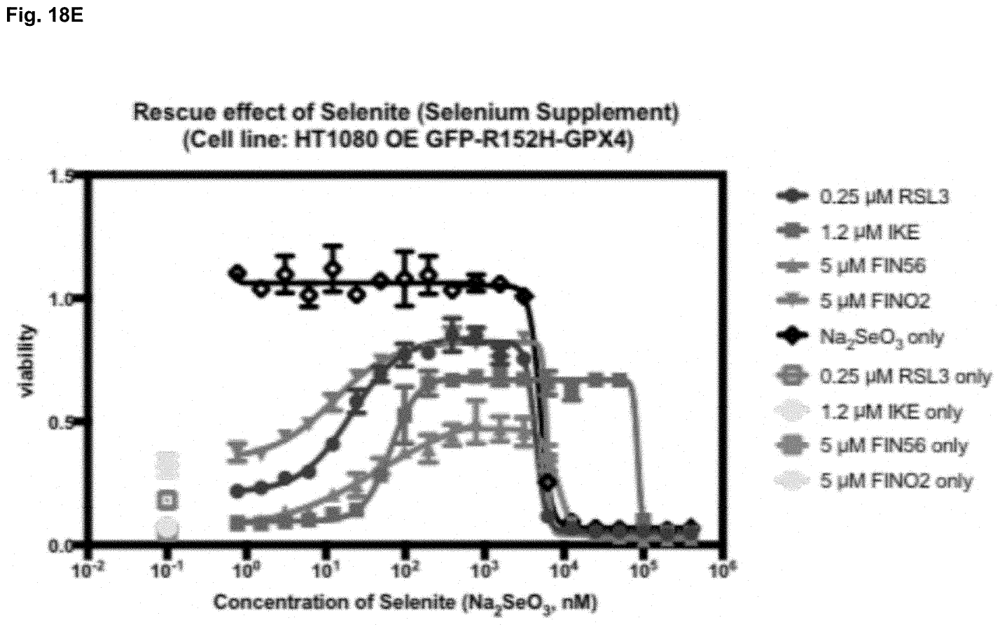

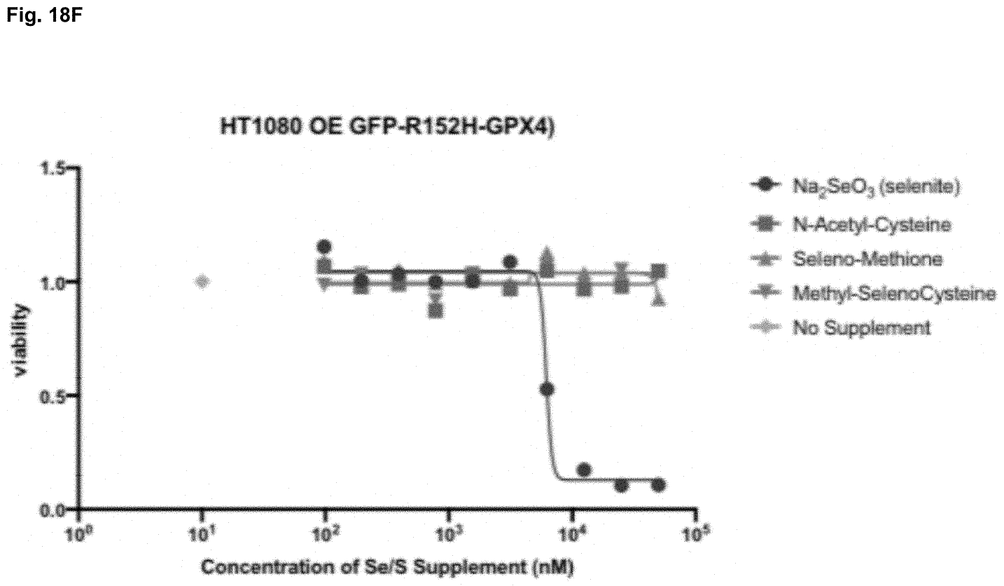

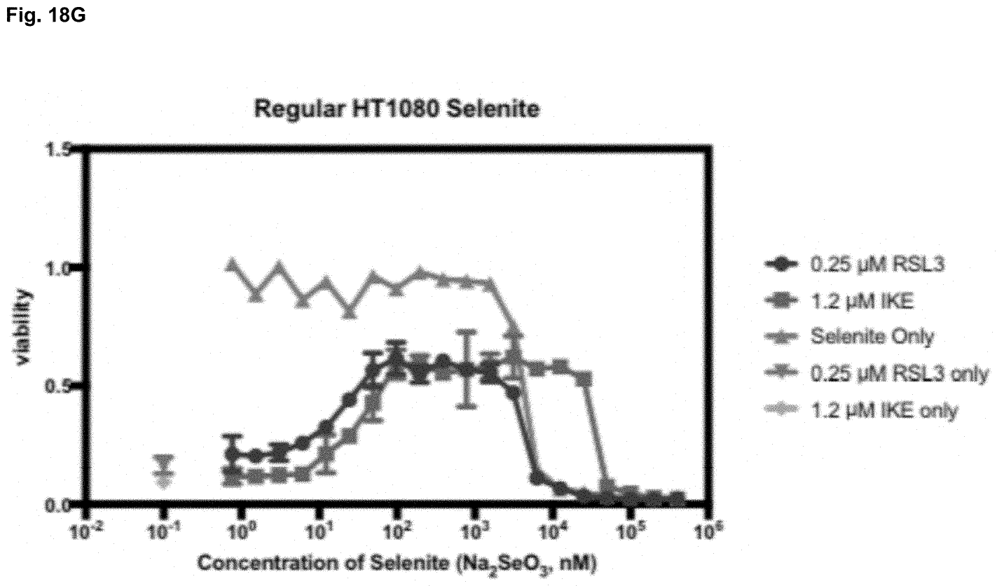

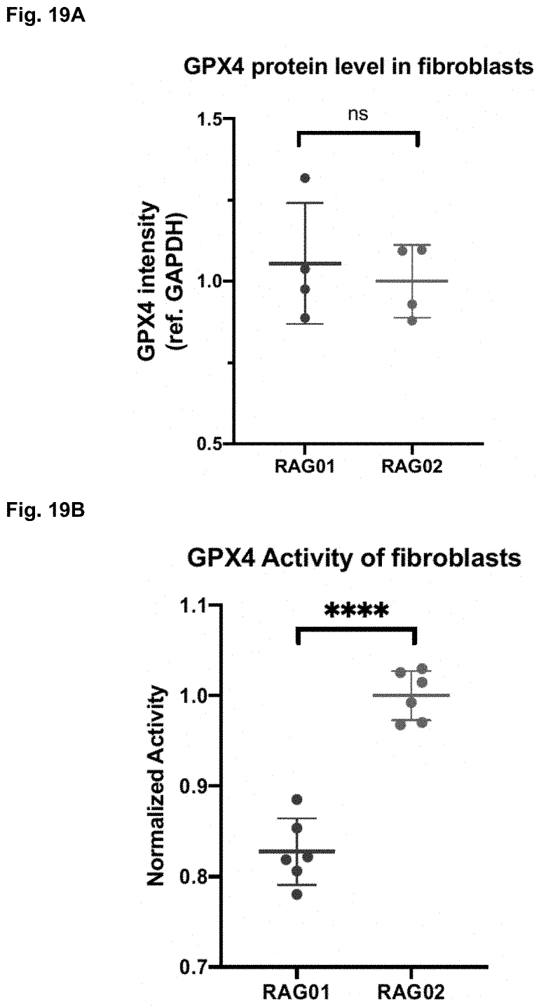

We tested the hypothesis on a HT-1080 OE GFP-GPX4^{R152H} cell model, which was transfected to overexpress GFP-GPX4^{R152H} and only produced a relatively negligible amount of endogenous GPX4^{WT} after pro-longed selections. Sodium selenite, selenomethione, and (methyl-)selenocysteine were tested as sources of selenium, while N-acetyl-cysteine was included as a control, which might also benefit the patients as a metabolic precursor of GPX4 cofactor GSH (Yang and Stockwell, 2016). After a pre-supplementation of selenium-containing compounds or cysteine at titrated concentrations for 24 h, HT-1080 OE GFP-GPX4^{R152H} cells were then uniformly treated with RSL3 at IC10 for 48 h to simulate a patient-related scenario, featuring a suppressed GPX4 activity and a vulnerability to phospholipid peroxidation and ferroptosis (FIG. 18A). The subsequent cell viability data showed limited protection effect by N-acetyl-cysteine supplementation, which suggested that the elevation of GSH level in cells might not be directly helpful in boosting the activity of the GPX4 variant when GPX4 is overwhelmingly inhibited by RSL3. Selenomethione, and (methyl-)selenocysteine showed moderate rescue effect, which were fully protective at the micromolar level. The results of selenocysteine and cysteine suggested the contributions of selenium supplementation. Furthermore, sodium selenite was shown to be the most effective treatment, with full protection observed at nanomolar concentrations, which might be due to its smaller size and greater cell permeability.

We also tested the protective effect of selenium-containing compounds and cysteine against other classes of ferroptosis inducers, which indirectly suppressed GPX4 activity via diverse pathways (FIGS. 18B-18D). We found that N-acetyl-cysteine only slightly protected cells from IKE, which is inhibitor of the cystine/glutamate antiporter system x_{c}⁻ (FIG. 18B). Nonetheless, all of the three selenium-containing compounds were able to fully rescue HT-1080 OE GFP-GPX4^{R152H} from all tested ferroptosis inducers, with sodium selenite being the most effective (FIGS. 18B-18D).

We repeated the above analysis of sodium selenite at an extended range of concentrations, and determined its EC₅₀ value of rescue effect against RSL3 (19 nM), IKE (66 nM), FIN56 (34 nM), and FINO₂ (12 nM, FIG. 18E). While only sodium selenite, among the tested supplemental compounds, provided substantial protection at nanomolar concentrations, it is also the only one that exhibited cellular toxicity when its concentration reached 5 μM (FIG. 18F). However, we observed an unexpected result that the toxicity of sodium selenite was significantly suppressed by IKE, while other ferroptosis inducers didn't show such an effect (FIG. 18E). While the IC50 of sodium selenite alone in the cell model is 5 μM, addition of 1.2 μM IKE shifted its IC₅₀ to 85 μM, inducing a 17-fold change. In other words, cells still survived with a combination of 1.2 μM IKE and 50 μM sodium selenite, which separately would have killed the cells. We also observed the same toxicity suppression effect on parental HT-1080 cells (FIG. 18G). Since inhibition of the cystine/glutamate antiporter system xc—can suppress the toxicity of selenium, this suggested that this system or cysteine/cystine is involved in the toxicity mechanism of selenium overdose. Additionally, this also indicated that, to alleviate or avoid the toxicity of selenium supplementation, we might limit the uptake of cysteine, in addition to a strict control of selenium dosage.

In summary, selenium supplementation, as a proof-of-concept treatment, was shown to be effective at nanomolar concentration in a cell model of a patient-derived GPX4 variant and therefore may benefit such patients.

### Proof-of-Concept Treatments in Patient Fibroblasts

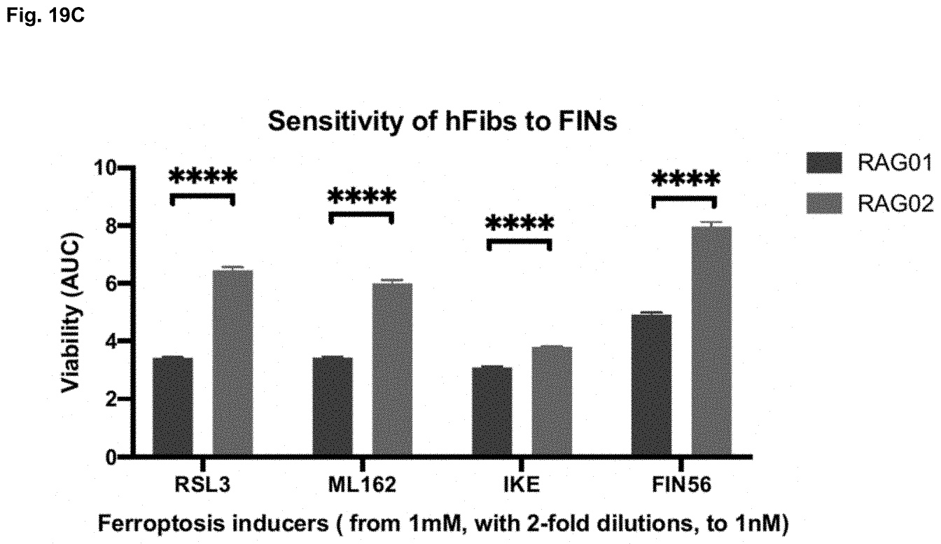

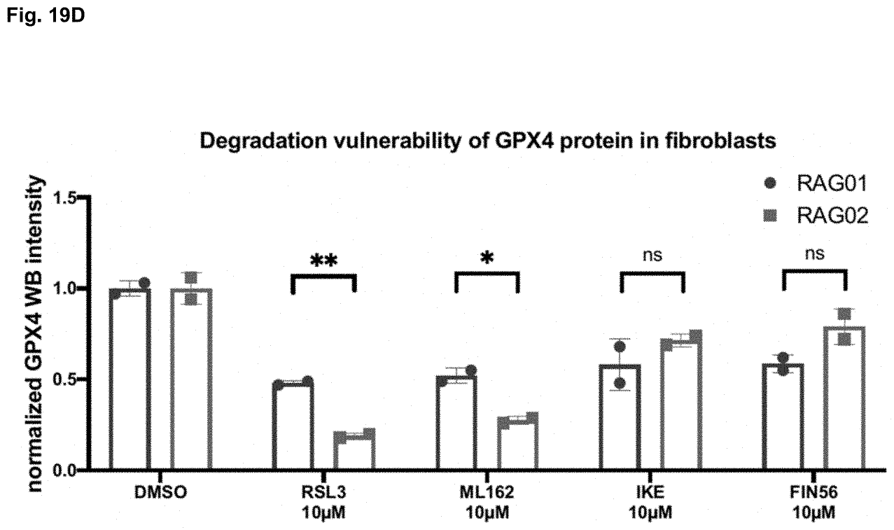

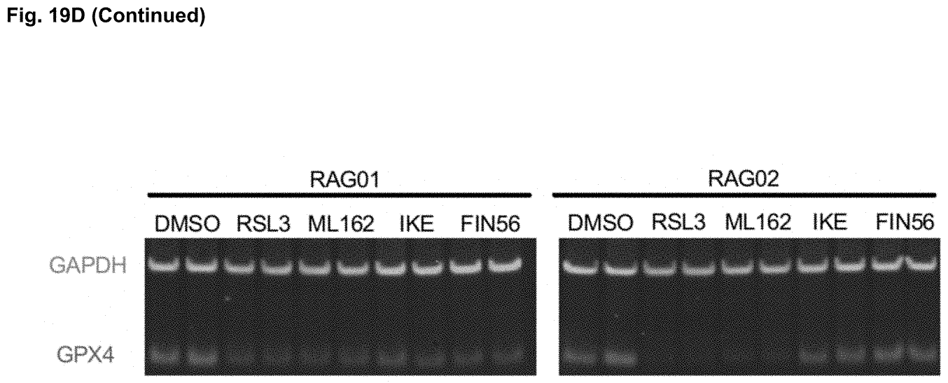

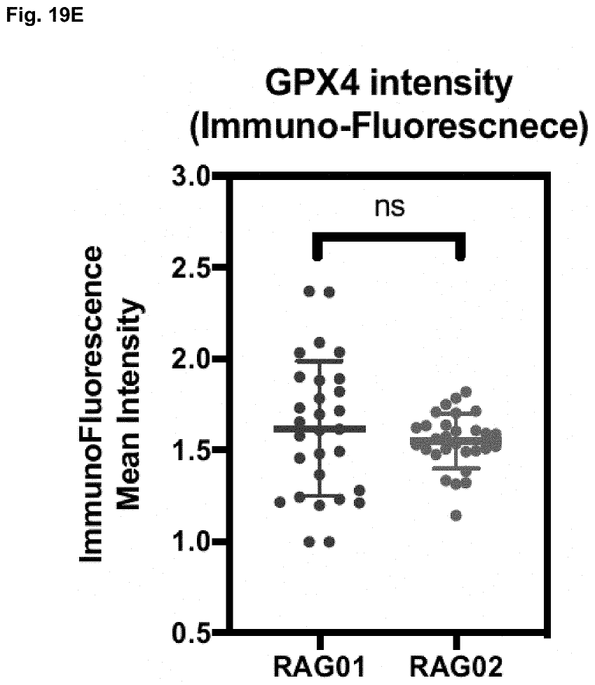

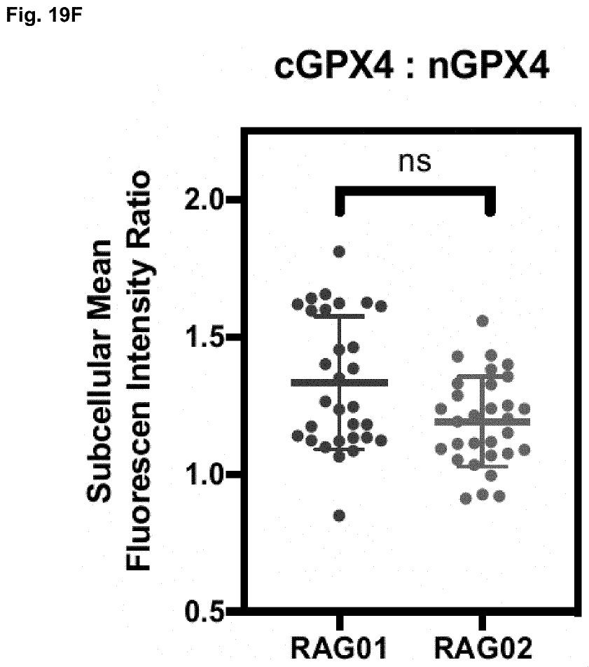

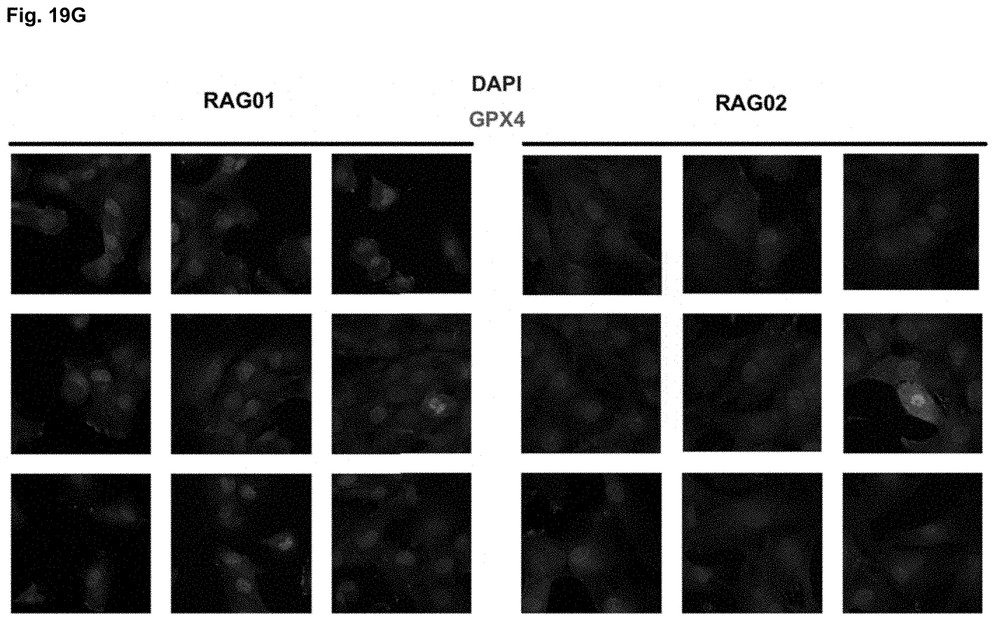

To validate these observations on molecular and cellular models, fibroblasts developed from a patient with homozygous mutations (RAG01, GPX4^{R152H/R152H}) and his parent as a healthy control with heterozygous mutations (RAG02, GPX4^{R152H/WT}) were tested for GPX4 protein level and enzymatic activity. Although the two human fibroblast cell lines expressed an equivalent level of GPX4 protein based on western blot analysis, RAG01 exhibited a significantly lower level of GPX4 enzymatic activity than RAG02, confirming that the patient-derived R152H variant in GPX4 caused substantial loss of function (FIGS. 19A and 19B). As expected, RAG01 with a partial loss of GPX4 activity was shown to be more sensitive than RAG02 to lipid peroxidation and ferroptosis induced by RSL3, ML162, IKE, and FIN56 (FIG. 19C). We then further tested the degradation vulnerability of GPX4 protein in both fibroblast cells, and found the mutation altered the degradation of GPX4 so that GPX4^{R152H} was more resistant than GPX4^{WT} to the degradation induced by RSL3 and ML162, which is consistent with our observations in the HT-1080 cell model and further suggested the involvement of ubiquitin-proteasome system in GPX4 degradation (FIG. 19D). Moreover, imaging of fibroblast cells with GPX4 immunofluorescence and DAPI staining under confocal microscope showed an indistinguishable level of cellular GPX4 intensity in RAG01 and RAG02 (FIG. 19E). Additionally, the ratio of cytoplasm GPX4 over nuclear GPX4 was comparable in RAG01 and RAG02, indicating that alteration of GPX4 subcellular localization was not observed for this patient-derived mutation (FIGS. 19F and 19G).

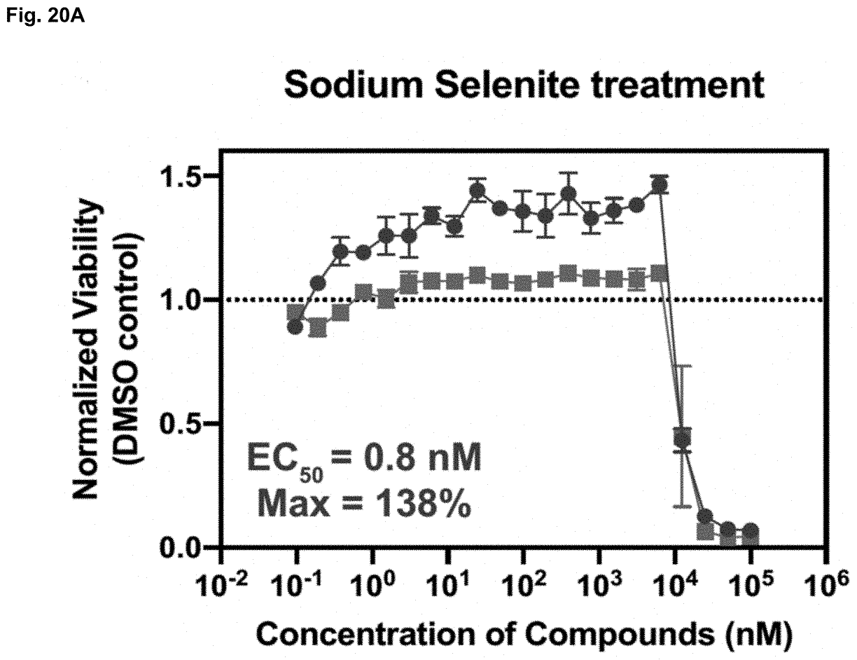

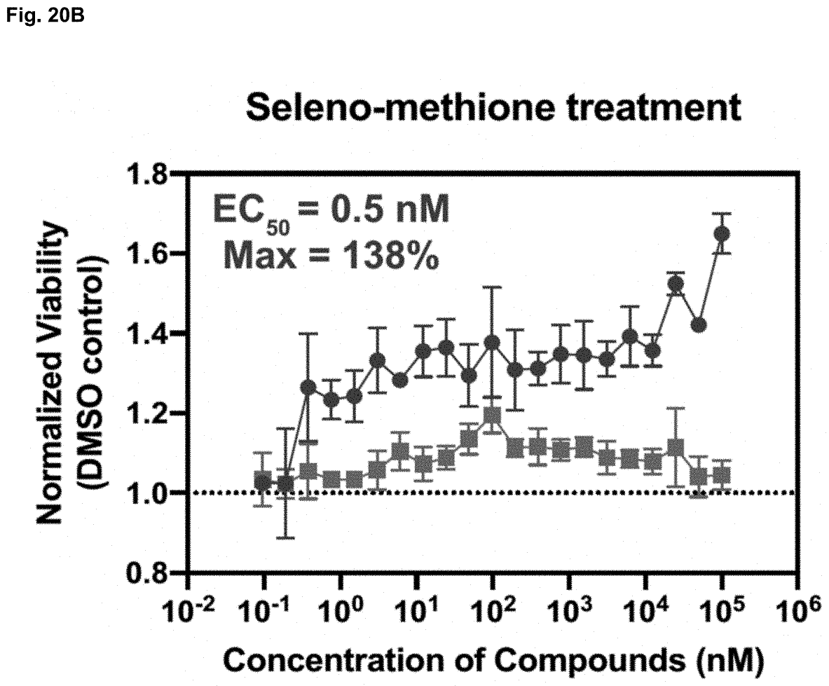

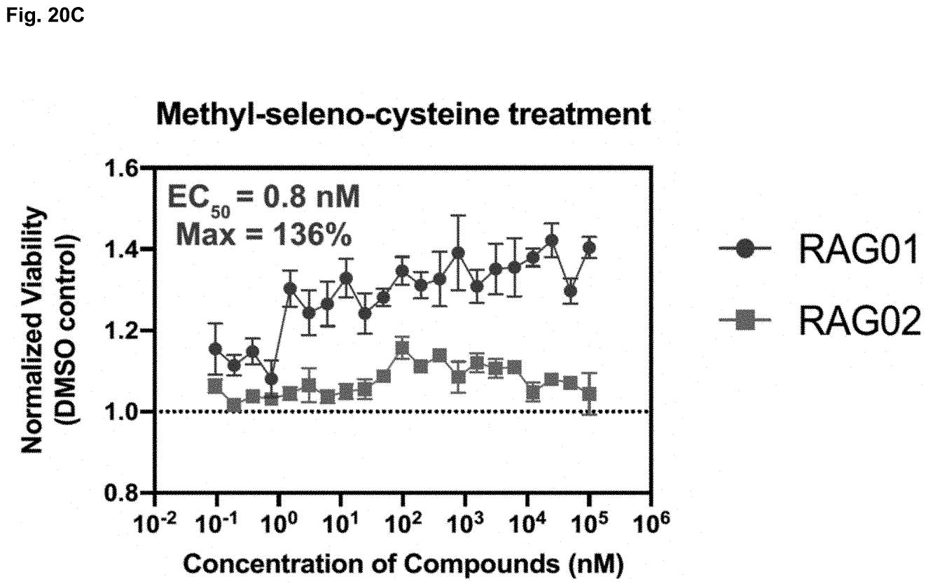

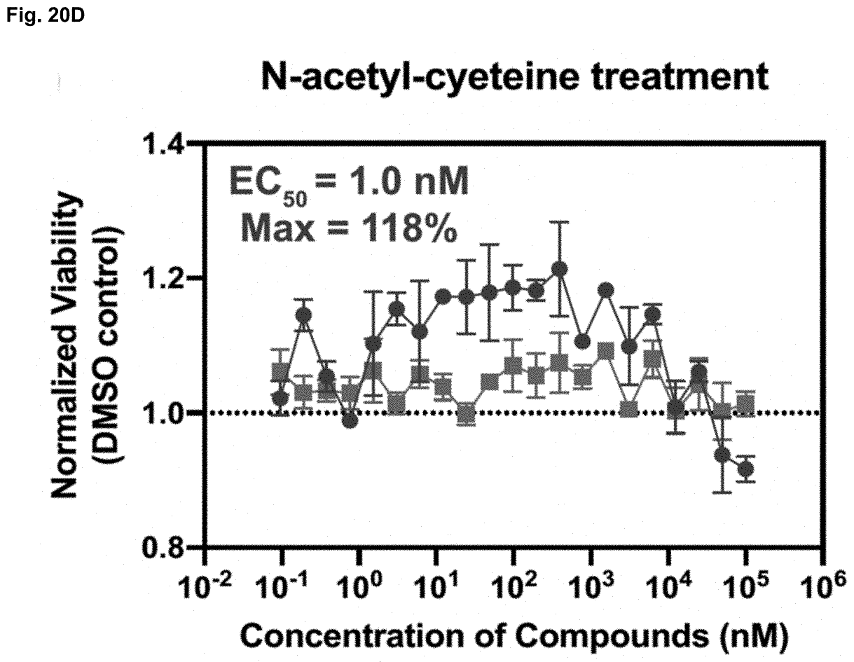

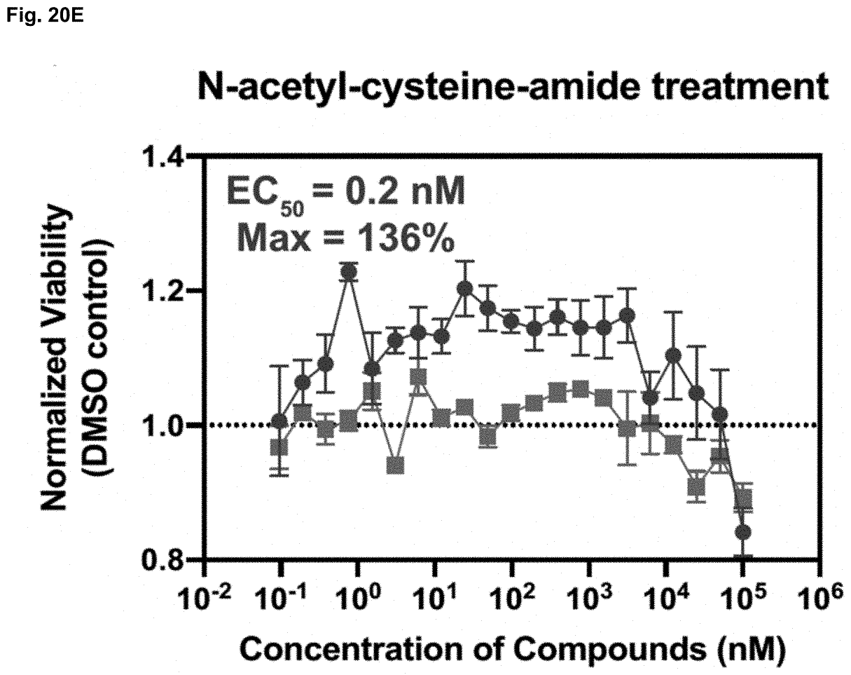

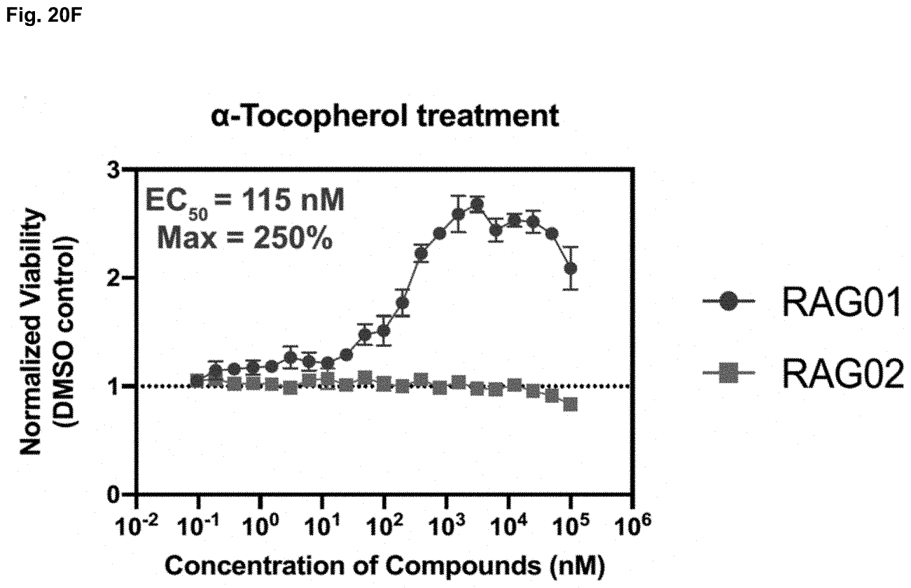

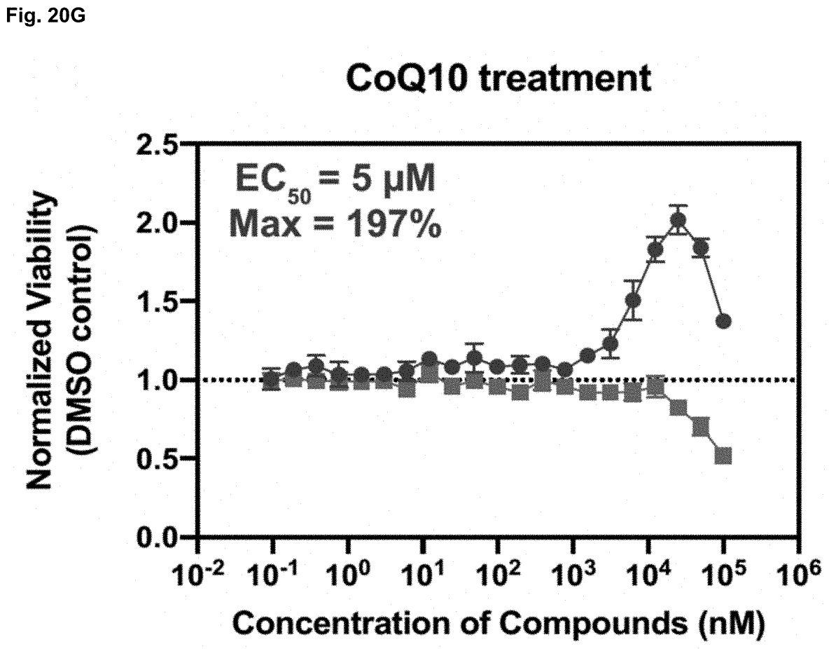

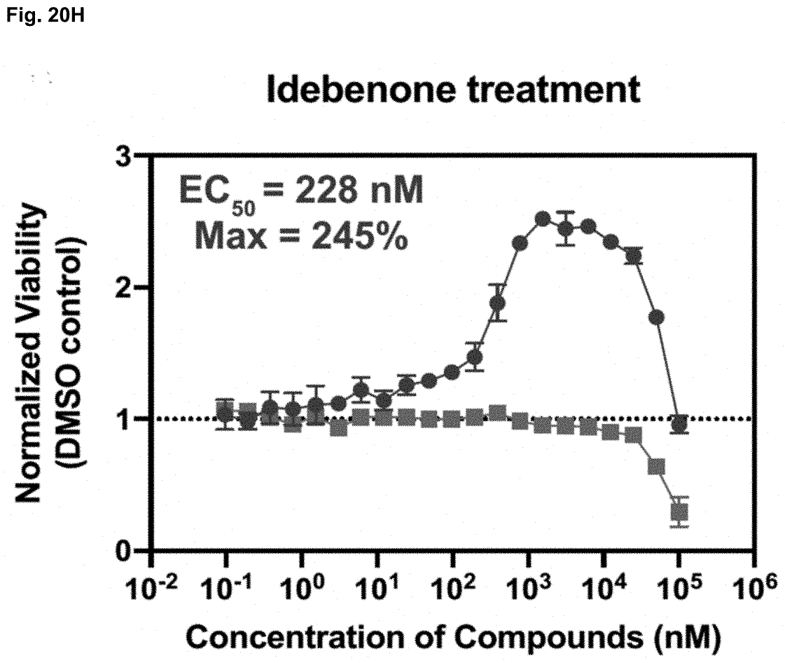

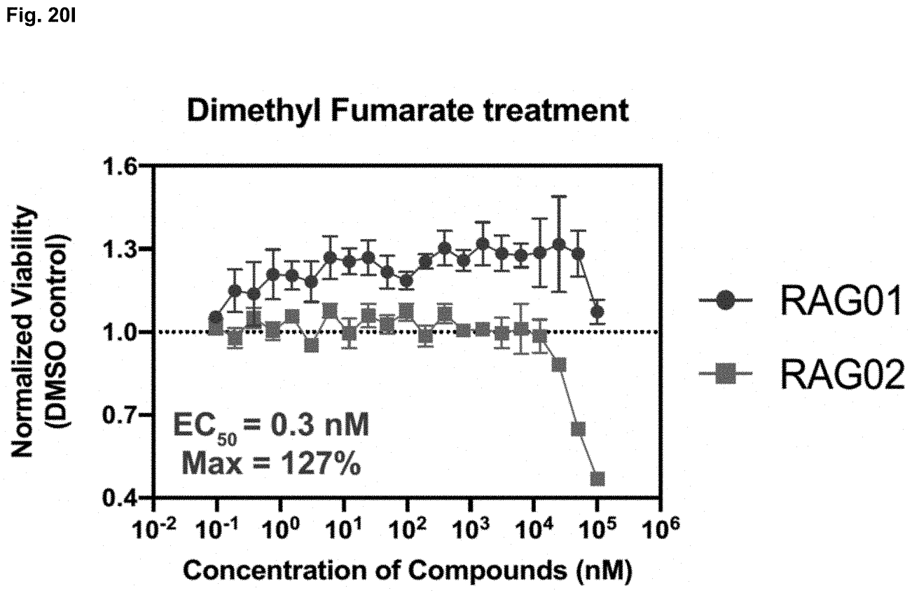

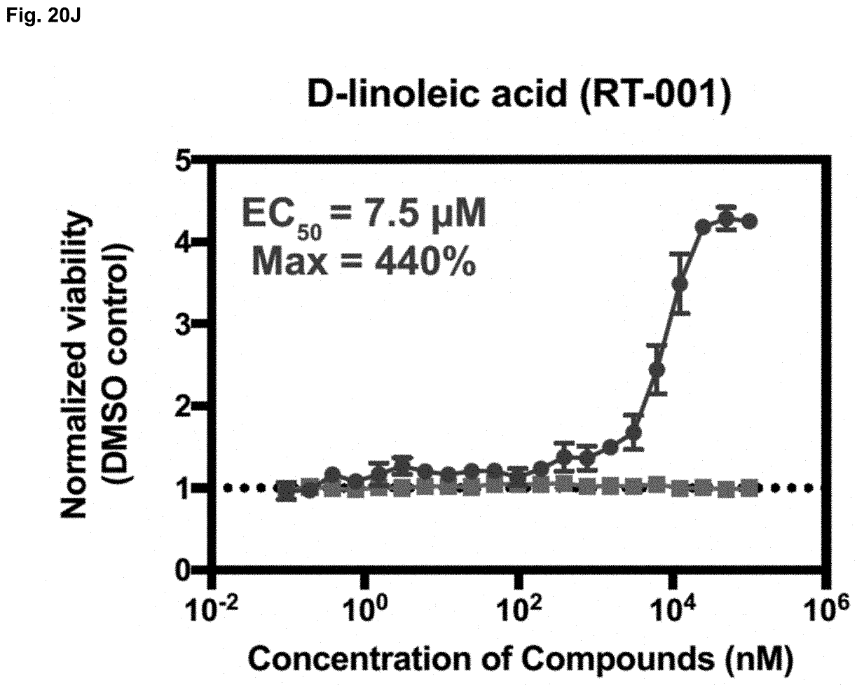

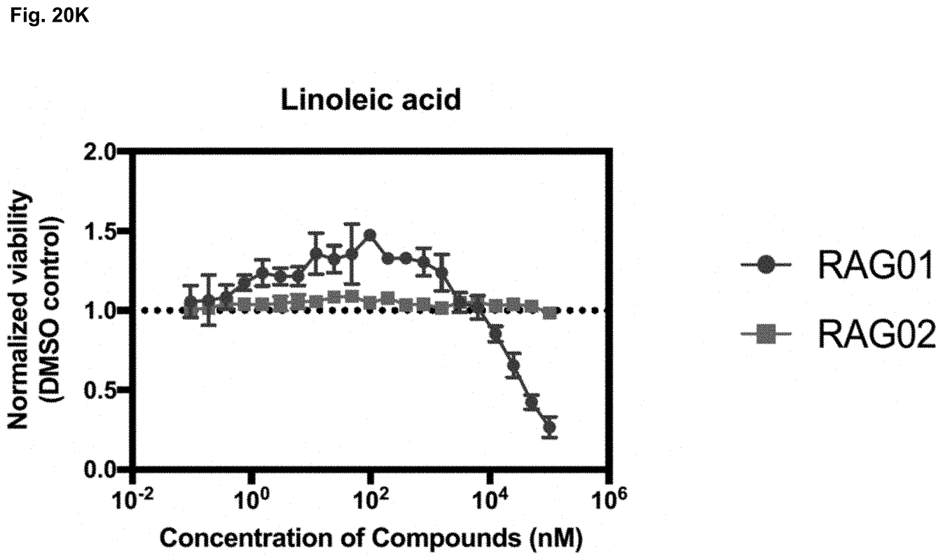

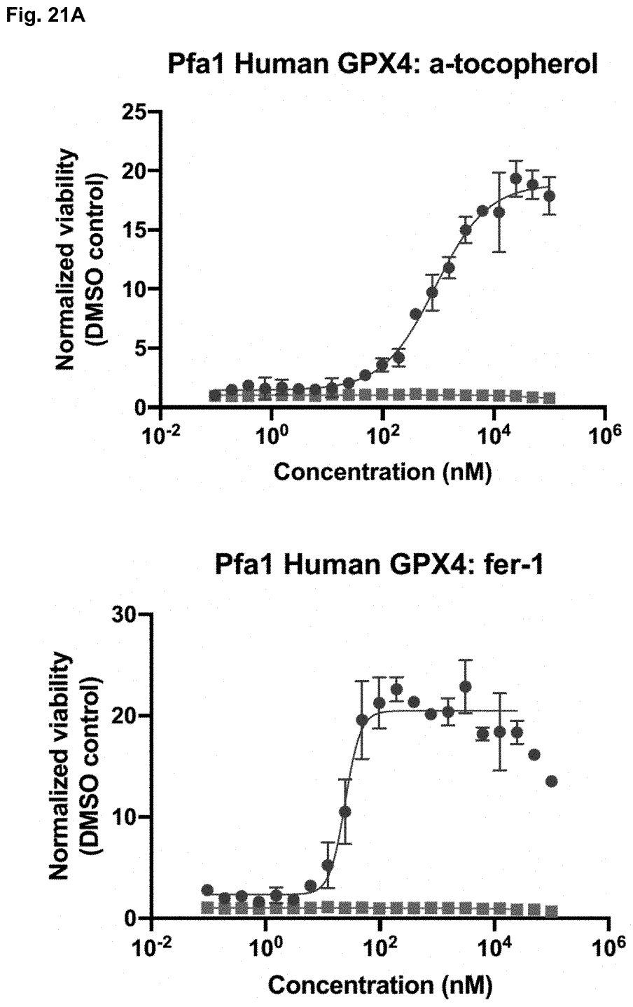

As fibroblast data validated the pathology analysis on the HT-1080 cell model, we further tested selenium supplementation and other proof-of-concept therapeutic treatments on fibroblasts. In contrast to the HT-1080 cell model, since patient fibroblasts already exhibited comprised GPX4 activity and a poor viability in the regular media, we didn't add ferroptosis inducers to the fibroblasts for these rescue experiments, but instead we directly treated fibroblast cells with various concentrations of selenium supplementation or antioxidants and then monitored the increase of cell numbers over control fibroblast cells treated with DMSO only as a readout. Accordingly, we found that selenium supplementation with sodium selenite, seleno-methione, and methyl-seleno-cysteine can boost the viability of RAG01 to 138.0%, 137.9%, and 136.3%, with an EC₅₀ value of 0.8 nM, 0.5 nM, and 0.8 nM, respectively (FIGS. 20A-20C). While the similarity in viability-increase extents of three seleno-compounds suggested the consistency of selenium supplementation as a beneficial treatment, the shared low EC₅₀ values indicated their high potencies as treatments and a precise effect on the GPX4 variant of the patient. As the IC₅₀ of sodium selenite toxicity (12 μM) is 15,000-fold higher than the its EC₅₀, the avoidance of its toxicity should be manageable, while seleno-methione and methyl-seleno-cysteine might be preferred. It is also noteworthy that the rescue effect of selenium is highly specific for RAG01, with relatively minimal effect on the control RAG02 line, which might come from its partial mutation of GPX4. Moreover, we found that N-acetyl-cysteine and N-acetyl-cysteine-amide can also boost the viability of RAG01 to 118% and 136.3%, which are less effective than selenol-cysteine and not effective beyond 1 μM (FIGS. 20D and 20E). Again, this suggested the utility of selenium for patients with compromised GPX4 activity.

Additionally, we found that the antioxidants α-Tocopherol, CoQ₁₀ and idebenone (a soluble analog of CoQ₁₀) can also boost the viability of RAG01 to 250%, 197%, and 245%, with an EC₅₀ value of 115 nM, 5 μM, and 228 nM, respectively (FIGS. 20F-20H). We also found that dimethyl fumarate, an Nrf2 activator that promotes an antioxidant response, boosted the viability of RAG01 to 127% (Wang et al. 2015) (FIG. 201). Furthermore, we found that treatment with deuterium-reinforced linoleic acid (RT-001), a polyunsaturated fatty acid (PUFA) with deuterium at its bis-allylic site to inhibit lipid autoxidation, provided the most pronounced rescue effect on RAG01 (Hatami et al. 2018) (440%, FIG. 20J). On the contrary, regular linoleic acid only exhibited a toxicity effect to RAG01 at high concentrations, which clearly demonstrated the involvement of lipid peroxidation in the pathology of the patient (FIG. 20K).

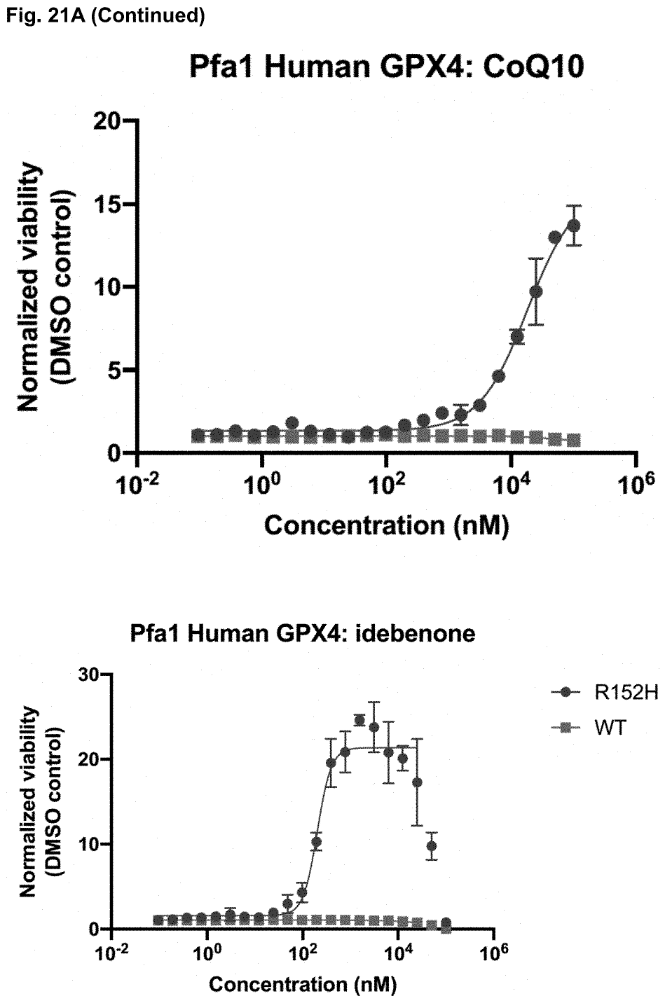

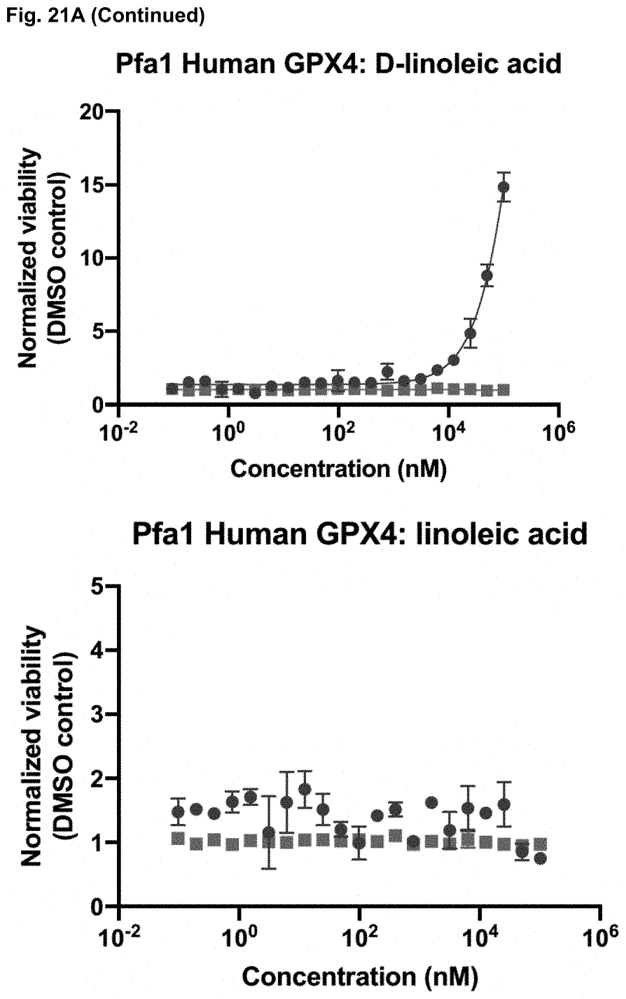

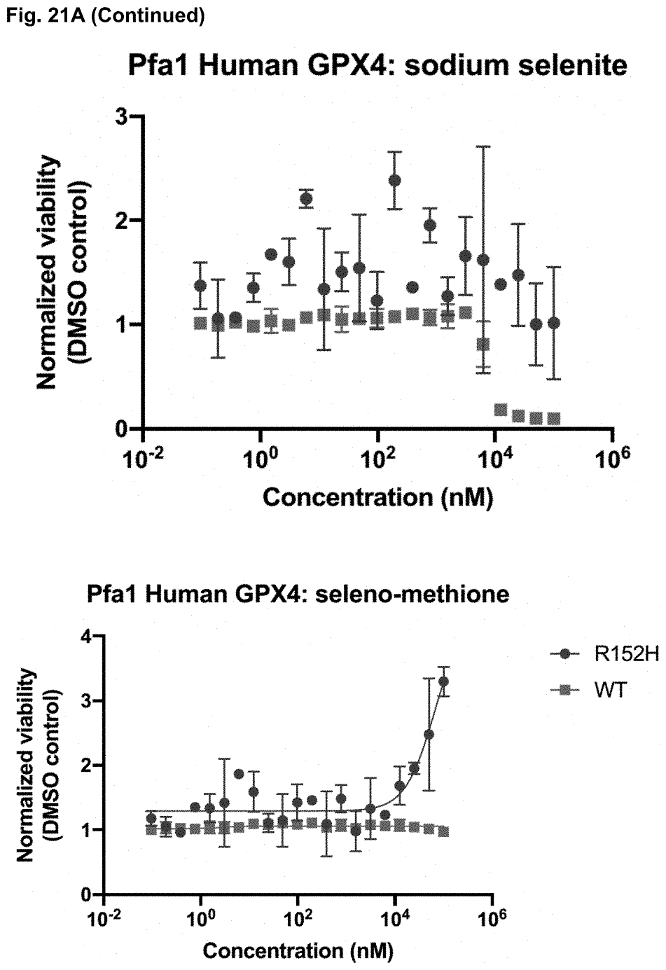

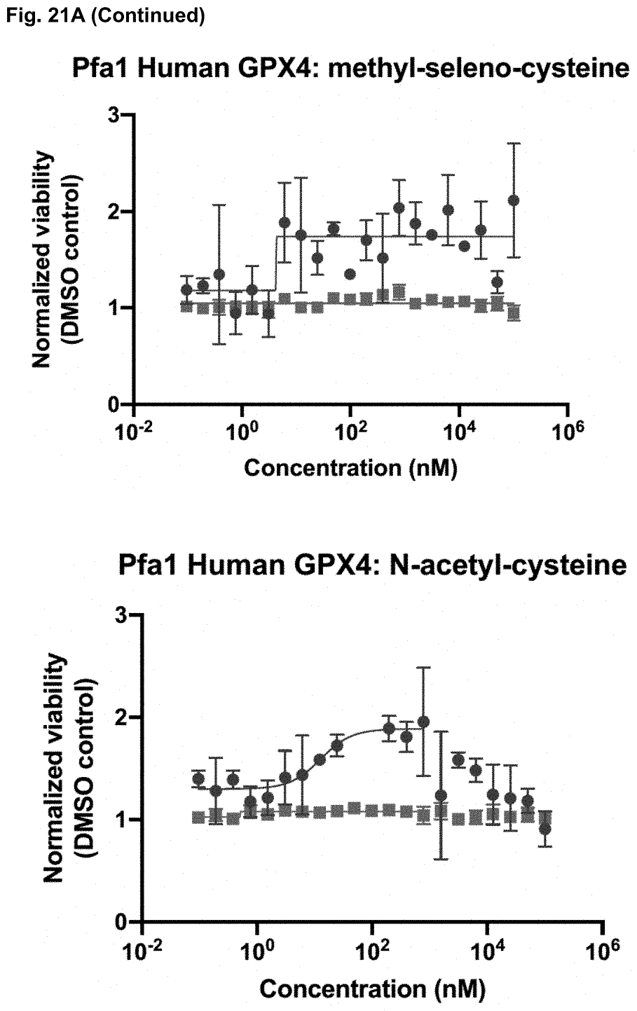

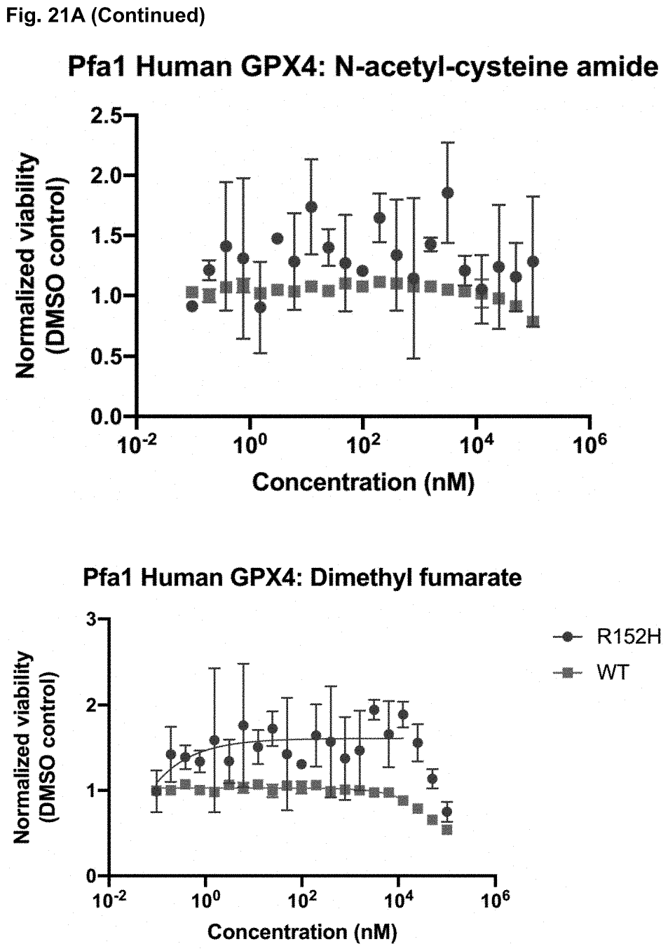

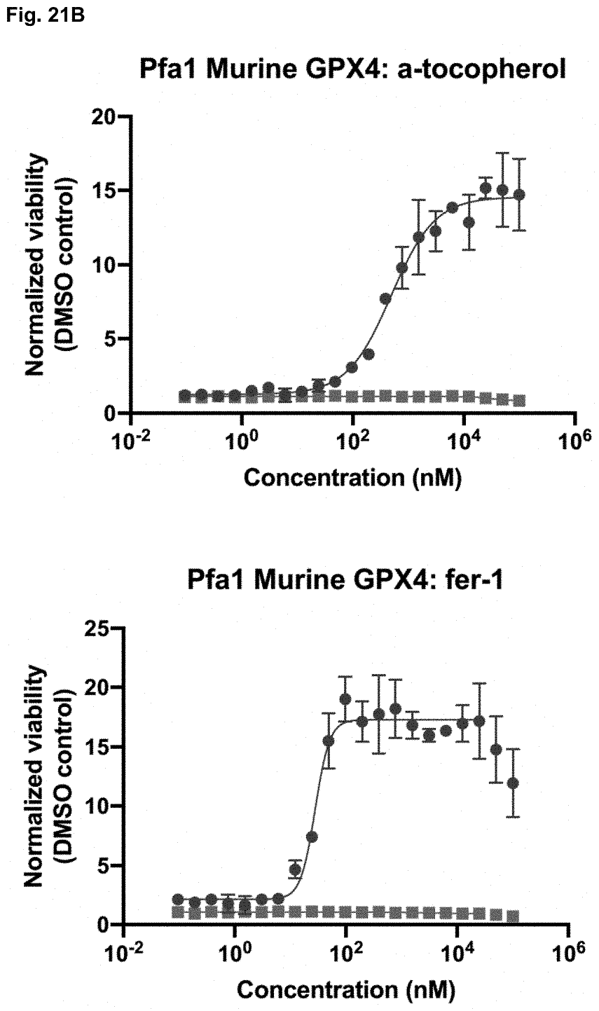

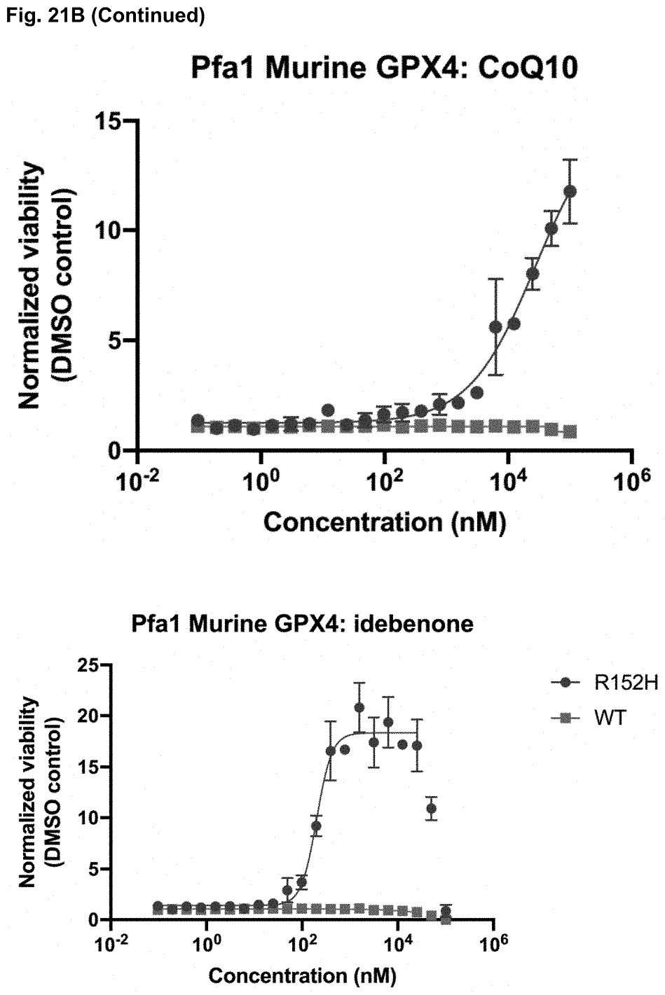

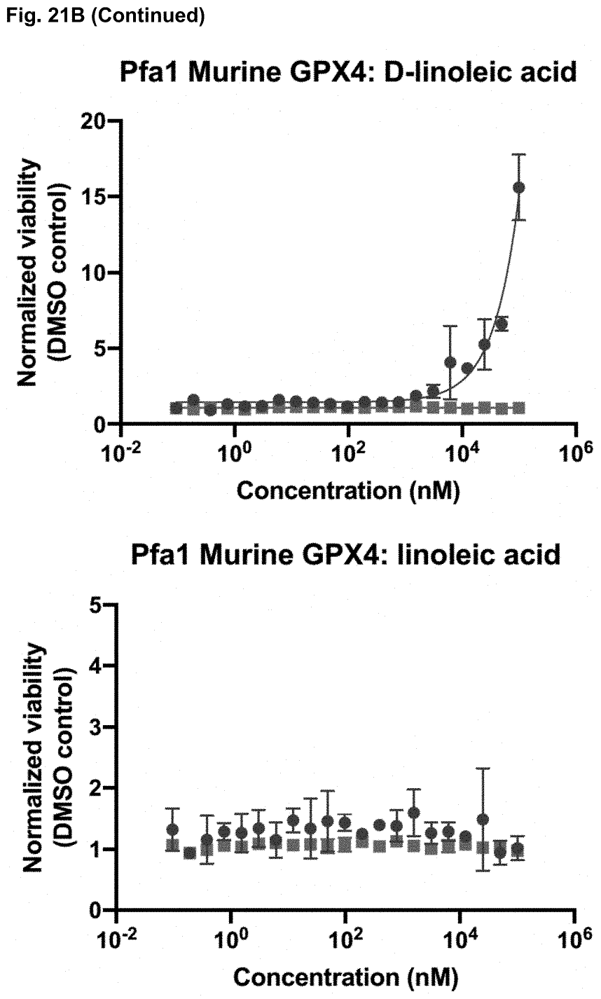

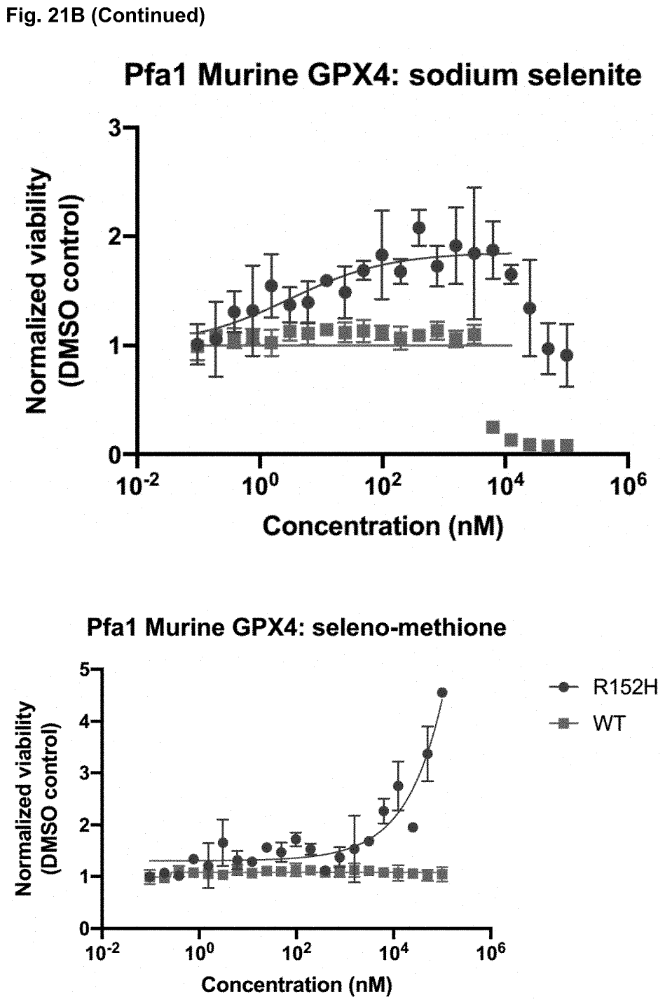

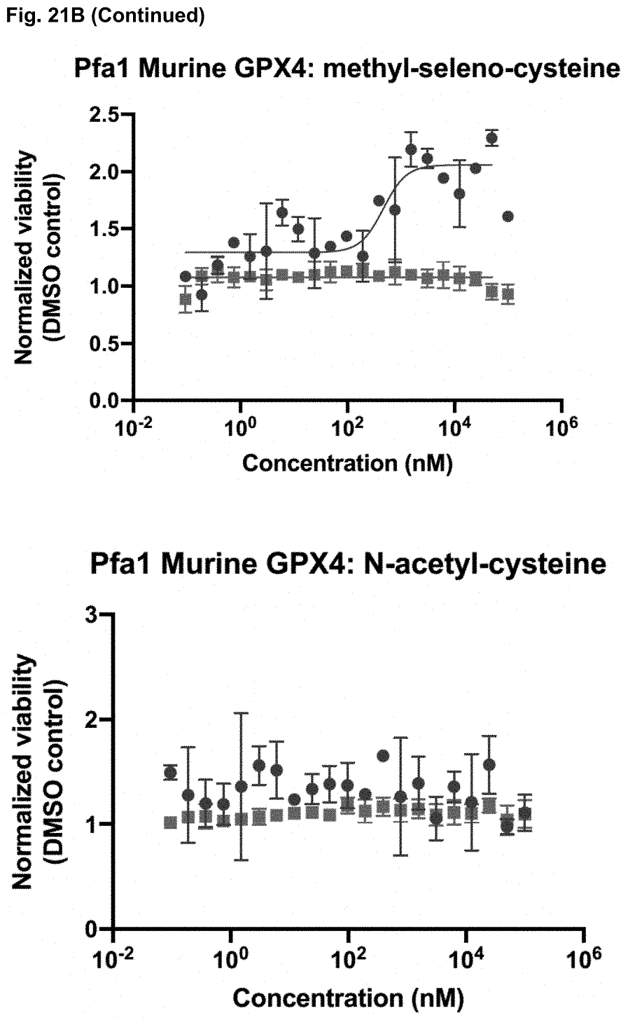

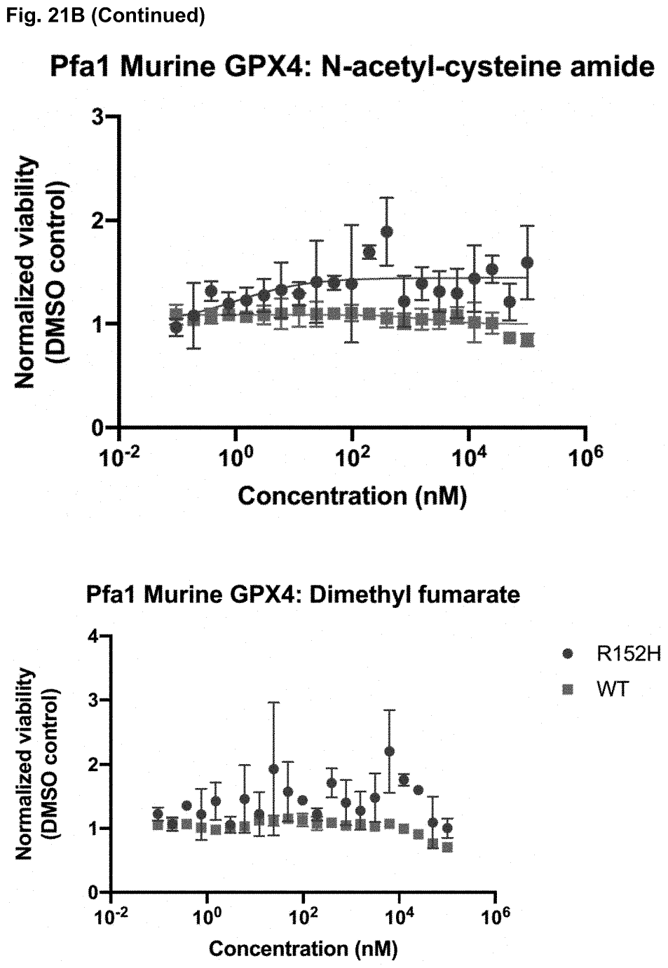

To further evaluate the potency of the proof-of-concept treatments and compare effects on GPX4^{R152H} to a mock control solely expressing wild-type GPX4 (instead of RAG02 expressing both variants), we tested all treatments on Gpx4-knockout Pfa-1 cells that are transfected to overexpress human/murine GPX4^{WT} or GPX4^{R152H} (FIG. 21). Fer-1, an specific inhibitor of ferroptosis, was also included in the test; its significant and selective rescue effect on cells solely expressing GPX4^{R152H} demonstrated the involvement of ferroptosis in the R152H pathology at cellular level (FIG. 21). In general, proof-of-concept treatments effective on the patient fibroblast cells also exhibited consistent rescue effects on the Pfa-1 cells, especially for α-Tocopherol, CoQ₁₀, idebenone, and D-linoleic acid. Their higher apparent rescue indexes on the Gpx4-knockout Pfa-1, compared with their effects on the fibroblasts, are likely due to the lower viability of the Pfa-1 at the particular intervention time point, 7 days after the removal of α-Tocopherol from media. When there was no intervention at this time point, Gpx4-knockout Pfa-1 expressing human GPX4^{R152H} exhibited lower than 5% viability, while it was approximately 10% viability for Pfa-1 expressing murine GPX4^{R152H}. This is accordance with the observation that the antioxidants exhibited higher apparent rescue indexes on the former cells than the latter. On the other hand, different from the anti-oxidants, the better effects of all selenium supplementation on the cells with relative higher viability before intervention indicated that selenium supplementation would work better at an early intervention time point (FIG. 21).

In summary, the observation that selenium supplementation, ferroptosis inhibitors, and antioxidants can increase the cell number and viability of patient fibroblasts and our R152H cell models further confirmed our pathological mechanism analysis, whereby the partial loss-of-function mutation in GPX4 sensitize the patient's cells to lipid peroxidation and ferroptosis. Therefore, we would expect that a combinational treatment of selenium supplementation to boost GPX4 level and antioxidants to suppress lipid peroxidation (α-Tocopherol and CoQ₁₀) might be the most effective treatment, which are all widely available as OTC supplements.

An outstanding phenomenon from the immunofluorescence imaging of the fibroblast cells might further support the potency of these proof-of-principle therapeutic treatments for patients, as we found cells undergoing mitosis exhibited a much higher fluorescence intensity of GPX4 than non-mitotic cells (FIG. 19G). This indicated that, during proliferation, fibroblast cells expressed a high level of GPX4 protein to protect themselves against lipid peroxidation. The loss-of-activity mutation in GPX4 might suppress mitosis, as evidenced by the lower number of RAG01 cells than RAG02 when seeded equivalently and the patient-selective viability-boosting effects of selenium supplements and anti-oxidants. Therefore, in summary, we propose that a combinational treatment of selenium supplementation and general antioxidants would be therapeutically beneficial for patients with the R152H variant in GPX4.

### Discussion

Prior to this study, only three previously reported infants with SSMD were identified to have predicted loss of function variants based on in silico data, although these variants were not studied in vitro (Smith et al. 2014; Fedida et al. 2020). Our report of the patient with biallelic missense variants from an unrelated family with typical findings of SSMD, combined with the functional characterization of the R152H missense variant, provided further evidence of the association of biallelic variants in GPX4 with SSMD. In addition, this report extends the phenotype associated with bi-allelic variants in GPX4 beyond SSMD to include long-term survival beyond infancy and other skeletal and neurological findings.

This study began by examining the effect of the R152H missense mutation on GPX4, which adversely changed the protein structure and caused a partial loss of its antioxidant activity. As a hypomorphic allele, we therefore hypothesize that there is sufficient enzymatic function to allow for survival beyond infancy but results in increased susceptibility to ferroptosis culminating in cell death, tissue damage, and neurodegeneration. This finding would be in keeping with observations of the adult condition Gpx4 knockout mouse which developed seizures, ataxia and progressive neuronal loss (Yoo et al. 2012).

Interestingly, we also reported a second family harboring the R152H missense variant in a homozygous state with concordant neurological phenotypes but the affected individuals (patients 2 and 3) had atypical skeletal findings for SSMD. The reasons for the disparate skeletal phenotypes despite the identical genotype are unclear. We speculate that the individuals in family 2 may harbor other genetic variants that are protective against the impact of GPX4 variation on developing chondrocytes. Identification and study of other individuals with biallelic variants in GPX4, regardless of the skeletal phenotype, as well as genotyping of other reported individuals with typical SSMD may improve our understanding of the phenotypic spectrum of GPX4 associated disease.

Through real-time communications with patients along our research timeline, living patients with the identified mutation have begun taking selenium supplementation and antioxidant treatments, including vitamin E, N-acetyl-cysteine, and CoQ₁₀. With 50 mg BID CoQ₁₀, 100 mcg selenium per day, and 150 mg BID N-acetyl-cysteine, patient 1 has developed enough strength to lift his head from a prone position for a brief time, and his skeletal X-ray indicated his bone is normalizing.

During this analysis, we used a structure-based computational modeling approach to study the effect of the variant on protein structure in silico, the predictions from which were confirmed by protein crystal structures and cellular assays. These experiments suggested that structure-based modeling of variant protein structures is an important and reliable approach to dissect the impact of patient-derived variants. The low-cost and high-throughput of computational modeling would potentially benefit more patients with orphan disease or variation in key genes.

This variant unexpectedly revealed the structural basis of GPX4's enzymatic activity and the regulation of its degradation: Lys48 was found to modulate GPX4 enzymatic activity and Lys125/Lys127 were revealed as sites of ubiquitin ligation. In addition, this also suggested Arg152 as an allosteric site indirectly regulating GPX4 activity. Since recent studies highlighted GPX4 protein as an Achilles's heel of drug-resistant and metastatic cancers, which are exceptionally dependent on the GPX4 lipid peroxide repair pathway, we would expect biochemical therapeutic strategies targeting the essential residue Lys48 and allosteric site Arg152 or taking advantage of its degradation mechanism, to be developed as a high priority and benefit additional patients (Viswanathan et al. 2017; Hangauer et al. 2017).

### Methods

### Cell Lines

HT-1080 cells were obtained from ATCC and grown in DMEM with glutamine and sodium pyruvate (Corning 10-013) supplemented with 10% FBS, 1% non-essential amino acids (Invitrogen) and 1% penicillin-streptomycin mix (Invitrogen). Human fibroblast cell line RAG01 and RAG02 were developed from patient with homozygous R152H variant and his parent with heterozygous R152H variant. RAG01 and RAG02 were grown in DMEM with glutamine and sodium pyruvate (Corning 10-013) supplemented with 15% FBS, 1% non-essential amino acids (Invitrogen) and 1% penicillin-streptomycin mix (Invitrogen). RAG01 and RAG02 cell lines are available for both commercial and academic use through CureGPX4.org, a patient organization dedicated to finding a treatment for SSMD disease. Cells were maintained in a humidified environment at 37° C. and 5% CO₂ in a tissue incubator.

### Computational Modeling and Molecular Dynamics (MD) Simulation

In silico residue mutation analysis and molecular dynamics (MD) simulations were performed in Maestro (Schrödinger Suite).

The crystal structure of GPX4^{U46C} (PDB: 2OBI) was imported into Maestro, preprocessed to remove water and add hydrogens, optimized at pH 7 for H-bond assignment, and minimized in the OPLS3e force field using Protein Preparation Wizard. Substitution of the Arg152 residue in the structure by His was followed by a global minimization of the whole structure in an implicit solvent to generate a GPX4^{R152H} model. As a control, GPX4^{R152R} was also generated by a synonymous mutation of Arg152 to Arg using the same algorithm and minimization method. SiteMap was then run under the default setting. A structural comparison between GPX4^{R152H} and GPX4^{R152R} was performed thereafter.

For MD simulation, each of the above structures was set up in an orthorhombic box with 0.15 M NaCl in SPC solvent and OPLS3e force field. MD simulations of the system for 100 ns with 4.8 ps per step at 300 K and 1.01325 bar were performed with random seeding in Desmond Molecular Dynamics. Simulation quality and event analysis were also done by Desmond. Videos for representative simulation process were exported.

### Generation of Cells Expressing Tagged GPX4

A pBabe-puro vector incorporated with the cDNA of GFP-tagged-cyto-GPX4^{WT} was prepared in previous work (Yang et al. 2014). With the vector as template, the following mutagenesis primers were designed using the Agilent QuikChange Primer Design application: R152H (5′-CTG CGT GGT GAA GCA CTA CGG ACC CAT GG-3′ (SEQ ID NO: 6), 5′-CCA TGG GTC CGT AGT GCT TCA CCA CGC AG-3′ (SEQ ID NO: 7)), K48A (5′-GGC CTC CCA GTG AGG CGC GAC CGA AGT AAA CTA C-3′ (SEQ ID NO: 8), 5′-GTA GTT TAC TTC GGT CGC GCC TCA CTG GGA GGC C-3′ (SEQ ID NO: 9)), K125R_K127R (5′-TGG ATG AAG ATC CAA CCC AGG GGC AGG GGC ATC CTG-3′ (SEQ ID NO: 10), 5′-CAG GAT GCC CCT GCC CCT GGG TTG GAT CTT CAT CCA-3′ (SEQ ID NO: 11)). Primers were purchased from Integrated DNA Technologies. Site-directed mutagenesis kit (QuickChange II, Agilent 200521) was then used to acquire pBP-GFP-cGPX4^{R152H}, pBP-GFP-cGPX4^{K48A}, and pBP-GFP-cGPX4^{K125R_K127R}. All mutations and the resulted plasmids were confirmed by sequencing at GENEWIZ.

HT-1080 cells were seeded into a 6-well dish at a density of 300,000 cells/well the night before lipofection. 2.5 μg DNA (empty pBabe-puro vector and the above four GFP-GPX4 expressing pBabe-puro vectors, separately), 7.5 μL Lipofectamine 3000 (Invitrogen, L3000015), and 250 μL Opti-MEM were incubated for 5 min at room temperature before adding to the HT-1080 cells. Following transfection, cells were passaged several times in media containing 1.5 mg/mL puromycin and grown in this media for all experiments performed. Expression of the exogenous GFP-tagged-GPX4 was confirmed with fluorescence microscope and Western Blot with both GFP and GPX4 antibodies.

### Western Blot

For the quantification of GPX4 protein level, the biological duplicates of each transfected HT-1080 cell lines subject to the GPX4-specific activity were tested by Western Blot in technical duplicates, which made a total of 4 samples for each cell line. In particular, cells were harvested with trypsin (Invitrogen, 25200-114), pelleted, and lysed by LCW lysis buffer (0.5% TritonX-100, 0.5% sodium deoxycholate salt, 150 mM NaCl, 20 mM Tris-HCl pH 7.5, 10 mM EDTA, 30 mM Na-pyrophosphate, and complete protease inhibitor cocktail). While part of the cell lysates was blotted for protein quantification, the other part of lysates was used for the GPX4-specific activity assay.

For the GPX4 degradation study, HT-1080 cells were seeded at 800,000 per well in a 60-mm plate and allowed to adhere overnight. Cells were then treated with 100 μM α-tocopherol and either 1 μM RSL3, 1 μM ML162, 10 μM IKE, 10 μM FIN56, or vehicle for 10 h. Cells were harvested with trypsin (Invitrogen, 25200-114), pelleted, and lysed with RIPA buffer.

For both experiments, cell lysates were blotted and imaged as previously described (Yang et al. 2014). Antibodies used were GPX4 (Abcam, ab125066, 1:250 dilution), actin (Cell Signaling, D18C11, 1:3,000 dilution), and GAPDH (Santa Cruz, sc-47724, 1:10,000 dilution). Results were quantified using a LI-COR Odyssey CLx IR scanner and GraphPad Prism 7.

### Immunofluorescence Study and Quantification

Human fibroblast cells RAG01 and RAG02 were separately seeded on poly-lysine-coated coverslips (Sigma Aldrich P4832) in a 24-well plate (100,000 per coverslip and three coverslips for each cell line) and allowed to grow overnight. Medium was removed and the cells were gently washed with PBS²⁺ (PBS with 1 mM CaCl₂ and 0.5 mM MgCl₂) gently twice. The cells were fixed and permeabilized by adding 200 μL/well of 4% paraformaldehyde (PFA) in PBS with 0.1% Triton X-100 (PBS-T), and incubated at room temperature for 20 min. The cells were then washed with PBS-T three times. Then the cells were blocked with 5% goat serum (ThermoFisher 50197Z) in PBS-T for 1 h at room temperature. The cells were then incubated with monoclonal mouse GPX4 antibody (Santa Cruz, sc-166570, 1:500 dilution) in PBS-T with 1% BSA and 5% goat serum overnight at 4° C. The cells were washed with PBT for 5 min three times. The cells were incubated with goat anti-mouse IgG (H+L) Highly Cross-Adsorbed Secondary Antibody, Alexa Fluor 594 (Thermo Fisher Scientific Cat #A-11032, RRID:AB_2534091, 1:200 dilution) at room temperature for 1 h. The cells were washed with PBS-T for 5 min three times. ProLong Diamond anti-fade mountant with DAPI (ThermoFisher P36962) was added to stain the nucleus. All images were captured on a Zeiss LSM 800 confocal microscope at Plan-Apochromat 63×/1.40 oil DIC objective with constant laser intensity for all samples. When applicable, line-scan analysis was performed on representative confocal microscopy images using Zeiss LSM software to qualitatively visualize fluorescence overlap.

The quantification of the intensity of antibodies was analyzed using CellProfiler 3.1.8 (Carpenter et al. 2006) (CellProfiler Image Analysis Software, RRID:SCR_007358). Nuclei were first identified as primary objects using global minimum cross entropy strategy, based on DAPI fluorescence signal. The whole cells were then identified as secondary objects based on primary objects by propagation using global minimum cross entropy strategy, based on GPX4 fluorescence (Alexa Fluor 594) signal. The cytoplasm were then identified as the tertiary objects as the part of each cell excluding the nucleus. Then mean intensities of GPX4 fluorescence of nuclei, cytoplasm and whole cell were measured and reported. Graphs were created in Prism 8.

### Determination of GPX4-Specific Activity

We applied a NADPH-coupled cellular GPX4 enzymatic activity assay as previously reported (Roveri et al. 1994). Oxidized glutathione, generated by GPX4 during reducing its specific phospholipid hydroperoxides substrate, was reduced by Glutathione Reductase at the expense of NADPH, the decrease in the characteristic absorbance of which at 340 nm was monitored and quantified as GPX4 activity. The GPX4-specific substrate PCOOH was prepared by enzymatic hydroperoxidation of phosphatidylcholine by soybean lipoxidase type IV: 22 mL of 0.2 M Tris-HCl, pH 8.8, containing 3 mM sodium deoxycholate and 0.3 mM phosphatidylcholine was incubated at room temperature, under continuous stirring, for 30 min with 0.7 mg of soybean lipoxidase type IV. The mixture was loaded on a Sep-Pak CI8 cartridge (Waters-Millipore) washed with methanol and equilibrated with water. After washing with 10 volumes of water, phosphatidylcholine hydroperoxides were eluted in 2 mL of methanol. 48 million HT-1080 cells were harvested and lysed by LCW lysis buffer (0.5% TritonX-100, 0.5% sodium deoxycholate salt, 150 mM NaCl, 20 mM Tris-HCl pH 7.5, 10 mM EDTA, 30 mM Na-pyrophosphate, and complete protease inhibitor cocktail). The concentration of protein in the lysate was determined using BCA assay kit using BSA as standards. Then, on a 96-well plate, 250 μL 1.5 μg/μL cell lysate was incubated in the GPX4 activity assay buffer (0.1% Triton X-100, 100 mM Tris-HCl pH 7.4, 10 mM NaN₃, 5 mM EDTA, 0.6 IU/mL Glutathione reductase, 0.5 mM NADPH) at 37° C. for 10 min. PCOOH was then added to the mixture to initiate GPX4 reaction. Absorbance of NADPH at 340 nm was determined kinetically at 1 min interval over the 20 min time. Experiments using lysis buffer instead of cell lysate and controls without addition of PCOOH were also done to measure the particular activity of GPX4 to reduce phospholipid hydroperoxides. Total GPX4 activity of each sample were normalized to their specific GPX4 level based on Western Blot for unit GPX4 enzymatic activity for comparison. Results were quantified using GraphPad Prism 7.

### Cellular Viability Assay

For dose response curves, 1000 cells of each transfected HT-1080 cell lines subject to the GPX4-specific activity were plated 36 μL per well of a 384-well plate on day 1. The remaining cells were immediately tested for Western Blot and activity assay. Compounds were dissolved in DMSO and a 12-point, twofold dilution series was prepared. The compounds were then diluted 1:50 in media and 4 μL was added to each well of the plates on day 2. Final concentrations of the compounds on the 384-well plate started from 2 μM for RSL3/ML162 and 20 μM for IKE/IN56. After 48 h of treatment, the viability of cells was measured using 1:1 dilution of the CellTiter-Glo luminescent reagent (Promega G7573) with media, which was read on a Victor 5 plate reader after 10 min of shaking at room temperature on day 4. The intensity of luminescence was normalized to that of DMSO control. Results were quantified using GraphPad Prism 7.

### Protein Purification

Bacterial expression vector pOE30-His-tagged-c-GPX4^{U46C} was described in the previous work (Yang et al. 2016). With the vector as template, the following mutagenesis primers were designed using the Agilent QuikChange Primer Design application: R152H (F: 5′-CTG CGT GGT GAA GCA CTA CGG ACC CAT GG-3′ (SEQ ID NO: 12), R: 5′-CCA TGG GTC CGT AGT GCT TCA CCA CGC AG-3′ (SEQ ID NO: 13)), K48A (F: 5′-GGC CTC CCA GTG TGG CGC GAC CGA AGT AAA CTA C-3′ (SEQ ID NO: 14), R: 5′-GTA GTT TAC TTC GGT CGC GCC ACA CTG GGA GGC C-3′ (SEQ ID NO: 15)), K48L (F: 5′-CGT GGC CTC CCA GTG TGG CCT AAC CGA AGT AAA CTA CAC TC-3′ (SEQ ID NO: 16), 5′-GAG TGT AGT TTA CTT CGG TTA GGC CAC ACT GGG AGG CCA CG-3′ (SEQ ID NO: 17)). Primers were purchased from Integrated DNA Technologies. Site-directed mutagenesis kit (QuickChange II, Agilent 200521) was then used to acquire pOE30-c-GPX4^{U46C_R152H}, pOE30-c-GPX4^{U46C_K48A}, and pOE30-c-GPX4^{U46C_K48L}. All mutations and the resulted plasmids were confirmed by sequencing at GENEWIZ.

Isolated colonies with each plasmid were separately transferred to 8 mL of LB medium with 100 μg/mL ampicillin, and the inoculated culture was incubated while being shaken (225 rpm) at 37° C. for 16 h. 3 mL of the starter culture was added to 1 L of fresh LB medium with 100 μg/mL ampicillin. The culture was incubated while being shaken at 37° C. and 225 rpm until the OD600 reached 0.9. The temperature was then decreased to 15° C. Cells were incubated with 1 mM isopropyl β-D-1-thiogalactopyranoside (IPTG) while being shaken at 15° C. and 225 rpm overnight. The next day, the bacteria were harvested by centrifugation at 4000 g for 20 min at 4° C. and the pellet obtained was ready for purification or stored at −20° C. The pellet was resuspended in 25 mL of chilled lysis buffer (100 mM Tris pH 8.0, 300 mM NaCl, 20 mM imidazole, 3 mM TCEP, and Roche protease inhibitor cocktail). The bacteria were lysed by sonication on ice for κ min, and the lysate was centrifuged at 10000 rpm for 20 min at 4° C. to remove cell debris. The clarified lysate was incubated with Ni Sepharose 6 Fast Flow beads (GE Life Sciences) on a rotator at 4° C. for at least 1 h. The beads were washed with wash buffer (100 mM Tris pH 8.0, 300 mM NaCl, 50 mM imidazole, and 3 mM TCEP) to remove nonspecific binding. The protein was eluted with 100 mM Tris pH 8.0, 300 mM NaCl, 100 mM imidazole, and 3 mM TCEP. The protein was further purified using a gel filtration Superdex 200 column in FPLC buffer containing 100 mM Tris pH 8.0, 300 mM NaCl, and 3 mM TCEP. The fractions containing GPX4 were pooled together and analyzed by sodium dodecyl sulfate-polyacrylamide gel electrophoresis (SDS-PAGE).

### Crystallization and Structure Determination

Protein sample of GPX4^{U46C} was initially screened at the High-Throughput Crystallization Screening Center (Luft et al. 2003) of the Hauptman-Woodward Medical Research Institute (HWI) (https://hwi.buffalo.edu/high-throughput-crystallization-center/). The most promising crystal hits were reproduced using under oil micro batch method in a COY anaerobic glove box at 23° C. Plate-like crystals of GPX4^{U46C} were grown using a crystallization reagent comprising 0.056 M sodium phosphate monobasic monohydrate, pH 8.2 and 1.344 M potassium phosphate with protein to crystallization reagent ratio of 2:1 μl. All crystals were subsequently transferred into a similar crystallization reagent that was supplemented by 20% (v/v) glycerol and flash-frozen in liquid nitrogen in the glove box. A native dataset was collected on a crystal of GPX4^{U46C} at the NE-CAT24-ID-C beam line of Advanced Photon Source in Lemont, Ill. Crystals of GPX4^{U46C} were subsequently used as seeds for growing crystals of GPX4^{U46C-R152H}, GPX4^{U46C-K48L}, GPX4^{U46C-K48A}, and GPX4^{U46C}-sulfone outside of the glove box, albeit different crystallization conditions were used for growing these crystals. Crystals of GPX4^{U46C-R152H} were grown in a crystallization condition comprising 0.2 M sodium thiocyanate, pH 6.9, and 20% (w/v) PEG 8000, while those of K48L and K48A were grown in a condition consisting of 0.1 M sodium chloride, 0.1 M MES, pH 6, and 40% (w/v) PEG 8000. Crystals of the fully oxidized GPX4^{U46C}-sulfone were grown in 0.1 M potassium thiocyanate, 0.1 M sodium acetate, pH 5, and 20% (w/v) PEG 8000, and were harvested after one month. In each case, crystals were transferred into the respective crystallization reagent, which was supplemented by 20% (v/v) ethylene glycol.

Crystals of GPX4^{U46C}, GPX4^{U46C-R152H}, GPX4^{U46C-K48L}, GPX4^{U46C-K48A}, and GPX4^{U46C}-sulfone diffracted X-ray at NE-CAT24-ID-C beam line to resolution 1.40 Å, 1.61 Å, 2.10 Å, 1.57 Å, and 1.58 Å, respectively. The images were respectively processed and scaled in space group P21, P21, P21, P212121, and P21, using XDS (Kabsch, 2010). The structure of each protein was determined by molecular replacement method using MOLREP (Vagin and Teplyakov, 2010) program and the crystal structure of GPX4^{U46C} (PDB id: 2OBI) was used as a search model for structure determination of GPX4^{U46C}. The latter structure was used as the search model for subsequent structure determination of other crystal structures. The geometry of each crystal structure was fixed using programs XtalView (McRee, 1999) and COOT (Emsley et al. 2010), and refined by Phenix (Adams et al. 2010). There is one protomer of GPX4 in the asymmetric unit (ASU) of the crystal with space group P2₁. While the ASU of GPX4-U46C-K48A with space group P2₁2₁2₁ contains two protomers. The crystallographic statistics is shown in Table 3.

TABLE 3
Crystallography data collection and refinement statistics.

| | GPX4 | GPX4 | GPX4 | GPX4 | GPX4 |
|---|---|---|---|---|---|
| Data collection | U46C | U46C-R152H | U46C-K48L | U460-K48A | U46C-sulfone |
| Space group | P2₁ | P2₁ | P2₁ | P2₁2₁2₁ | P2₁ |
| Cell dimensions | | | | | |
| a, b, c (Å) | 32.8, 68.9, 35.7 | 32.7, 70.1, 35.7 | 32.8. 69.8, 35.8 | 62.8, 68.6, 81.6 | 32.8, 69.0. 35.8 |
| α, β, γ (°) | 90, 116.1, 90 | 90, 116.3, 90 | 90, 116.2, 90 | 90, 90, 90 | 90, 115.9, 90 |
| Resolution (Å) | 34.5-1.40 | 35.1-1.60 | 69.8-2.10 | 52.48-1.57 | 69.04-1.58 |
| | (1.43-1.40)* | (1.65-1.61)* | (2.15-2.10)* | (1.59-1.57)* | (1.55-1.58)* |
| R_{merge} (%) | 3.3 (21.7) | 18.5 (57.6) | 15.7 (64.5) | 11.7 (73.6) | 3.7 (49.0) |
| l/σl | 18.9 (4.3) | 10.5 (2.6) | 7.1 (2.2) | 13.2 (2.4) | 16.0 (2.7) |
| Completeness (%) | 98.1 (93.1) | 92.1 (93.3) | 99.5 (98.0) | 100 (100) | 99.0 (93.6) |
| Redundancy | 3.3 (3.0) | 3.3 (2.7) | 6.7 (6.3) | 12.9 (9.7) | 3.3 (3.2) |
| CC1/2 | 0.99 (0.84) | 9.97 (0.56) | 0.99 (0.93) | 0.99 (0.92) | 0.99 (0.91) |
| Refinement | | | | | |
| Resolution (Å) | 34.5-1.40 | 35.1-1.61 | 34.9-2.10 | 49.8-1.57 | 34.52-1.58 |
| | (1.43-1.40) | (1.66-1.61)* | (2.23-2.10) | (1.59-1.57) | (1.62-1.58) |
| No. reflections | 27,601 (1,295) | 17.072 (1,158) | 6,375 (1,250) | 49,831 (1,477) | 19,265 (1,237) |
| R_{work}/R_{free} (%) | 16.2 (18.7)/ | 18.0 (18.6)/ | 17.1 (24.4)/ | 15.3 (22.6)/ | 14.5 (19.9)/ |
| | 17.2 (18.8) | 22.3 (23.8) | 23.3 (30.1) | 17.7 (25.7) | 17.2 (20.2) |
| Ramachantran | | | | | |
| Plot (%) | | | | | |
| Outliers | 0.00 | 0.00 | 0.00 | 0.00 | 0.00 |
| Allowed | 123 | 0.68 | 1.23 | 0.31 | 0.00 |
| Favored | 98.77 | 99.35 | 98.77 | 99.69 | 100.00 |
| No. atoms | | | | | |
| Protein | 1,322 | 1,275 | 1,273 | 2,633 | 1,333 |
| Ligand/ion | 11 | 3 | 0 | 0 | 8 |
| Water | 140 | 142 | 55 | 441 | 199 |
| B-factors | | | | | |
| Protein | 18.3 | 12.1 | 34.6 | 19.1 | 20.1 |
| Ligand/ion | 28.3 | 14.7 | | | 22.8 |
| Water | 31.1 | 29.9 | 38.5 | 35.9 | 34.9 |
| R.m.s deviations | | | | | |
| Bond lengths (Å) | 0.005 | 0.006 | 0.006 | 0.005 | 0.006 |
| Bond angles (°) | 0.8 | 0.8 | 0.8 | 0.8 | 0.8 |

*Highest resolution shell is shown in parenthesis.

### DOCUMENTS CITED

1. Agmon E, Solon J, Bassereau P, Stockwell B R. Modeling the effects of lipid peroxidation during ferroptosis on membrane properties. Sci Rep. 2018; 8(1):5155. Epub 2018/03/28. doi: 10.1038/s41598-018-23408-0. PubMed PMID: 29581451; PMCID: PMC5979948.
2. Agmon, E. & Stockwell, B. R. Lipid homeostasis and regulated cell death. Curr Opin Chem Biol 39, 83-89, doi:10.1016/j.cbpa.2017.06.002 (2017).
3. Allimuthu, D. & Adams, D. J. 2-Chloropropionamide As a Low-Reactivity Electrophile for Irreversible Small-Molecule Probe Identification. ACS Chem Biol 12, 2124-2131, doi:10.1021/acschembio.7b00424 (2017).
4. Arai, M. et al. Import into mitochondria of phospholipid hydroperoxide glutathione peroxidase requires a leader sequence. Biochem Biophys Res Commun 227, 433-439, doi:10.1006/bbrc.1996.1525 (1996).
5. Backus, K. M. et al. Proteome-wide covalent ligand discovery in native biological systems. Nature 534, 570-574, doi:10.1038/nature18002 (2016).
6. Baillie, T. A. Targeted Covalent Inhibitors for Drug Design. Angew Chem Int Ed Engl 55, 13408-13421, doi:10.1002/anie.201601091 (2016).
7. Bar-Peled, L. et al. Chemical Proteomics Identifies Druggable Vulnerabilities in a Genetically Defined Cancer. Cell 171, 696-709.e623, doi:https://doi.org/10.1016/j.cell.2017.08.051 (2017).
8. Blanchard, J. E. et al. High-throughput screening identifies inhibitors of the SARS coronavirus main proteinase. Chem Biol 11, 1445-1453, doi:10.1016/j.chembiol.2004.08.011 (2004).
9. Bodnar, M., Szczyglowska, M., Konieczka, P. & Namiesnik, J. Methods of Selenium Supplementation: Bioavailability and Determination of Selenium Compounds. Crit Rev Food Sci Nutr 56, 36-55, doi:10.1080/10408398.2012.709550 (2016).
10. Borchert, A. et al. Crystal structure and functional characterization of selenocysteine-containing glutathione peroxidase 4 suggests an alternative mechanism of peroxide reduction. Biochim Biophys Acta Mol Cell Biol Lipids 1863, 1095-1107, doi:10.1016/j.bbalip.2018.06.006 (2018).
11. Brigelius-Flohe, R. & Maiorino, M. Glutathione peroxidases. Biochim Biophys Acta 1830, 3289-3303, doi:10.1016/j.bbagen.2012.11.020 (2013).
12. Cao J Y, Poddar A, Magtanong L, Lumb J H, Mileur T R, Reid M A, Dovey C M, Wang J, Locasale J W, Stone E, Cole S P C, Carette J E, Dixon S J. A Genome-wide Haploid Genetic Screen Identifies Regulators of Glutathione Abundance and Ferroptosis Sensitivity. Cell reports. 2019; 26(6):1544-56 e8. Epub 2019/02/07. doi: 10.1016/j.celrep.2019.01.043. PubMed PMID: 30726737; PMCID: PMC6424331.
13. Conrad, M. et al. The nuclear form of phospholipid hydroperoxide glutathione peroxidase is a protein thiol peroxidase contributing to sperm chromatin stability. Mol Cell Biol 25, 7637-7644, doi:10.1128/MCB.25.17.7637-7644.2005 (2005).
14. Dillekas, H., Rogers, M. S. & Straume, O. Are 90% of deaths from cancer caused by metastases? Cancer Med 8, 5574-5576, doi:10.1002/cam4.2474 (2019).
15. Dixon S J, Patel D N, Welsch M E, Skouta R, Lee E D, Hayano M, Thomas A G, Gleason C E, Tatonetti N, Slusher B S, Stockwell B R. Pharmacological inhibition of cystine-glutamate exchange induces endoplasmic reticulum stress and ferroptosis. eLife. 2014(May 20):doi: 10.7554/eLife.02523.
16. Dixon S J, Stockwell B R. The Hallmarks of Ferroptosis. Annual Review of Cancer Biology. 2019; 3:35-54.
17. Dixon, S. J. et al. Ferroptosis: an iron-dependent form of nonapoptotic cell death. Cell 149, 1060-1072, doi:10.1016/j.cell.2012.03.042 (2012).
18. Eaton, J. K. et al. Selective covalent targeting of GPX4 using masked nitrile-oxide electrophiles. Nat Chem Biol 16, 497-506, doi:10.1038/s41589-020-0501-5 (2020).
19. Eaton, J. K. et al. Targeting a Therapy-Resistant Cancer Cell State Using Masked Electrophiles as GPX4 Inhibitors. bioRxiv, 376764, doi:10.1 101/376764 (2018).
20. Eaton, J. K., Furst, L., Cai, L. L., Viswanathan, V. S. & Schreiber, S. L. Structure-activity relationships of GPX4 inhibitor warheads. Bioorg Med Chem Lett 30, 127538, doi:10.1016/j.bmcl.2020.127538 (2020).
21. Eaton, J. K., Ruberto, R. A., Kramm, A., Viswanathan, V. S. & Schreiber, S. L. Diacylfuroxans Are Masked Nitrile Oxides That Inhibit GPX4 Covalently. J Am Chem Soc 141, 20407-20415, doi:10.1021/jacs.9b10769 (2019).
22. Elcioglu, N. & Hall, C. M. Spondylometaphyseal dysplasia-Sedaghatian type. Am J Med Genet 76, 410-414 (1998).
23. Fedida, A. et al. Sedaghatian-type spondylometaphyseal dysplasia: Whole exome sequencing in neonatal dry blood spots enabled identification of a novel variant in GPX4. Eur J Med Genet 63, 104020, doi:10.1016/j.ejmg.2020.104020 (2020).
24. Feldmann F, Schenk B, Martens S, Vandenabeele P, Fulda S. Sorafenib inhibits therapeutic induction of necroptosis in acute leukemia cells. Oncotarget. 2017; 8(40):68208-20. Epub 2017/10/06. doi: 10.18632/oncotarget.19919. PubMed PMID: 28978109; PMCID: PMC5620249.
25. Feng, H. & Stockwell, B. R. Unsolved mysteries: How does lipid peroxidation cause ferroptosis? PLoS Biol 16, e2006203, doi:10.1371/journal.pbio.2006203 (2018).
26. Fischer, K. R. et al. Epithelial-to-mesenchymal transition is not required for lung metastasis but contributes to chemoresistance. Nature 527, 472-476, doi:10.1038/nature15748 (2015).
27. Gao, J. et al. Selenium-Encoded Isotopic Signature Targeted Profiling. ACS Cent Sci 4, 960-970, doi:10.1021/acscentsci.8b00112 (2018).
28. Gaschler, M. M. et al. FINO2 initiates ferroptosis through GPX4 inactivation and iron oxidation. Nat Chem Biol 14, 507-515, doi:10.1038/s41589-018-0031-6 (2018).
29. Hangauer, M. J. et al. Drug-tolerant persister cancer cells are vulnerable to GPX4 inhibition. Nature 551, 247-250, doi:10.1038/nature24297 (2017).
30. Hatami, A. et al. Deuterium-reinforced linoleic acid lowers lipid peroxidation and mitigates cognitive impairment in the Q140 knock in mouse model of Huntington's disease. FEBS J 285, 3002-3012, doi:10.1111/febs.14590 (2018).
31. lelasi L, Tovoli F, Piscaglia F. Lenvatinib mesylate to treat hepatocellular carcinoma. Drugs Today (Barc). 2019; 55(5):305-13. Epub 2019/05/28. doi: 10.1358/dot.2019.55.5.2969817. PubMed PMID: 31131841.
32. Imai, H. & Nakagawa, Y. Biological significance of phospholipid hydroperoxide glutathione peroxidase (PHGPx, GPx4) in mammalian cells. Free Radic Biol Med 34, 145-169, doi:10.1016/s0891-5849(02)01197-8 (2003).
33. Ingold, I. et al. Selenium Utilization by GPX4 Is Required to Prevent Hydroperoxide-Induced Ferroptosis. Cell 172, 409-422 e421, doi:10.1016/j.cell.2017.11.048 (2018).
34. Ipek, M. S. & Akin, A. Sedaghatian-Type Spondylometaphyseal Dysplasia: A Case of Rapid Demise with Evidence of Myocardial Injury. J Genet Genome Res 3, 25-27, doi:10.23937/2378-3648/1410025 (2016).
35. Janes, M. R. et al. Targeting KRAS Mutant Cancers with a Covalent G12C-Specific Inhibitor. Cell 172, 578-589.e517, doi:https://doi.org/10.1016/j.cell.2018.01.006 (2018).
36. Janowski, R., Scanu, S., Niessing, D. & Madi, T. Crystal and solution structural studies of mouse phospholipid hydroperoxide glutathione peroxidase 4. Acta Crystallogr F Struct Biol Commun 72, 743-749, doi:10.1107/S2053230X16013686 (2016).
37. Jiang L, Kon N, Li T, Wang S J, Su T, Hibshoosh H, Baer R, Gu W. Ferroptosis as a p53-mediated activity during tumour suppression. Nature. 2015; 520(7545):57-62. doi: 10.1038/nature14344. PubMed PMID: 25799988; PMCID: 4455927.
38. Jung K A, Kwak M K. Enhanced 4-hydroxynonenal resistance in KEAP1 silenced human colon cancer cells. Oxidative medicine and cellular longevity. 2013; 2013:423965. doi: 10.1155/2013/423965. PubMed PMID: 23766854; PMCID: 3674683.
39. Kaplan, A. et al. Small molecule-induced oxidation of protein disulfide isomerase is neuroprotective. Proc Natl Acad Sci USA 112, E2245-2252, doi:10.1073/pnas.1500439112 (2015).
40. Kraft V A N, Bezjian C T, Pfeiffer S, Ringelstetter L, Muller C, Zandkarimi F, Merl-Pham J, Bao X, Anastasov N, Kossl J, Brandner S, Daniels J D, Schmitt-Kopplin P, Hauck S M, Stockwell B R, Hadian K, Schick J A. GTP Cyclohydrolase 1/Tetrahydrobiopterin Counteract Ferroptosis through Lipid Remodeling. ACS Cent Sci. 2020; 6(1):41-53. Epub 2020/01/29. doi: 10.1021/acscentsci.9b01063. PubMed PMID: 31989025; PMCID: PMC6978838 equity in and consults with Inzen Therapeutics and is an inventor on patents and patent applications related to IKE and ferroptosis. J.A.S. holds patents related to ferroptosis. The other authors declare no competing interests.
41. Kuhn, H. & Borchert, A. Regulation of enzymatic lipid peroxidation: the interplay of peroxidizing and peroxide reducing enzymes. Free Radic Biol Med 33, 154-172, doi:10.1016/s0891-5849(02)00855-9 (2002).
42. Lachaier E, Louandre C, Godin C, Saidak Z, Baert M, Diouf M, Chauffert B, Galmiche A. Sorafenib induces ferroptosis in human cancer cell lines originating from different solid tumors. Anticancer Res. 2014; 34(11):6417-22. PubMed PMID: 25368241.
43. Li, C. et al. Novel Allosteric Activators for Ferroptosis Regulator Glutathione Peroxidase 4. J Med Chem 62, 266-275, doi:10.1021/acs.jmedchem.8b00315 (2019).
44. Liang, H. et al. Short form glutathione peroxidase 4 is the essential isoform required for survival and somatic mitochondrial functions. J Biol Chem 284, 30836-30844, doi:10.1074/jbc.M109.032839 (2009).
45. Liu, H., Schreiber, S. L. & Stockwell, B. R. Targeting Dependency on the GPX4 Lipid Peroxide Repair Pathway for Cancer Therapy. Biochemistry 57, 2059-2060, doi:10.1021/acs.biochem.8b00307 (2018).
46. Lo, M. C. et al. Evaluation of fluorescence-based thermal shift assays for hit identification in drug discovery. Anal Biochem 332, 153-159, doi:10.1016/j.ab.2004.04.031 (2004).
47. Louandre C, Ezzoukhry Z, Godin C, Barbare J C, Maziere J C, Chauffert B, Galmiche A. Iron-dependent cell death of hepatocellular carcinoma cells exposed to sorafenib. Int J Cancer. 2013; 133(7):1732-42. doi: 10.1002/ijc.28159. PubMed PMID: 23505071.
48. Martens S, Jeong M, Tonnus W, Feldmann F, Hofmans S, Goossens V, Takahashi N, Brasen J H, Lee E W, Van der Veken P, Joossens J, Augustyns K, Fulda S, Linkermann A, Song J, Vandenabeele P. Sorafenib tosylate inhibits directly necrosome complex formation and protects in mouse models of inflammation and tissue injury. Cell Death Dis. 2017; 8(6):e2904. Epub 2017/07/01. doi: 10.1038/cddis.2017.298. PubMed PMID: 28661484; PMCID: PMC5520944.
49. Mauri, P. et al. Versatility of selenium catalysis in PHGPx unraveled by LC/ESI-MS/MS. Biol Chem 384, 575-588, doi:10.1515/BC.2003.065 (2003).
50. Medavaram S, Zhang Y. Emerging therapies in advanced hepatocellular carcinoma. Exp Hematol Oncol. 2018; 7:17. Epub 2018/08/09. doi: 10.1186/s40164-018-0109-6. PubMed PMID: 30087805; PMCID: PMC6076403.
51. Nural, M. S., Diren, H. B., Sakarya, O., Yalin, T. & Dagdemir, A. Kozlowski type spondylometaphyseal dysplasia: a case report with literature review. Diagn Interv Radiol 12, 70-73 (2006).
52. Romanowska, M. et al. Effects of selenium supplementation on expression of glutathione peroxidase isoforms in cultured human lung adenocarcinoma cell lines. Lung Cancer 55, 35-42, doi:10.1016/j.lungcan.2006.09.007 (2007).
53. Roveri, A., Maiorino, M. & Ursini, F. Enzymatic and immunological measurements of soluble and membrane-bound phospholipid-hydroperoxide glutathione peroxidase. Methods Enzymol 233, 202-212, doi:10.1016/s0076-6879(94)33023-9 (1994).
54. Sakamoto, K. et al. Discovery of GPX4 inhibitory peptides from random peptide T7 phage display and subsequent structural analysis. Biochem Biophys Res Commun 482, 195-201, doi:10.1016/j.bbrc.2016.11.035 (2017).
55. Sayin V I, Ibrahim M X, Larsson E, Nilsson J A, Lindahl P, Bergo M O. Antioxidants accelerate lung cancer progression in mice. Sci Transl Med. 2014; 6(221):221 ra15. doi: 10.1126/scitranslmed.3007653. PubMed PMID: 24477002.
56. Scheerer, P. et al. Structural basis for catalytic activity and enzyme polymerization of phospholipid hydroperoxide glutathione peroxidase-4 (GPx4). Biochemistry 46, 9041-9049, doi:10.1021/bi700840d (2007).
57. Seiler, A. et al. Glutathione peroxidase 4 senses and translates oxidative stress into 12/15-lipoxygenase dependent- and AIF-mediated cell death. Cell Metab 8, 237-248, doi:10.1016/j.cmet.2008.07.005 (2008).
58. Shimada K, Hayano M, Pagano N C, Stockwell B R. Cell-Line Selectivity Improves the Predictive Power of Pharmacogenomic Analyses and Helps Identify NADPH as Biomarker for Ferroptosis Sensitivity. Cell Chem Biol. 2016; 23(2):225-35. doi: 10.1016/j.chembiol.2015.11.016. PubMed PMID: 26853626; PMCID: 4792701.
59. Shimada, K. et al. Global survey of cell death mechanisms reveals metabolic regulation of ferroptosis. Nat Chem Biol 12, 497-503, doi:10.1038/nchembio.2079 (2016).
60. Singh A K, Kumar R, Pandey A K. Hepatocellular Carcinoma: Causes, Mechanism of Progression and Biomarkers. Curr Chem Genom Transl Med. 2018; 12:9-26. Epub 2018/08/03. doi: 10.2174/2213988501812010009. PubMed PMID: 30069430; PMCID: PMC6047212.
61. Smith, A. C. et al. Mutations in the enzyme glutathione peroxidase 4 cause Sedaghatian-type spondylometaphyseal dysplasia. J Med Genet 51, 470-474, doi:10.1136/jmedgenet-2013-102218 (2014).
62. Speer R E, Karuppagounder S S, Basso M, Sleiman S F, Kumar A, Brand D, Smirnova N, Gazaryan I, Khim S J, Ratan R R. Hypoxia-inducible factor prolyl hydroxylases as targets for neuroprotection by “antioxidant” metal chelators: From ferroptosis to stroke. Free Radic Biol Med. 2013; 62:26-36. Epub 2013/02/05. doi: 10.1016/j.freeradbiomed.2013.01.026. PubMed PMID: 23376032; PMCID: PMC4327984.
63. Stockwell B R, Friedmann Angeli J P, Bayir H, Bush A I, Conrad M, Dixon S J, Fulda S, Gascon S, Hatzios S K, Kagan V E, Noel K, Jiang X, Linkermann A, Murphy M E, Overholtzer M, Oyagi A, Pagnussat G C, Park J, Ran Q, Rosenfeld C S, Salnikow K, Tang D, Torti F M, Torti S V, Toyokuni S, Woerpel K A, Zhang D D. Ferroptosis: A Regulated Cell Death Nexus Linking Metabolism, Redox Biology, and Disease. Cell. 2017; 171(2):273-85. Epub 2017/10/07. doi: 10.1016/j.cell.2017.09.021. PubMed PMID: 28985560; PMCID: PMC5685180.
64. Sun X, Ou Z, Chen R, Niu X, Chen, Kang R, Tang D. Activation of the p62-Keap1-NRF2 Pathway Protects against Ferroptosis in Hepatocellular Carcinoma Cells. Hepatology. 2015. doi: 10.1002/hep.28251. PubMed PMID: 26403645.
65. Sutanto, F., Konstantinidou, M. & Dömling, A. Covalent inhibitors: a rational approach to drug discovery. RSC Medicinal Chemistry 11, 876-884, doi:10.1039/DOMD00154F (2020).
66. Thomas, J. P., Geiger, P. G., Maiorino, M., Ursini, F. & Girotti, A. W. Enzymatic reduction of phospholipid and cholesterol hydroperoxides in artificial bilayers and lipoproteins. Biochim Biophys Acta 1045, 252-260, doi:10.1016/0005-2760(90)90128-k (1990).
67. Tosatto, S. C. et al. The catalytic site of glutathione peroxidases. Antioxid Redox Signal 10, 1515-1526, doi:10.1089/ars.2008.2055 (2008).
68. Tsurusaki S, Tsuchiya Y, Koumura T, Nakasone M, Sakamoto T, Matsuoka M, Imai H, Yuet-Yin Kok C, Okochi H, Nakano H, Miyajima A, Tanaka M. Hepatic ferroptosis plays an important role as the trigger for initiating inflammation in nonalcoholic steatohepatitis. Cell Death Dis. 2019; 10(6):449. Epub 2019/06/19. doi: 10.1038/s41419-019-1678-y. PubMed PMID: 31209199; PMCID: PMC6579767.
69. Ursini, F. et al. Dual function of the selenoprotein PHGPx during sperm maturation. Science 285, 1393-1396, doi:10.1126/science.285.5432.1393 (1999).
70. Vadrevu, R., Falzone, C. J. & Matthews, C. R. Partial NMR assignments and secondary structure mapping of the isolated alpha subunit of Escherichia coli tryptophan synthase, a 29-kD TIM barrel protein. Protein Sci 12, 185-191, doi:10.1110/ps.0221103 (2003).
71. Viswanathan, V. S. et al. Dependency of a therapy-resistant state of cancer cells on a lipid peroxidase pathway. Nature 547, 453-457, doi:10.1038/nature23007 (2017).
72. Wang, Q. et al. Dimethyl Fumarate Protects Neural Stem/Progenitor Cells and Neurons from Oxidative Damage through Nrf2-ERK1/2 MAPK Pathway. Int J Mol Sci 16, 13885-13907, doi:10.3390/ijms160613885 (2015).
73. Wang, X., Zhang, H. & Chen, X. Drug resistance and combating drug resistance in cancer. Cancer Drug Resistance 2, 141-160, doi:10.20517/cdr.2019.10 (2019).
74. Weiwer, M. et al. Development of small-molecule probes that selectively kill cells induced to express mutant RAS. Bioorg Med Chem Lett 22, 1822-1826, doi:10.1016/j.bmcl.2011.09.047 (2012).
75. Wu J, Minikes A M, Gao M, Bian H, Li Y, Stockwell B R, Chen Z N, Jiang X. Intercellular interaction dictates cancer cell ferroptosis via NF2-YAP signalling. Nature. 2019; 572(7769):402-6. Epub 2019/07/26. doi: 10.1038/s41586-019-1426-6. PubMed PMID: 31341276; PMCID: PMC6697195.
76. Wu, Z. et al. Chaperone-mediated autophagy is involved in the execution of ferroptosis. Proc Natl Acad Sci USA 116, 2996-3005, doi:10.1073/pnas.1819728116 (2019).
77. Yang, W. S. & Stockwell, B. R. Ferroptosis: Death by Lipid Peroxidation. Trends Cell Biol 26, 165-176, doi:10.1016/j.tcb.2015.10.014 (2016).
78. Yang, W. S. & Stockwell, B. R. Synthetic lethal screening identifies compounds activating iron-dependent, nonapoptotic cell death in oncogenic-RAS-harboring cancer cells. Chem Biol 15, 234-245, doi:10.1016/j.chembiol.2008.02.010 (2008).
79. Yang, W. S. et al. Peroxidation of polyunsaturated fatty acids by lipoxygenases drives ferroptosis. Proc Natl Acad Sci USA 113, E4966-4975, doi:10.1073/pnas.1603244113 (2016).
80. Yang, W. S. et al. Regulation of ferroptotic cancer cell death by GPX4. Cell 156, 317-331, doi:10.1016/j.cell.2013.12.010 (2014).
81. Yant, L. J. et al. The selenoprotein GPX4 is essential for mouse development and protects from radiation and oxidative damage insults. Free Radic Biol Med 34, 496-502, doi:10.1016/s0891-5849(02)01360-6 (2003).
82. Yoo, S. E. et al. Gpx4 ablation in adult mice results in a lethal phenotype accompanied by neuronal loss in brain. Free Radic Biol Med 52, 1820-1827, doi:10.1016/j.freeradbiomed.2012.02.043 (2012).
83. Yu, Y. et al. Characterization and structural analysis of human selenium-dependent glutathione peroxidase 4 mutant expressed in Escherichia coli. Free Radic Biol Med 71, 332-338, doi:10.1016/j.freeradbiomed.2014.03.032 (2014).
84. Zhang, Y. et al. Imidazole Ketone Erastin Induces Ferroptosis and Slows Tumor Growth in a Mouse Lymphoma Model. Cell Chem Biol 26, 623-633 e629, doi:10.1016/j.chembiol.2019.01.008 (2019).
85. Zhou Z, Ye T J, DeCaro E, Buehler B, Stahl Z, Bonavita G, Daniels M, You M. Intestinal SIRT1 Deficiency Protects Mice from Ethanol-Induced Liver Injury by Mitigating Ferroptosis. Am J Pathol. 2019. Epub 2019/10/15. doi: 10.1016/j.ajpath.2019.09.012. PubMed PMID: 31610175.
86. Zhu, K. et al. Docking covalent inhibitors: a parameter free approach to pose prediction and scoring. J Chem Inf Model 54, 1932-1940, doi:10.1021/ci500118s (2014).
87. Zou, Y. et al. A GPX4-dependent cancer cell state underlies the clear-cell morphology and confers sensitivity to ferroptosis. Nat Commun 10, 1617, doi:10.1038/s41467-019-09277-9 (2019).
88. Zou, Y. et al. Plasticity of ether lipids promotes ferroptosis susceptibility and evasion. Nature 585, 603-608, doi:10.1038/s41586-020-2732-8 (2020).

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

1. (canceled)

2. A compound according to formula (2): 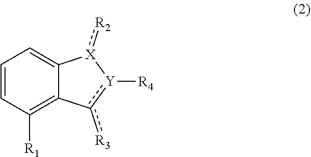
wherein:
a dashed line indicates the presence of an optional double bond;
X is C; Y is N;
R₁ is H, ether, C₁₋₆alkyl, wherein the ether or C₁₋₆alkyl may be optionally substituted with an atom or a group selected from the group consisting of N, O, Sn, halo, C₁₋₄alkyl, CF₃, and combinations thereof:
R₂ and R₃ are O; and
R₄ is selected from the group consisting of no atom, H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 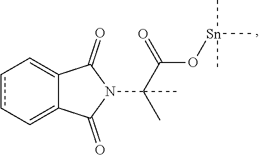
or an N-oxide, crystalline form, hydrate, or pharmaceutically acceptable salt thereof.

3. The compound according to claim 2 having a structure selected from the group consisting of: 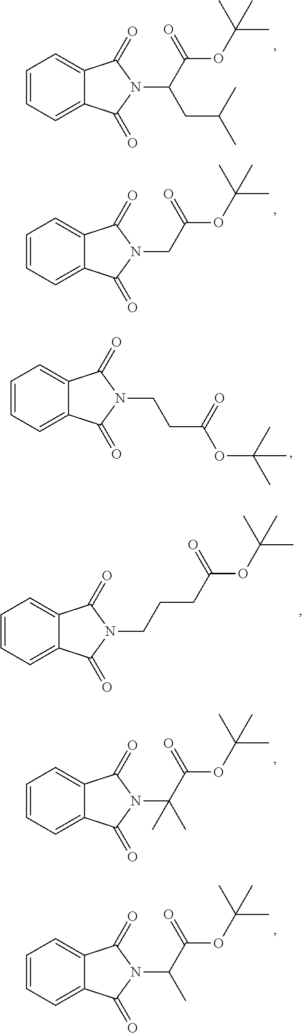 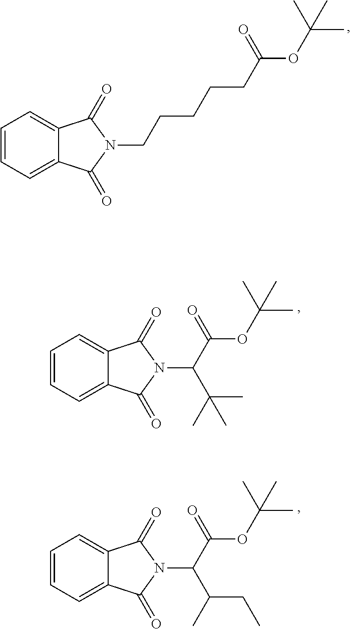 [SMILES: CC(C)(C)OC(=O)CCCCCN1C(=O)c2ccccc2C1=O ; CC(C)(C)OC(=O)C(N1C(=O)c2ccccc2C1=O)C(C)(C)C ; CCC(C)C(C(=O)OC(C)(C)C)N1C(=O)c2ccccc2C1=O]
or an N-oxide, crystalline form, hydrate, or pharmaceutically acceptable salt thereof

4. (canceled)

5. (canceled)

6. A composition comprising one or more compounds of claim 2 and a pharmaceutically acceptable carrier, adjuvant or vehicle.

7. A method for treating or ameliorating the effects of a glutathione peroxidase 4 (GPX4)-associated disease, modulating the activity of glutathione peroxidase 4 (GPX4), increasing the level of peroxide, or treating or ameliorating the effects of a cancer in a subject in need thereof or inducing ferroptosis in a cell, comprising administering to the subject or contacting the cell with an effective amount of one or more compounds of claim 2, or one or more compounds of formula (1), (3), or (4): 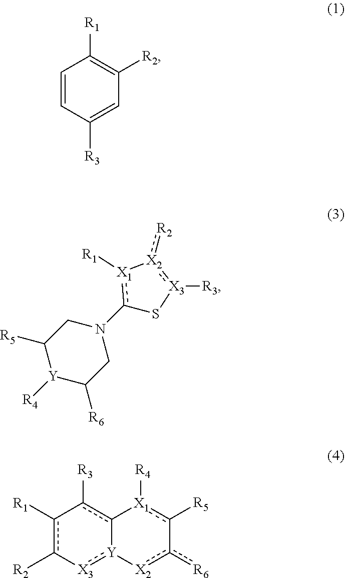
wherein for formula (1):
R₁, R₂, and R₃ are independently selected from the group consisting of H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 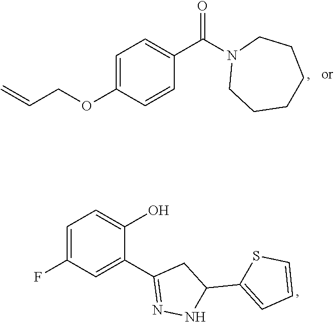 [SMILES: C=CCOc1ccc(C(=O)N2CCCCCC2)cc1 ; Oc1ccc(F)cc1C1=NNC(c2cccs2)C1]
or an N-oxide, crystalline form, hydrate, or pharmaceutically acceptable salt thereof;
wherein for formula (3):
a dashed line indicates the presence of an optional double bond;
X₁, X₂, X₃ and Y are independently selected from the group consisting of C, N, S and O;
R₁, R₂, R₃, R₄, R₅, and R₆ are independently selected from the group consisting of no atom H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 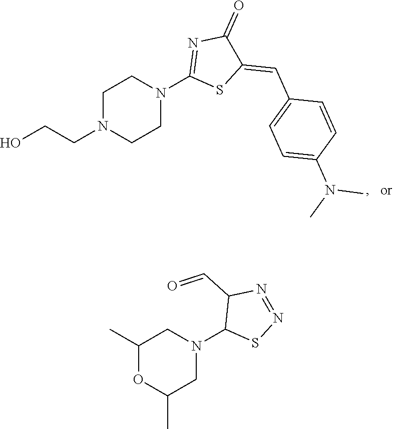
or an N-oxide, crystalline form, hydrate, or pharmaceutically acceptable salt thereof; and
wherein for formula (4):
a dashed line indicates the presence of an optional double bond;
X₁, X₂, and X₃ are independently selected from the group consisting of C, N, S and O;
Y is C or N;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₂carbocycle that may be optionally substituted with an atom or a group selected from the group consisting of O, N, halo, C₁₋₄alkyl, CF₃, and combinations thereof;
R₃ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
R₄ and R₅ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₄ and R₅ may together form a C₃₋₁₂carbocycle that may be optionally substituted with an atom or a group selected from the group consisting of O, N, halo, C₁₋₄alkyl, CF₃, and combinations thereof;
R₆ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 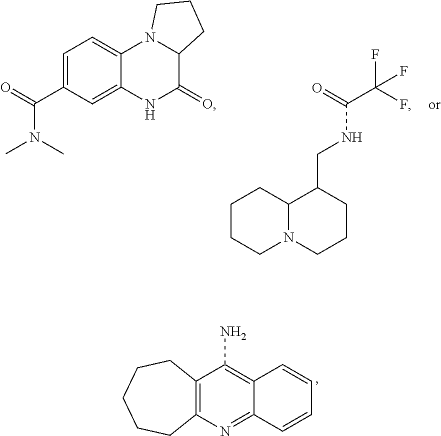
or an N-oxide, crystalline form, hydrate, or pharmaceutically acceptable salt thereof.

8. The method of claim 7, wherein the GPX4-associated disease is selected from the group consisting of a cancer, a neurotic disorder, a neurodegenerative disorder, spondylometaphyseal dysplasia, mixed cerebral palsy, pontocerebellar hypoplasia, and male infertility.

9. The method of 7, wherein the GPX4-associated disease is a cancer.

10. The method of claim 9, wherein the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma.

11. The method of claim 9, wherein the cancer is hepatocellular carcinoma.

12. The method of claim 7, wherein the subject is a mammal.

13.-15. (canceled)

16. The method of claim 9, wherein the cancer is under epithelial-to-mesenchymal (EMT) transition, the cancer is hypersensitive to ferroptosis, or the cancer is refractory to standard cancer treatment.

17.-24. (canceled)

25. The method of claim 7, wherein the cell has abberant lipid accumulation.

26. The method of claim 7, wherein the cell is a cancer cell.

27.-34. (canceled)

35. The method of claim 7, wherein the treating or ameliorating the effects of a cancer in a subject in need thereof, further comprises administering to the subject an effective amount of a second anti-cancer agent.

36.-47. (canceled)

48. A kit for treating or ameliorating the effects of a glutathione peroxidase 4 (GPX4)-associated disease in a subject in need thereof, comprising an effective amount of one or more compounds of any one of claim 2, or one or more compounds of formula (1), (3), or (4): 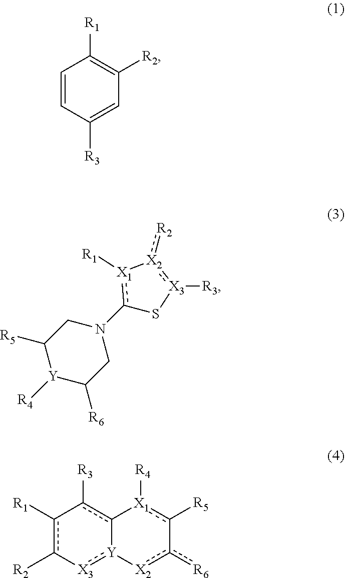
wherein for formula (1):
R₁, R₂, and R₃ are independently selected from the group consisting of H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 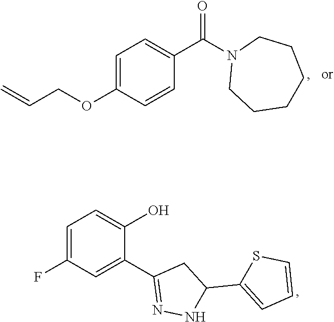 [SMILES: C=CCOc1ccc(C(=O)N2CCCCCC2)cc1 ; Oc1ccc(F)cc1C1=NNC(c2cccs2)C1]
or an N-oxide, crystalline form, hydrate, or pharmaceutically acceptable salt thereof;
wherein for formula (3):
a dashed line indicates the presence of an optional double bond;
X₁, X₂, X₃ and Y are independently selected from the group consisting of C, N, S and O;
R₁, R₂, R₃, R₄, R₅, and R₆ are independently selected from the group consisting of no atom H, D, O, N, halo, ether, ester, amide, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 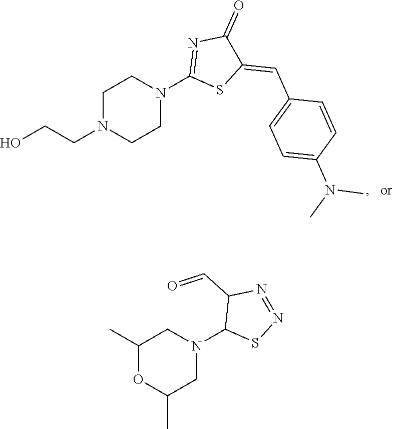
or an N-oxide, crystalline form, hydrate, or pharmaceutically acceptable salt thereof; and
wherein for formula (4):
a dashed line indicates the presence of an optional double bond;
X₁, X₂, and X₃ are independently selected from the group consisting of C, N, S and O;
Y is C or N;
R₁ and R₂ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₁ and R₂ may together form a C₃₋₁₂carbocycle that may be optionally substituted with an atom or a group selected from the group consisting of O, N, halo, C₁₋₄alkyl, CF₃, and combinations thereof;
R₃ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
R₄ and R₅ are independently selected from the group consisting of no atom, H, D, —OH, N, halo, C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl, wherein the C₁₋₆alkyl, C₁₋₆alkyl-aryl, C₁₋₆alkyl-heteroaryl, C₂₋₆alkenyl, C₂₋₆alkenyl-aryl, and C₂₋₆alkenyl-heteroaryl may be optionally substituted with an atom or a group selected from the group consisting of N, epoxy, —OH, halo, C₁₋₄alkyl, CF₃, and combinations thereof, or R₄ and R₅ may together form a C₃₋₁₂carbocycle that may be optionally substituted with an atom or a group selected from the group consisting of O, N, halo, C₁₋₄alkyl, CF₃, and combinations thereof;
R₆ is selected from the group consisting of H, D, O, N, halo, ether, ester, amide, amino, C(O), (O)C(R), C(O)O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 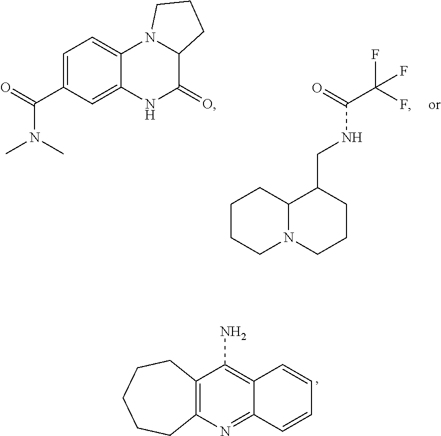
or an N-oxide, crystalline form, hydrate, or pharmaceutically acceptable salt thereof, packaged with its instructions for use.

49. A method for treating or ameliorating the effects of a glutathione peroxidase 4 (GPX4)-associated disease, increasing the level of peroxide, treating or ameliorating the effects of a cancer in a subject in need thereof or inducing ferroptosis in a cell, comprising administering to the subject or contacting the cell with an effective amount of one or more compounds having a structure selected from the group consisting of: 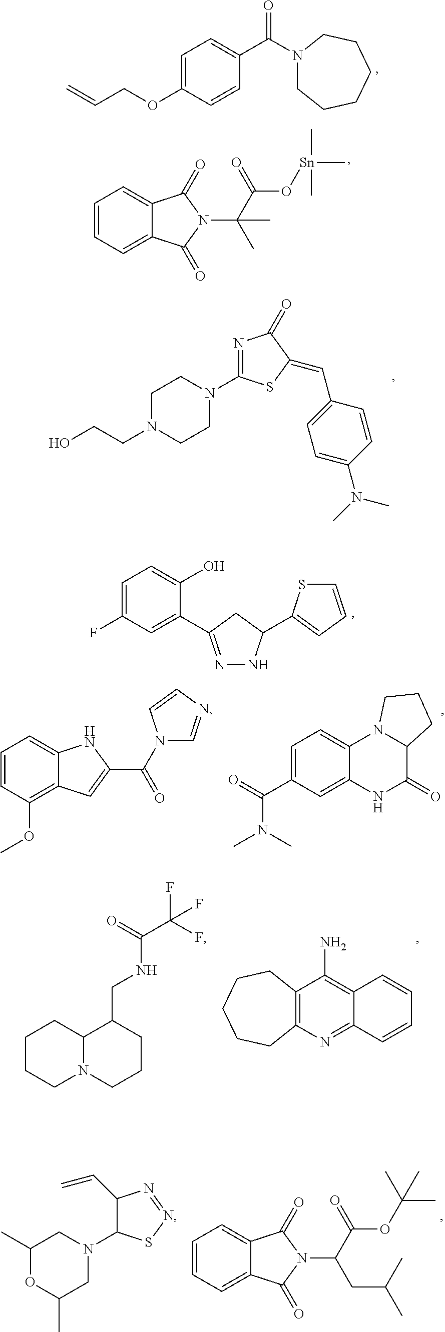 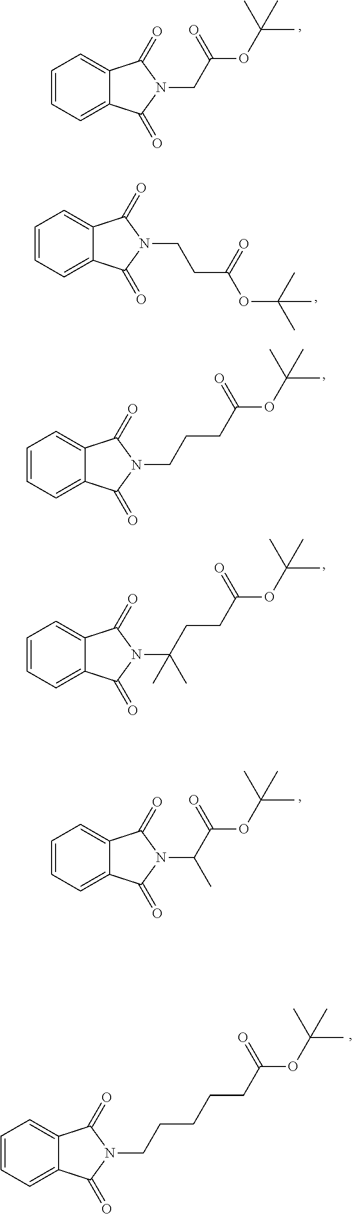 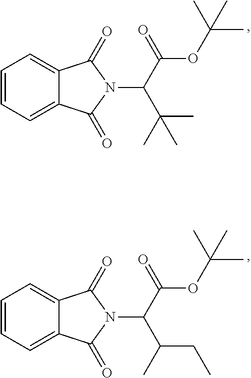 [SMILES: CC(C)(C)OC(=O)C(N1C(=O)c2ccccc2C1=O)C(C)(C)C ; CCC(C)C(C(=O)OC(C)(C)C)N1C(=O)c2ccccc2C1=O]
and combinations thereof, or an N-oxide, crystalline form, hydrate, or pharmaceutically acceptable salt thereof.

50.-54. (canceled)
